(19) United States
(12) Patent Application Publication
(10) Pub. No.: US 2015/0376172 A1
(43) Pub. Date: Dec. 31, 2015

(54) FLUORO-[1,3]OXAZINES AS BACE1 INHIBITORS

(71) Applicants: Thomas WOLTERING, (US); HOFFMANN-LA ROCHE INC., Nutley, NJ (US); SIENA BIOTECH S.P.A., Siena (IT)

(72) Inventors: Wolfgang Guba, Muellheim (DE); Hans Hilpert, Muenchenstein (CH); Andreas Kuglstatter, Loerrach (DE); Anja Limberg, Basel (CH); Ulrike Obst Sander, Reinach (CH); Emmanuel Pinard, Linsdorf (FR); Wolfgang Wostl, Grenzach-Wyhlen (DE); Thomas Woltering, Freiburg (DE)

(21) Appl. No.: 14/759,980

(22) PCT Filed: Jan. 15, 2014
(86) PCT No.: PCT/EP2014/050645

§ 371 Date: Jul. 9, 2015

(30) Foreign Application Priority Data
Jan. 22, 2013 (EP) 13152213.8

(51) Int. Cl. C07D 413/14; C07D 413/10; C07D 413/12

(52) U.S. Cl. CPC C07D 413/14; C07D 413/12; C07D 413/10

(57) ABSTRACT

The present invention provides a compound of formula I having BACE1 inhibitory activity, their manufacture, pharmaceutical compositions containing them and their use as therapeutically active substances. The active compounds of the present invention are useful in the therapeutic and/or prophylactic treatment of e.g. Alzheimer's disease.

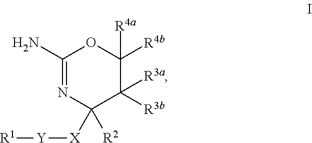

### BACKGROUND ART

Alzheimer's disease (AD) is a neurodegenerative disorder of the central nervous system and the leading cause of a progressive dementia in the elderly population. Its clinical symptoms are impairment of memory, cognition, temporal and local orientation, judgment and reasoning but also severe emotional disturbances. There are currently no treatments available which can prevent the disease or its progression or stably reverse its clinical symptoms. AD has become a major health problem in all societies with high life expectancies and also a significant economic burden for their health systems.

AD is characterized by 2 major pathologies in the central nervous system (CNS), the occurrence of amyloid plaques and neurofibrillar tangles (Hardy et al.¹ and Selkoe²). Both pathologies are also commonly observed in patients with Down's syndrome (trisomy 21), which also develop AD-like symptoms in early life. Neurofibrillar tangles are intracellular aggregates of the microtubule-associated protein tau (MAPT). Amyloid plaques occur in the extracellular space; their principal components are Aβ-peptides. The latter are a group of proteolytic fragments derived from the β-amyloid precursor protein (APP) by a series of proteolytic cleavage steps. Several forms of APP have been identified of which the most abundant are proteins of 695, 751 and 770 amino acids length. They all arise from a single gene through differential splicing. The Aβ-peptides are derived from the same domain of the APP but differ at their N- and C-termini, the main species are of 40 and 42 amino-acid length. There are several lines of evidence which strongly suggest that aggregated Aβ-peptides are the essential molecules in the pathogenesis of AD: 1) amyloid plaques formed of Aβ-peptides are invariably part of the AD pathology; 2) Aβ-peptides are toxic for neurons; 3) in Familial Alzheimer's Disease (FAD) the mutations in the disease genes APP, PSN1, PSN2 lead to increased levels of Aβ-peptides and early brain amyloidosis; 4) transgenic mice which express such FAD genes develop a pathology which bears many resemblances to the human disease. Aβ-peptides are produced from APP through the sequential action of 2 proteolytic enzymes termed β- and γ-secretase. β-Secretase cleaves first in the extracellular domain of APP approximately 28 amino acids outside of the trans-membrane domain (TM) to produce a C-terminal fragment of APP containing the TM- and the cytoplasmatic domain (CTFβ). CTFβ is the substrate for γ-secretase which cleaves at several adjacent positions within the TM to produce the Aβ peptides and the cytoplasmic fragment. The γ-secretase is a complex of at least 4 different proteins, its catalytic subunit is very likely a presenilin protein (PSEN1, PSEN2). The β-secretase (BACE1, Asp2; BACE stands for β-site APP-cleaving enzyme) is an aspartyl protease which is anchored into the membrane by a transmembrane domain (Vassar et al.³). It is expressed in many tissues of the human organism but its level is especially high in the CNS. Genetic ablation of the BACE1 gene in mice has clearly shown that its activity is essential for the processing of APP which leads to the generation of Aβ-peptides, in the absence of BACE1 no Aβ-peptides are produced (Luo et al.⁴ and Roberds et al.⁵). Mice which have been genetically engineered to express the human APP gene and which form extensive amyloid plaques and Alzheimer's disease like pathologies during aging fail to do so when 3-secretase activity is reduced by genetic ablation of one of the BACE1 alleles (McConlogue et al.⁶). It is thus presumed that inhibitors of BACE1 activity can be useful agents for therapeutic intervention in Alzheimer's disease (AD).

Furthermore, the formation, or formation and deposition, of β-amyloid peptides in, on or around neurological tissue (e.g., the brain) are inhibited by the present compounds, i.e. inhibition of the Aβ-production from APP or an APP fragment.

Inhibitors of BACE1 can in addition be used to treat the following diseases: IBM (inclusion body myositis) (Vattemi G. et al.⁷), Down's Syndrome (Barbiero L. et al.⁸), Wilson's Disease (Sugimoto I. et al.⁹), Whipple's disease (Desnues B. et al.¹⁰), SpinoCerebellar Ataxia 1 and SpinoCerebellar Ataxia 7 (Gatchel J. R. et al.¹¹ Dermatomyositis (Greenberg S. A. et al¹² and Greenberg S. A. et al.¹³), Kaposi Sarcoma (Lagos D. et al.¹⁴), Glioblastoma multiforme⁵, Rheumatoid arthritis (Ungethuem U. et al.¹⁶), Amyotrophic lateral sclerosis (Koistinen H. et al.¹⁷ and Li Q. X. et al.¹⁸), Huntington's Disease (Kim Y. J. et al.¹⁹ and Hodges A. et al.²⁰), Multiple Mieloma (Kihara Y. et al.²), Malignant melanoma (Talantov D. et al.²²) Sjogren syndrome (Basset C. et al.²³), Lupus erythematosus (Grewal P. K. et al.²⁴), Macrophagic myofasciitis, juvenile idiopathic arthritis, granulomatous arthritis, Breast cancer (Hedlund M. et al.²⁵ and Kondoh K. et al.²⁶), Gastrointestinal diseases (Hoffmeister A. et al.²⁷), Autoimmune/inflammatory diseases (Woodard-Grice A. V. et al.²⁸), Rheumatoid Arthritis (Toegel S. et al.²⁹), Inflammatory reactions (Lichtenthaler S. F. et al.³⁰), Arterial Thrombosis (Merten M. et al.³¹), Cardiovascular diseases such as Myocardial infarction and stroke (Maugeri N. et al.³² and Graves disease (Kiljafiski J. et al.³³).

WO2013110622, WO2012168175, WO2012168164 and WO2012156284³⁴ describe certain oxazines as BACE1 and/or BACE2 inhibitors.

The present invention provides novel compounds of formula I, their manufacture, medicaments based on a compound in accordance with the invention and their production as well as the use of compounds of formula I in the control or prevention of illnesses such as Alzheimer's disease. Furthermore the use of compounds of formula I in the treatment of amyotrophic lateral sclerosis (ALS), arterial thrombosis, autoimmune/inflammatory diseases, cancer such as breast cancer, cardiovascular diseases such as myocardial infarction and stroke, dermatomyositis, Down's Syndrome, gastrointestinal diseases, Glioblastoma multiforme, Graves Disease, Huntington's Disease, inclusion body myositis (IBM), inflammatory reactions, Kaposi Sarcoma, Kostmann Disease, lupus erythematosus, macrophagic myofasciitis, juvenile idiopathic arthritis, granulomatous arthritis, malignant melanoma, multiple mieloma, rheumatoid arthritis, Sjogren syndrome, SpinoCerebellar Ataxia 1, SpinoCerebellar Ataxia 7, Whipple's Disease and Wilson's Disease. The novel compounds of formula I have improved pharmacological properties.

### FIELD OF THE INVENTION

The present invention provides fluoro-[1,3]oxazines having BACE1 inhibitory properties, their manufacture, pharmaceutical compositions containing them and their use as therapeutically active substances.

### SUMMARY OF THE INVENTION

The present invention provides a compound of formula I,

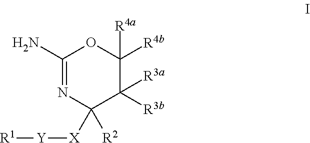

wherein the substituents and variables are as described below and in the claims, or a pharmaceutically acceptable salt thereof.

The present compounds have Asp2 (β-secretase, BACE1 or Memapsin-2) inhibitory activity and may therefore be used in the therapeutic and/or prophylactic treatment of diseases and disorders characterized by elevated β-amyloid levels and/or β-amyloid oligomers and/or β-amyloid plaques and further deposits, particularly Alzheimer's disease.

### DETAILED DESCRIPTION OF THE INVENTION

The present invention provides a compound of formula I and its pharmaceutically acceptable salts thereof, the preparation of the above mentioned compounds, medicaments containing them and their manufacture as well as the use of the above mentioned compounds in the therapeutic and/or prophylactic treatment of diseases and disorders which are associated with inhibition of BACE1, such as Alzheimer's disease. Furthermore, the formation, or formation and deposition, of β-amyloid plaques in, on or around neurological tissue (e.g., the brain) are inhibited by the present compounds by inhibiting the Aβ production from APP or an APP fragment.

The following definitions of the general terms used in the present description apply irrespectively of whether the terms in question appear alone or in combination with other groups.

The term “C₁₋₆-alkyl”, alone or in combination with other groups, stands for a hydrocarbon radical which may be linear or branched, with single or multiple branching, wherein the alkyl group in general comprises 1 to 6 carbon atoms, for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl (2-methyl-butyl), 1,2-dimethyl-propyl and the like. Particular “C₁₋₆-alkyl” are “C₁₋₃-alkyl”. Specific groups are methyl and ethyl. Most specific is methyl.

The term “halogen-C₁₋₆-alkyl”, alone or in combination with other groups, refers to C₁₋₆-alkyl as defined herein, which is substituted by one or multiple halogen, particularly 1-5 halogen, more particularly 1-3 halogen. Particular halogen is fluoro. Particular “halogen-C₁₋₆-alkyl” is fluoro-C₁₋₆-alkyl and a particular “halogen-C₁₋₃-alkyl” is fluoro-C₁₋₃-alkyl. Examples are trifluoromethyl, difluoromethyl, fluoromethyl and the like. A specific group is trifluoromethyl.

The term “C₁₋₆-alkoxy-C₁₋₆-alkyl”, alone or in combination with other groups, refers to C₁₋₆-alkyl as defined herein, which is substituted by one or multiple C₁₋₆-alkoxy, as defined herein, particularly 1 C₁₋₆-alkoxy. Particular “C₁₋₆-alkoxy-C₁₋₆-alkyl” is methoxy-C₁₋₆-alkyl. Examples are methoxymethyl, methoxyethyl and the like.

The term “cyano”, alone or in combination with other groups, refers to N≡C—(NC—).

The term “halogen”, alone or in combination with other groups, denotes chloro (Cl), iodo (I), fluoro (F) and bromo (Br). Particular “halogen” is Cl and F. A specific group is F.

The term “heteroaryl”, alone or in combination with other groups, refers to an aromatic carbocyclic group of having a single 4 to 8 membered ring, in particular 5 to 8, or multiple condensed rings comprising 6 to 14, in particular 6 to 10 ring atoms and containing 1, 2 or 3 heteroatoms individually selected from N, O and S, in particular 1N or 2N, in which group at least one heterocyclic ring is aromatic. Examples of “heteroaryl” include benzofuryl, benzoimidazolyl, 1H-benzoimidazolyl, benzooxazinyl, benzoxazolyl, benzothiazinyl, benzothiazolyl, benzothienyl, benzotriazolyl, furyl, imidazolyl, indazolyl, 1H-indazolyl, indolyl, isoquinolinyl, isothiazolyl, isoxazolyl, oxazolyl, pyrazinyl, pyrazolyl (pyrazyl), 1H-pyrazolyl, pyrazolo[1,5-a]pyridinyl, pyridazinyl, pyridinyl, pyrimidinyl, pyrrolyl, quinolinyl, tetrazolyl, thiazolyl, thienyl, triazolyl, 6,7-dihydro-5H-[1]pyrindinyl and the like. Particular “heteroaryl” are pyridinyl, pyrazinyl, 1H-pyrazolyl, 6,7-dihydro-5H-cyclopenta[b]pyridinyl, benzo[d]oxazolyl, 1,3,4-oxadiazolyl, 1H-tetrazolyl, isothiazolyl, thiazolyl, 1H-1,2,3-triazolyl and pyrimidinyl. Specific “heteroaryl” are pyridin-2-yl, pyridin-3-yl, pyridin-4-yl and pyrazin-2-yl, 1H-pyrazole-4-yl, 1H-pyrazol-3-yl, 6,7-dihydro-5H-cyclopenta[b]pyridinyl, benzo[d]oxazol-2-yl, 1,3,4-oxadiazol-2-yl, 1H-tetrazol-5-yl, isothiazol-5-yl, thiazol-5-yl, 1H-1,2,3-triazol-1-yl and pyrimidin-5-yl.

The term “C₁₋₆-alkoxy”, alone or in combination with other groups, stands for an —O—C₁₋₆-alkyl radical which may be linear or branched, with single or multiple branching, wherein the alkyl group in general comprises 1 to 6 carbon atoms, for example, methoxy (OMe, MeO), ethoxy (OEt), propoxy, isopropoxy (i-propoxy), n-butoxy, i-butoxy (iso-butoxy), 2-butoxy (sec-butoxy), t-butoxy (tert-butoxy), isopentyloxy (i-pentyloxy) and the like. Particular “C₁₋₆-alkoxy” are groups with 1 to 4 carbon atoms. Specific is methoxy.

The term “halogen-C₁₋₆-alkoxy”, alone or in combination with other groups, refers to C₁₋₆-alkoxy as defined herein, which is substituted by one or multiple halogens, in particular fluoro. Particular “halogen-C₁₋₆-alkoxy” are fluoro-C₁₋₆-alkoxy. Specific “halogen-C₁₋₆-alkoxy” is trifluoromethoxy.

The term “C₂₋₆-alkynyl-C₁₋₆-alkoxy”, alone or in combination with other groups, refers to C₁₋₆-alkoxy as defined herein, which is substituted by one or multiple C₂₋₆-alkynyl as defined herein, in particular 1 C₂₋₆-alkynyl.

The term “C₂₋₆-alkynyl”, alone or in combination with other groups, denotes a monovalent linear or branched saturated hydrocarbon group of 2 to 6 carbon atoms, in particular from 2 to 4 carbon atoms, and comprising one, two or three triple bonds. Examples of C₂₋₆-alkynyl include ethynyl, propynyl, and n-butynyl.

The term “aryl” denotes a monovalent aromatic carbocyclic mono- or bicyclic ring system comprising 6 to 10 carbon ring atoms. Examples of aryl moieties include phenyl and naphthyl. Specific “aryl” is phenyl.

The term “pharmaceutically acceptable salts” refers to salts that are suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid (sulphuric acid), tartaric acid, trifluoroacetic acid and the like. Particular acids are formic acid, trifluoroacetic acid and hydrochloric acid. Specific acids are hydrochloric acid, trifluoroacetic acid and fumaric acid.

The terms “pharmaceutically acceptable carrier” and “pharmaceutically acceptable auxiliary substance” refer to carriers and auxiliary substances such as diluents or excipients that are compatible with the other ingredients of the formulation.

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

The term “inhibitor” denotes a compound which competes with, reduces or prevents the binding of a particular ligand to particular receptor or which reduces or prevents the inhibition of the function of a particular protein.

The term “half maximal inhibitory concentration” (IC₅₀) denotes the concentration of a particular compound required for obtaining 50% inhibition of a biological process in vitro. IC₅₀ values can be converted logarithmically to pIC₅₀ values (−log IC₅₀), in which higher values indicate exponentially greater potency. The IC₅₀ value is not an absolute value but depends on experimental conditions e.g. concentrations employed. The IC₅₀ value can be converted to an absolute inhibition constant (Ki) using the Cheng-Prusoff equation³⁵. The term “inhibition constant” (Ki) denotes the absolute binding affinity of a particular inhibitor to a receptor. It is measured using competition binding assays and is equal to the concentration where the particular inhibitor would occupy 50% of the receptors if no competing ligand (e.g. a radioligand) was present. Ki values can be converted logarithmically to pKi values (−log Ki), in which higher values indicate exponentially greater potency.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The term “as defined herein” and “as described herein” when referring to a variable incorporates by reference the broad definition of the variable as well as particularly, more particularly and most particularly definitions, if any.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “protecting group” denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups. The term “amino-protecting group” (here also P¹) denotes groups intended to protect an amino group and includes benzyl, benzyloxycarbonyl (carbobenzyloxy, CBZ), 9-fluorenylmethoxycarbonyl (FMOC), p-methoxybenzyloxycarbonyl, p-nitrobenzyloxycarbonyl, tert-butoxycarbonyl (BOC), and trifluoroacetyl. Further examples of these groups are found in several books.³⁶. The term “protected amino group” refers to an amino group substituted by an amino-protecting groups. Particular amino-protecting groups are tert-butoxycarbonyl group and dimethoxytrityl.

The term “leaving group” denotes the group with the meaning conventionally associated with it in synthetic organic chemistry, i.e., an atom or group displaceable under substitution reaction conditions. Examples of leaving groups include halogen, in particular bromo, alkane- or arylenesulfonyloxy, such as methanesulfonyloxy, ethanesulfonyloxy, thiomethyl, benzenesulfonyloxy, tosyloxy, dihalophosphinoyloxy, optionally substituted benzyloxy, isopropyloxy, and acyloxy.

The term “aromatic” denotes the conventional idea of aromaticity as defined in the literature³⁷.

The term “pharmaceutically acceptable excipient” denotes any ingredient having no therapeutic activity and being non-toxic such as disintegrators, binders, fillers, solvents, buffers, tonicity agents, stabilizers, antioxidants, surfactants or lubricants used in formulating pharmaceutical products.

Whenever a chiral carbon is present in a chemical structure, it is intended that all stereoisomers associated with that chiral carbon are encompassed by the structure as pure stereoisomers as well as mixtures thereof.

The invention also provides pharmaceutical compositions, methods of using, and methods of preparing the aforementioned compounds.

All separate embodiments may be combined.

One embodiment of the invention provides a compound of formula I,

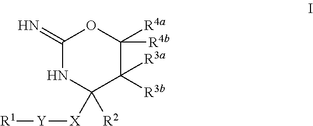

wherein
Y is a bond and X is selected from the group consisting of

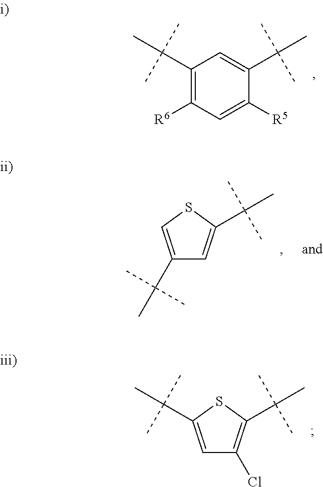

or
Y is selected from the group consisting of

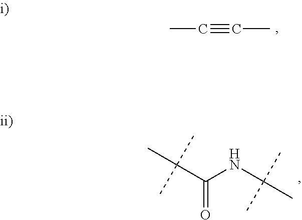

iii) —(CH₂)ₙ— with n=1, 2 or 3,
iv) —NH—, and
and X is

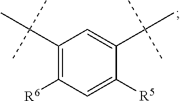

### or Y is

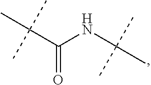

### and X is

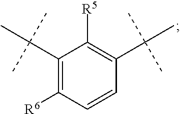

R¹ is selected from the group consisting of
i) heteroaryl, and
ii) heteroaryl, substituted by one or two substituents individually selected from the group consisting of amino, cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl, C₁₋₆-alkyl, aryl, which aryl is optionally substituted by cyano, halogen, halogen-C₁₋₆-alkyl, halogen-C₁₋₆-alkoxy, C₁₋₆-alkoxy or C₁₋₆-alkyl, and heteroaryl, which heteroaryl is optionally substituted by cyano, halogen, halogen-C₁₋₆-alkyl, halogen-C₁₋₆-alkoxy, C₁₋₆-alkoxy or C₁₋₆-alkyl,
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iv) aryl, when R^{4a} is halogen-C₁₋₆-alkyl;
   R² is selected from the group consisting of
i) C₁₋₆-alkyl, and
ii) halogen-C₁₋₆-alkyl;
   R^{3a} is selected from the group consisting of
i) hydrogen and
ii) C₁₋₆-alkyl;
   R^{3b} is selected from the group consisting of
i) hydrogen,
ii) halogen, and
iii) halogen-C₁₋₆-alkoxy;
   R^{4a} is selected from the group consisting of
i) halogen-C₁₋₆-alkyl, and
ii) C₁₋₆-alkyl;
   R^{4b} is selected from the group consisting of
i) hydrogen and
ii) C₁₋₆-alkyl;
   R⁵ is halogen;
   R⁶ is selected from the group consisting of
i) hydrogen and
ii) halogen;
   or pharmaceutically acceptable salts thereof.

A certain embodiment of the invention provides a compound of formula I, wherein

Y is a bond and X is selected from the group consisting of

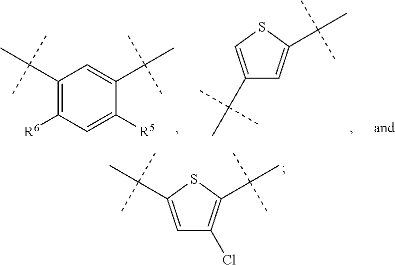

or

Y is selected from the group consisting of
—C≡C— and

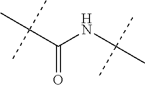

### and X is

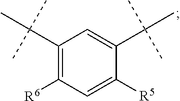

R¹ is selected from the group consisting of
i) heteroaryl, and
ii) heteroaryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl.

R² is selected from the group consisting of
i) C₁₋₆-alkyl, and
ii) halogen-C₁₋₆-alkyl;

R^{3a} is selected from the group consisting of
i) hydrogen and
ii) C₁₋₆-alkyl;

R^{3b} is selected from the group consisting of
i) hydrogen,
ii) halogen, and
iii) halogen-C₁₋₆-alkoxy;

R^{4a} is selected from the group consisting of
i) halogen-C₁₋₆-alkyl, and
ii) C₁₋₆-alkyl;

R^{4b} is selected from the group consisting of
i) hydrogen and
ii) C₁₋₆-alkyl;

R⁵ is halogen;

R⁶ is selected from the group consisting of
i) hydrogen and
ii) halogen;

or pharmaceutically acceptable salts thereof.

A certain embodiment of the invention provides a compound of formula I, which is of formula I′

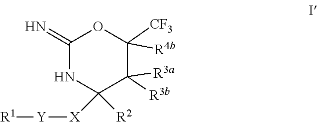

wherein
Y is a bond and X is selected from the group consisting of

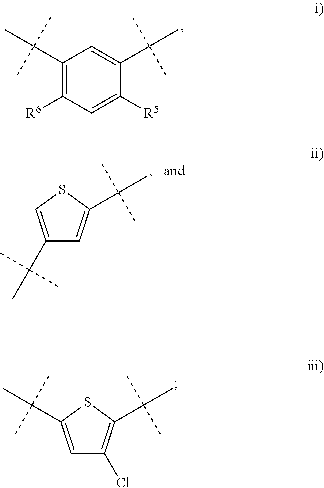

or
Y is selected from the group consisting of

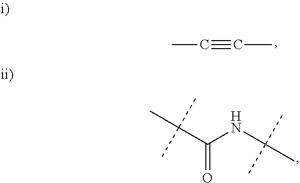

iii) —(CH₂)ₙ— with n=1, 2 or 3,
iv) —NH—, and

### and X is

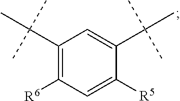

### or Y is

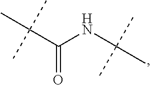

### and X is

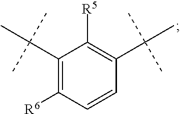

R¹ is selected from the group consisting of
i) heteroaryl, and
ii) heteroaryl, substituted by one or two substituents individually selected from the group consisting of amino, cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl, C₁₋₆-alkyl, aryl, which aryl is optionally substituted by cyano, halogen, halogen-C₁₋₆-alkyl, halogen-C₁₋₆-alkoxy, C₁₋₆-alkoxy or C₁₋₆-alkyl, and heteroaryl, which heteroaryl is optionally substituted by cyano, halogen, halogen-C₁₋₆-alkyl, halogen-C₁₋₆-alkoxy, C₁₋₆-alkoxy or C₁₋₆-alkyl,
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and iv) aryl, when R^{4a} is halogen-C₁₋₆-alkyl;
   R² is selected from the group consisting of
i) C₁₋₆-alkyl, and
ii) halogen-C₁ 6-alkyl;
   R^{3a} is selected from the group consisting of
i) hydrogen and
ii) C₁₋₆-alkyl;
   R^{3b} is selected from the group consisting of
i) hydrogen,
ii) halogen, and
iii) halogen-C₁₋₆-alkoxy;
   R^{4a} is selected from the group consisting of
i) halogen-C₁₋₆-alkyl, and
ii) C₁₋₆-alkyl;

R^{4b} is selected from the group consisting of
i) hydrogen and
ii) C₁₋₆-alkyl;
   R⁵ is halogen;
   R⁶ is selected from the group consisting of
i) hydrogen and
ii) halogen;
   or pharmaceutically acceptable salts thereof.

A certain embodiment of the invention provides a compound of formula I, which is of formula I′, wherein

Y is a bond and X is selected from the group consisting of

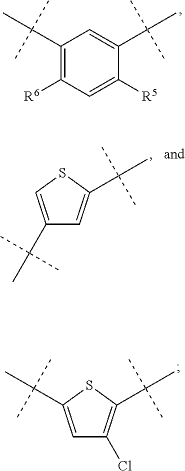

or

Y is selected from the group consisting of
—C≡C— and

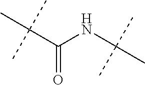

### and X is

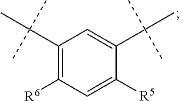

R¹ is selected from the group consisting of
i) heteroaryl, and
ii) heteroaryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl.

R² is selected from the group consisting of
i) C₁₋₆-alkyl, and
ii) halogen-C₁₋₆-alkyl;

R^{3a} is selected from the group consisting of
i) hydrogen and
ii) C₁₋₆-alkyl;

R^{3b} is selected from the group consisting of
i) hydrogen,
ii) halogen, and
iii) halogen-C₁₋₆-alkoxy;

R^{4a} is selected from the group consisting of
i) halogen-C₁₋₆-alkyl, and
ii) C₁₋₆-alkyl;

R^{4b} is selected from the group consisting of
i) hydrogen and
ii) C₁₋₆-alkyl;

R⁵ is halogen;

R⁶ is selected from the group consisting of
i) hydrogen and
ii) halogen;

or pharmaceutically acceptable salts thereof.

A certain embodiment of the invention provides a compound of formula I, which is of formula Ia′

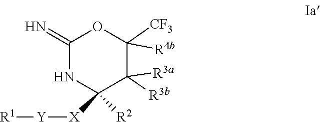

wherein
Y is a bond and X is selected from the group consisting of

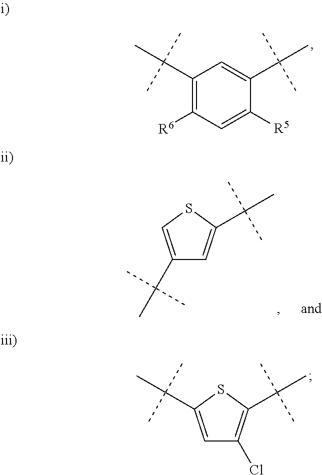

or
Y is selected from the group consisting of
i) —C≡C—,

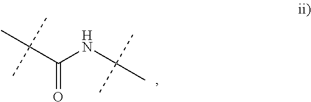

iii) —(CH₂)ₙ— with n=1, 2 or 3,
iv) —NH—, and

### and X is

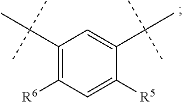

### or Y is

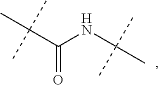

### and X is

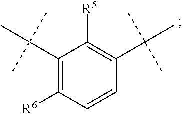

R¹ is selected from the group consisting of
i) heteroaryl, and
ii) heteroaryl, substituted by one or two substituents individually selected from the group consisting of amino, cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl, C₁₋₆-alkyl, aryl, which aryl is optionally substituted by cyano, halogen, halogen-C₁₋₆-alkyl, halogen-C₁₋₆-alkoxy, C₁₋₆-alkoxy or C₁₋₆-alkyl, and heteroaryl, which heteroaryl is optionally substituted by cyano, halogen, halogen-C₁₋₆-alkyl, halogen-C₁₋₆-alkoxy, C₁₋₆-alkoxy or C₁₋₆-alkyl,
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iv) aryl, when R^{4a} is halogen-C₁₋₆-alkyl;
   R² is selected from the group consisting of
i) C₁₋₆-alkyl, and
ii) halogen-C₁₋₆-alkyl;
   R^{3a} is selected from the group consisting of
i) hydrogen and
ii) C₁₋₆-alkyl;
   R^{3b} is selected from the group consisting of
i) hydrogen,
ii) halogen, and
iii) halogen-C₁₋₆-alkoxy;
   R^{4a} is selected from the group consisting of
i) halogen-C₁₋₆-alkyl, and
ii) C₁₋₆-alkyl;
   R^{4b} is selected from the group consisting of
i) hydrogen and
ii) C₁₋₆-alkyl;
   R⁵ is halogen;
   R⁶ is selected from the group consisting of
i) hydrogen and
ii) halogen;
   or pharmaceutically acceptable salts thereof.

A certain embodiment of the invention provides a compound of formula I, which is of formula Ia′, wherein

Y is a bond and X is selected from the group consisting of

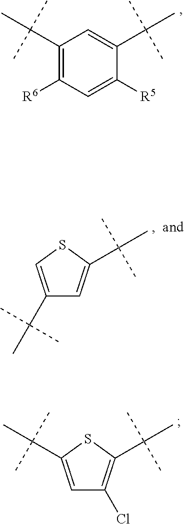

or

Y is selected from the group consisting of
—C═C— and

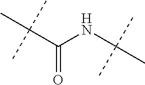

### and X is

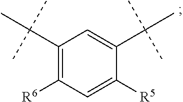

R¹ is selected from the group consisting of
i) heteroaryl, and
ii) heteroaryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl.

R² is selected from the group consisting of
i) C₁₋₆-alkyl, and
ii) halogen-C₁₋₆-alkyl;

R^{3a} is selected from the group consisting of
i) hydrogen and
ii) C₁₋₆-alkyl;

R^{3b} is selected from the group consisting of
i) hydrogen,
ii) halogen, and
iii) halogen-C₁₋₆-alkoxy;

R^{4a} is selected from the group consisting of
i) halogen-C₁₋₆-alkyl, and
ii) C₁₋₆-alkyl;

R^{4b} is selected from the group consisting of
i) hydrogen and
ii) C₁₋₆-alkyl;

R⁵ is halogen;

R⁶ is selected from the group consisting of
i) hydrogen and
ii) halogen;

or pharmaceutically acceptable salts thereof.

In a certain embodiment the invention relates to a compound of formula I, wherein Y is a bond and X is

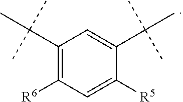

In a certain embodiment the invention relates to a compound of formula I, wherein Y is a bond, X is

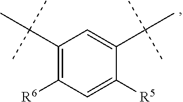

### R² is —CH₃, R^{3a} is H, R^{3b} is H, R^{4a} is —CF₃, R^{4b} is H, R⁶ is H and R⁵ is F.

In a certain embodiment the invention relates to a compound of formula I, wherein Y is a bond, X is

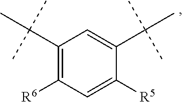

R² is —CH₃, R^{3a} is H, R^{3b} is H, R^{4a} is —CF₃, R^{4b} is H, R⁶ is H and R⁵ is F, and R¹ is heteroaryl, optionally substituted by cyano, halogen, C₂₋₆-alkynyl or halogen-C₁₋₆-alkyl.

In a certain embodiment the invention relates to a compound of formula I, wherein Y is a bond and X is

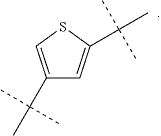

In a certain embodiment the invention relates to a compound of formula I, wherein Y is a bond, X is

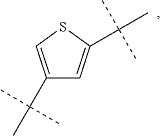

### R² is —CH₃, R^{3a} is H, R^{3b} is H, R^{4a} is —CF₃, R^{4b} is H.

In a certain embodiment the invention relates to a compound of formula I, wherein Y is a bond, X is

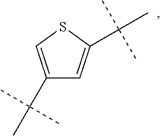

R² is —CH₃, R^{3a} is H, R^{3b} is H, R^{4a} is —CF₃, R^{4b} is H and R¹ is phenyl substituted by halogen-C₁₋₆-alkyl.

In a certain embodiment the invention relates to a compound of formula I, wherein Y is a bond, X is

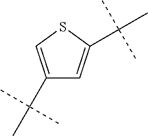

R² is —CH₃, R^{3a} is H, R^{3b} is H, R^{4a} is —CF₃, R^{4b} is H and R¹ is pyridinyl substituted by cyano, halogen, C₂₋₆-alkynyl or halogen-C₁₋₆-alkyl.

In a certain embodiment the invention relates to a compound of formula I, wherein Y is a bond and X is

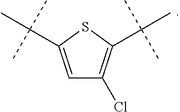

In a certain embodiment the invention relates to a compound of formula I, wherein Y is a bond, X is

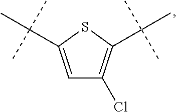

### R² is —CH₃, R^{3a} is H, R^{3b} is H, R^{4a} is —CF₃, R^{4b} is H.

In a certain embodiment the invention relates to a compound of formula I, wherein Y is a bond, X is

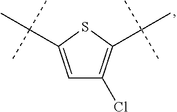

R² is —CH₃, R^{3a} is H, R^{3b} is H, R^{4a} is —CF₃, R^{4b} is H and R¹ is phenyl substituted by cyano.

In a certain embodiment the invention relates to a compound of formula I, wherein Y is a bond, X is

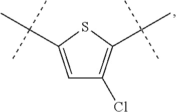

R² is —CH₃, R^{3a} is H, R^{3b} is H, R^{4a} is —CF₃, R^{4b} is H and R¹ is pyridinyl substituted by cyano, halogen or C₂₋₆-alkynyl.

In a certain embodiment the invention relates to a compound of formula I, wherein Y is

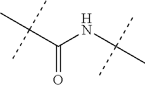

### and X is

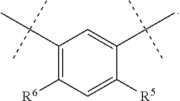

In a certain embodiment the invention relates to a compound of formula I, wherein Y is

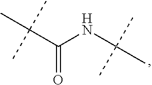

### X is

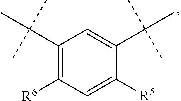

### R² is —CH₃, R^{3a} is H, R^{3b} is F, R^{4a} is —CF₃, R^{4b} is H, R⁶ is H and R⁵ is F.

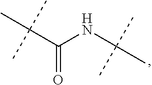

### X is

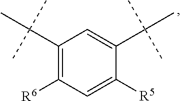

### R² is —CH₃, R^{3a} is Me, R^{3b} is H, R^{4a} is —CF₃, R^{4b} is H, R⁶ is H and R⁵ is F.

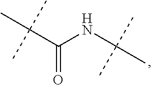

### X is

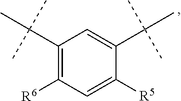

### R² is —CH₃, R^{3a} is H, R^{3b} is —OCH₂CF₃, R^{4a} is —CF₃, R^{4b} is H, R⁶ is H and R⁵ is F.

In a certain embodiment the invention relates to a compound of formula I, wherein Y is

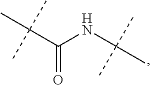

### X is

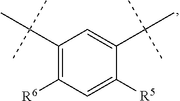

### R² is —CH₃, R^{3a} is Me, R^{3b} is —OCH₂CF₃, R^{4a} is —CF₃, R^{4b} is H, R⁶ is H and R⁵ is F.

In a certain embodiment the invention relates to a compound of formula I, wherein Y is

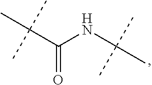

### X is

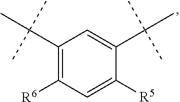

### R² is —CH₃, R^{3a} is H, R^{3b} is H, R^{4a} is —CH₃, R^{4b} is H, R⁶ is H and R⁵ is F.

In a certain embodiment the invention relates to a compound of formula I, wherein Y is

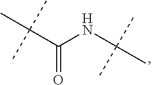

### X is

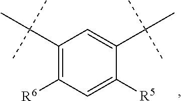

R² is —CH₃, R^{3a} is H, R^{3b} is H, R^{4a} is —CF₃, R^{4b} is H, R⁶ is H and R⁵ is F, and R¹ is heteroaryl, optionally substituted by cyano, halogen, C₂₋₆-alkynyl or halogen-C₁₋₆-alkyl.

In a certain embodiment the invention relates to a compound of formula I, wherein Y is —C≡C— and X is

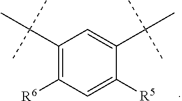

In a certain embodiment the invention relates to a compound of formula I, wherein Y is —C≡C—, X is

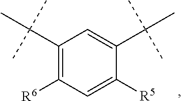

### R² is —CH₃, R^{3a} is H, R^{3b} is F, R^{4a} is —CF₃, R^{4b} is H, R⁶ is H and R⁵ is F.

In a certain embodiment the invention relates to a compound of formula I, wherein Y is —C≡C—, X is

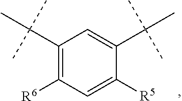

### R² is —CH₃, R^{3a} is Me, R^{3b} is H, R^{4a} is —CF₃, R^{4b} is H, R⁶ is H and R⁵ is F.

In a certain embodiment the invention relates to a compound of formula I, wherein Y is —C≡C—, X is

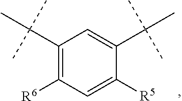

### R² is —CH₃, R^{3a} is H, R^{3b} is —OCH₂CF₃, R^{4a} is —CF₃, R^{4b} is H, R⁶ is H and R⁵ is F.

In a certain embodiment the invention relates to a compound of formula I, wherein Y is —C≡C—, X is

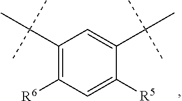

### R² is —CH₃, R^{3a} is Me, R^{3b} is —OCH₂CF₃, R^{4a} is —CF₃, R^{4b} is H, R⁶ is H and R⁵ is F.

In a certain embodiment the invention relates to a compound of formula I, wherein Y is —C≡C—, X is

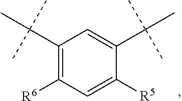

### R² is —CH₃, R^{3a} is H, R^{3b} is H, R^{4a} is —CH₃, R^{4b} is H, R⁶ is H and R⁵ is F.

In a certain embodiment the invention relates to a compound of formula I, wherein Y is —C≡C—, X is

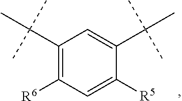

R² is —CH₃, R^{3a} is H, R^{3b} is H, R^{4a} is —CF₃, R^{4b} is H, R⁶ is H and R⁵ is F, and R¹ is heteroaryl, optionally substituted by cyano, halogen, C₂₋₆-alkynyl or halogen-C₁₋₆-alkyl.

In a certain embodiment the invention relates to a compound of formula I, wherein Y is a bond and X is selected from the group consisting of

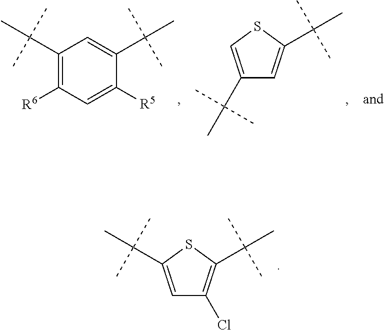

In a certain embodiment the invention relates to a compound of formula I, wherein X is

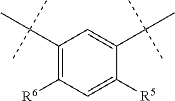

and Y is selected from the group consisting of —C≡C— and

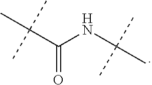

In a certain embodiment the invention relates to a compound of formula I, wherein Y is —C≡C—.

In a certain embodiment the invention relates to a compound of formula I, wherein R⁵ is fluoro.

In a certain embodiment the invention relates to a compound of formula I, wherein R⁶ is hydrogen.

In a certain embodiment the invention relates to a compound of formula I, wherein R¹ is selected from the group consisting of
i) heteroaryl,
ii) heteroaryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl.

In a certain embodiment the invention relates to a compound of formula I, wherein R¹ is heteroaryl.

In a certain embodiment the invention relates to a compound of formula I, wherein R¹ is heteroaryl substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl.

In a certain embodiment the invention relates to a compound of formula I, wherein R¹ is aryl substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl.

In a certain embodiment the invention relates to a compound of formula I, wherein R¹ is selected from the group consisting of phenyl, 1H-pyrazolyl, pyridinyl, pyrazinyl and pyrimidinyl, each unsubstituted or individually substituted by difluoromethyl, chloro, fluoro, cyano, trifluoromethyl, prop-1-ynyl, but-2-ynyloxy or methoxy.

In a certain embodiment the invention relates to a compound of formula I, wherein R¹ is phenyl substituted by cyano or trifluoromethyl.

In a certain embodiment the invention relates to a compound of formula I, wherein R¹ is phenyl substituted by cyano.

In a certain embodiment the invention relates to a compound of formula I, wherein R¹ is phenyl substituted trifluoromethyl.

In a certain embodiment the invention relates to a compound of formula I, wherein R¹ is 1H-pyrazolyl substituted by difluoromethyl.

In a certain embodiment the invention relates to a compound of formula I, wherein R¹ is pyridinyl, unsubstituted or substituted by cyano, chloro, fluoro or prop-1-ynyl.

In a certain embodiment the invention relates to a compound of formula I, wherein R¹ is pyridinyl.

In a certain embodiment the invention relates to a compound of formula I, wherein R¹ is pyridinyl, substituted by cyano.

In a certain embodiment the invention relates to a compound of formula I, wherein R¹ is pyridinyl, substituted by chloro.

In a certain embodiment the invention relates to a compound of formula I, wherein R¹ is pyridinyl, substituted by fluoro.

In a certain embodiment the invention relates to a compound of formula I, wherein R¹ is pyridinyl, substituted by prop-1-ynyl.

In a certain embodiment the invention relates to a compound of formula I, wherein R¹ is pyrazinyl substituted by but-2-ynyloxy, methoxy, difluoromethyl or chloro.

In a certain embodiment the invention relates to a compound of formula I, wherein R¹ is pyrazinyl substituted by but-2-ynyloxy.

In a certain embodiment the invention relates to a compound of formula I, wherein R¹ is pyrazinyl substituted by methoxy.

In a certain embodiment the invention relates to a compound of formula I, wherein R¹ is pyrazinyl substituted by difluoromethyl.

In a certain embodiment the invention relates to a compound of formula I, wherein R¹ is pyrazinyl substituted by chloro.

In a certain embodiment the invention relates to a compound of formula I, wherein R¹ is pyrimidinyl, unsubstituted or substituted by chloro or methoxy.

In a certain embodiment the invention relates to a compound of formula I, wherein R² is methyl.

In a certain embodiment the invention relates to a compound of formula I, wherein R² is —CH₂F.

In a certain embodiment the invention relates to a compound of formula I, wherein R^{3a} is hydrogen.

In a certain embodiment the invention relates to a compound of formula I, wherein R^{3a} is methyl.

In a certain embodiment the invention relates to a compound of formula I, R^{3b} is hydrogen.

In a certain embodiment the invention relates to a compound of formula I, R^{3b} is —OCH₂CF₃.

In a certain embodiment the invention relates to a compound of formula I, wherein R^{3b} is fluoro.

In a certain embodiment the invention relates to a compound of formula I, wherein R^{4a} is —CF₃.

In a certain embodiment the invention relates to a compound of formula I, wherein R^{4a} is methyl.

In a certain embodiment the invention relates to a compound of formula I, wherein R^{4b} is hydrogen.

In a certain embodiment the invention relates to a compound of formula I, wherein R^{4b} is methyl.

In a certain embodiment the invention relates to a compound of formula I, which is selected from the group consisting of:
(4S,6S)-4-(2,4-Difluoro-5-(2-fluoropyridin-3-yl)phenyl)-4-methyl-6-(trifluoro-methyl)-5,6-dihydro-4H-1,3-oxazin-2-amine,
(4S,6S)-4-(2,4-Difluoro-5-(5-(prop-1-ynyl)pyridin-3-yl)phenyl)-4-methyl-6-(trifluoromethyl)-5,6-dihydro-4H-1,3-oxazin-2-amine,
(4S,6S)-4-(2,4-Difluoro-5-(pyrimidin-5-yl)phenyl)-4-methyl-6-(trifluoromethyl)-5,6-dihydro-4H-1,3-oxazin-2-amine,
(4S,6S)-4-(2-fluoro-5-((3-methylisothiazol-5-yl)ethynyl)phenyl)-4-(fluoromethyl)-6-(trifluoromethyl)-5,6-dihydro-4H-1,3-oxazin-2-amine,
(4S,6S)-4-(2-fluoro-5-((3-methylisothiazol-5-yl)ethynyl)phenyl)-4-methyl-6-(trifluoromethyl)-5,6-dihydro-4H-1,3-oxazin-2-amine,
(4S,6S)-4-(2-Fluoro-5-((5-methoxypyrazin-2-yl)ethynyl)phenyl)-4-methyl-6-(trifluoromethyl)-5,6-dihydro-4H-1,3-oxazin-2-amine,
(4S,6S)-4-(2-Fluoro-5-((5-methoxypyrimidin-2-yl)ethynyl)phenyl)-4-methyl-6-(trifluoromethyl)-5,6-dihydro-4H-1,3-oxazin-2-amine,
(4S,6S)-4-(2-fluoro-5-(4-fluoropyridin-3-yl)-4-methylphenyl)-4-methyl-6-(trifluoromethyl)-5,6-dihydro-4H-1,3-oxazin-2-amine,
(4S,6S)-4-(2-fluoro-5-(5-(5-methyl-1H-pyrazol-3-yl)pyridin-3-yl)phenyl)-4-(fluoromethyl)-6-(trifluoromethyl)-5,6-dihydro-4H-1,3-oxazin-2-amine,
(4S,6S)-4-(2-Fluoro-5-(5-(prop-1-ynyl)pyridin-3-yl)phenyl)-4-methyl-6-(trifluoromethyl)-5,6-dihydro-4H-1,3-oxazin-2-amine,
(4S,6S)-4-(2-Fluoro-5-(pyridin-3-ylethynyl)phenyl)-4-methyl-6-(trifluoromethyl)-5,6-dihydro-4H-1,3-oxazin-2-amine,
(4S,6S)-4-(2-fluoro-5-(pyrimidin-5-yl)phenyl)-4-methyl-6-(trifluoromethyl)-5,6-dihydro-4H-1,3-oxazin-2-amine,
(4S,6S)-4-(2-fluoro-5-(pyrimidin-5-ylethynyl)phenyl)-4-methyl-6-(trifluoromethyl)-5,6-dihydro-4H-1,3-oxazin-2-amine,
(4S,6S)-4-(3-Chloro-5-(5-(prop-1-ynyl)pyridin-3-yl)thiophen-2-yl)-4-methyl-6-(trifluoromethyl)-5,6-dihydro-4H-1,3-oxazin-2-amine,
(4S,6S)-4-(3-Chloro-5-(5-chloropyridin-3-yl)thiophen-2-yl)-4-methyl-6-(trifluoromethyl)-5,6-dihydro-4H-1,3-oxazin-2-amine,
(4S,6S)-4-(4-(2-Fluoropyridin-3-yl)thiophen-2-yl)-4-methyl-6-(trifluoromethyl)-5,6-dihydro-4H-1,3-oxazin-2-amine,
(4S,6S)-4-(4-fluorobiphenyl-3-yl)-4-methyl-6-(trifluoromethyl)-5,6-dihydro-4H-1,3-oxazin-2-amine,
(4S,6S)-4-(5-((1-ethyl-1H-pyrazol-4-yl)ethynyl)-2-fluorophenyl)-4-(fluoromethyl)-6-(trifluoromethyl)-5,6-dihydro-4H-1,3-oxazin-2-amine,
(4S,6S)-4-(5-((2-aminopyrimidin-5-yl)ethynyl)-2-fluorophenyl)-4-methyl-6-(trifluoromethyl)-5,6-dihydro-4H-1,3-oxazin-2-amine,
(4S,6S)-4-(5-((2-Chloropyridin-4-yl)ethynyl)-2-fluorophenyl)-4-methyl-6-(trifluoromethyl)-5,6-dihydro-4H-1,3-oxazin-2-amine,
(4S,6S)-4-(5-((4-chloro-1-(difluoromethyl)-1H-pyrazol-3-yl)ethynyl)-2-fluorophenyl)-4-methyl-6-(trifluoromethyl)-5,6-dihydro-4H-1,3-oxazin-2-amine,
(4S,6S)-4-(5-((4-chloro-1-(difluoromethyl)-1H-pyrazol-3-yl)ethynyl)-2-fluoro-4-methylphenyl)-4-methyl-6-(trifluoromethyl)-5,6-dihydro-4H-1,3-oxazin-2-amine,
(4S,6S)-4-(5-((4-chlorophenyl)ethynyl)-2-fluorophenyl)-4-methyl-6-(trifluoromethyl)-5,6-dihydro-4H-1,3-oxazin-2-amine,
(4S,6S)-4-(5-((5-chloropyridin-3-yl)ethynyl)-2-fluorophenyl)-4-(fluoromethyl)-6-(trifluoromethyl)-5,6-dihydro-4H-1,3-oxazin-2-amine,
(4S,6S)-4-(5-((5-chloropyridin-3-yl)ethynyl)-2-fluorophenyl)-4-methyl-6-(trifluoromethyl)-5,6-dihydro-4H-1,3-oxazin-2-amine,
(4S,6S)-4-(5-((5-chloropyrimidin-2-yl)ethynyl)-2-fluoro-4-methylphenyl)-4-methyl-6-(trifluoromethyl)-5,6-dihydro-4H-1,3-oxazin-2-amine,
(4S,6S)-4-(5-((5-Chloropyrimidin-2-yl)ethynyl)-2-fluorophenyl)-4-methyl-6-(trifluoromethyl)-5,6-dihydro-4H-1,3-oxazin-2-amine,
(4S,6S)-4-(5-((6-aminopyridin-3-yl)ethynyl)-2-fluorophenyl)-4-methyl-6-(trifluoromethyl)-5,6-dihydro-4H-1,3-oxazin-2-amine,
(4S,6S)-4-(5-(1-(Difluoromethyl)-1H-pyrazol-4-yl)-2-fluorophenyl)-4-methyl-6-(trifluoromethyl)-5,6-dihydro-4H-1,3-oxazin-2-amine,
(4S,6S)-4-(5-(5-(1H-tetrazol-5-yl)pyridin-3-yl)-2-fluorophenyl)-4-(fluoromethyl)-6-(trifluoromethyl)-5,6-dihydro-4H-1,3-oxazin-2-amine,
(4S,6S)-4-(5-(5-(4-chlorophenyl)-1,3,4-oxadiazol-2-yl)-2-fluorophenyl)-4-methyl-6-(trifluoromethyl)-5,6-dihydro-4H-1,3-oxazin-2-amine,
(4S,6S)-4-(5-(5-Chloropyridin-3-yl)-2-fluorophenyl)-4-methyl-6-(trifluoromethyl)-5,6-dihydro-4H-1,3-oxazin-2-amine,
(4S,6S)-4-(5-(6-chlorobenzo[d]oxazol-2-yl)-2-fluorophenyl)-4-(fluoromethyl)-6-(trifluoromethyl)-5,6-dihydro-4H-1,3-oxazin-2-amine,
(4S,6S)-4-(5-(6-chlorobenzo[d]oxazol-2-yl)-2-fluorophenyl)-4-methyl-6-(trifluoromethyl)-5,6-dihydro-4H-1,3-oxazin-2-amine,
(4S,6S)-4-(5-(6-Chloropyrazin-2-yl)-2-fluorophenyl)-4-methyl-6-(trifluoromethyl)-5,6-dihydro-4H-1,3-oxazin-2-amine,
(4S,6S)-4-[2-fluoro-5-[2-(2-methoxypyrimidin-5-yl)ethynyl]phenyl]-4-methyl-6-(trifluoromethyl)-5,6-dihydro-1,3-oxazin-2-amine,
(4S,6S)-4-[5-[2-(5-chloropyrimidin-2-yl)ethynyl]-2-fluorophenyl]-4-(fluoromethyl)-6-(trifluoromethyl)-5,6-dihydro-1,3-oxazin-2-amine,
(4S,6S)-4-[5-[2-(6-chloropyridin-3-yl)ethynyl]-2-fluorophenyl]-4-methyl-6-(trifluoromethyl)-5,6-dihydro-1,3-oxazin-2-amine,
(4S,6S)-4-Methyl-4-(4-(5-(prop-1-ynyl)pyridin-3-yl)thiophen-2-yl)-6-(trifluoromethyl)-5,6-dihydro-4H-1,3-oxazin-2-amine,
(4S,6S)-4-methyl-4-(4-(pyrimidin-5-yl)thiophen-2-yl)-6-(trifluoromethyl)-5,6-dihydro-4H-1,3-oxazin-2-amine,
(4S,6S)-4-Methyl-6-(trifluoromethyl)-4-(4-(3-(trifluoromethyl)phenyl)thiophen-2-yl)-5,6-dihydro-4H-1,3-oxazin-2-amine,
2-((3-((4S,6S)-2-amino-4-methyl-6-(trifluoromethyl)-5,6-dihydro-4H-1,3-oxazin-4-yl)-4-fluorophenyl)ethynyl)thiazole-5-carbonitrile,
3-(5-((4S,6S)-2-Amino-4-methyl-6-(trifluoromethyl)-5,6-dihydro-4H-1,3-oxazin-4-yl)-4-chlorothiophen-2-yl)benzonitrile,
4-((3-((4S,6S)-2-amino-4-(fluoromethyl)-6-(trifluoromethyl)-5,6-dihydro-4H-1,3-oxazin-4-yl)-4-fluorophenyl)ethynyl)benzonitrile,
4-((3-((4S,6S)-2-amino-4-methyl-6-(trifluoromethyl)-5,6-dihydro-4H-1,3-oxazin-4-yl)-4-fluorophenyl)ethynyl)benzonitrile,
5-((3-((4S,6S)-2-amino-4-methyl-6-(trifluoromethyl)-5,6-dihydro-4H-1,3-oxazin-4-yl)-4-fluorophenyl)ethynyl)picolinonitrile,
5′-((4S,6S)-2-amino-4-methyl-6-(trifluoromethyl)-5,6-dihydro-4H-1,3-oxazin-4-yl)-2,4′-difluoro-2′-methylbiphenyl-3-carbonitrile,
5-(3-((4S,6S)-2-Amino-4-methyl-6-(trifluoromethyl)-5,6-dihydro-4H-1,3-oxazin-4-yl)-4-fluorophenyl)nicotinonitrile,
5-(5-((4S,6S)-2-Amino-4-methyl-6-(trifluoromethyl)-5,6-dihydro-4H-1,3-oxazin-4-yl)thiophen-3-yl)nicotinonitrile,
5-(5-((4S,6S)-2-Amino-4-methyl-6-(trifluoromethyl)-5,6-dihydro-4H-1,3-oxazin-4-yl)-4-chlorothiophen-2-yl)nicotinonitrile,
5-(5-((4S,6S)-2-amino-4-methyl-6-(trifluoromethyl)-5,6-dihydro-4H-1,3-oxazin-4-yl)thiophen-3-yl)pyrimidine-2-carbonitrile,
5-[2-[3-[(4S,6S)-2-amino-4-methyl-6-(trifluoromethyl)-5,6-dihydro-1,3-oxazin-4-yl]-4-fluorophenyl]ethynyl]pyrimidine-2-carbonitrile,
5-[2-[3-[(4S,6S)-2-amino-4-methyl-6-(trifluoromethyl)-5,6-dihydro-1,3-oxazin-4-yl]-4-fluorophenyl]ethynyl]pyridine-3-carbonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)-5-methoxynicotinonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)-5-chloronicotinonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)nicotinonitrile,
6-((3-((4S,6S)-2-Amino-4-methyl-6-(trifluoromethyl)-5,6-dihydro-4H-1,3-oxazin-4-yl)-4-fluorophenyl)ethynyl)nicotinonitrile,
6-((5-((4S,6S)-2-amino-4-methyl-6-(trifluoromethyl)-5,6-dihydro-4H-1,3-oxazin-4-yl)thiophen-3-yl)ethynyl)nicotinonitrile,
6-((5-((4S,6S)-2-amino-4-methyl-6-(trifluoromethyl)-5,6-dihydro-4H-1,3-oxazin-4-yl)-4-fluoro-2-methylphenyl)ethynyl)-5-chloronicotinonitrile,
6-((5-((4S,6S)-2-amino-4-methyl-6-(trifluoromethyl)-5,6-dihydro-4H-1,3-oxazin-4-yl)-4-fluoro-2-methylphenyl)ethynyl)nicotinonitrile,
6-(3-((4S,6S)-2-amino-4-methyl-6-(trifluoromethyl)-5,6-dihydro-4H-1,3-oxazin-4-yl)-4-fluorophenethyl)nicotinonitrile,
6-(4-(3-((4S,6S)-2-amino-4-methyl-6-(trifluoromethyl)-5,6-dihydro-4H-1,3-oxazin-4-yl)-4-fluorophenyl)-1H-1,2,3-triazol-1-yl)nicotinonitrile,
7-(3-((4S,6S)-2-amino-4-methyl-6-(trifluoromethyl)-5,6-dihydro-4H-1,3-oxazin-4-yl)-4-fluorophenylamino)-6,7-dihydro-5H-cyclopenta[b]pyridine-3-carbonitrile,
N-(3-((4R,5R,6R)-2-Amino-5-fluoro-4,5-dimethyl-6-(trifluoromethyl)-5,6-dihydro-4H-1,3-oxazin-4-yl)-4-fluorophenyl)-5-cyanopicolinamide,
N-(3-((4R,5R,6R)-2-Amino-5-fluoro-4-methyl-6-(trifluoromethyl)-5,6-dihydro-4H-1,3-oxazin-4-yl)-4-fluorophenyl)-5-cyanopicolinamide,
N-(3-((4R,5R,6R)-2-Amino-5-fluoro-4-methyl-6-(trifluoromethyl)-5,6-dihydro-4H-1,3-oxazin-4-yl)-4-fluorophenyl)-5-chloropicolinamide,
N-(3-((4R,5R,6S)-2-Amino-4-methyl-5-(2,2,2-trifluoroethoxy)-6-(trifluoromethyl)-5,6-dihydro-4H-1,3-oxazin-4-yl)-4-fluorophenyl)-5-cyanopicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-cyanopicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-chloropicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-methoxypyrazine-2-carboxamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-(difluoromethyl)pyrazine-2-carboxamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-(but-2-ynyloxy)pyrazine-2-carboxamide,
N-(3-((4S,6S)- and (4R,6R)-2-Amino-4-(fluoromethyl)-6-methyl-6-(trifluoro-methyl)-5,6-dihydro-4H-1,3-oxazin-4-yl)-4-fluorophenyl)-5-cyanopicolinamide,
N-(3-((4S,6S)-2-amino-4-methyl-6-(trifluoromethyl)-5,6-dihydro-4H-1,3-oxazin-4-yl)-2-fluorophenyl)-5-cyanopicolinamide,
N-(3-((4S,6S)-2-amino-4-methyl-6-(trifluoromethyl)-5,6-dihydro-4H-1,3-oxazin-4-yl)-2,4-difluorophenyl)-5-cyanopicolinamide,
N-(5-((4S,6S)-2-amino-4-methyl-6-(trifluoromethyl)-5,6-dihydro-4H-1,3-oxazin-4-yl)thiophen-3-yl)-5-chloropicolinamide, and
N-(5-((4S,6S)-2-amino-4-methyl-6-(trifluoromethyl)-5,6-dihydro-4H-1,3-oxazin-4-yl)thiophen-3-yl)-5-cyanopicolinamide,
   or pharmaceutically acceptable salts thereof.

In a certain embodiment the invention relates to a compound of formula I, which is selected from the group consisting of:
(4S,6S)-4-(2-fluoro-5-((3-methylisothiazol-5-yl)ethynyl)phenyl)-4-(fluoromethyl)-6-(trifluoromethyl)-5,6-dihydro-4H-1,3-oxazin-2-amine,
(4S,6S)-4-(2-fluoro-5-((3-methylisothiazol-5-yl)ethynyl)phenyl)-4-methyl-6-(trifluoromethyl)-5,6-dihydro-4H-1,3-oxazin-2-amine,
(4S,6S)-4-(2-Fluoro-5-((5-methoxypyrazin-2-yl)ethynyl)phenyl)-4-methyl-6-(trifluoromethyl)-5,6-dihydro-4H-1,3-oxazin-2-amine,
(4S,6S)-4-(2-Fluoro-5-((5-methoxypyrimidin-2-yl)ethynyl)phenyl)-4-methyl-6-(trifluoromethyl)-5,6-dihydro-4H-1,3-oxazin-2-amine,
(4S,6S)-4-(2-Fluoro-5-(pyridin-3-ylethynyl)phenyl)-4-methyl-6-(trifluoromethyl)-5,6-dihydro-4H-1,3-oxazin-2-amine,
(4S,6S)-4-(2-fluoro-5-(pyrimidin-5-ylethynyl)phenyl)-4-methyl-6-(trifluoromethyl)-5,6-dihydro-4H-1,3-oxazin-2-amine,
(4S,6S)-4-(5-((1-ethyl-1H-pyrazol-4-yl)ethynyl)-2-fluorophenyl)-4-(fluoromethyl)-6-(trifluoromethyl)-5,6-dihydro-4H-1,3-oxazin-2-amine,
(4S,6S)-4-(5-((2-aminopyrimidin-5-yl)ethynyl)-2-fluorophenyl)-4-methyl-6-(trifluoromethyl)-5,6-dihydro-4H-1,3-oxazin-2-amine,
(4S,6S)-4-(5-((2-Chloropyridin-4-yl)ethynyl)-2-fluorophenyl)-4-methyl-6-(trifluoromethyl)-5,6-dihydro-4H-1,3-oxazin-2-amine,
(4S,6S)-4-(5-((4-chloro-1-(difluoromethyl)-1H-pyrazol-3-yl)ethynyl)-2-fluorophenyl)-4-methyl-6-(trifluoromethyl)-5,6-dihydro-4H-1,3-oxazin-2-amine,
(4S,6S)-4-(5-((4-chloro-1-(difluoromethyl)-1H-pyrazol-3-yl)ethynyl)-2-fluoro-4-methylphenyl)-4-methyl-6-(trifluoromethyl)-5,6-dihydro-4H-1,3-oxazin-2-amine,
(4S,6S)-4-(5-((4-chlorophenyl)ethynyl)-2-fluorophenyl)-4-methyl-6-(trifluoromethyl)-5,6-dihydro-4H-1,3-oxazin-2-amine,
(4S,6S)-4-(5-((5-chloropyridin-3-yl)ethynyl)-2-fluorophenyl)-4-(fluoromethyl)-6-(trifluoromethyl)-5,6-dihydro-4H-1,3-oxazin-2-amine,
(4S,6S)-4-(5-((5-chloropyridin-3-yl)ethynyl)-2-fluorophenyl)-4-methyl-6-(trifluoromethyl)-5,6-dihydro-4H-1,3-oxazin-2-amine,
(4S,6S)-4-(5-((5-chloropyrimidin-2-yl)ethynyl)-2-fluoro-4-methylphenyl)-4-methyl-6-(trifluoromethyl)-5,6-dihydro-4H-1,3-oxazin-2-amine,
(4S,6S)-4-(5-((5-Chloropyrimidin-2-yl)ethynyl)-2-fluorophenyl)-4-methyl-6-(trifluoromethyl)-5,6-dihydro-4H-1,3-oxazin-2-amine,
(4S,6S)-4-(5-((6-aminopyridin-3-yl)ethynyl)-2-fluorophenyl)-4-methyl-6-(trifluoromethyl)-5,6-dihydro-4H-1,3-oxazin-2-amine,
(4S,6S)-4-[2-fluoro-5-[2-(2-methoxypyrimidin-5-yl)ethynyl]phenyl]-4-methyl-6-(trifluoromethyl)-5,6-dihydro-1,3-oxazin-2-amine,
(4S,6S)-4-[5-[2-(5-chloropyrimidin-2-yl)ethynyl]-2-fluorophenyl]-4-(fluoromethyl)-6-(trifluoromethyl)-5,6-dihydro-1,3-oxazin-2-amine,
(4S,6S)-4-[5-[2-(6-chloropyridin-3-yl)ethynyl]-2-fluorophenyl]-4-methyl-6-(trifluoromethyl)-5,6-dihydro-1,3-oxazin-2-amine,
2-((3-((4S,6S)-2-amino-4-methyl-6-(trifluoromethyl)-5,6-dihydro-4H-1,3-oxazin-4-yl)-4-fluorophenyl)ethynyl)thiazole-5-carbonitrile,
4-((3-((4S,6S)-2-amino-4-(fluoromethyl)-6-(trifluoromethyl)-5,6-dihydro-4H-1,3-oxazin-4-yl)-4-fluorophenyl)ethynyl)benzonitrile,
4-((3-((4S,6S)-2-amino-4-methyl-6-(trifluoromethyl)-5,6-dihydro-4H-1,3-oxazin-4-yl)-4-fluorophenyl)ethynyl)benzonitrile,
5-((3-((4S,6S)-2-amino-4-methyl-6-(trifluoromethyl)-5,6-dihydro-4H-1,3-oxazin-4-yl)-4-fluorophenyl)ethynyl)picolinonitrile,
5-[2-[3-[(4S,6S)-2-amino-4-methyl-6-(trifluoromethyl)-5,6-dihydro-1,3-oxazin-4-yl]-4-fluorophenyl]ethynyl]pyrimidine-2-carbonitrile,
5-[2-[3-[(4S,6S)-2-amino-4-methyl-6-(trifluoromethyl)-5,6-dihydro-1,3-oxazin-4-yl]-4-fluorophenyl]ethynyl]pyridine-3-carbonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)-5-methoxynicotinonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)-5-chloronicotinonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)nicotinonitrile,
6-((3-((4S,6S)-2-Amino-4-methyl-6-(trifluoromethyl)-5,6-dihydro-4H-1,3-oxazin-4-yl)-4-fluorophenyl)ethynyl)nicotinonitrile,
6-((5-((4S,6S)-2-amino-4-methyl-6-(trifluoromethyl)-5,6-dihydro-4H-1,3-oxazin-4-yl)thiophen-3-yl)ethynyl)nicotinonitrile,
6-((5-((4S,6S)-2-amino-4-methyl-6-(trifluoromethyl)-5,6-dihydro-4H-1,3-oxazin-4-yl)-4-fluoro-2-methylphenyl)ethynyl)-5-chloronicotinonitrile, and
6-((5-((4S,6S)-2-amino-4-methyl-6-(trifluoromethyl)-5,6-dihydro-4H-1,3-oxazin-4-yl)-4-fluoro-2-methylphenyl)ethynyl)nicotinonitrile,
   or pharmaceutically acceptable salts thereof.

In a certain embodiment the invention relates to a compound of formula I, which is selected from the group consisting of:
N-(3-((4R,5R,6R)-2-Amino-5-fluoro-4-methyl-6-(trifluoromethyl)-5,6-dihydro-4H-1,3-oxazin-4-yl)-4-fluorophenyl)-5-cyanopicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-cyanopicolinamide,
N-(3-((4R,5R,6R)-2-Amino-5-fluoro-4-methyl-6-(trifluoromethyl)-5,6-dihydro-4H-1,3-oxazin-4-yl)-4-fluorophenyl)-5-chloropicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-chloropicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-methoxypyrazine-2-carboxamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-(difluoromethyl)pyrazine-2-carboxamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-(but-2-ynyloxy)pyrazine-2-carboxamide,
N-(3-((4S,6S)- and (4R,6R)-2-Amino-4-(fluoromethyl)-6-methyl-6-(trifluoro-methyl)-5,6-dihydro-4H-1,3-oxazin-4-yl)-4-fluorophenyl)-5-cyanopicolinamide,
(4S,6S)-4-(2-fluoro-5-(pyrimidin-5-yl)phenyl)-4-methyl-6-(trifluoromethyl)-5,6-dihydro-4H-1,3-oxazin-2-amine,
(4S,6S)-4-(5-(5-Chloropyridin-3-yl)-2-fluorophenyl)-4-methyl-6-(trifluoromethyl)-5,6-dihydro-4H-1,3-oxazin-2-amine,
(4S,6S)-4-(2-Fluoro-5-(5-(prop-1-ynyl)pyridin-3-yl)phenyl)-4-methyl-6-(trifluoromethyl)-5,6-dihydro-4H-1,3-oxazin-2-amine,
(4S,6S)-4-(5-(6-Chloropyrazin-2-yl)-2-fluorophenyl)-4-methyl-6-(trifluoromethyl)-5,6-dihydro-4H-1,3-oxazin-2-amine,
(4S,6S)-4-(5-(1-(Difluoromethyl)-1H-pyrazol-4-yl)-2-fluorophenyl)-4-methyl-6-(trifluoromethyl)-5,6-dihydro-4H-1,3-oxazin-2-amine,
(4S,6S)-4-(5-((2-Chloropyridin-4-yl)ethynyl)-2-fluorophenyl)-4-methyl-6-(trifluoromethyl)-5,6-dihydro-4H-1,3-oxazin-2-amine,
(4S,6S)-4-(2-Fluoro-5-(pyridin-3-ylethynyl)phenyl)-4-methyl-6-(trifluoromethyl)-5,6-dihydro-4H-1,3-oxazin-2-amine,
(4S,6S)-4-(5-((5-Chloropyrimidin-2-yl)ethynyl)-2-fluorophenyl)-4-methyl-6-(trifluoromethyl)-5,6-dihydro-4H-1,3-oxazin-2-amine,
(4S,6S)-4-(2-Fluoro-5-((5-methoxypyrimidin-2-yl)ethynyl)phenyl)-4-methyl-6-(trifluoromethyl)-5,6-dihydro-4H-1,3-oxazin-2-amine,
(4S,6S)-4-(2-Fluoro-5-((5-methoxypyrazin-2-yl)ethynyl)phenyl)-4-methyl-6-(trifluoromethyl)-5,6-dihydro-4H-1,3-oxazin-2-amine,
(4S,6S)-4-(4-(2-Fluoropyridin-3-yl)thiophen-2-yl)-4-methyl-6-(trifluoromethyl)-5,6-dihydro-4H-1,3-oxazin-2-amine,
(4S,6S)-4-Methyl-6-(trifluoromethyl)-4-(4-(3-(trifluoromethyl)phenyl)thiophen-2-yl)-5,6-dihydro-4H-1,3-oxazin-2-amine,
(4S,6S)-4-Methyl-4-(4-(5-(prop-1-ynyl)pyridin-3-yl)thiophen-2-yl)-6-(trifluoromethyl)-5,6-dihydro-4H-1,3-oxazin-2-amine,
(4S,6S)-4-(3-Chloro-5-(5-(prop-1-ynyl)pyridin-3-yl)thiophen-2-yl)-4-methyl-6-(trifluoromethyl)-5,6-dihydro-4H-1,3-oxazin-2-amine,
(4S,6S)-4-(3-Chloro-5-(5-chloropyridin-3-yl)thiophen-2-yl)-4-methyl-6-(trifluoromethyl)-5,6-dihydro-4H-1,3-oxazin-2-amine,
N-(3-((4R,5R,6S)-2-Amino-4-methyl-5-(2,2,2-trifluoroethoxy)-6-(trifluoromethyl)-5,6-dihydro-4H-1,3-oxazin-4-yl)-4-fluorophenyl)-5-cyanopicolinamide,
(4S,6S)-4-(2,4-Difluoro-5-(pyrimidin-5-yl)phenyl)-4-methyl-6-(trifluoromethyl)-5,6-dihydro-4H-1,3-oxazin-2-amine,
(4S,6S)-4-(2,4-Difluoro-5-(2-fluoropyridin-3-yl)phenyl)-4-methyl-6-(trifluoro-methyl)-5,6-dihydro-4H-1,3-oxazin-2-amine,
(4S,6S)-4-(2,4-Difluoro-5-(5-(prop-1-ynyl)pyridin-3-yl)phenyl)-4-methyl-6-(trifluoromethyl)-5,6-dihydro-4H-1,3-oxazin-2-amine,
N-(3-((4R,5R,6R)-2-Amino-5-fluoro-4,5-dimethyl-6-(trifluoromethyl)-5,6-dihydro-4H-1,3-oxazin-4-yl)-4-fluorophenyl)-5-cyanopicolinamide,
5-(3-((4S,6S)-2-Amino-4-methyl-6-(trifluoromethyl)-5,6-dihydro-4H-1,3-oxazin-4-yl)-4-fluorophenyl)nicotinonitrile,
6-((3-((4S,6S)-2-Amino-4-methyl-6-(trifluoromethyl)-5,6-dihydro-4H-1,3-oxazin-4-yl)-4-fluorophenyl)ethynyl)nicotinonitrile,
5-(5-((4S,6S)-2-Amino-4-methyl-6-(trifluoromethyl)-5,6-dihydro-4H-1,3-oxazin-4-yl)thiophen-3-yl)nicotinonitrile,
5-(5-((4S,6S)-2-Amino-4-methyl-6-(trifluoromethyl)-5,6-dihydro-4H-1,3-oxazin-4-yl)-4-chlorothiophen-2-yl)nicotinonitrile, and
3-(5-((4S,6S)-2-Amino-4-methyl-6-(trifluoromethyl)-5,6-dihydro-4H-1,3-oxazin-4-yl)-4-chlorothiophen-2-yl)benzonitrile,
   or pharmaceutically acceptable salts thereof.

A certain embodiment of the invention provides a compound of formula I as described herein, whenever prepared by a process as defined herein.

A certain embodiment of the invention provides a compound of formula I as described herein for use as therapeutically active substance.

A certain embodiment of the invention provides a compound of formula I as described herein for the use as inhibitor of BACE1 activity.

A certain embodiment of the invention provides a compound of formula I as described herein for the use as therapeutically active substance for the therapeutic and/or prophylactic treatment of diseases and disorders characterized by elevated β-amyloid levels and/or β-amyloid oligomers and/or β-amyloid plaques and further deposits or Alzheimer's disease.

A certain embodiment of the invention provides a compound of formula I as described herein for the use as therapeutically active substance for the therapeutic and/or prophylactic treatment of Alzheimer's disease.

A certain embodiment of the invention provides a compound of formula I as described herein for the use as therapeutically active substance for the therapeutic and/or prophylactic treatment of amyotrophic lateral sclerosis (ALS), arterial thrombosis, autoimmune/inflammatory diseases, cancer such as breast cancer, cardiovascular diseases such as myocardial infarction and stroke, dermatomyositis, Down's Syndrome, gastrointestinal diseases, Glioblastoma multiforme, Graves Disease, Huntington's Disease, inclusion body myositis (IBM), inflammatory reactions, Kaposi Sarcoma, Kostmann Disease, lupus erythematosus, macrophagic myofasciitis, juvenile idiopathic arthritis, granulomatous arthritis, malignant melanoma, multiple mieloma, rheumatoid arthritis, Sjogren syndrome, SpinoCerebellar Ataxia 1, SpinoCerebellar Ataxia 7, Whipple's Disease or Wilson's Disease.

A certain embodiment of the invention provides a pharmaceutical composition comprising a compound of formula I as described herein and a pharmaceutically acceptable carrier and/or a pharmaceutically acceptable auxiliary substance.

A certain embodiment of the invention provides the use of a compound of formula I as described herein for the manufacture of a medicament for the use in inhibition of BACE1 activity.

A certain embodiment of the invention provides the use of a compound of formula I as described herein for the manufacture of a medicament for the therapeutic and/or prophylactic treatment of diseases and disorders characterized by elevated β-amyloid levels and/or β-amyloid oligomers and/or β-amyloid plaques and further deposits or Alzheimer's disease.

A certain embodiment of the invention provides the use of a compound of formula I as described herein for the manufacture of a medicament for the therapeutic and/or prophylactic treatment of Alzheimer's disease.

A certain embodiment of the invention provides the use of a compound of formula I as described herein for the manufacture of a medicament for the therapeutic and/or prophylactic treatment of amyotrophic lateral sclerosis (ALS), arterial thrombosis, autoimmune/inflammatory diseases, cancer such as breast cancer, cardiovascular diseases such as myocardial infarction and stroke, dermatomyositis, Down's Syndrome, gastrointestinal diseases, Glioblastoma multiforme, Graves Disease, Huntington's Disease, inclusion body myositis (IBM), inflammatory reactions, Kaposi Sarcoma, Kostmann Disease, lupus erythematosus, macrophagic myofasciitis, juvenile idiopathic arthritis, granulomatous arthritis, malignant melanoma, multiple mieloma, rheumatoid arthritis, Sjogren syndrome, SpinoCerebellar Ataxia 1, SpinoCerebellar Ataxia 7, Whipple's Disease or Wilson's Disease.

A certain embodiment of the invention provides the use of a compound of formula I as described herein for the manufacture of a medicament for the therapeutic and/or prophylactic treatment of Alzheimer's disease.

A certain embodiment of the invention provides a compound of formula I as described herein for the use in inhibition of BACE1 activity.

A certain embodiment of the invention provides a compound of formula I as described herein for the use in the therapeutic and/or prophylactic treatment of diseases and disorders characterized by elevated β-amyloid levels and/or β-amyloid oligomers and/or β-amyloid plaques and further deposits or Alzheimer's disease.

A certain embodiment of the invention provides a compound of formula I as described herein for the use in the therapeutic and/or prophylactic treatment of Alzheimer's disease.

A certain embodiment of the invention provides a compound of formula I as described herein for the use in the therapeutic and/or prophylactic treatment of amyotrophic lateral sclerosis (ALS), arterial thrombosis, autoimmune/inflammatory diseases, cancer such as breast cancer, cardiovascular diseases such as myocardial infarction and stroke, dermatomyositis, Down's Syndrome, gastrointestinal diseases, Glioblastoma multiforme, Graves Disease, Huntington's Disease, inclusion body myositis (IBM), inflammatory reactions, Kaposi Sarcoma, Kostmann Disease, lupus erythematosus, macrophagic myofasciitis, juvenile idiopathic arthritis, granulomatous arthritis, malignant melanoma, multiple mieloma, rheumatoid arthritis, Sjogren syndrome, SpinoCerebellar Ataxia 1, SpinoCerebellar Ataxia 7, Whipple's Disease or Wilson's Disease.

A certain embodiment of the invention provides a method for the use in inhibition of BACE1 activity, particularly for the therapeutic and/or prophylactic treatment of diseases and disorders characterized by elevated β-amyloid levels and/or β-amyloid oligomers and/or β-amyloid plaques and further deposits or Alzheimer's disease, which method comprises administering compound of formula I as described herein to a human being or animal.

A certain embodiment of the invention provides a method for the use in the therapeutic and/or prophylactic treatment of Alzheimer's disease, which method comprises administering a compound of formula I as described herein to a human being or animal.

A certain embodiment of the invention provides a method for the use in the therapeutic and/or prophylactic treatment of amyotrophic lateral sclerosis (ALS), arterial thrombosis, autoimmune/inflammatory diseases, cancer such as breast cancer, cardiovascular diseases such as myocardial infarction and stroke, dermatomyositis, Down's Syndrome, gastrointestinal diseases, Glioblastoma multiforme, Graves Disease, Huntington's Disease, inclusion body myositis (IBM), inflammatory reactions, Kaposi Sarcoma, Kostmann Disease, lupus erythematosus, macrophagic myofasciitis, juvenile idiopathic arthritis, granulomatous arthritis, malignant melanoma, multiple mieloma, rheumatoid arthritis, Sjogren syndrome, SpinoCerebellar Ataxia 1, SpinoCerebellar Ataxia 7, Whipple's Disease or Wilson's Disease, which method comprises administering a compound of formula I as described herein to a human being or animal.

Furthermore, the invention includes all optical isomers, i.e. diastereoisomers, diastereomeric mixtures, racemic mixtures, all their corresponding enantiomers and/or tautomers as well as their solvates of the compounds of formula I.

The skilled person in the art will recognize that the compounds of formula I can exist in tautomeric form

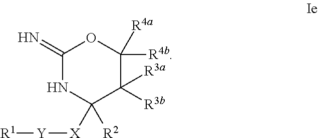

All tautomeric forms are encompassed in the present invention.

The compounds of formula I may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. Stereoisomers of compounds of formula I are compounds of formula Ia or compounds of formula Ib, in particular compounds of formula Ia, wherein the residues have the meaning as described in any of the embodiments.

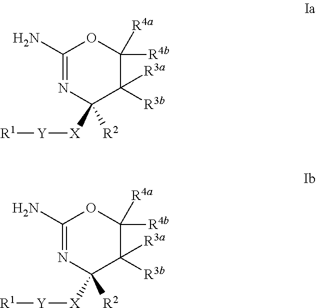

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

The compounds of formula I may be prepared in accordance with the following schemes. The starting material is commercially available or may be prepared in accordance with known methods. Any previously defined residues and variables will continue to have the previously defined meaning unless otherwise indicated.

Compounds of formula I-1 may be prepared in accordance with Scheme 1 as follows:

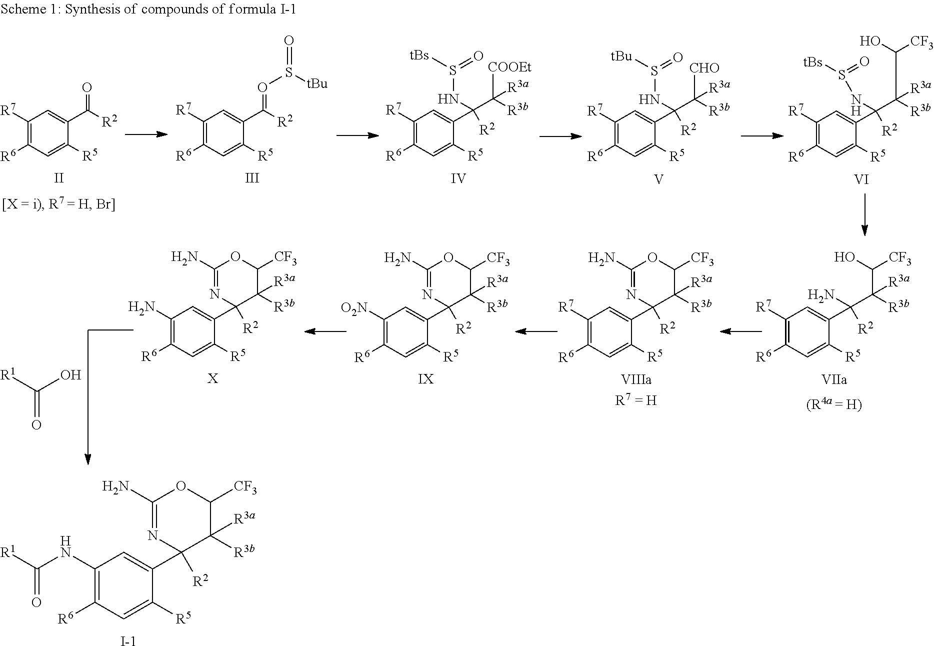

Sulfinyl imines of formula III can be prepared in analogy to T. P. Tang & J. A. Ellman³⁸, by condensation of an aryl ketone of formula II and a sulfinamide, e.g. an alkyl sulfinamide, most particularly (R)-tert-butylsulfinamide or (S)-tert-butylsulfinamide, in the presence of a Lewis acid such as e.g. a titanium(IV)alkoxide, more particularly titanium(IV)ethoxide, in a solvent such as an ether, e.g. diethyl ether or more particularly tetrahydrofuran.

The conversion of sulfinyl imines of formula III to sulfinamide esters of formula IV proceeds stereoselectively by the chiral directing group as described by Tang & Ellman³⁸. The sulfinyl imines of formula III can be reacted in a Reformatsky reaction with a zinc enolate, generated from an alkyl acetate substituted by halogen, e.g. particularly ethyl bromoacetate, and activated zinc powder at ambient to elevated temperature, particularly at 23 to 60° C., in a solvent such as an ether, e.g. diethyl ether or more particularly tetrahydrofuran, in presence of a copper(I) salt, preferably copper(I) chloride.

Aldehydes of formula V can be prepared by the reduction of ethyl esters of formula IV with an alkali hydride, e.g. lithium aluminum hydride in presence of diethylamine or sodium dihydrobis(2-methoxyethoxy)aluminate (Red-Al), preferably with diisobutylaluminum hydride (DIBAH) in an inert solvent such as an ether, e.g. diethyl ether or more particularly tetrahydrofuran, or in a chlorinated solvent, such as dichloromethane, at temperatures between −78° C. and ambient temperature.

Alcohols of formula VI can be obtained by the reaction of aldehydes of formula V with a trifluoromethylating agent, preferably trifluoromethyltrimethylsilane (Ruppert-Prakash reagent), in presence of tetrabutylammonium fluoride in a solvent such as an ether, e.g. diethyl ether or more particularly tetrahydrofuran, at temperatures between −10° C. and ambient temperature.

Hydrolysis of the chiral directing group in sulfinamide alcohols of formula VI to give aminoalcohols of formula VIIa can be accomplished with a mineral acid, e.g. sulfuric acid or particularly hydrochloric acid, in a solvent such as an ether, e.g. diethyl ether, tetrahydrofuran or more particularly 1,4-dioxane.

Aminooxazines of formula VIIIa can be prepared by reaction of aminoalcohols of formula VIIa with cyanogen bromide in a solvent such as an alcohol, particularly ethanol.

The nitro derivatives of formula IX can be prepared by nitration of oxazines of formula VIIIa, wherein R⁷ is hydrogen, following a standard procedure involving neat sulfuric acid and fuming nitric acid without using a solvent.

The reduction of the nitro group in compounds of formula IX to give anilines of formula X can be accomplished by hydrogenation using a catalyst, such as palladium on carbon, in protic solvents, such as alcohols, in particular ethanol or methanol.

Selective reaction of anilines of formula X with carboxylic acids of formula XI to give amides of formula I-1 can be effected with 4-(4,6-dimethoxy[1.3.5]triazin-2-yl)-4-methylmorpholinium chloride hydrate (DMTMM) as the condensating agent in a solvent such as methanol at temperatures between 0° C. and ambient temperature. Alternatively, 2,4,6-tripropyl-1,3,5,2,4,6-trioxatriphosphorinane-2,4,6-trioxide (T₃P®) can be used as the condensating agent in an inert solvent like e.g. ethyl acetate, at temperatures between 0° C. and ambient temperature.

Compounds of formula I-2 and I-3 may be prepared in accordance with Scheme 2 as follows:

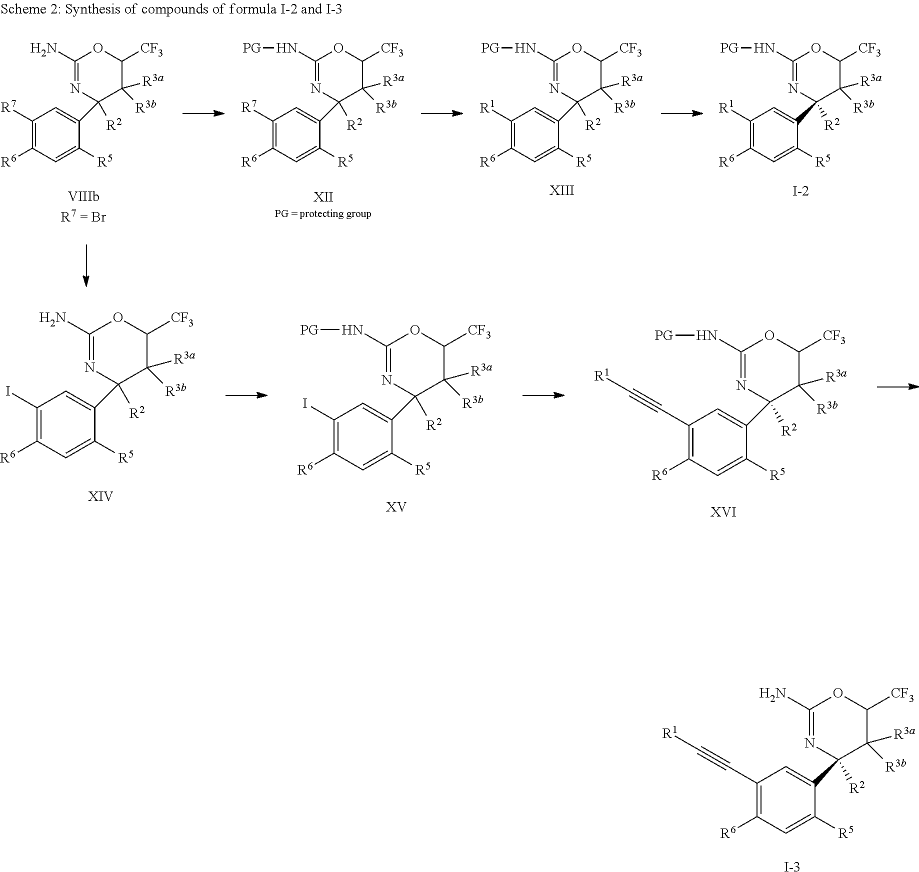

Protection of the amino group in compounds of formula VIIIb, wherein R⁷ is Br, to produce aryl bromides of formula XII can be performed with triarylmethyl chlorides, such as triphenylmethyl chloride (Tr-Cl), p-methoxyphenyldiphenylmethyl chloride (MMTr-Cl), di(p-methoxyphenyl)phenylmethyl chloride (DMTr-Cl) or tri(p-methoxyphenyl)methyl chloride (TMTr-Cl), preferably DMTr-Cl, under basic conditions, e.g. in the presence of an amine, such as triethylamine or diisopropylethylamine, in a chlorinated solvent, such as dichloromethane or chloroform, at temperatures between 0° C. and ambient temperature.

Palladium-catalyzed cross coupling between organoboronic acids or esters thereof and compounds of formula XII under conditions (Suzuki-Miyaura-coupling) known to those skilled in the art yields compounds of formula XIII.

Deprotection of the dimethoxytrityl protected amines of formula XIII to the target amine of formula I-2 can be accomplished involving a strong carbonic acid, e.g. trifluoroacetic acid, in a halogenated solvent, e.g. dichloromethane, at temperatures between 0° C. and 23° C.

Compounds of formula I-3 can be prepared as follows:

The conversion of arylbromides of formula VIIIb into the corresponding iodides of formula XIV can be accomplished utilizing a catalyst system comprising copper(I)iodide and a 1,2- or 1,3-diamine ligand as described by A. Klapars and S. L. Buchwald³⁹.

Protection of the amino group in compounds of formula XIV to produce compounds of formula XV can be performed with triarylmethyl chlorides, such as triphenylmethyl chloride (Tr-Cl), p-methoxyphenyldiphenylmethyl chloride (MMTr-Cl), di(p-methoxyphenyl)phenylmethyl chloride (DMTr-Cl) or tri(p-methoxyphenyl)methyl chloride (TMTr-Cl), preferably DMTr-Cl, under basic conditions, e.g. in the presence of an amine, such as triethylamine or diisopropylethylamine, in a chlorinated solvent, such as dichloromethane or chloroform, at temperatures between 0° C. and ambient temperature.

Sonogashira coupling of terminal alkynes with aryl iodides of formula XV to yield compounds of formula XVI can be achieved with a palladium catalyst, e.g. bis(triphenyphosphine)palladium(II) chloride, a copper(I) co-catalyst, e.g. copper(I) iodide, and an amine base, e.g. triethylamine under conditions known to those skilled in the art

Deprotection of the dimethoxytrityl protected amines of formula XVI to the target amine of formula I-3 can be accomplished involving a strong carbonic acid, e.g. trifluoroacetic acid, in a halogenated solvent, e.g. dichloromethane, at temperatures between 0° C. and 23° C.

Compounds of formula I-4 may be prepared in accordance with Scheme 3 as follows:

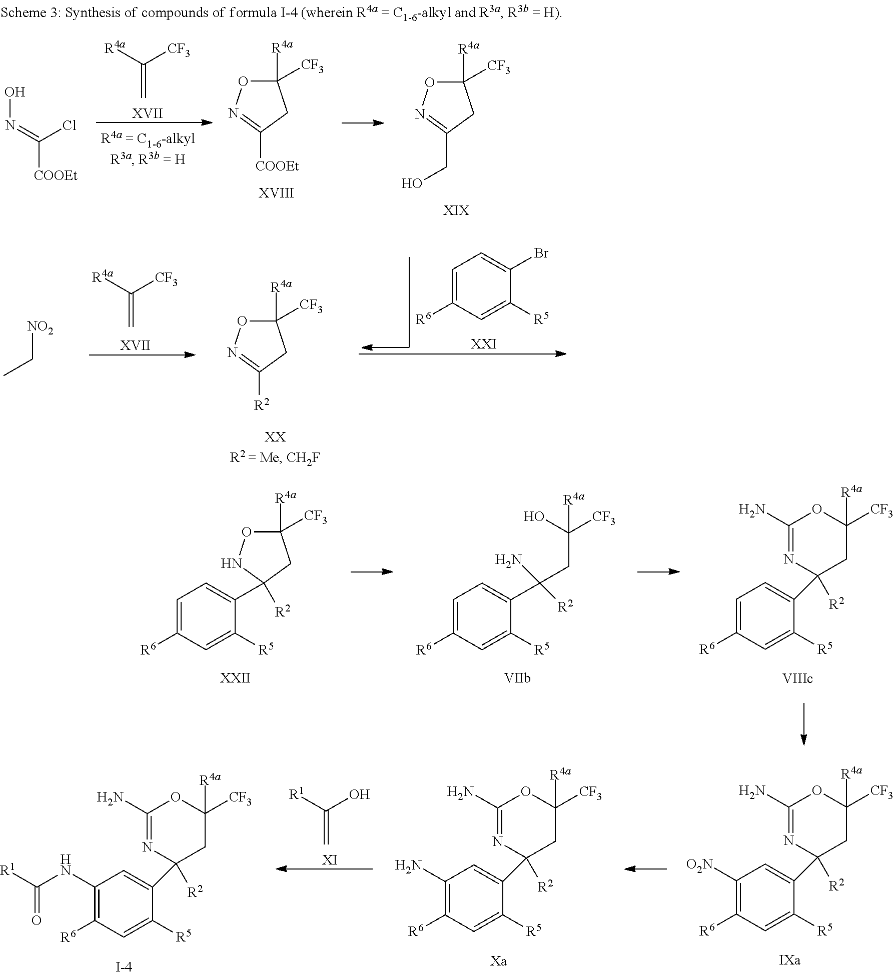

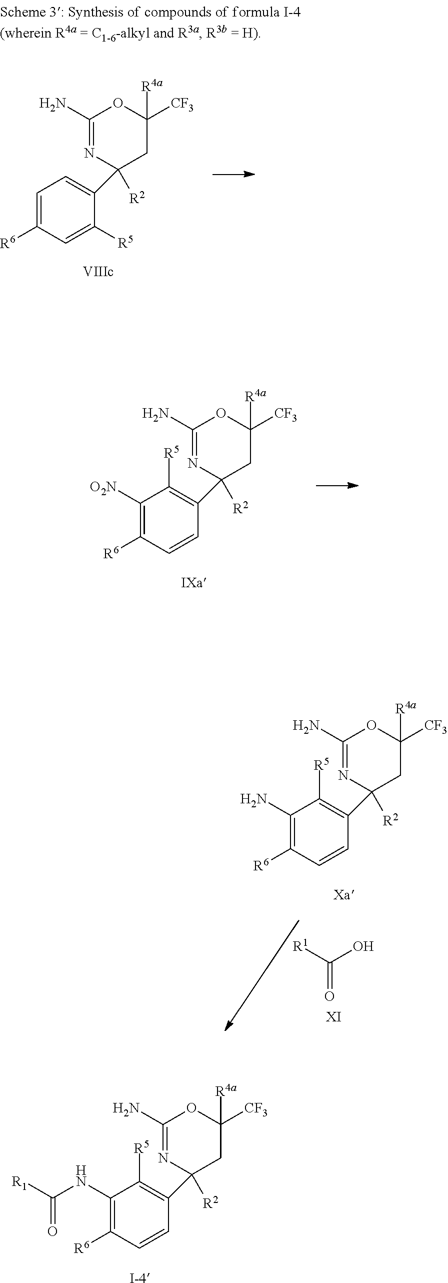

An alkyl-2-chloro-2-(hydroxyimino)acetate is reacted with an olefin of formula XVII in the presence of a base such as an alkyl amine, more particular TEA (triethyamine) or an alkali carbonate, more particular NaHCO₃, in a solvent such as a chlorinated alkane, in particular DCM (dichloromethane), or an ester, in particular EtOAc (ethyl acetate) to yield an ester of formula XVIII.

The ester of formula XVIII is reduced with a hydride, in particular NaBH₄ (sodium borohydride) in a solvent such as an alcohol, in particular EtOH (ethanol) to give the alcohol of formula XIX.

Optionally, resolution of the racemic dihydroisoxazoles of formula XIX to give the chiral dihydroisoxazoles can be done by chiral high-performance liquid chromatography (HPLC) using a Chiralpack AD or Reprosil NR column in a mixture of n-heptane and ethanol or isopropanol as the eluent.

For the synthesis of dihydroisoxazoles of formula XX, wherein R² is alkyl, particularly methyl, a nitro compound is reacted with an olefin of formula XVII in the presence of an activating reagent such as e.g. an isocyanate, in particular phenylisocyanate, and a catalytic amount of a base, in particular an alkyl amine, more particular TEA, in a solvent such as benzene or toluene, in particular benzene, or an alkyl ether, in particular diethyl ether.

Dihydroisoxazoles of formula XX, wherein R² is halogen-alkyl, particularly fluoromethyl, can be obtained from alcohols of formula XIX by reaction with a fluorinating agent like e.g. morpholinosulfur trifluoride in an inert solvent like a chlorinated alkane, preferably dichloromethane, at temperatures between −78° C. and ambient temperature.

Arylation of dihydroisoxazoles of formula XX to give isoxazolidines of formula XXII is performed by reacting an arylhalogenide, in particular an arylbromide of formula XXI, with an alkyl lithium reagent, in particular n-butyl lithium. The resulting aryllithium species can be reacted with dihydroisoxazoles of formula XX in the presence of a Lewis base, preferably boron trifluoride etherate in a solvent mixture consisting of an ether, in particular THF (tetrahydrofuran) and toluene at −100° C. to −20° C., in particular at −78° C. to yield the isoxazolidines of formula XXII, wherein R² is halogen-alkyl, particularly fluoromethyl.

Optionally, resolution of the racemic isoxazolidines of formula XXII to give the chiral isoxazolidines can be done by chiral high-performance liquid chromatography (HPLC) using a Chiralpack AD or Reprosil NR column in a mixture of n-heptane and ethanol or isopropanol as the eluent.

Hydrogenolysis of the isoxazolidines of formula XXII to the aminoalcohols of formula VIIb can be accomplished best by transfer hydrogenolysis using a palladium catalyst, in particular palladium on carbon and a hydrogen source, e.g. a salt of formic acid, in particular ammonium formate, in a protic solvent such as an alcohol, in particular ethanol.

Oxazines of formula VIIIc can be prepared by reaction of aminoalcohols of formula VIIb with cyanogen bromide in a solvent such as an alcohol, in particular ethanol, at elevated temperature. Alternatively, the reaction can be carried out in a two step sequence using cyanogen bromide and a buffer, such as e.g. sodium acetate, in the presence of a solvent, such as e.g. acetonitrile, followed by cyclisation of the intermediate in the presence of a mineral acid, in particular hydrochloric acid, in a solvent such as an ether, in particular 1,4-dioxane.

The nitration of oxazines of formula VIIIc to give the nitro-oxazines of formula IXa follows a standard procedure involving neat sulfuric acid and fuming nitric acid without using a solvent.

The reduction of the nitro group in intermediates of formula IXa to give anilines of formula Xa can be accomplished by hydrogenation using a catalyst such as palladium on carbon in protic solvents, such as alcohols, in particular ethanol or methanol.

Selective amide coupling of anilines of formula Xa and a carboxylic acid XI to give amides of formula I-4 can be effected with 4-(4,6-dimethoxy[1.3.5]triazin-2-yl)-4-methylmorpholinium chloride (DMTMM) hydrate as the condensating agent in a solvent such as an alcohol, in particular methanol.

Compounds of formula I-5 may be prepared in accordance with Scheme 4 as follows:

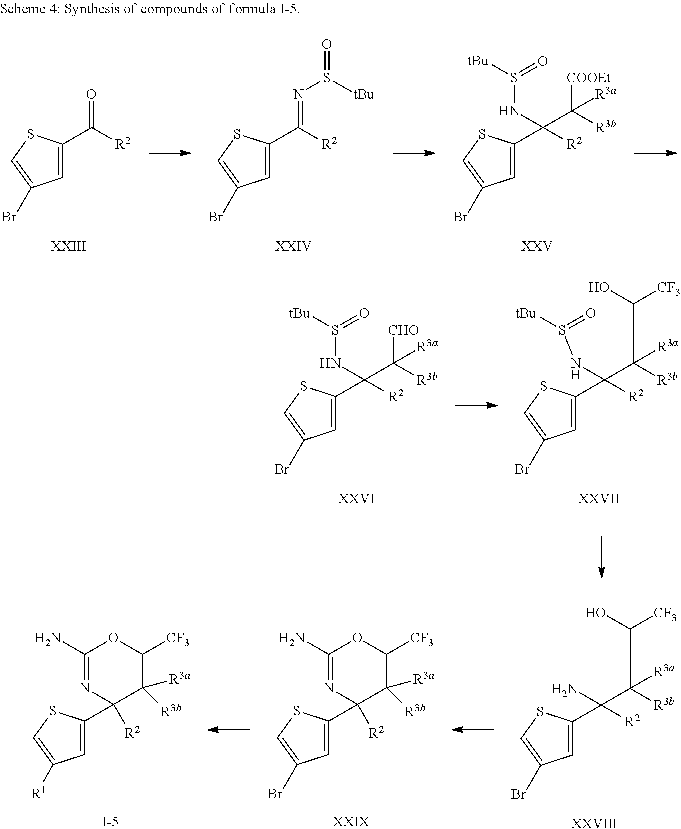

Compounds of formula I-6 may be prepared in accordance with Scheme 5 as follows:

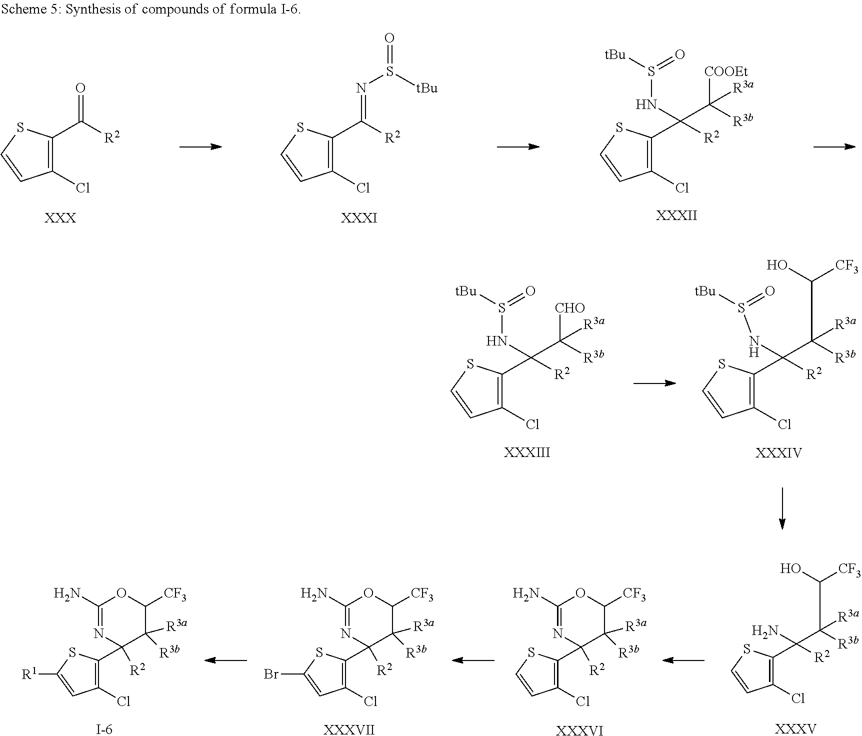

Sulfinyl imines of formula XXIV or of formula XXXI can be prepared in analogy to T. P. Tang & J. A. Ellman³⁸, by condensation of an aryl ketone of formula XXIII or of formula XXX and a sulfinamide, e.g. an alkyl sulfinamide, most particularly (R)-tert-butylsulfinamide or (S)-tert-butylsulfinamide, in the presence of a Lewis acid such as e.g. a titanium(IV)alkoxide, more particularly titanium(IV)ethoxide, in a solvent such as an ether, e.g. diethyl ether or more particularly tetrahydrofuran.

The conversion of sulfinyl imines of formula XXIV or of formula XXXI to sulfinamide esters of formula XXV or of formula XXXII proceeds stereoselectively by the chiral directing group as described by Tang & Ellman³⁸. The sulfinyl imines of formula XXIV or of formula XXXI can be reacted in a Reformatsky reaction with a zinc enolate, generated from an alkyl acetate substituted by halogen, e.g. particularly ethyl bromoacetate, and activated zinc powder at ambient to elevated temperature, particularly at 23 to 60° C., in a solvent such as an ether, e.g. diethyl ether or more particularly tetrahydrofuran, in presence of a copper(I) salt, preferably copper(I) chloride.

Aldehydes of formula XXVI or of formula XXXIII can be prepared by the reduction of ethyl esters of formula XXV or of formula XXXII with an alkali hydride, e.g. lithium aluminum hydride in presence of diethylamine or sodium dihydrobis(2-methoxyethoxy)aluminate (Red-Al), preferably with diisobutylaluminum hydride (DIBAH) in an inert solvent such as an ether, e.g. diethyl ether or more particularly tetrahydrofuran, or in a chlorinated solvent, such as dichloromethane, at temperatures between −78° C. and ambient temperature.

Alcohols of formula XXVII or of formula XXXIV can be obtained by the reaction of aldehydes of formula XXVI or of formula XXXIII with a trifluoromethylating agent, preferably trifluoromethyltrimethylsilane (Ruppert-Prakash reagent), in presence of tetrabutylammonium fluoride in a solvent such as an ether, e.g. diethyl ether or more particularly tetrahydrofuran, at temperatures between −10° C. and ambient temperature.

Hydrolysis of the chiral directing group in sulfinamide alcohols of formula XXVI or of formula XXXIII to give aminoalcohols of formula XXVIII or of formula XXXV can be accomplished with a mineral acid, e.g. sulfuric acid or particularly hydrochloric acid, in a solvent such as an ether, e.g. diethyl ether, tetrahydrofuran or more particularly 1,4-dioxane.

Aminooxazines of formula XXIX or of formula XXXVI can be prepared by reaction of aminoalcohols of formula XXVIII or of formula XXXV with cyanogen bromide in a solvent such as an alcohol, particularly ethanol.

Palladium-catalyzed cross coupling between organoboronic acids or esters thereof and aminooxazines of formula XXIX under conditions (Suzuki-Miyaura-coupling) known to those skilled in the art yields compounds of formula I-5.

Bromination of aminooxazines of formula XXXVI yields compounds of formula XXXVII. As bromination agents can be used N-bromosuccinimide in a solvent such as a chlorinated alkane, in particular dichloromethane at room temperature or a solution of bromine in acetic acid in a solvent such as acetic acid at room temperature.

Palladium-catalyzed cross coupling between organoboronic acids or esters thereof and aminooxazines of formula XXXVII under conditions (Suzuki-Miyaura-coupling) known to those skilled in the art yields compounds of formula I-6.

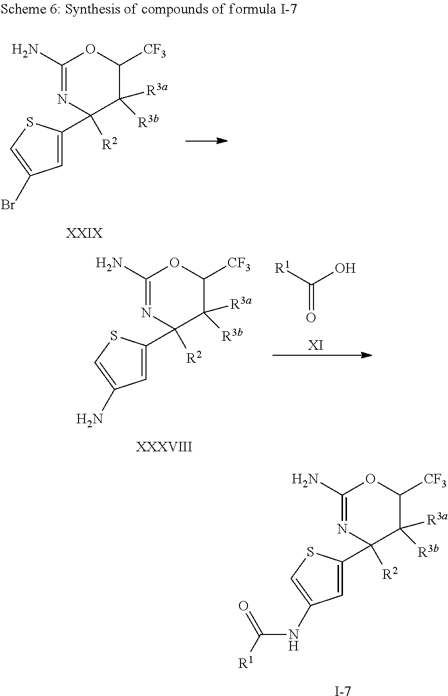

The conversion of the bromo group in formula XXIX to the amine group in formula XXXVIII can be performed by reaction with an azide, in particular sodium azide and a copper(I)halogenide in particular copper(I)iodide in the presence of L-ascorbate and an alkyl-1,2-diamine in particular trans-N,N′-dimethylcyclohexane-1,2-diamine in a protic solvent such as an alcohol in particular ethanol and water at elevated temperature preferably approximately 70° C.

The coupling of the aromatic amine XXXVIII with a carboxylic acids XI to give amides of formula I-7 can be effected with T3P in an aprotic solvent such as EtOAc at ambient temperature.

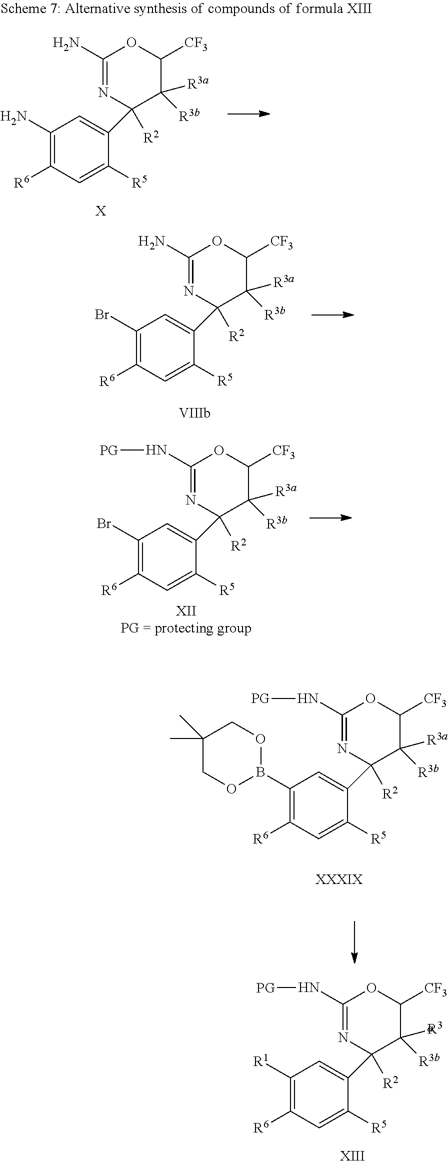

The conversion of the amino group in formula X to the bromo group in formula VIIIb can be performed with an alkyl nitrite, in particular with t-butyl nitrite and a copper(II)halogenide, in particular copper(II)bromide in a solvent like acetonitrile at elevated temperature, in particular 60-70° C.

Protection of the amino group in compounds of formula VIIIb to produce aryl bromides of formula XII can be performed with triarylmethyl chlorides, such as triphenylmethyl chloride (Tr-Cl), p-methoxyphenyldiphenylmethyl chloride (MMTr-Cl), di(p-methoxyphenyl)phenylmethyl chloride (DMTr-Cl) or tri(p-methoxyphenyl)methyl chloride (TMTr-Cl), preferably DMTr-Cl, under basic conditions, e.g. in the presence of an amine, such as triethylamine or diisopropylethylamine, in a chlorinated solvent, such as dichloromethane or chloroform, at temperatures between 0° C. and ambient temperature.

The conversion of the bromo group in formula XII to the boronic ester in formula XXXIX can be performed with an dioxaborinane, in particular 2-(5,5-dimethyl-1,3,2-dioxaborinan-2-yl)-5,5-dimethyl-1,3,2-dioxaborinane and a catalyst such as for example bis(triphenylphosphine)palladium(II)dichloride and a alkali acetate for example potassium acetate in a solvent such as an ether, in particular 1,4-dioxane at elevated temperature, in particular at 100-110° C.
The coupling of the boronic ester in formula XXXIX and an aromatic halogenide to the compound of formula XIII can be effected with a ferrocen derived catalyst, in particular 1,1′-bis(diphenylphosphino)-ferrocene-palladium(II)dichloride complex with dichloromethane and a metal carbonate, in particular cesium carbonate in a solvent mixture of an ether and water, in particular THF and water at elevated temperature, in particular between 80-90° C.

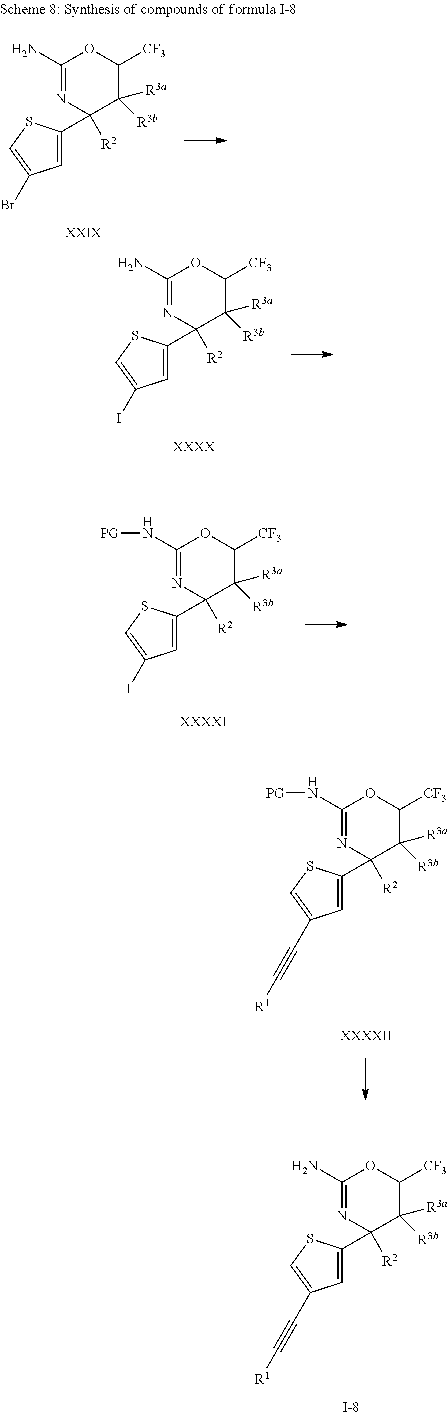

The synthesis of compounds of formula I-8 is described in Scheme 8: Bromides XXIX can be converted to the corresponding iodides of formula XXXX by utilizing a catalyst system comprising copper(I)iodide and a 1,2- or 1,3-diamine ligand as described by A. Klapars and S. L. Buchwald 40. Protection of the amino group in compounds of formula XXXX to produce compounds of formula XXXXI can be performed with triarylmethyl chlorides, such as triphenylmethyl chloride (Tr-Cl), p-methoxyphenyldiphenylmethyl chloride (MMTr-Cl), di(p-methoxyphenyl)phenylmethyl chloride (DMTr-Cl) or tri(p-methoxyphenyl)methyl chloride (TMTr-Cl), preferably DMTr-Cl, under basic conditions, e.g. in the presence of an amine, such as triethylamine or diisopropylethylamine, in a chlorinated solvent, such as dichloromethane or chloroform, at temperatures between 0° C. and ambient temperature. Sonogashira coupling of terminal alkynes with iodides of formula XXXXI to yield compounds of formula XXXXI can be achieved with a palladium catalyst, e.g. bis(triphenyphosphine)palladium(II) chloride, a copper(I) co-catalyst, e.g. copper(I) iodide, and an amine base, e.g. triethylamine under conditions known to those skilled in the art. Deprotection of dimethoxytrityl protected amines of formula XXXXI to the target amine of formula I-8 can be accomplished involving a strong carbonic acid, e.g. trifluoroacetic acid, in a halogenated solvent, e.g. dichloromethane, at temperatures between 0° C. and 23° C.

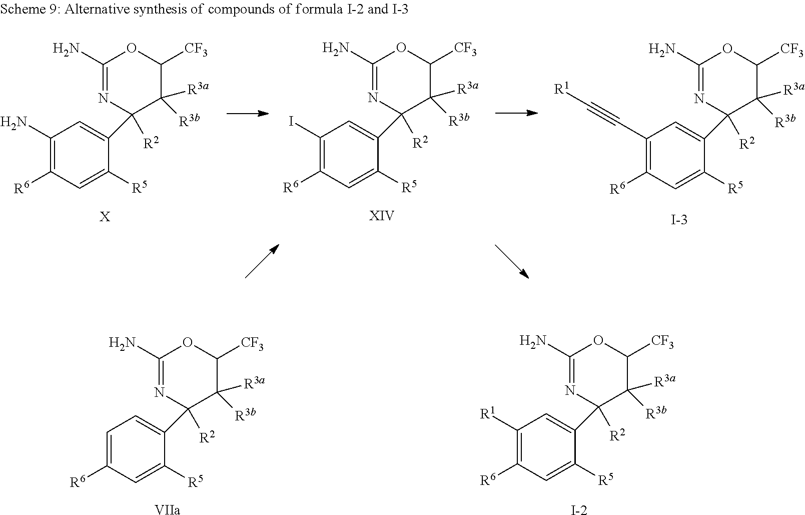

An alternative synthesis of compounds of formula I-2 and I-3 is described in Scheme 9. Anilines X can be converted to iodides XIV using a Sandmeyer reaction which can be performed by treating X with tert-butyl nitrite in the presence of copper (I) iodide in a solvent such as acetonitrile at elevated temperature. Iodides XIV can also be obtained by direct iodination of compounds VIIIa using an iodinating agent such as N-iodosuccinimide in the presence of an acid such as trifluoromethanesulfonic acid or tetrafluoroboric acid in a solvent such as dichloromethane at a temperature between 0° C. and reflux temperature of the solvent. Sonogashira coupling of terminal alkynes with aryl iodides of formula XIV to yield compounds of formula I-3 can be achieved with a palladium catalyst, e.g. bis(triphenyphosphine)palladium(II) chloride, a copper(I) co-catalyst, e.g. copper(I) iodide, and an amine base, e.g. triethylamine under conditions known to those skilled in the art. The coupling of the iodide XIV with a boronic acid or a boronic ester to the compound of formula I-2 can be effected with a ferrocen derived catalyst, in particular 1,1′-bis(diphenylphosphino)-ferrocene-palladium(II)dichloride complex with dichloromethane and a metal carbonate, in particular cesium carbonate in a solvent mixture of an ether and water, in particular THF and water at elevated temperature, in particular between 80-90° C.

The corresponding pharmaceutically acceptable salts with acids can be obtained by standard methods known to the person skilled in the art, e.g. by dissolving the compound of formula I in a suitable solvent such as e.g. dioxane or tetrahydrofuran and adding an appropriate amount of the corresponding acid. The products can usually be isolated by filtration or by chromatography. The conversion of a compound of formula I into a pharmaceutically acceptable salt with a base can be carried out by treatment of such a compound with such a base. One possible method to form such a salt is e.g. by addition of 1/n equivalents of a basic salt such as e.g. M(OH)ₙ, wherein M=metal or ammonium cation and n=number of hydroxide anions, to a solution of the compound in a suitable solvent (e.g. ethanol, ethanol-water mixture, tetrahydrofuran-water mixture) and to remove the solvent by evaporation or lyophilisation. Particular salts are hydrochloride, formate and trifluoroacetate. Specific is hydrochloride.

Insofar as their preparation is not described in the examples, the compounds of formula I as well as all intermediate products can be prepared according to analogous methods or according to the methods set forth herein. Starting materials are commercially available, known in the art or can be prepared by methods known in the art or in analogy thereto.

It will be appreciated that the compounds of general formula I in this invention may be derivatised at functional groups to provide derivatives which are capable of conversion back to the parent compound in vivo.

### Pharmacological Tests

The compounds of formula I and their pharmaceutically acceptable salts possess valuable pharmacological properties. It has been found that the compounds of the present invention are associated with inhibition of BACE1 activity. The compounds were investigated in accordance with the test given hereinafter.

Cellular Aβ-lowering assay:

The Abeta 40 AlphaLISA Assay can be used. The HEK293 APP cells were seeded in 96 well Microtiter plates in cell culture medium (Iscove's, plus 10% (v/v) fetal bovine serum, penicillin/streptomycin) to about 80% confluency and the compounds were added at a 3× concentration in ⅓ volume of culture medium (final DMSO concentration was kept at 1% v/v). After 18-20 hrs incubation at 37° C. and 5% CO₂ in a humidified incubator, the culture supernatants were harvested for the determination of Aβ 40 concentrations using Perkin-Elmer Human Amyloid beta 1-40 (high specificity) Kit (Cat#AL275C).

In a Perkin-Elmer White Optiplate-384 (Cat#6007290), 2 ul culture supernatants were combined with 2 μl of a 10× AlphaLISA Anti-hAβAcceptor beads+Biotinylated Antibody Anti-Aβ1-40 Mix (50 μg/mL/5 nM). After 1 hour room temperature incubation, 16 μl of a 1.25× preparation of Streptavidin (SA) Donor beads (25 μg/mL) were added and incubated for 30 minutes in the Dark. Light Emission at 615 nm was then recorded using EnVision-Alpha Reader. Levels of Aβ 40 in the culture supernatants were calculated as percentage of maximum signal (cells treated with 1% DMSO without inhibitor). The IC₅₀ values were calculated using the Excel XLfit software.

TABLE 1
IC₅₀ values of selected examples

| | | BACE1 cell |
|---|---|---|
| | | act. Aβ40 |
| Exam. | Structure | IC₅₀ [μM] |
| 1 | 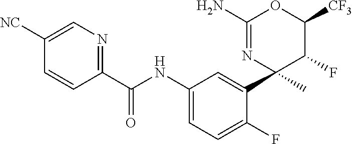 [SMILES: C[C@]1(c2cc(NC(=O)c3ccc(C#N)cn3)ccc2F)N=C(N)O[C@@H](C(F)(F)F)[C@@H]1F] | 0.150 |
| 2 | 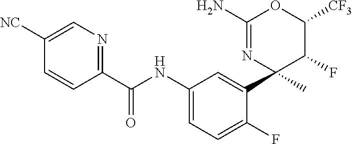 [SMILES: C[C@]1(c2cc(NC(=O)c3ccc(C#N)cn3)ccc2F)N=C(N)O[C@H](C(F)(F)F)[C@@H]1F] | 0.010 |
| 3 | 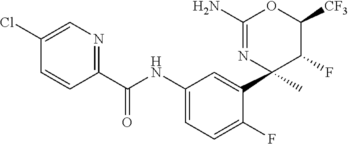 [SMILES: C[C@]1(c2cc(NC(=O)c3ccc(Cl)cn3)ccc2F)N=C(N)O[C@@H](C(F)(F)F)[C@@H]1F] | 0.490 |
| 4 | 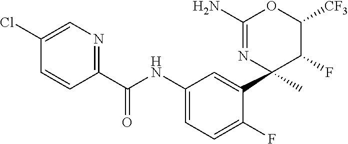 [SMILES: C[C@]1(c2cc(NC(=O)c3ccc(Cl)cn3)ccc2F)N=C(N)O[C@H](C(F)(F)F)[C@@H]1F] | 0.014 |
| 5 | 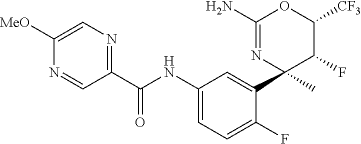 [SMILES: COc1cnc(C(=O)Nc2ccc(F)c([C@@]3(C)N=C(N)O[C@H](C(F)(F)F)[C@@H]3F)c2)cn1] | 0.012 |
| 6 | 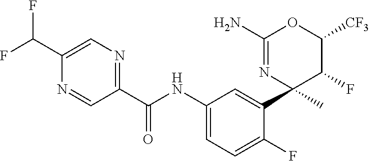 [SMILES: C[C@]1(c2cc(NC(=O)c3cnc(C(F)F)cn3)ccc2F)N=C(N)O[C@H](C(F)(F)F)[C@@H]1F] | 0.058 |
| 7 | 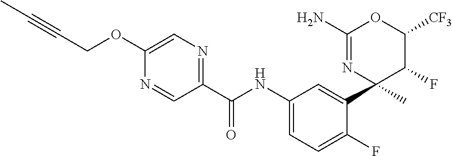 [SMILES: CC#CCOc1cnc(C(=O)Nc2ccc(F)c([C@@]3(C)N=C(N)O[C@H](C(F)(F)F)[C@@H]3F)c2)cn1] | 0.002 |
| 8 | 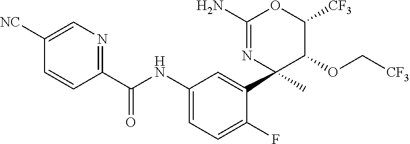 [SMILES: C[C@]1(c2cc(NC(=O)c3ccc(C#N)cn3)ccc2F)N=C(N)O[C@H](C(F)(F)F)[C@@H]1OCC(F)(F)F] | 0.002 |
| 9 | 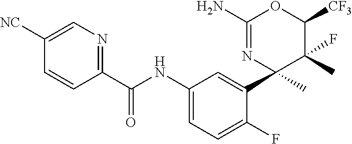 [SMILES: C[C@]1(F)[C@H](C(F)(F)F)OC(N)=N[C@]1(C)c1cc(NC(=O)c2ccc(C#N)cn2)ccc1F] | 0.440 |
| 10 | 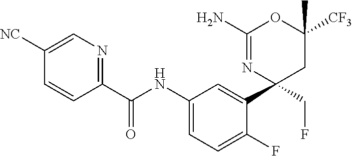 [SMILES: C[C@@]1(C(F)(F)F)C[C@@](CF)(c2cc(NC(=O)c3ccc(C#N)cn3)ccc2F)N=C(N)O1] rac | — |
| 11 | 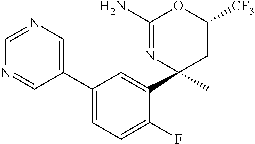 [SMILES: C[C@@]1(c2cc(-c3cncnc3)ccc2F)C[C@@H](C(F)(F)F)OC(N)=N1] | 0.420 |
| 12 | 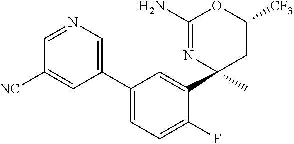 [SMILES: C[C@@]1(c2cc(-c3cncc(C#N)c3)ccc2F)C[C@@H](C(F)(F)F)OC(N)=N1] | 0.900 |
| 13 | 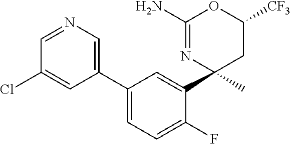 [SMILES: C[C@@]1(c2cc(-c3cncc(Cl)c3)ccc2F)C[C@@H](C(F)(F)F)OC(N)=N1] | 0.150 |
| 14 | 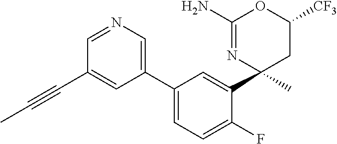 [SMILES: CC#Cc1cncc(-c2ccc(F)c([C@]3(C)C[C@@H](C(F)(F)F)OC(N)=N3)c2)c1] | 0.250 |
| 15 | 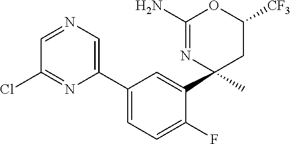 [SMILES: C[C@@]1(c2cc(-c3cncc(Cl)n3)ccc2F)C[C@@H](C(F)(F)F)OC(N)=N1] | 0.950 |
| 16 | 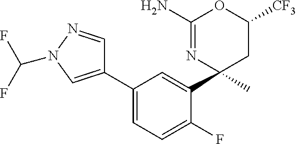 [SMILES: C[C@@]1(c2cc(-c3cnn(C(F)F)c3)ccc2F)C[C@@H](C(F)(F)F)OC(N)=N1] | 1.100 |
| 17 | 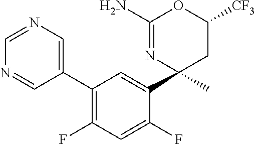 [SMILES: C[C@@]1(c2cc(-c3cncnc3)c(F)cc2F)C[C@@H](C(F)(F)F)OC(N)=N1] | 0.190 |
| 18 | 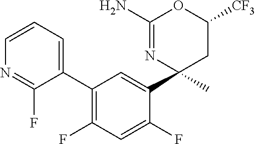 [SMILES: C[C@@]1(c2cc(-c3cccnc3F)c(F)cc2F)C[C@@H](C(F)(F)F)OC(N)=N1] | 0.120 |
| 19 | 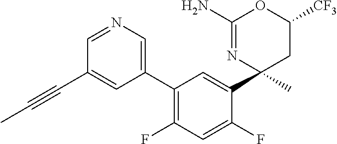 [SMILES: CC#Cc1cncc(-c2cc([C@]3(C)C[C@@H](C(F)(F)F)OC(N)=N3)c(F)cc2F)c1] | 0.570 |
| 20 | 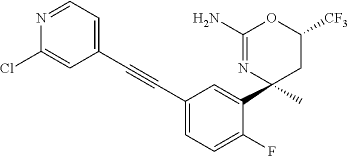 [SMILES: C[C@@]1(c2cc(C#Cc3ccnc(Cl)c3)ccc2F)C[C@@H](C(F)(F)F)OC(N)=N1] | 0.250 |
| 21 | 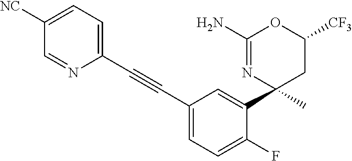 [SMILES: C[C@@]1(c2cc(C#Cc3ccc(C#N)cn3)ccc2F)C[C@@H](C(F)(F)F)OC(N)=N1] | 0.059 |
| 22 | 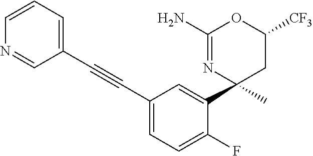 [SMILES: C[C@@]1(c2cc(C#Cc3cccnc3)ccc2F)C[C@@H](C(F)(F)F)OC(N)=N1] | 0.470 |
| 23 | 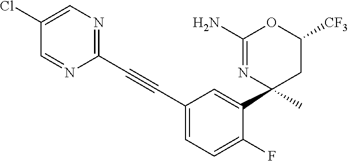 [SMILES: C[C@@]1(c2cc(C#Cc3ncc(Cl)cn3)ccc2F)C[C@@H](C(F)(F)F)OC(N)=N1] | 0.077 |
| 24 | 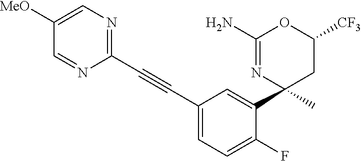 [SMILES: COc1cnc(C#Cc2ccc(F)c([C@]3(C)C[C@@H](C(F)(F)F)OC(N)=N3)c2)nc1] | 1.670 |
| 25 | 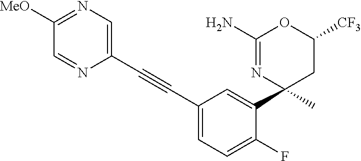 [SMILES: COc1cnc(C#Cc2ccc(F)c([C@]3(C)C[C@@H](C(F)(F)F)OC(N)=N3)c2)cn1] | 1.280 |
| 26 | 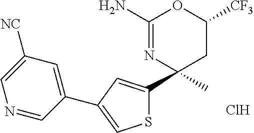 [SMILES: C[C@@]1(c2cc(-c3cncc(C#N)c3)cs2)C[C@@H](C(F)(F)F)OC(N)=N1 ; Cl] | 0.700 |
| 27 | 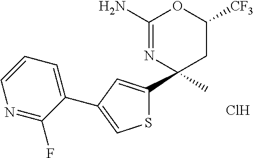 [SMILES: C[C@@]1(c2cc(-c3cccnc3F)cs2)C[C@@H](C(F)(F)F)OC(N)=N1 ; Cl] | 1.380 |
| 28 | 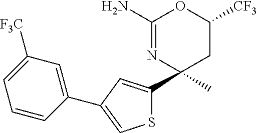 [SMILES: C[C@@]1(c2cc(-c3cccc(C(F)(F)F)c3)cs2)C[C@@H](C(F)(F)F)OC(N)=N1] | 0.330 |
| 29 | 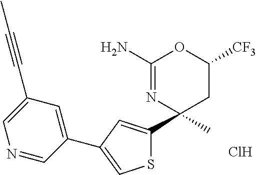 [SMILES: CC#Cc1cncc(-c2csc([C@]3(C)C[C@@H](C(F)(F)F)OC(N)=N3)c2)c1 ; Cl] | 0.310 |
| 30 | 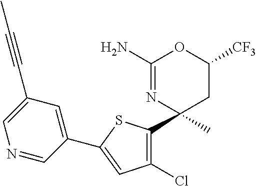 [SMILES: CC#Cc1cncc(-c2cc(Cl)c([C@]3(C)C[C@@H](C(F)(F)F)OC(N)=N3)s2)c1] | 0.620 |
| 31 | 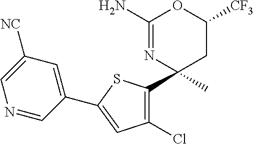 [SMILES: C[C@@]1(c2sc(-c3cncc(C#N)c3)cc2Cl)C[C@@H](C(F)(F)F)OC(N)=N1] | 0.340 |
| 32 | 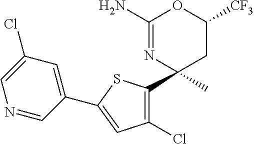 [SMILES: C[C@@]1(c2sc(-c3cncc(Cl)c3)cc2Cl)C[C@@H](C(F)(F)F)OC(N)=N1] | 0.740 |
| 33 | 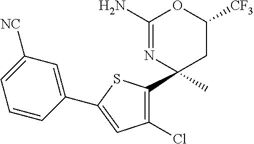 [SMILES: C[C@@]1(c2sc(-c3cccc(C#N)c3)cc2Cl)C[C@@H](C(F)(F)F)OC(N)=N1] | 0.510 |
| 34 | 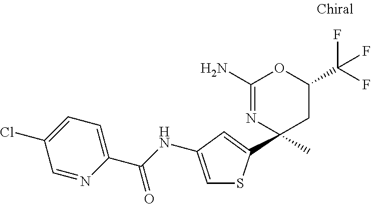 [SMILES: C[C@@]1(c2cc(NC(=O)c3ccc(Cl)cn3)cs2)C[C@@H](C(F)(F)F)OC(N)=N1] | 0.33 |
| 35 | 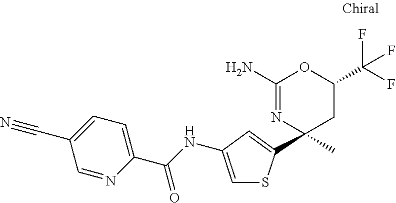 [SMILES: C[C@@]1(c2cc(NC(=O)c3ccc(C#N)cn3)cs2)C[C@@H](C(F)(F)F)OC(N)=N1] | 0.088 |
| 36 | 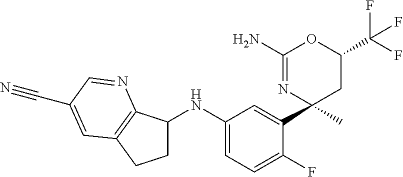 [SMILES: C[C@@]1(c2cc(NC3CCc4cc(C#N)cnc43)ccc2F)C[C@@H](C(F)(F)F)OC(N)=N1] | 0.033 |
| 37 | 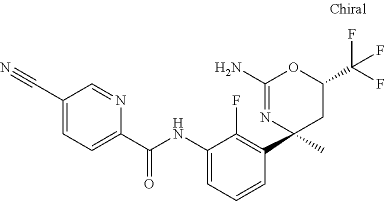 [SMILES: C[C@@]1(c2cccc(NC(=O)c3ccc(C#N)cn3)c2F)C[C@@H](C(F)(F)F)OC(N)=N1] | 0.14 |
| 38 | 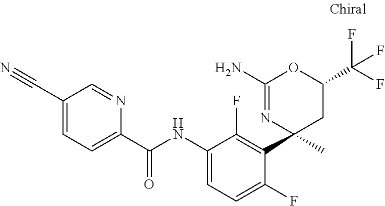 [SMILES: C[C@@]1(c2c(F)ccc(NC(=O)c3ccc(C#N)cn3)c2F)C[C@@H](C(F)(F)F)OC(N)=N1] | 0.013 |
| 39 | 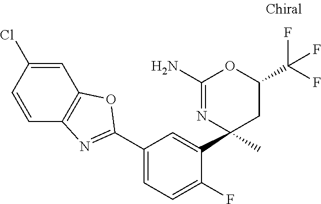 [SMILES: C[C@@]1(c2cc(-c3nc4ccc(Cl)cc4o3)ccc2F)C[C@@H](C(F)(F)F)OC(N)=N1] | 0.53 |
| 40 | 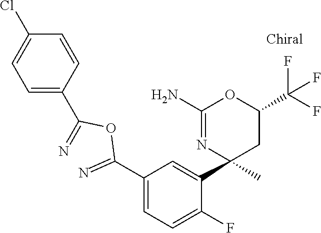 [SMILES: C[C@@]1(c2cc(C(=N)OC(=N)c3ccc(Cl)cc3)ccc2F)C[C@@H](C(F)(F)F)OC(N)=N1] | >40 |
| 41 | 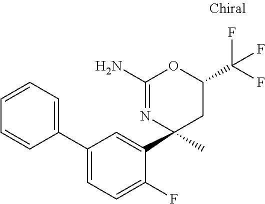 [SMILES: C[C@@]1(c2cc(-c3ccccc3)ccc2F)C[C@@H](C(F)(F)F)OC(N)=N1] | 6.42 |
| 42 | 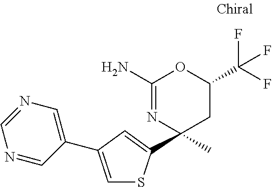 [SMILES: C[C@@]1(c2cc(-c3cncnc3)cs2)C[C@@H](C(F)(F)F)OC(N)=N1] | 2.52 |
| 43 | 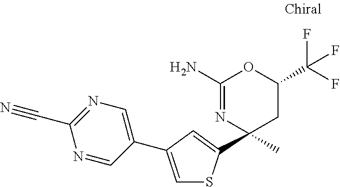 [SMILES: C[C@@]1(c2cc(-c3cnc(C#N)nc3)cs2)C[C@@H](C(F)(F)F)OC(N)=N1] | |
| 44 | 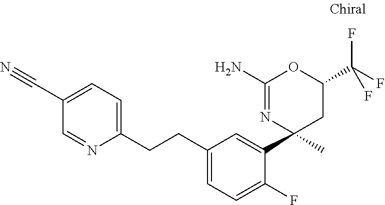 [SMILES: C[C@@]1(c2cc(CCc3ccc(C#N)cn3)ccc2F)C[C@@H](C(F)(F)F)OC(N)=N1] | 2.02 |
| 45 | 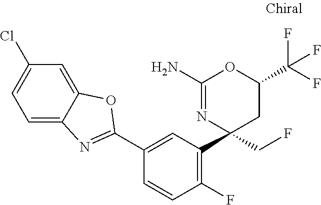 [SMILES: NC1=N[C@](CF)(c2cc(-c3nc4ccc(Cl)cc4o3)ccc2F)C[C@@H](C(F)(F)F)O1] | 1.64 |
| 46 | 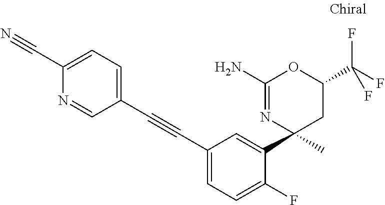 [SMILES: C[C@@]1(c2cc(C#Cc3ccc(C#N)nc3)ccc2F)C[C@@H](C(F)(F)F)OC(N)=N1] | 0.45 |
| 47 | 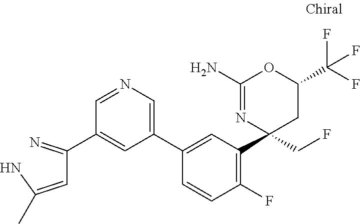 [SMILES: C/C(N)=C/C(=N)c1cncc(-c2ccc(F)c([C@]3(CF)C[C@@H](C(F)(F)F)OC(N)=N3)c2)c1] | 10.63 |
| 48 | 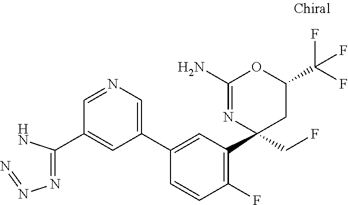 | |
| 49 | 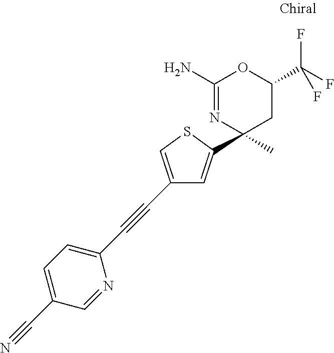 [SMILES: C[C@@]1(c2cc(C#Cc3ccc(C#N)cn3)cs2)C[C@@H](C(F)(F)F)OC(N)=N1] | |
| 50 | 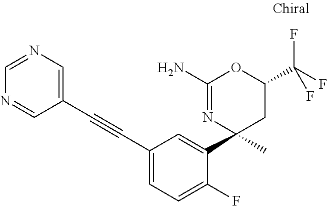 [SMILES: C[C@@]1(c2cc(C#Cc3cncnc3)ccc2F)C[C@@H](C(F)(F)F)OC(N)=N1] | 2.66 |
| 51 | 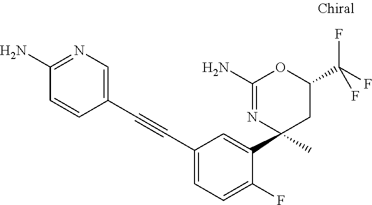 [SMILES: C[C@@]1(c2cc(C#Cc3ccc(N)nc3)ccc2F)C[C@@H](C(F)(F)F)OC(N)=N1] | |
| 52 | 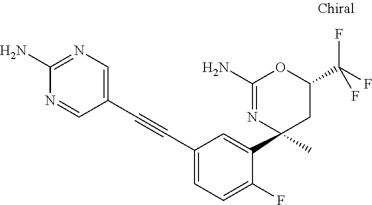 [SMILES: C[C@@]1(c2cc(C#Cc3cnc(N)nc3)ccc2F)C[C@@H](C(F)(F)F)OC(N)=N1] | |
| 53 | 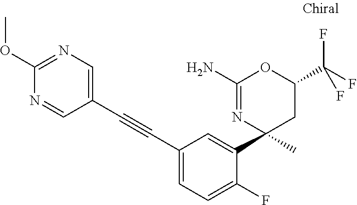 [SMILES: COc1ncc(C#Cc2ccc(F)c([C@]3(C)C[C@@H](C(F)(F)F)OC(N)=N3)c2)cn1] | 5.65 |
| 54 | 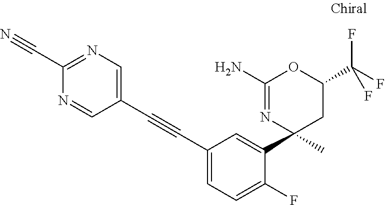 [SMILES: C[C@@]1(c2cc(C#Cc3cnc(C#N)nc3)ccc2F)C[C@@H](C(F)(F)F)OC(N)=N1] | 1.36 |
| 55 | 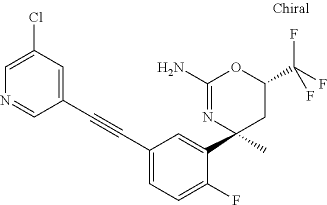 [SMILES: C[C@@]1(c2cc(C#Cc3cncc(Cl)c3)ccc2F)C[C@@H](C(F)(F)F)OC(N)=N1] | 1.17 |
| 56 | 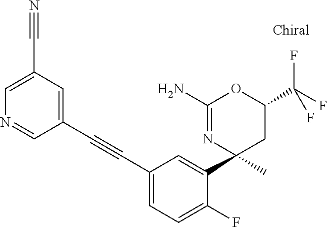 [SMILES: C[C@@]1(c2cc(C#Cc3cncc(C#N)c3)ccc2F)C[C@@H](C(F)(F)F)OC(N)=N1] | 1.65 |
| 57 | 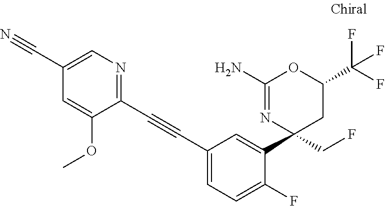 [SMILES: COc1cc(C#N)cnc1C#Cc1ccc(F)c([C@]2(CF)C[C@@H](C(F)(F)F)OC(N)=N2)c1] | 0.52 |
| 58 | 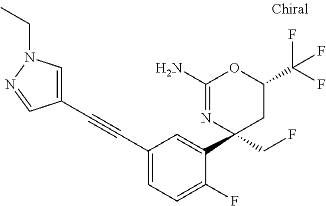 [SMILES: CCn1cc(C#Cc2ccc(F)c([C@]3(CF)C[C@@H](C(F)(F)F)OC(N)=N3)c2)cn1] | 25.74 |
| 59 | 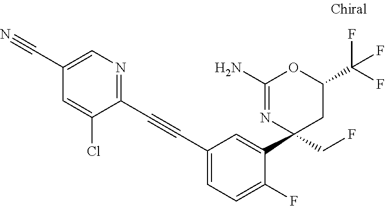 [SMILES: N#Cc1cnc(C#Cc2ccc(F)c([C@]3(CF)C[C@@H](C(F)(F)F)OC(N)=N3)c2)c(Cl)c1] | 0.79 |
| 60 | 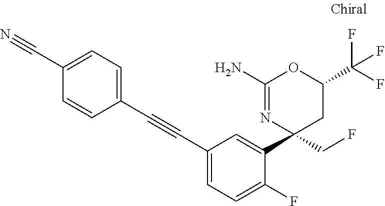 [SMILES: N#Cc1ccc(C#Cc2ccc(F)c([C@]3(CF)C[C@@H](C(F)(F)F)OC(N)=N3)c2)cc1] | 0.65 |
| 61 | 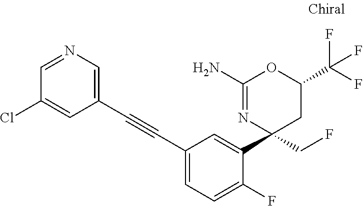 [SMILES: NC1=N[C@](CF)(c2cc(C#Cc3cncc(Cl)c3)ccc2F)C[C@@H](C(F)(F)F)O1] | 3.49 |
| 62 | 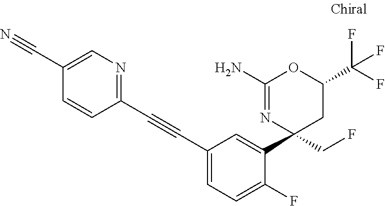 [SMILES: N#Cc1ccc(C#Cc2ccc(F)c([C@]3(CF)C[C@@H](C(F)(F)F)OC(N)=N3)c2)nc1] | 0.4 |
| 63 | 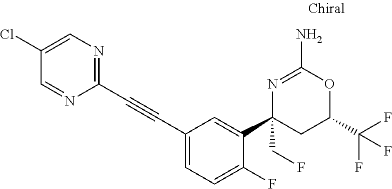 [SMILES: NC1=N[C@](CF)(c2cc(C#Cc3ncc(Cl)cn3)ccc2F)C[C@@H](C(F)(F)F)O1] | 0.43 |
| 64 | 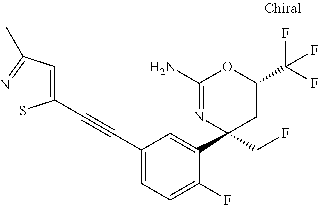 [SMILES: CC(=N)/C=C(\S)C#Cc1ccc(F)c([C@]2(CF)C[C@@H](C(F)(F)F)OC(N)=N2)c1] | 0.27 |
| 65 | 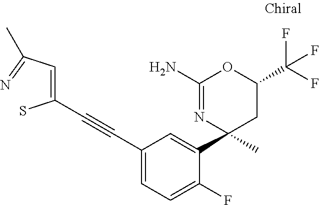 [SMILES: CC(=N)/C=C(\S)C#Cc1ccc(F)c([C@]2(C)C[C@@H](C(F)(F)F)OC(N)=N2)c1] | 0.059 |
| 66 | 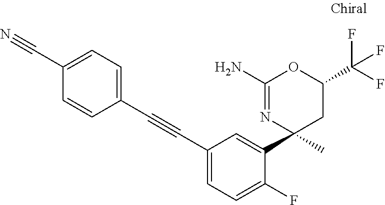 [SMILES: C[C@@]1(c2cc(C#Cc3ccc(C#N)cc3)ccc2F)C[C@@H](C(F)(F)F)OC(N)=N1] | 0.2 |
| 67 | 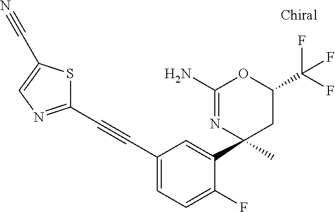 [SMILES: C[C@@]1(c2cc(C#Cc3ncc(C#N)s3)ccc2F)C[C@@H](C(F)(F)F)OC(N)=N1] | 0.95 |
| 68 | 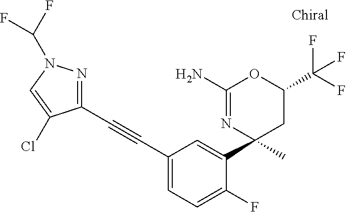 [SMILES: C[C@@]1(c2cc(C#Cc3nn(C(F)F)cc3Cl)ccc2F)C[C@@H](C(F)(F)F)OC(N)=N1] | 0.16 |
| 69 | 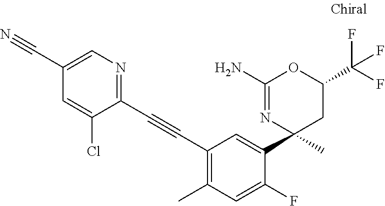 [SMILES: Cc1cc(F)c([C@]2(C)C[C@@H](C(F)(F)F)OC(N)=N2)cc1C#Cc1ncc(C#N)cc1Cl] | 0.13 |
| 70 | 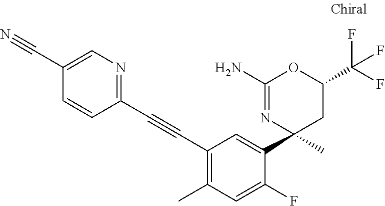 [SMILES: Cc1cc(F)c([C@]2(C)C[C@@H](C(F)(F)F)OC(N)=N2)cc1C#Cc1ccc(C#N)cn1] | 0.57 |
| 71 | 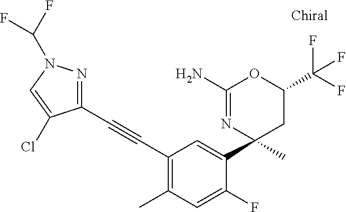 [SMILES: Cc1cc(F)c([C@]2(C)C[C@@H](C(F)(F)F)OC(N)=N2)cc1C#Cc1nn(C(F)F)cc1Cl] | 0.18 |
| 72 | 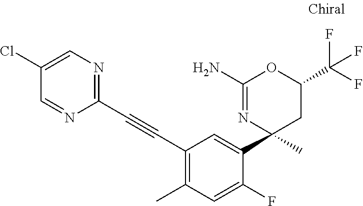 [SMILES: Cc1cc(F)c([C@]2(C)C[C@@H](C(F)(F)F)OC(N)=N2)cc1C#Cc1ncc(Cl)cn1] | 0.11 |
| 73 | 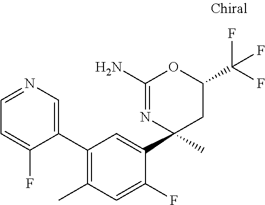 [SMILES: Cc1cc(F)c([C@]2(C)C[C@@H](C(F)(F)F)OC(N)=N2)cc1-c1cnccc1F] | 1.3 |
| 74 | 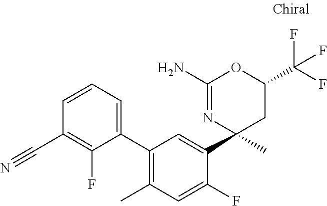 [SMILES: Cc1cc(F)c([C@]2(C)C[C@@H](C(F)(F)F)OC(N)=N2)cc1-c1cccc(C#N)c1F] | 3.56 |
| 75 | 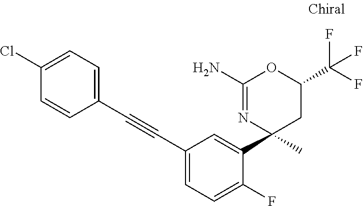 [SMILES: C[C@@]1(c2cc(C#Cc3ccc(Cl)cc3)ccc2F)C[C@@H](C(F)(F)F)OC(N)=N1] | 1.43 |
| 76 | 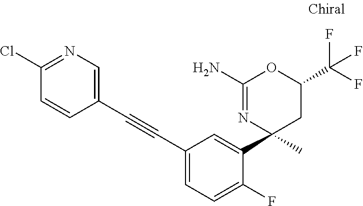 [SMILES: C[C@@]1(c2cc(C#Cc3ccc(Cl)nc3)ccc2F)C[C@@H](C(F)(F)F)OC(N)=N1] | 1.92 |
| 77 | 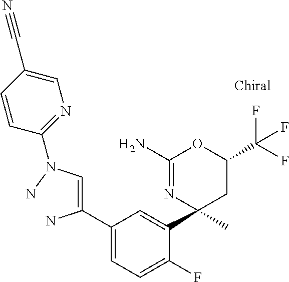 [SMILES: C[C@@]1(c2cc(/C(N)=C/N(N)c3ccc(C#N)cn3)ccc2F)C[C@@H](C(F)(F)F)OC(N)=N1] | 0.18 |

### Pharmaceutical Compositions

The compounds of formula I and the pharmaceutically acceptable salts can be used as therapeutically active substances, e.g. in the form of pharmaceutical preparations. The pharmaceutical preparations can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The compounds of formula I and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula I or a pharmaceutically acceptable salt thereof and a therapeutically inert carrier are also provided by the present invention, as is a process for their production, which comprises bringing one or more compounds of formula I and/or pharmaceutically acceptable salts thereof and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 1-500 mg, particularly 1-100 mg, of a compound of formula I. Examples of compositions according to the invention are:

### Example A

### Tablets of the Following Composition are Manufactured in the Usual Manner

TABLE 2
possible tablet composition

mg/tablet

| ingredient | 5 | 25 | 100 | 500 |
|---|---|---|---|---|
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B

### Capsules of the Following Composition are Manufactured

TABLE 3
possible capsule ingredient composition

mg/capsule

| ingredient | 5 | 25 | 100 | 500 |
|---|---|---|---|---|
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula I, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

### Soft Gelatin Capsules of the Following Composition are Manufactured

TABLE 4
possible soft gelatin capsule ingredient composition

| ingredient | mg/capsule |
|---|---|
| Compound of formula I | 5 |
| Yellow wax | 8 |
| Hydrogenated Soya bean oil | 8 |
| Partially hydrogenated plant oils | 34 |
| Soya bean oil | 110 |
| Total | 165 |

TABLE 5
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula I is dissolved in a warm melting of the other ingredients and the mixture is filled into soft gelatin capsules of appropriate size. The filled soft gelatin capsules are treated according to the usual procedures.

### Example C

### Suppositories of the Following Composition are Manufactured

TABLE 6
possible suppository composition

| ingredient | mg/supp. |
|---|---|
| Compound of formula I | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula I is added thereto and stirred until it has dispersed completely. The mixture is poured into suppository moulds of suitable size, left to cool; the suppositories are then removed from the moulds and packed individually in wax paper or metal foil.

### Example D

### Injection Solutions of the Following Composition are Manufactured

TABLE 7
possible injection solution composition

| ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

### Manufacturing Procedure

The compound of formula I is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

### Sachets of the Following Composition are Manufactured

TABLE 8
possible sachet composition

| ingredient | mg/sachet |
|---|---|
| Compound of formula I | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula I is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### Experimental Part

The following examples are provided for illustration of the invention. They should not be considered as limiting the scope of the invention, but merely as being representative thereof.

Abbreviations.

DCM, dichloromethane; Deoxo-Fluor®, bis(2-methoxyethyl)aminosulfur trifluoride; DIBAH, diisobutylaluminum hydride; DMF, N,N-dimethylformamide, DMSO, dimethyl sulfoxide; DMTMM, 4-(4,6-dimethoxy[1.3.5]triazin-2-yl)-4-methylmorpholinium chloride hydrate; EtOAc, ethyl acetate; EtOH, ethanol; MeOH, methanol; rt, room temperature; TBME, tert-butylmethylether; TEA, triethylamine; T₃P®, (2,4,6-tripropyl-1,3,5,2,4,6-trioxatriphosphorinane-2,4,6-trioxide; TBAF, tetrabutylammonium fluoride; THF, tetrahydrofuran.

General Procedure A: Synthesis of the Intermediate Aldehydes V

In a dry flask under an inert atmosphere a solution of the sulfonamide ester (IV) (9.11 mmol) in toluene (15 ml) was cooled to −76° C. and DIBAH (1M in toluene; 10.0 ml, 10.0 mmol) was added dropwise during 9 min while keeping the temperature between −75 and −68° C. After complete addition the reaction mixture was stirred at −76° C. for 1-3 h. The progress of the reaction was followed by TLC. The incomplete reaction was stopped by quenching the mixture at −76° C. with a saturated solution of NH₄Cl, then left to warm to r.t. Water (20 ml) and EtOAc (50 ml) were added under stirring. The emulsion was filtered through a layer of Dicalite®. Brine was added to the filtrate, the layers were separated, and the aqueous layer was extracted three times with EtOAc. The combined organic layers were washed with brine, dried over Na₂SO₄, then evaporated. The crude product was purified by chromatography on silica gel using mixtures of heptane and EtOAc as the eluent.

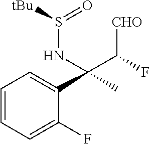 [SMILES: CC(C)(C)[S@@](=O)N[C@](C)(c1ccccc1F)[C@@H](F)C=O]

Intermediate V-1: Starting from (2R,3R)-ethyl 3-((R)-1,1-dimethylethylsulfinamido)-2-fluoro-3-(2-fluorophenyl) butanoate (IV-1) [H. Hilpert et al.⁴¹], the product (R)—N-((2R,3R)-3-fluoro-2-(2-fluorophenyl)-4-oxobutan-2-yl)-2-methylpropane-2-sulfinamide (V-1) was obtained as a light brown oil, (51% yield) after flash chromatography on silica gel (eluent: heptane-EtOAc; gradient: 0-50% EtOAc). MS: m/z=303.1 (M+H)⁺.

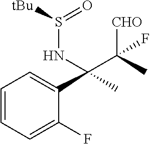 [SMILES: CC(C)(C)[S@@](=O)N[C@](C)(c1ccccc1F)[C@@](C)(F)C=O]

Intermediate V-2: Starting from (2R,3R)-ethyl 3-((R)-1,1-dimethylethylsulfinamido)-2-fluoro-3-(2-fluorophenyl)-2-methylbutanoate (IV-2) [H. Hilpert et al.¹], the product (R)—N-((2R,3R)-3-fluoro-2-(2-fluorophenyl)-3-methyl-4-oxobutan-2-yl)-2-methylpropane-2-sulfinamide (V-2) was obtained as a light brown oil (80% yield) after flash chromatography on silica gel (eluent: heptane-EtOAc; gradient: 50-100% EtOAc). MS: m/z=318.4 (M+H)⁺.

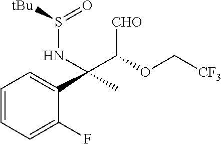 [SMILES: CC(C)(C)[S@@](=O)N[C@](C)(c1ccccc1F)[C@H](C=O)OCC(F)(F)F]

Intermediate V-3: Starting from (2R,3R)-ethyl 3-((R)-1,1-dimethylethylsulfinamido)-3-(2-fluorophenyl)-2-(2,2,2-trifluoroethoxy)butanoate (IV-3) [H. Hilpert et al.¹], the product (R)—N-((2R,3R)-2-(2-fluorophenyl)-3-(neopentyloxy)-4-oxobutan-2-yl)-2-methylpropane-2-sulfinamide (IV-3) was obtained as a light yellow oil (54% yield) after flash chromatography on silica gel (eluent: heptane-EtOAc; gradient: 50-100% EtOAc). MS: m/z=384.5 (M+H)⁺.

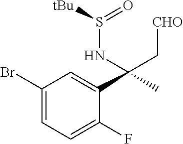 [SMILES: CC(C)(C)[S@@](=O)N[C@@](C)(CC=O)c1cc(Br)ccc1F]

Intermediate V-4: Starting from (S)-ethyl 3-(5-bromo-2-fluorophenyl)-3-((R)-1,1-dimethylethylsulfinamido)butanoate (IV-4) [D. Banner et al.⁴²], the product (R)—N—((S)-2-(5-bromo-2-fluorophenyl)-4-oxobutan-2-yl)-2-methylpropane-2-sulfinamide (V-4) was obtained as a colorless viscous oil (67% yield) after flash chromatography on silica gel (eluent: heptane-EtOAc; gradient: 0-66% EtOAc). MS: m/z=364 (M+H)⁺, 366 (M+2+H)⁺, 386 (M+Na)⁺, 388 (M+2+Na)⁺.

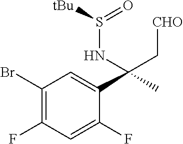 [SMILES: CC(C)(C)[S@@](=O)N[C@@](C)(CC=O)c1cc(Br)c(F)cc1F]

Intermediate V-5: Starting from (S)-ethyl 3-(5-bromo-2,4-difluorophenyl)-3-((R)-1,1-dimethylethylsulfinamido)butanoate (IV-5) [J. E. Audia et al.⁴³], the product (R)—N—((S)-2-(5-bromo-2,4-difluorophenyl)-4-oxobutan-2-yl)-2-methylpropane-2-sulfinamide (V-5) was obtained as a colorless viscous oil (56% yield) after flash chromatography on silica gel (eluent: heptane-EtOAc; gradient: 0-66% EtOAc). MS: m/z=382 (M+H)⁺, 384 (M+2+H)⁺.

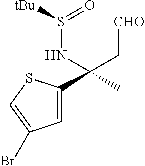 [SMILES: CC(C)(C)[S@@](=O)N[C@@](C)(CC=O)c1cc(Br)cs1]

Intermediate XVI-1: Starting from (S)-ethyl 3-(4-bromothiophen-2-yl)-3-((R)-1,1-dimethylethylsulfinamido)-butanoate (XXV-1), the product (R)—N—((S)-2-(4-bromothiophen-2-yl)-4-oxobutan-2-yl)-2-methylpropane-2-sulfinamide (XXVI-1) was obtained as a yellow oil (55% yield) after chromatography on silica gel (eluent: heptane-EtOAc; gradient: 0-80% EtOAc).

The (S)-ethyl 3-(4-bromothiophen-2-yl)-3-((R)-1,1-dimethylethylsulfinamido)-butanoate (XXV-1) was obtained in close analogy to the preparation of the corresponding methyl ester described by Stamford et al.⁴⁴

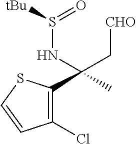 [SMILES: CC(C)(C)[S@@](=O)N[C@@](C)(CC=O)c1sccc1Cl]

Intermediate XXXIII-1: Starting from (S)-ethyl 3-(3-chlorothiophen-2-yl)-3-((R)-1,1-dimethylethylsulfinamido)butanoate (XXXII-1), the product (R)—N—((S)-2-(3-chlorothiophen-2-yl)-4-oxobutan-2-yl)-2-methylpropane-2-sulfinamide (XXXIII-1) was obtained as a yellow oil (41% yield) after chromatography on silica gel (eluent: heptane-EtOAc; gradient: 0-80% EtOAc). MS: m/z=330.4 (M+Na)⁺, 332.4 (M+2+Na)⁺.

The (S)-ethyl 3-(3-chlorothiophen-2-yl)-3-((R)-1,1-dimethylethylsulfinamido)butanoate (XXXII-1) was obtained in close analogy to the preparation of the corresponding methyl ester described by Stamford et al⁴.

General Procedure B: Synthesis of the Intermediate Alcohols VI

In a dry flask under an inert atmosphere a solution of aldehyde V (4.33 mmol) in THF (30 ml) was cooled to 0° C. and treated with trifluoromethyltrimethylsilane (Ruppert's reagent) (1.24 g, 1.29 ml, 8.66 mmol). Thereafter TBAF (433 μl, 433 μmol, Eq: 0.1) was added dropwise within 6 min. The reaction mixture was stirred at 0° C. for 1 h before TBAF (3.9 ml, 3.9 mmol, Eq: 0.9) was added dropwise. After 1 h stirring at 0° C. the reaction mixture was quenched with a saturated solution of NH₄Cl. The mixture was extracted three times with EtOAc, the combined organic layers were washed with brine, dried over Na₂SO₄ and evaporated at reduced pressure. The crude product was purified by flash chromatography on silica gel using mixtures of heptane and EtOAc as the eluent.

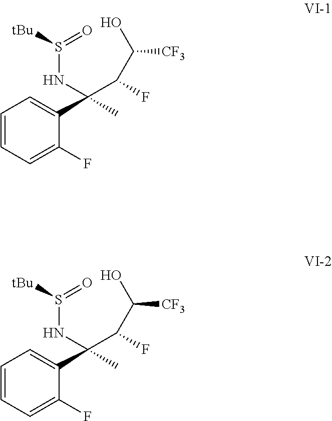 [SMILES: CC(C)(C)[S@@](=O)N[C@](C)(c1ccccc1F)[C@@H](F)[C@H](O)C(F)(F)F ; CC(C)(C)[S@@](=O)N[C@](C)(c1ccccc1F)[C@@H](F)[C@@H](O)C(F)(F)F]

Intermediates VI-1 and VI-2: Starting from (R)—N-((2R,3R)-3-fluoro-2-(2-fluorophenyl)-4-oxobutan-2-yl)-2-methylpropane-2-sulfinamide (V-1), the resulting isomers were obtained as follows: After flash chromatography on silica gel (eluent: heptane-EtOAc; gradient: 0-100% EtOAc) the (R)-2-methyl-N-((2R,3R,4S)-3,5,5,5-tetrafluoro-2-(2-fluorophenyl)-4-hydroxypentan-2-yl)propane-2-sulfinamide (VI-1) was obtained as the first eluting isomer (light yellow solid, 22% yield) and the (R)-2-methyl-N-((2R,3R,4R)-3,5,5,5-tetrafluoro-2-(2-fluorophenyl)-4-hydroxypentan-2-yl)propane-2-sulfinamide (VI-2) (light orange solid, 35% yield) as the later eluting isomer. VI-1: MS: m/z=374.6 [M+H]. VI-2: MS: m/z=372.6 [M−H]⁻.

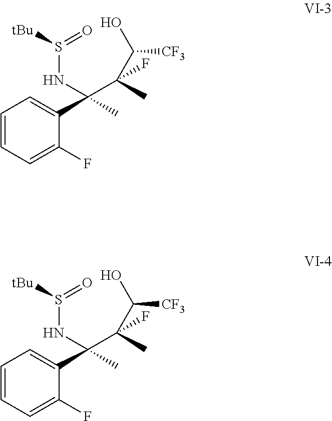 [SMILES: CC(C)(C)[S@@](=O)N[C@](C)(c1ccccc1F)[C@@](C)(F)[C@H](O)C(F)(F)F ; CC(C)(C)[S@@](=O)N[C@](C)(c1ccccc1F)[C@@](C)(F)[C@@H](O)C(F)(F)F]

Intermediate VI-3 and VI-4: Starting from (R)—N-((2R,3R)-3-fluoro-2-(2-fluorophenyl)-3-methyl-4-oxobutan-2-yl)-2-methylpropane-2-sulfinamide (V-2), the resulting isomers were obtained as follows: After 2 consecutive flash chromatographies on silica gel (eluent: heptane-EtOAc; gradient: 0-50% and 0-40% EtOAc) the (R)-2-methyl-N-((2R,3R,4S)-3,5,5,5-tetrafluoro-2-(2-fluorophenyl)-4-hydroxy-3-methylpentan-2-yl)propane-2-sulfinamide (VI-3) was obtained as the first eluting isomer (yellow, oil, 5% yield) and the (R)-2-methyl-N-((2R,3R,4R)-3,5,5,5-tetrafluoro-2-(2-fluorophenyl)-4-hydroxy-3-methylpentan-2-yl)propane-2-sulfinamide (VI-4) as the later eluting isomer (yellow oil, 15% yield). VI-4: MS: m/z=388.6 [M+H]⁺.

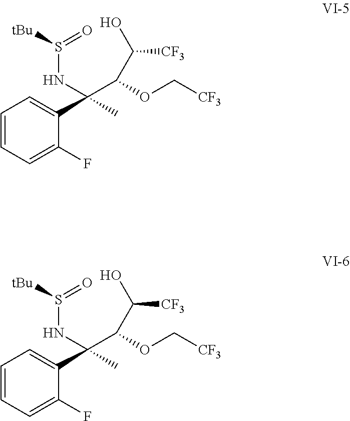 [SMILES: CC(C)(C)[S@@](=O)N[C@](C)(c1ccccc1F)[C@@H](OCC(F)(F)F)[C@H](O)C(F)(F)F ; CC(C)(C)[S@@](=O)N[C@](C)(c1ccccc1F)[C@@H](OCC(F)(F)F)[C@@H](O)C(F)(F)F]

Intermediates VI-5 and VI-6: Starting from (R)—N-((2R,3R)-2-(2-fluorophenyl)-3-(neopentyloxy)-4-oxobutan-2-yl)-2-methylpropane-2-sulfinamide (V-3), the resulting isomers were obtained as follows: After flash chromatography on silica gel (eluent: heptane-EtOAc; gradient: 0-100% EtOAc) the (R)-2-methyl-N-((2R,3R,4S)-5,5,5-trifluoro-2-(2-fluorophenyl)-4-hydroxy-3-(2,2,2-trifluoroethoxy)pentan-2-yl)propane-2-sulfinamide (VI-5) was obtained as the first eluting isomer (brown oil, 25% yield) and the (R)-2-methyl-N-((2R,3R,4R)-5,5,5-trifluoro-2-(2-fluorophenyl)-4-hydroxy-3-(2,2,2-trifluoroethoxy)pentan-2-yl)propane-2-sulfinamide (VI-6) (brown foam, 40% yield) as the later eluting isomer. VI-5: MS: m/z=454.6 [M+H]⁺. VI-6: MS: m/z=454.6 [M+H]⁺.

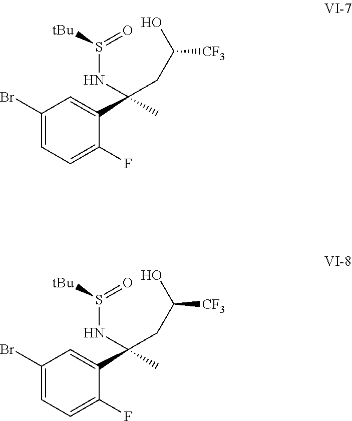 [SMILES: CC(C)(C)[S@@](=O)N[C@@](C)(C[C@H](O)C(F)(F)F)c1cc(Br)ccc1F ; CC(C)(C)[S@@](=O)N[C@@](C)(C[C@@H](O)C(F)(F)F)c1cc(Br)ccc1F]

Intermediates VI-7 and VI-8: Starting from (R)—N—((S)-2-(5-bromo-2-fluorophenyl)-4-oxobutan-2-yl)-2-methylpropane-2-sulfinamide (V-4), the resulting isomers were obtained as follows: After flash chromatography on silica gel (eluent: heptane-EtOAc; gradient: 33-100% EtOAc) the (R)—N-((2S,4S)-2-(5-bromo-2-fluorophenyl)-5,5,5-trifluoro-4-hydroxypentan-2-yl)-2-methylpropane-2-sulfinamide (VI-7) as the first eluting isomer (yellow solid, 29% yield) and the (R)—N-((2S,4R)-2-(5-bromo-2-fluorophenyl)-5,5,5-trifluoro-4-hydroxypentan-2-yl)-2-methylpropane-2-sulfinamide (VI-8) (yellow foam, 16% yield) as the later eluting isomer. VI-7: MS: m/z=432 [M−H]⁻, 434.5 [M+2−H]⁻; VI-8: MS: m/z=432.7 [M−H]⁻, 434.6 [M+2−H]⁻.

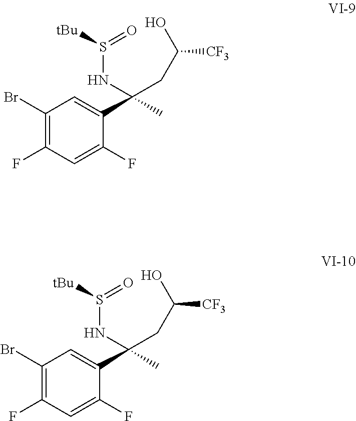 [SMILES: CC(C)(C)[S@@](=O)N[C@@](C)(C[C@H](O)C(F)(F)F)c1cc(Br)c(F)cc1F ; CC(C)(C)[S@@](=O)N[C@@](C)(C[C@@H](O)C(F)(F)F)c1cc(Br)c(F)cc1F]

Intermediates VI-9 and VI-10: Starting from (R)—N—((S)-2-(5-bromo-2,4-difluorophenyl)-4-oxobutan-2-yl)-2-methylpropane-2-sulfinamide (V-5), the resulting isomers were obtained as follows: After flash chromatography on silica gel (eluent: heptane-EtOAc; gradient: 33-100% EtOAc) the (R)—N-((2S,4S)-2-(5-bromo-2,4-difluorophenyl)-5,5,5-trifluoro-4-hydroxypentan-2-yl)-2-methylpropane-2-sulfinamide (VI-9) (yellow solid, 24% yield) and the (R)—N-((2S,4R)-2-(5-bromo-2,4-difluorophenyl)-5,5,5-trifluoro-4-hydroxypentan-2-yl)-2-methylpropane-2-sulfinamide (VI-10) (yellow solid, 17% yield) as the later eluting isomer. VI-9: MS: m/z=450 [M−H]⁻, 452.4 [M+2−H]⁻; VI-10: MS: m/z=450.4 [M−H]⁻, 452.4 [M+2−H]⁻.

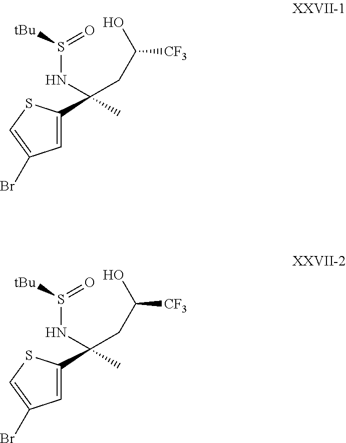 [SMILES: CC(C)(C)[S@@](=O)N[C@@](C)(C[C@H](O)C(F)(F)F)c1cc(Br)cs1 ; CC(C)(C)[S@@](=O)N[C@@](C)(C[C@@H](O)C(F)(F)F)c1cc(Br)cs1]

Intermediates XXVII-1 and XXVII-2: Starting from (R)—N—((S)-2-(4-bromothiophen-2-yl)-4-oxobutan-2-yl)-2-methylpropane-2-sulfinamide (XXVI-1), the resulting isomers were obtained as follows: After flash chromatography on silica gel (eluent: heptane-EtOAc; gradient: 0-80% EtOAc) the (R)—N-((2S,4S)-2-(4-bromothiophen-2-yl)-5,5,5-trifluoro-4-hydroxypentan-2-yl)-2-methylpropane-2-sulfinamide (XXVII-1) (yellow foam, 29% yield) and the (R)—N-((2S,4R)-2-(4-bromothiophen-2-yl)-5,5,5-trifluoro-4-hydroxypentan-2-yl)-2-methylpropane-2-sulfinamide (XXVII-2) (yellow oil, 21% yield) as the later eluting isomer. XXVII-1: MS: m/z=420.5 [M−H]⁻, 422.5 [M+2−H]⁻; XXVII-2: MS: m/z=420.5 [M−H]⁻, 422.5 [M+2−H]⁻.

The relative configuration was tentatively assigned retrospectively based on NMR-analysis of intermediates XXIX-1 and XXIX-2.

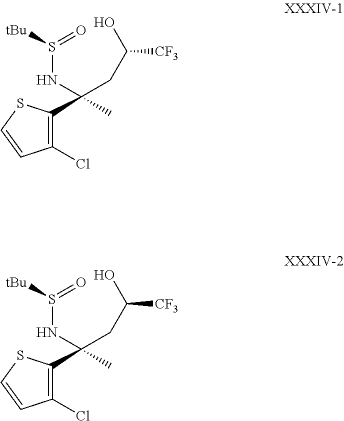 [SMILES: CC(C)(C)[S@@](=O)N[C@@](C)(C[C@H](O)C(F)(F)F)c1sccc1Cl ; CC(C)(C)[S@@](=O)N[C@@](C)(C[C@@H](O)C(F)(F)F)c1sccc1Cl]

Intermediates XXXIV-1 and XXXIV-2: Starting from (R)—N—((S)-2-(3-chlorothiophen-2-yl)-4-oxobutan-2-yl)-2-methylpropane-2-sulfinamide (XXXIII-1), the resulting isomers were obtained as follows: After flash chromatography on silica gel (eluent: heptane-EtOAc; gradient: 0-33%-66%-80% EtOAc) the (R)—N-((2S,4S)-2-(3-chlorothiophen-2-yl)-5,5,5-trifluoro-4-hydroxypentan-2-yl)-2-methylpropane-2-sulfinamide (XXXIV-1) (yellow solid, 38% yield) and the (R)—N-((2S,4R)-2-(3-chlorothiophen-2-yl)-5,5,5-trifluoro-4-hydroxypentan-2-yl)-2-methylpropane-2-sulfinamide (XXXIV-2) together with XXXIV-1 as a 4:3-mixture (yellow oil, 8% yield) as the later eluting isomer. XXXIV-1: MS: m/z=376.0 [M−H]⁻, 378.0 [M+2−H]⁻.

The relative configuration was tentatively assigned based on NMR-analyses of intermediates XXXIV-1 and XXXIV-2.

General Procedure C: Synthesis of the Intermediate Amino Alcohols VIIa

A solution of the sulfinamide alcohol VI (3.4 mmol) in MeOH (12 ml) was treated at 0° C. with a solution of HCl in dioxane (4M; 17.1 mmol). The reaction mixture was left to warm and kept at room temperature for 3-16 h. The progress of the reaction was checked by TLC. For the workup, the reaction mixture was evaporated at reduced pressure. The residue was partitioned between water (10 ml) and EtOAc (25 ml). The aqueous layer was separated, again extracted with EtOAc (25 ml). The combined organic layers were washed with water (5 ml), the aqueous layers combined and treated with an aqueous solution of Na₂CO₃ to adjust the pH to 9-10. Thereafter, the aqueous layer was extracted with EtOAc (3×35 ml). The combined organic layers were dried over Na₂SO₄ and evaporated at reduced pressure. The crude product was purified by chromatography on silica gel using mixtures of heptane and EtOAc as the eluent or was directly used in the next step without further purification.

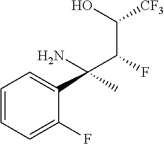 [SMILES: C[C@@](N)(c1ccccc1F)[C@@H](F)[C@H](O)C(F)(F)F]

Intermediate VIIa-1: Starting from (R)-2-methyl-N-((2R,3R,4S)-3,5,5,5-tetrafluoro-2-(2-fluorophenyl)-4-hydroxypentan-2-yl)propane-2-sulfinamide (VI-1), the product (2S,3R,4R)-4-amino-1,1,1,3-tetrafluoro-4-(2-fluorophenyl)pentan-2-ol (VIIa-1) was obtained as a light brown solid (quant. yield) and was used in the following step without further purification. MS: m/z=270.4 [M+H]⁺.

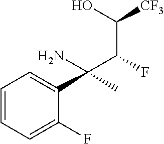 [SMILES: C[C@@](N)(c1ccccc1F)[C@@H](F)[C@@H](O)C(F)(F)F]

Intermediate VIIa-2: Starting from (R)-2-methyl-N-((2R,3R,4R)-3,5,5,5-tetrafluoro-2-(2-fluorophenyl)-4-hydroxypentan-2-yl)propane-2-sulfinamide (VI-2), the product (2R,3R,4R)-4-amino-1,1,1,3-tetrafluoro-4-(2-fluorophenyl)pentan-2-ol (VIIa-2) was obtained as a brown oil (95% yield); after chromatography on silica gel (eluent: heptane-EtOAc; gradient: 0-33% EtOAc) as a light brown oil (49% yield). MS: m/z=270.4 [M+H]⁺.

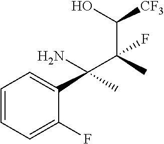 [SMILES: C[C@](F)([C@@H](O)C(F)(F)F)[C@](C)(N)c1ccccc1F]

Intermediate VIIa-3: Starting from (R)-2-methyl-N-((2R,3R,4R)-3,5,5,5-tetrafluoro-2-(2-fluorophenyl)-4-hydroxy-3-methylpentan-2-yl)propane-2-sulfinamide (VI-4), the product (2R,3R,4R)-4-amino-1,1,1,3-tetrafluoro-4-(2-fluorophenyl)-3-methylpentan-2-ol (VIIa-3) was obtained as light brown oil (80% yield) and was used in the following step without further purification. MS: m/z=284.5 [M+H]⁺.

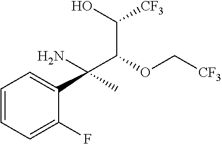 [SMILES: C[C@@](N)(c1ccccc1F)[C@@H](OCC(F)(F)F)[C@H](O)C(F)(F)F]

Intermediate VIIa-4: Starting from (R)-2-methyl-N-((2R,3R,4S)-5,5,5-trifluoro-2-(2-fluorophenyl)-4-hydroxy-3-(2,2,2-trifluoroethoxy)pentan-2-yl)propane-2-sulfinamide (VI-5), the product (2S,3R,4R)-4-amino-1,1,1-trifluoro-4-(2-fluorophenyl)-3-(2,2,2-trifluoroethoxy)pentan-2-ol (VIIa-4) was obtained as an orange solid (84% yield) and was used in the following step without further purification. MS: m/z=350.5 [M+H]⁺.

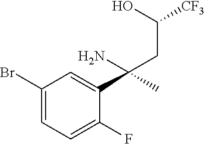 [SMILES: C[C@](N)(C[C@H](O)C(F)(F)F)c1cc(Br)ccc1F]

Intermediate VIIa-5: Starting from (R)—N-((2S,4S)-2-(5-bromo-2-fluorophenyl)-5,5,5-trifluoro-4-hydroxypentan-2-yl)-2-methylpropane-2-sulfinamide (VI-7), the product (2S,4S)-4-amino-4-(5-bromo-2-fluorophenyl)-1,1,1-trifluoropentan-2-ol (VIIa-5) was obtained as a yellow foam (quant. yield) and was used in the following step without further purification. MS: m/z=330.3 [M+H]⁺, 332 [M+2+H]⁺.

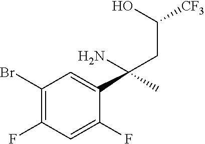 [SMILES: C[C@](N)(C[C@H](O)C(F)(F)F)c1cc(Br)c(F)cc1F]

Intermediate VIIa-6: Starting from (R)—N-((2S,4S)-2-(5-bromo-2,4-difluorophenyl)-5,5,5-trifluoro-4-hydroxypentan-2-yl)-2-methylpropane-2-sulfinamide (VI-9), the product (2S,4S)-4-amino-4-(5-bromo-2,4-difluorophenyl)-1,1,1-trifluoropentan-2-ol (VIIa-6) was obtained as a brown solid (quant. yield) and was used in the following step without further purification. MS: m/z=348 [M+H]⁺, 350.4 [M+2+H]⁺.

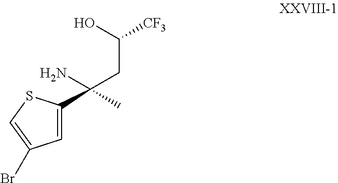 [SMILES: C[C@](N)(C[C@H](O)C(F)(F)F)c1cc(Br)cs1]

Intermediate XXVIII-1: Starting from (R)—N-((2S,4S)-2-(4-bromothiophen-2-yl)-5,5,5-trifluoro-4-hydroxypentan-2-yl)-2-methylpropane-2-sulfinamide (XXVII-1), the product (2S,4S)-4-amino-4-(4-bromothiophen-2-yl)-1,1,1-trifluoropentan-2-ol (XXVIII-1) was obtained as a yellow oil (quant. yield) and was used in the following step without further purification. LC-HRMS: m/z=316.9684 [calcd for C₉H₁₁BrF₃NOS, 316.9697].

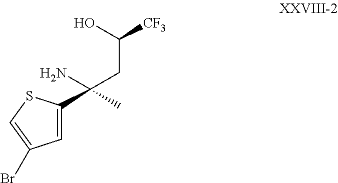 [SMILES: C[C@](N)(C[C@@H](O)C(F)(F)F)c1cc(Br)cs1]

Intermediate XXVIII-2: Starting from (R)—N-((2S,4R)-2-(4-bromothiophen-2-yl)-5,5,5-trifluoro-4-hydroxypentan-2-yl)-2-methylpropane-2-sulfinamide (XXVII-1), the product (2S,4R)-4-amino-4-(4-bromothiophen-2-yl)-1,1,1-trifluoropentan-2-ol (XXVIII-2) was obtained as a yellow oil (99% yield) and was used in the following step without further purification. LC-HRMS: m/z=316.9680 [calcd for C₉H₁₁BrF₃NOS, 316.9697].

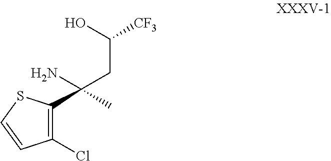 [SMILES: C[C@](N)(C[C@H](O)C(F)(F)F)c1sccc1Cl]

Intermediate XXXV-1: Starting from (R)—N-((2S,4S)-2-(3-chlorothiophen-2-yl)-5,5,5-trifluoro-4-hydroxypentan-2-yl)-2-methylpropane-2-sulfinamide (XXXIV-1), the product (2S,4S)-4-amino-4-(3-chlorothiophen-2-yl)-1,1,1-trifluoropentan-2-ol (XXXV-1) was obtained as a brown solid (quant. yield) and was used in the following step without further purification. MS: m/z=274.5 [M+H]⁺, 276.5 [M+2+H]⁺.

General Procedure D: Synthesis of the Intermediate Oxazines VIIIa

A dried tube was charged with a mixture of the amino alcohol (18.8 mmol), cyanogen bromide or a solution of cyanogen bromide (5 M in CH₃CN) (33.9 mmol) and EtOH (61 ml). The tube was sealed and heated at 80-95° C. for 15-20 h. For the workup, the reaction mixture was cooled and evaporated at reduced pressure. The residue was partitioned between EtOAc (150 ml) and a saturated aqueous solution of Na₂CO₃ (50 ml). The aqueous layer was separated and re-extracted with EtOAc (2×50 ml). The organic layers were washed with brine (50 ml), then combined, dried over Na₂SO₄ and evaporated at reduced pressure. The crude product was purified by chromatography on silica gel or on silica-NH₂ gel using mixtures of heptane and EtOAc as the eluent or it was directly used in the next step without further purification.

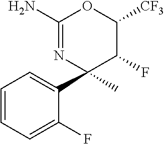 [SMILES: C[C@]1(c2ccccc2F)N=C(N)O[C@H](C(F)(F)F)[C@@H]1F]

Intermediate VIIIa-1: Starting from (2S,3R,4R)-4-amino-1,1,1,3-tetrafluoro-4-(2-fluorophenyl)pentan-2-ol (VIIa-1), the product (4R,5R,6S)-5-fluoro-4-(2-fluorophenyl)-4-methyl-6-(trifluoromethyl)-5,6-dihydro-4H-1,3-oxazin-2-amine (VIIIa-1) was obtained after chromatography on silica gel (eluent: heptane-EtOAc 1:1) as a light yellow oil (76% yield). MS: m/z=295.4 [M+H]⁺.

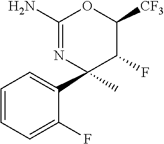 [SMILES: C[C@]1(c2ccccc2F)N=C(N)O[C@@H](C(F)(F)F)[C@@H]1F]

Intermediate VIIIa-2: Starting from (2R,3R,4R)-4-amino-1,1,1,3-tetrafluoro-4-(2-fluorophenyl)pentan-2-ol (VIIa-2), the product (4R,5R,6R)-5-fluoro-4-(2-fluorophenyl)-4-methyl-6-(trifluoromethyl)-5,6-dihydro-4H-1,3-oxazin-2-amine (VIIIa-2) was obtained after chromatography on silica gel (eluent: heptane-EtOAc; gradient: 0-100% EtOAc) as a yellow oil (56% yield). MS: m/z=295.4 [M+H]⁺.

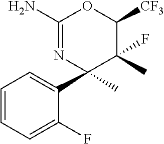 [SMILES: C[C@]1(F)[C@H](C(F)(F)F)OC(N)=N[C@]1(C)c1ccccc1F]

Intermediate VIIIa-3: Starting from (2R,3R,4R)-4-amino-1,1,1,3-tetrafluoro-4-(2-fluorophenyl)-3-methylpentan-2-ol (VIIa-3), the product (4R,5R,6R)-5-fluoro-4-(2-fluorophenyl)-4,5-dimethyl-6-(trifluoromethyl)-5,6-dihydro-4H-1,3-oxazin-2-amine (VIIIa-3) was obtained after chromatography on silica gel (eluent: heptane-EtOAc; gradient: 0-50% EtOAc) as a light brown oil (51% yield). MS: m/z=309.5 [M+H]⁺.

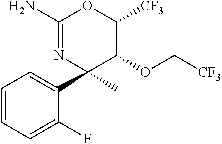 [SMILES: C[C@]1(c2ccccc2F)N=C(N)O[C@H](C(F)(F)F)[C@@H]1OCC(F)(F)F]

Intermediate VIIIa-4: Starting from (2S,3R,4R)-4-amino-1,1,1-trifluoro-4-(2-fluorophenyl)-3-(2,2,2-trifluoroethoxy)pentan-2-ol (VIIa-4), the product (4R,5R,6S)-4-(2-fluorophenyl)-4-methyl-5-(2,2,2-trifluoroethoxy)-6-(trifluoromethyl)-5,6-dihydro-4H-1,3-oxazin-2-amine (VIIIa-4) was obtained after chromatography on silica gel (eluent: heptane-EtOAc; gradient: 0-50% EtOAc) as a brown oil (41% yield). MS: m/z=375.1 [M+H]⁺.

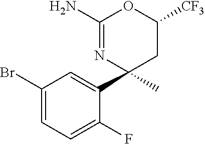 [SMILES: C[C@@]1(c2cc(Br)ccc2F)C[C@@H](C(F)(F)F)OC(N)=N1]

Intermediate VIIIb-1: Starting from (2S,4S)-4-amino-4-(5-bromo-2-fluorophenyl)-1,1,1-trifluoropentan-2-ol (VIIa-5), the product (4S,6S)-4-(5-bromo-2-fluorophenyl)-4-methyl-6-(trifluoromethyl)-5,6-dihydro-4H-1,3-oxazin-2-amine (VIIIb-1) was obtained as a 2:3-mixture together with starting material VIIa-5, difficult to separate by chromatography on silica gels (eluent: mixtures of heptane and EtOAc) (light brown oil, 77% yield total). The mixture was used as such in the following step. MS: m/z=355.4 [M+H]⁺, 357.5 [M+2+H]⁺.

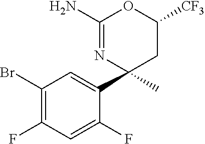 [SMILES: C[C@@]1(c2cc(Br)c(F)cc2F)C[C@@H](C(F)(F)F)OC(N)=N1]

Intermediate VIIIb-2: Starting from (2S,4S)-4-amino-4-(5-bromo-2,4-difluorophenyl)-1,1,1-trifluoropentan-2-ol (VIIa-6), the product (4S,6S)-4-(5-bromo-2,4-difluorophenyl)-4-methyl-6-(trifluoromethyl)-5,6-dihydro-4H-1,3-oxazin-2-amine (VIIIb-2) was obtained as a 3:4-mixture together with starting material VIIa-6, difficult to separate by chromatography on silica gels (eluent: mixtures of heptane and EtOAc) (light brown foam, 69% yield total). The mixture was used as such in the following step. MS: m/z=373.4 [M+H]⁺, 375 [M+2+H]⁺.

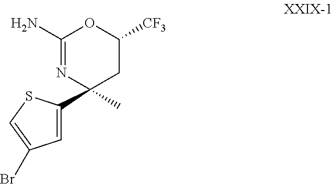 [SMILES: C[C@@]1(c2cc(Br)cs2)C[C@@H](C(F)(F)F)OC(N)=N1]

Intermediate XXIX-1: Starting from (2S,4S)-4-amino-4-(4-bromothiophen-2-yl)-1,1,1-trifluoropentan-2-ol (XXVIII-1), the product (4S,6S)-4-(4-bromothiophen-2-yl)-4-methyl-6-(trifluoromethyl)-5,6-dihydro-4H-1,3-oxazin-2-amine (XXIX-1) was obtained as a light brown oil (94% yield) and was used in the following step without further purification. MS: m/z=343 [M+H]⁺, 345 [M+2+H]⁺.

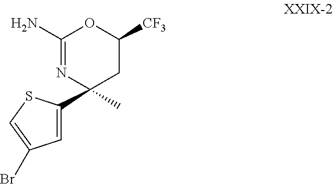 [SMILES: C[C@@]1(c2cc(Br)cs2)C[C@H](C(F)(F)F)OC(N)=N1]

Intermediate XXIX-2: Starting from (2S,4R)-4-amino-4-(4-bromothiophen-2-yl)-1,1,1-trifluoropentan-2-ol (XXVIII-2), the product (4S,6R)-4-(4-bromothiophen-2-yl)-4-methyl-6-(trifluoromethyl)-5,6-dihydro-4H-1,3-oxazin-2-amine (XXXIX-2) was obtained as a light brown oil (quant. yield) and was used in the following step without further purification. MS: m/z=343 [M+H]⁺, 345 [M+2+H]⁺.

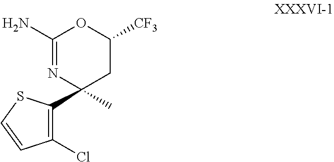 [SMILES: C[C@@]1(c2sccc2Cl)C[C@@H](C(F)(F)F)OC(N)=N1]

Intermediate XXXVI-1: Starting from (2S,4S)-4-amino-4-(3-chlorothiophen-2-yl)-1,1,1-trifluoropentan-2-ol (XXXV-1), the product (4S,6S)-4-(3-chlorothiophen-2-yl)-4-methyl-6-(trifluoromethyl)-5,6-dihydro-4H-1,3-oxazin-2-amine (XXXVI-1) was obtained after chromatography on silica gel (eluent: heptane-EtOAc; gradient: 0-35% EtOAc) as a yellow solid (59% yield). MS: m/z=299.0 [M+H]⁺.

Synthesis of Intermediate Bromo Derivative XXXVII-1

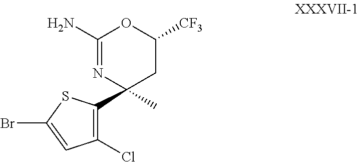 [SMILES: C[C@@]1(c2sc(Br)cc2Cl)C[C@@H](C(F)(F)F)OC(N)=N1]

A solution of (4S,6S)-4-(3-chlorothiophen-2-yl)-4-methyl-6-(trifluoromethyl)-5,6-dihydro-4H-1,3-oxazin-2-amine (XXXVI-1) (204 mg, 683 μmol) in acetic Acid (3 ml) was treated with a solution of bromine in acetic acid (1 M; 2.05 ml). The reaction mixture was stirred in a sealed tube at rt overnight. The incomplete reaction was concentrated at reduced pressure and the residue dried at high vacuum. The crude product was dissolved in EtOAc (5 ml) and washed with a saturated solution of Na₂CO₃ (1 ml). The organic layer was dried over Na₂SO₄ and evaporated at reduced pressure. The residue consisted of a 2:1-mixture of the product (4S,6S)-4-(5-bromo-3-chlorothiophen-2-yl)-4-methyl-6-(trifluoromethyl)-5,6-dihydro-4H-1,3-oxazin-2-amine (XXXVII-1) and the starting material (4S,6S)-4-(3-chlorothiophen-2-yl)-4-methyl-6-(trifluoromethyl)-5,6-dihydro-4H-1,3-oxazin-2-amine (XXXVI-1) (brown oil, 279 mg) and was engaged in the next step without further purification.

General Procedure E: Synthesis of the Intermediate Nitro Oxazines IX

A dispersion of the amino oxazine VIIIa (2.8 mmol) in cone. H₂SO₄ (22.1 g, 216 mmol) was cooled to 0° C. and stirring was continued until a complete solution was obtained. At 0° C. fuming HNO₃ (300 mg, 214 μl, 4.29 mmol) was added dropwise in 4 portions. After complete addition, the ice bath was removed and stirring continued for 30 min at room temperature. For the workup, the solution was added dropwise to a mixture of crushed ice (50 g) and water (50 g). With an aqueous solution of NaOH the pH was adjusted to 7-8. The aqueous layer was extracted twice with EtOAc, thereafter the combined organic layers were washed with brine, then dried over Na₂SO₄ and evaporated at reduced pressure. The crude product was purified by chromatography on silica gel using mixtures of heptane and EtOAc as the eluent or was directly used in the next step without further purification.

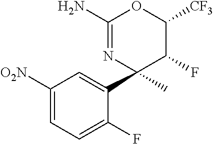 [SMILES: C[C@]1(c2cc([N+](=O)[O-])ccc2F)N=C(N)O[C@H](C(F)(F)F)[C@@H]1F]

Intermediate IX-1: Starting from (4R,5R,6S)-5-fluoro-4-(2-fluorophenyl)-4-methyl-6-(trifluoromethyl)-5,6-dihydro-4H-1,3-oxazin-2-amine (VIIIa-1), the product (4R,5R,6S)-5-fluoro-4-(2-fluoro-5-nitrophenyl)-4-methyl-6-(trifluoromethyl)-5,6-dihydro-4H-1,3-oxazin-2-amine (IX-1) was obtained after chromatography on silica gel (eluent: heptane-EtOAc; gradient: 0-50% EtOAc) as a white foam (67% yield). MS: m/z=340.4 [M+H]⁺.

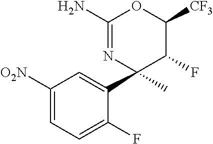 [SMILES: C[C@]1(c2cc([N+](=O)[O-])ccc2F)N=C(N)O[C@@H](C(F)(F)F)[C@@H]1F]

Intermediate IX-2: Starting from (4R,5R,6R)-5-fluoro-4-(2-fluorophenyl)-4-methyl-6-(trifluoromethyl)-5,6-dihydro-4H-1,3-oxazin-2-amine (VIIIa-2), the product (4R,5R,6R)-5-fluoro-4-(2-fluoro-5-nitrophenyl)-4-methyl-6-(trifluoromethyl)-5,6-dihydro-4H-1,3-oxazin-2-amine (IX-2) was obtained after chromatography on silica gel (eluent: heptane-EtOAc; gradient: 0-50% EtOAc) as a light yellow oil (68% yield). MS: m/z=340.4 [M+H]⁺.

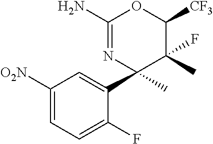 [SMILES: C[C@]1(F)[C@H](C(F)(F)F)OC(N)=N[C@]1(C)c1cc([N+](=O)[O-])ccc1F]

Intermediate IX-3: Starting from (4R,5R,6R)-5-fluoro-4-(2-fluorophenyl)-4,5-dimethyl-6-(trifluoromethyl)-5,6-dihydro-4H-1,3-oxazin-2-amine (VIIIa-3), the product (4R,5R,6R)-5-fluoro-4-(2-fluoro-5-nitrophenyl)-4,5-dimethyl-6-(trifluoromethyl)-5,6-dihydro-4H-1,33-oxazin-2-amine (IX-3) was obtained as a yellow solid (74% yield) and was used in the following step without further purification. MS: m/z=354.4 [M+H]⁺.

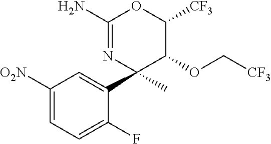 [SMILES: C[C@]1(c2cc([N+](=O)[O-])ccc2F)N=C(N)O[C@H](C(F)(F)F)[C@@H]1OCC(F)(F)F]

Intermediate IX-4: Starting from (4R,5R,6S)-4-(2-fluorophenyl)-4-methyl-5-(2,2,2-trifluoroethoxy)-6-(trifluoromethyl)-5,6-dihydro-4H-1,3-oxazin-2-amine (VIIIa-4), the product (4R,5R,6S)-4-(2-fluoro-5-nitrophenyl)-4-methyl-5-(2,2,2-trifluoroethoxy)-6-(trifluoromethyl)-5,6-dihydro-4H-1,3-oxazin-2-amine (IX-4) was obtained after chromatography on silica gel (eluent: heptane-EtOAc; gradient: 0-50% EtOAc) as a light yellow solid (80% yield). MS: m/z=420.4 [M+H]⁺.

General Procedure F: Synthesis of the Intermediate Anilines X

A solution of the nitro oxazine IX (3 mmol) in ethanol (31 ml) was, optionally, in presence of TEA (3 mmol), hydrogenated at atmospheric pressure using palladium (10% on carbon) (159 mg, 150 mol) as the catalyst. The progress of the reaction was followed by TLC. The reaction mixture was filtrated through a layer of Dicalite®, which was washed with ethanol (3×20 ml). The combined solutions of ethanol were evaporated at reduced pressure. The crude product was purified by chromatography on silica gel or silica-NH₂ phase using mixtures of heptane and EtOAc as the eluent or was directly used in the next step without further purification.

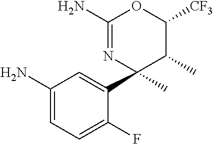 [SMILES: C[C@H]1[C@@H](C(F)(F)F)OC(N)=N[C@]1(C)c1cc(N)ccc1F]

Intermediate X-1: Starting from (4R,5R,6S)-5-fluoro-4-(2-fluoro-5-nitrophenyl)-4-methyl-6-(trifluoromethyl)-5,6-dihydro-4H-1,3-oxazin-2-amine (IX-1), the product (4R,5R,6S)-4-(5-amino-2-fluorophenyl)-5-fluoro-4-methyl-6-(trifluoromethyl)-5,6-dihydro-4H-1,3-oxazin-2-amine (X-1) was obtained as a white solid (99% yield) and was used in the next step without further purification. MS: m/z=310.5 [M+H]⁺.

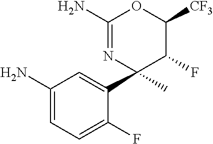 [SMILES: C[C@]1(c2cc(N)ccc2F)N=C(N)O[C@@H](C(F)(F)F)[C@@H]1F]

Intermediate X-2: Starting from (4R,5R,6R)-5-fluoro-4-(2-fluoro-5-nitrophenyl)-4-methyl-6-(trifluoromethyl)-5,6-dihydro-4H-1,3-oxazin-2-amine (IX-2), the product (4R,5R,6R)-4-(5-amino-2-fluorophenyl)-5-fluoro-4-methyl-6-(trifluoromethyl)-5,6-dihydro-4H-1,3-oxazin-2-amine (X-2) was obtained as a light yellow oil (87% yield) and was used in the next step without further purification. MS: m/z=310.4 [M+H]⁺.

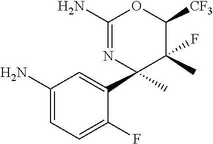 [SMILES: C[C@]1(F)[C@H](C(F)(F)F)OC(N)=N[C@]1(C)c1cc(N)ccc1F]

Intermediate X-3: Starting from (4R,5R,6R)-5-fluoro-4-(2-fluoro-5-nitrophenyl)-4,5-dimethyl-6-(trifluoromethyl)-5,6-dihydro-4H-1,3-oxazin-2-amine (IX-3), the product (4R,5R,6R)-4-(5-amino-2-fluorophenyl)-5-fluoro-4,5-dimethyl-6-(trifluoromethyl)-5,6-dihydro-4H-1,3-oxazin-2-amine (X-3) was obtained as a yellow foam (87% yield) and was used in the next step without further purification. MS: m/z=324.5 [M+H]⁺.

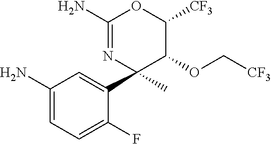 [SMILES: C[C@]1(c2cc(N)ccc2F)N=C(N)O[C@H](C(F)(F)F)[C@@H]1OCC(F)(F)F]

Intermediate X-4: Starting from (4R,5R,6S)-4-(2-fluoro-5-nitrophenyl)-4-methyl-5-(2,2,2-trifluoroethoxy)-6-(trifluoromethyl)-5,6-dihydro-4H-1,3-oxazin-2-amine (IX-4), the product (4R,5R,6S)-4-(5-amino-2-fluorophenyl)-4-methyl-5-(2,2,2-trifluoroethoxy)-6-(trifluoromethyl)-5,6-dihydro-4H-1,3-oxazin-2-amine (X-4) was obtained as a light yellow solid (91% yield) and was used in the next step without further purification. MS: m/z=390.5 [M+H]⁺.

General Procedure G: Synthesis of the Intermediate DMTr-Oxazines XII

A solution of the amino oxazines VIIIb (574 μmol) in dichloromethane (8 ml) was treated subsequently at 0° C. with N-ethyldiisopropylamine (195 μl, 1.15 mmol, 2 eq) and 4,4′-dimethoxytriphenylmethyl chloride (292 mg, 861 μmol, 1.5 eq). After 16 hours at 22° C. the reaction mixture was washed with H₂O, the organic layer was separated, dried over Na₂SO₄, and evaporated. The residue was purified by chromatography on silica gel (Telos Flash Silica) using mixtures of heptane and EtOAc as the eluent.

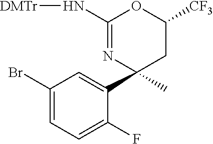

Intermediate XII-1: Starting from (4S,6S)-4-(5-bromo-2-fluorophenyl)-4-methyl-6-(trifluoromethyl)-5,6-dihydro-4H-1,3-oxazin-2-amine (VIIIb-1), the product (4S,6S)—N-(bis(4-methoxyphenyl)(phenyl)methyl)-4-(5-bromo-2-fluorophenyl)-4-methyl-6-(trifluoromethyl)-5,6-dihydro-4H-1,3-oxazin-2-amine (XII-1) was obtained after chromatography on silica gel (eluent: heptane-EtOAc; gradient: 0-14% EtOAc) as a white foam (51% yield). MS (FT): m/z=655.1 [M−H]⁻, 657.1 [M+2−H]⁻.

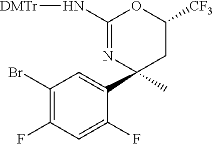

Intermediate XII-2: Starting from (4S,6S)-4-(5-bromo-2-fluorophenyl)-4-methyl-6-(trifluoromethyl)-5,6-dihydro-4H-1,3-oxazin-2-amine (VIIIb-2), the product (4S,6S)—N-(bis(4-methoxyphenyl)(phenyl)methyl)-4-(5-bromo-2,4-difluorophenyl)-4-methyl-6-(trifluoromethyl)-5,6-dihydro-4H-1,3-oxazin-2-amine (XII-2) was obtained after chromatography on silica gel (eluent: heptane-EtOAc; gradient: 0-14% EtOAc) as a white foam (45% yield). MS: m/z=675.1 [M+H]⁺, 677.1 [M+2+H]⁺.

General Procedure H: Synthesis of the Intermediate DMTr Bis-Aryl Derivatives XIII

A solution of the aryl-bromide XII (189 mol) and the boronic acid or the boronic acid ester (283 mol) in 1,2-dimethoxyethane (1.5 ml) was treated with a solution of Na₂CO₃ (2M, 0.3 ml) and triphenylphosphine (45.3 μmol). The solution was purged with argon, then palladium(II) acetate (27.2 μmol) was added and the mixture stirred at 100-110° C. for 16 h. For the workup, the reaction mixture was evaporated at reduced pressure and the residue purified by flash chromatography on silica gel or silica-NH₂ gel using mixtures of heptane and EtOAc as the eluent.

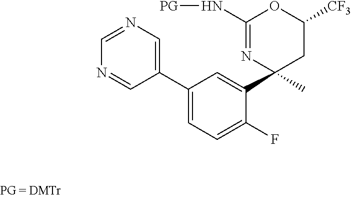

Intermediate XIII-1: Starting from (4S,6S)—N-(bis(4-methoxyphenyl)(phenyl)methyl)-4-(5-bromo-2-fluorophenyl)-4-methyl-6-(trifluoromethyl)-5,6-dihydro-4H-1,3-oxazin-2-amine (XII-1) and pyrimidine boronic acid, the product (4S,6S)—N-(bis(4-methoxyphenyl)(phenyl)methyl)-4-(2-fluoro-5-(pyrimidin-5-yl)phenyl)-4-methyl-6-(trifluoromethyl)-5,6-dihydro-4H-1,3-oxazin-2-amine (XIII-1) was obtained after chromatography on silica-NH₂ gel (eluent: heptane-EtOAc; gradient: 0-20% EtOAc) as a white solid (68% yield). MS: m/z=655.1 [M−H]⁻.

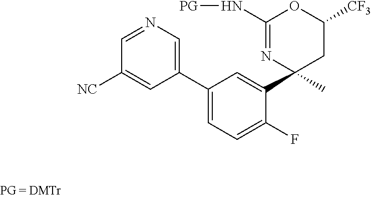

Intermediate XIII-2: Starting from (4S,6S)—N-(bis(4-methoxyphenyl)(phenyl)methyl)-4-(5-bromo-2-fluorophenyl)-4-methyl-6-(trifluoromethyl)-5,6-dihydro-4H-1,3-oxazin-2-amine (XII-1) and 5-(4,4,5,5-tetramethyl-1,3,2-dioxaborolan-2-yl)nicotinonitrile, the product 5-(3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)nicotinonitrile (XIII-2) was obtained after chromatography on silica-NH₂ gel (eluent: heptane-EtOAc; gradient: 0-20% EtOAc) as a white solid (81% yield). MS: m/z=681.5 [M+H]⁺.

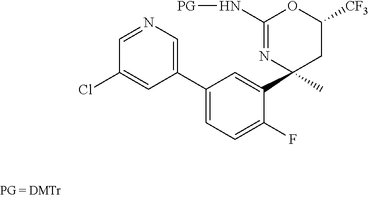

Intermediate XIII-3: Starting from (4S,6S)—N-(bis(4-methoxyphenyl)(phenyl)methyl)-4-(5-bromo-2-fluorophenyl)-4-methyl-6-(trifluoromethyl)-5,6-dihydro-4H-1,3-oxazin-2-amine (XII-1) and 5-chloropyridin-3-ylboronic acid, the product (4S,6S)—N-(bis(4-methoxyphenyl)(phenyl)methyl)-4-(5-(5-chloropyridin-3-yl)-2-fluorophenyl)-4-methyl-6-(trifluoromethyl)-5,6-dihydro-4H-1,3-oxazin-2-amine (XIII-3) was obtained after chromatography on silica-NH₂ gel (eluent: heptane-EtOAc; gradient: 0-10% EtOAc) as a white foam (70% yield). MS: m/z=690.4 [M+H]⁺.

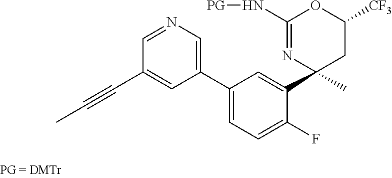

Intermediate XIII-4: Starting from (4S,6S)—N-(bis(4-methoxyphenyl)(phenyl)methyl)-4-(5-bromo-2-fluorophenyl)-4-methyl-6-(trifluoromethyl)-5,6-dihydro-4H-1,3-oxazin-2-amine (XII-1) and 5-(prop-1-ynyl)pyridin-3-ylboronic acid, the product (4S,6S)—N-(bis(4-methoxyphenyl)(phenyl)methyl)-4-(2-fluoro-5-(5-(prop-1-ynyl)pyridin-3-yl)phenyl)-4-methyl-6-(trifluoromethyl)-5,6-dihydro-4H-1,3-oxazin-2-amine (XIII-4) was obtained after chromatography on silica-NH₂ gel (eluent: heptane-EtOAc; gradient: 0-20% EtOAc) as a white foam (61% yield). MS: m/z=694.4 [M+H]⁺.

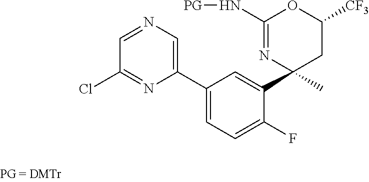

Intermediate XIII-5: Starting from (4S,6S)—N-(bis(4-methoxyphenyl)(phenyl)methyl)-4-(5-bromo-2-fluorophenyl)-4-methyl-6-(trifluoromethyl)-5,6-dihydro-4H-1,3-oxazin-2-amine (XII-1) and 2-chloro-6-(4,4,5,5-tetramethyl-1,3,2-dioxaborolan-2-yl)pyrazine, the product (4S,6S)—N-(bis(4-methoxyphenyl)(phenyl)methyl)-4-(5-(6-chloropyrazin-2-yl)-2-fluorophenyl)-4-methyl-6-(trifluoromethyl)-5,6-dihydro-4H-1,3-oxazin-2-amine (XIII-5) was obtained after chromatography on silica-NH₂ gel (eluent: heptane-EtOAc; gradient: 0-25% EtOAc) as a white foam (21% yield). MS: m/z=689.3 [M−H].

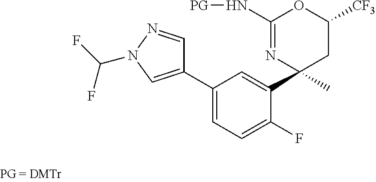

Intermediate XIII-6: Starting from (4S,6S)—N-(bis(4-methoxyphenyl)(phenyl)methyl)-4-(5-bromo-2-fluorophenyl)-4-methyl-6-(trifluoromethyl)-5,6-dihydro-4H-1,3-oxazin-2-amine (XII-1) and 1-(difluoromethyl)-4-(4,4,5,5-tetramethyl-1,3,2-dioxaborolan-2-yl)-1H-pyrazole, the product (4S,6S)—N-(bis(4-methoxyphenyl)(phenyl)methyl)-4-(5-(1-(difluoromethyl)-1H-pyrazol-4-yl)-2-fluorophenyl)-4-methyl-6-(trifluoromethyl)-5,6-dihydro-4H-1,3-oxazin-2-amine (XIII-6) was obtained after chromatography on silica-NH₂ gel (eluent: heptane-EtOAc; gradient: 0-14% EtOAc) as a white foam (50% yield). MS: m/z=695.3 [M+H]⁺.

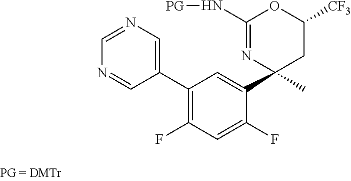

Intermediate XIII-7: Starting from (4S,6S)—N-(bis(4-methoxyphenyl)(phenyl)methyl)-4-(5-bromo-2,4-difluorophenyl)-4-methyl-6-(trifluoromethyl)-5,6-dihydro-4H-1,3-oxazin-2-amine (XII-2) and pyrimidine-5-boronic acid, the product (4S,6S)—N-(bis(4-methoxyphenyl)(phenyl)methyl)-4-(2,4-difluoro-5-(pyrimidin-5-yl)phenyl)-4-methyl-6-(trifluoromethyl)-5,6-dihydro-4H-1,3-oxazin-2-amine (XIII-7) was obtained after chromatography on silica-NH₂ gel (eluent: heptane-EtOAc; gradient: 0-20% EtOAc) as a white semisolid (64% yield).

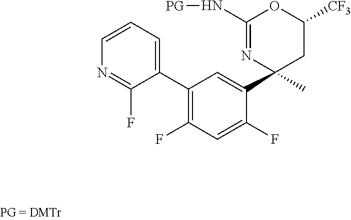

Intermediate XIII-8: Starting from (4S,6S)—N-(bis(4-methoxyphenyl)(phenyl)methyl)-4-(5-bromo-2,4-difluorophenyl)-4-methyl-6-(trifluoromethyl)-5,6-dihydro-4H-1,3-oxazin-2-amine (XII-2) and 2-fluoropyridin-3-ylboronic acid the product (4S,6S)—N-(bis(4-methoxyphenyl)(phenyl)methyl)-4-(2,4-difluoro-5-(2-fluoropyridin-3-yl)phenyl)-4-methyl-6-(trifluoromethyl)-5,6-dihydro-4H-1,3-oxazin-2-amine (XIII-8) was obtained after chromatography on silica-NH₂ gel (eluent: heptane-EtOAc; gradient: 0-20% EtOAc) as a colorless oil (29% yield). MS: m/z=690.3 [M−H]⁻.

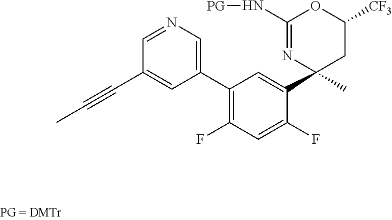

Intermediate XIII-9: Starting from (4S,6S)—N-(bis(4-methoxyphenyl)(phenyl)methyl)-4-(5-bromo-2,4-difluorophenyl)-4-methyl-6-(trifluoromethyl)-5,6-dihydro-4H-1,3-oxazin-2-amine (XII-2) and 5-(prop-1-ynyl)pyridin-3-ylboronic acid, the product (4S,6S)—N-(bis(4-methoxyphenyl)(phenyl)methyl)-4-(2,4-difluoro-5-(5-(prop-1-ynyl)pyridin-3-yl)phenyl)-4-methyl-6-(trifluoromethyl)-5,6-dihydro-4H-1,3-oxazin-2-amine (XIII-9) was obtained after chromatography on silica-NH₂ gel (eluent: heptane-EtOAc; gradient: 0-5% EtOAc) as a white foam (66% yield). MS: m/z=712.4 [M+H]⁺.

Synthesis of the Intermediate Iodo Derivative XIV

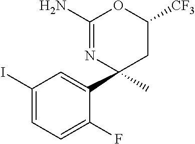 [SMILES: C[C@@]1(c2cc(I)ccc2F)C[C@@H](C(F)(F)F)OC(N)=N1]

A solution of the 2:3-mixture of (4S,6S)-4-(5-bromo-2-fluorophenyl)-4-methyl-6-(trifluoromethyl)-5,6-dihydro-4H-1,3-oxazin-2-amine (VIIIb-1) and (2S,4S)-4-amino-4-(5-bromo-2-fluorophenyl)-1,1,1-trifluoropentan-2-ol (VIIa-5) (1.67 g) in dioxane (40 ml) was treated consecutively with trans-N,N-dimethyl-1,2-cyclohexanediamine (152 mg, 168 μl, 1.03 mmol), CuI (101 mg, 0.518 mmol), and NaI (1.57 g, 10.4 mmol). The reaction mixture was stirred overnight at 110° C. The reaction mixture was evaporated at reduced pressure and the residue purified by flash chromatography on silica gel (eluent: heptane-EtOAc; gradient: 0-33% EtOAc). The product (4S,6S)-4-(2-fluoro-5-iodophenyl)-4-methyl-6-(trifluoromethyl)-5,6-dihydro-4H-1,3-oxazin-2-amine (XIV) was obtained as a 1:2-mixture together with (2S,4S)-4-amino-, 1,1-trifluoro-4-(2-fluoro-5-iodophenyl)pentan-2-ol as a light brown solid (1.608 g). The mixture was used as such in the following step.

In a small scale separation by flash chromatography on silica-NH₂ gel (eluent: heptane-EtOAc; gradient: 0-20% EtOAc) the 2 products could be separated yielding XIV as a light brown foam (36% yield) (MS: m/z=403.4 [M+H]⁺) and (2S,4S)-4-amino-1,1,1-trifluoro-4-(2-fluoro-5-iodophenyl)pentan-2-ol as a light brown foam (43% yield) (MS: m/z=378.4 [M+H]+).

Cyclization of the (2S,4S)-4-amino-1,1,1-trifluoro-4-(2-fluoro-5-iodophenyl)pentan-2-ol (150 mg, 0.398 mmol) following General Procedure D yielded the (4S,6S)-4-(2-fluoro-5-iodophenyl)-4-methyl-6-(trifluoromethyl)-5,6-dihydro-4H-1,3-oxazin-2-amine (XIV) as a light brown viscous oil.

Synthesis of the Intermediate DMTr-Iodo Derivative XV

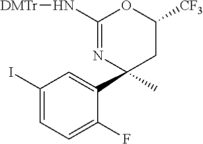

Following General Procedure G the reaction of the mixture of (4S,6S)-4-(2-fluoro-5-iodophenyl)-4-methyl-6-(trifluoromethyl)-5,6-dihydro-4H-1,3-oxazin-2-amine (XIV) and (2S,4S)-4-amino-1,1,1-trifluoro-4-(2-fluoro-5-iodophenyl)pentan-2-ol with 4,4′-dimethoxytriphenylmethyl chloride yielded the (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) as a white foam (60% yield) and unreacted (2S,4S)-4-amino-1,1,1-trifluoro-4-(2-fluoro-5-iodophenyl)pentan-2-ol as an off-white solid (20% yield).

General Procedure I: Synthesis of the Intermediate DMTr Acetylene Derivatives XVI

A dry flask was charged under argon with DMTr-iodo derivative XV (65.3 μmol), the (trimethylsilyl)ethynyl-derivative (91.4 μmol), and DMF (1 ml) (solution 1). Another dry flask was charged under argon with DMF (1 ml), bis(triphenylphosphin)palladium(II) dichloride (15% Pd; 3.27 mg, 4.57 μmol), triphenylphosphine (1.37 mg, 5.22 μmol), CuI (497 μg, 2.61 μmol), TEA (33.0 mg, 45.4 μl, 326 μmol), and tetrabutylammonium iodide (24.6 mg, 65.3 μmol). The mixture was warmed to 40° C. and solution 1 was added dropwise. After complete addition the temperature was raised to 60° C. and a solution of tetrabutylammonium fluoride (1 M in THF, dried over molecular sieve; 91.4 μl, 91.4 μmol) was added. The reaction mixture was stirred at 60° C. for 16 h, thereafter evaporated at reduced pressure. The residue was purified by flash chromatography on silica gel or silica-NH₂ gel using mixtures of heptane and EtOAc as the eluent.

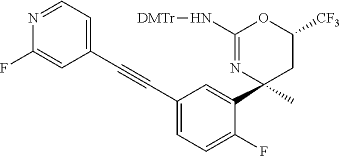

Intermediate XVI-1: Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 2-chloro-4-((trimethylsilyl)ethynyl)pyridine (e.g. Röhrig et al.⁴⁵), the product (4S,6S)—N-(bis(4-methoxyphenyl)(phenyl)methyl)-4-(5-((2-chloropyridin-4-yl)ethynyl)-2-fluorophenyl)-4-methyl-6-(trifluoromethyl)-5,6-dihydro-4H-1,3-oxazin-2-amine (XVI-1) was obtained after chromatography on silica gel (eluent: heptane-EtOAc; gradient: 0-20% EtOAc) as a light yellow foam (32% yield). MS: m/z=714.2 [M+H]⁺.

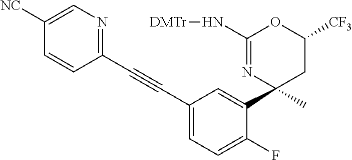

Intermediate XVI-2: Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 6-((trimethylsilyl)ethynyl)nicotinonitrile (Farahat et al.⁴⁶), the product 6-((3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)ethynyl)nicotinonitrile (XVI-2) was obtained after chromatography on silica gel (eluent: heptane-EtOAc; gradient: 0-33% EtOAc) as a light yellow foam (45% yield).

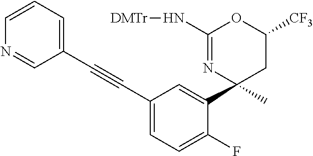

Intermediate XVI-3: Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 3-((trimethylsilyl)ethynyl)pyridine, the product (4S,6S)—N-(bis(4-methoxyphenyl)(phenyl)methyl)-4-(2-fluoro-5-(pyridin-3-ylethynyl)phenyl)-4-methyl-6-(trifluoromethyl)-5,6-dihydro-4H-1,3-oxazin-2-amine (XVI-3) was obtained after chromatography on silica gel (eluent: heptane-EtOAc; gradient: 0-50% EtOAc) as a light yellow foam (76% yield). MS: m/z=680.2 [M+H]⁺.

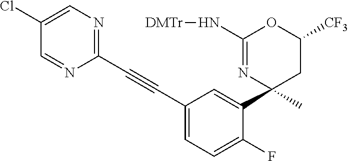

Intermediate XVI-4: Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 5-chloro-2-((trimethylsilyl)ethynyl)pyrimidine, the product (4S,6S)—N-(bis(4-methoxyphenyl)(phenyl)methyl)-4-(5-((5-chloropyrimidin-2-yl)ethynyl)-2-fluorophenyl)-4-methyl-6-(trifluoromethyl)-5,6-dihydro-4H-1,3-oxazin-2-amine (XVI-4) was obtained after chromatography on silica gel (eluent: heptane-EtOAc; gradient: 0-25% EtOAc) as a yellow foam (35% yield). MS: m/z=715.3 [M+H]⁺.

The 5-chloro-2-((trimethylsilyl)ethynyl)pyrimidine was obtained as follows:

A solution of 2-bromo-5-chloropyrimidine (201 mg, 1.04 mmol) in THF (3 ml) was treated consecutively with bis(triphenylphosphin)palladium(II) dichloride (52.1 mg, 72.7 mol), ethynyltrimethylsilane (129 mg, 184 μl, 1.29 mmol), and TEA (315 mg, 432 μl, 3.12 mmol). The mixture was degassed and under an atmosphere of argon CuI (5.94 mg, 31.2 mol) was added. After stirring at r.t. during 16 h the reaction mixture was evaporated at reduced pressure. The residue was purified by chromatography on silica gel (eluent: heptane-EtOAc; gradient: 0-10% EtOAc) and was obtained as a dark brown solid (31% yield). MS: m/z=210 [M]⁺.

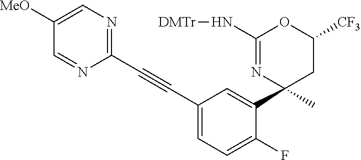

Intermediate XVI-5: Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 5-methoxy-2-((trimethylsilyl)ethynyl)pyrimidine, the product (4S,6S)—N-(bis(4-methoxyphenyl)(phenyl)methyl)-4-(2-fluoro-5-((5-methoxypyrimidin-2-yl)ethynyl)phenyl)-4-methyl-6-(trifluoromethyl)-5,6-dihydro-4H-1,3-oxazin-2-amine (XVI-5) was obtained after chromatography on silica gel (eluent: heptane-EtOAc; gradient: 0-50% EtOAc) as a light yellow foam (72% yield). MS: m/z=711.2 [M+H]⁺.

In close analogy to the procedure described for 5-chloro-2-((trimethylsilyl)ethynyl)pyrimidine, the 5-methoxy-2-((trimethylsilyl)ethynyl)pyrimidine was obtained starting from 2-bromo-5-methoxypyrimidine as a light brown oil (34% yield) after chromatography on silica gel (eluent: heptane-EtOAc; gradient: 0-10% EtOAc). MS: m/z=207.5 [M+H]⁺.

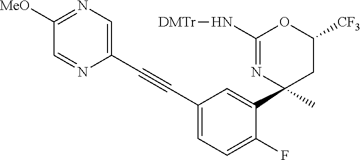

Intermediate XVI-6: Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 2-methoxy-5-((trimethylsilyl)ethynyl)pyrazine, the product (4S,6S)—N-(bis(4-methoxyphenyl)(phenyl)methyl)-4-(2-fluoro-5-((5-methoxypyrazin-2-yl)ethynyl)phenyl)-4-methyl-6-(trifluoromethyl)-5,6-dihydro-4H-1,3-oxazin-2-amine (XVI-6) was obtained after chromatography on silica gel (eluent: heptane-EtOAc; gradient: 0-20% EtOAc) as a light yellow foam (48% yield). MS: m/z=711.2 [M+H]⁺.

In close analogy to the procedure described for 5-chloro-2-((trimethylsilyl)ethynyl)pyrimidine, the 2-methoxy-5-((trimethylsilyl)ethynyl)pyrazine was obtained starting from 2-bromo-5-methoxypyrazine as a light yellow liquid (73% yield) after chromatography on silica gel (eluent: heptane-EtOAc; gradient: 0-10% EtOAc). MS: m/z=207.5 [M+H]⁺.

Synthesis of the Intermediate Ester XVIII-1

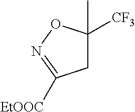

3,3,3-Trifluoro-2-methylprop-1-ene (14.5 g, 132 mmol) was condensed under an atmosphere of nitrogen at −78° C. via a gas inlet tube into a dispersion of NaHCO₃ (24.9 g, 297 mmol) in EtOAc (120 ml) during 95 min. A solution of (Z)-ethyl 2-chloro-2-(hydroxyimino)acetate (10 g, 66.0 mmol) in EtOAc (25 ml) was added dropwise during 15 min while the internal temperature was kept between −75° C. and −70° C. After complete addition the cooling bath was removed and the mixture was allowed to warm to rt and stirred for 4 d. For the workup, the reaction mixture was diluted with EtOAc (200 ml) and extracted with H₂O (100 ml). The aqueous layer was separated and washed with EtOAc (150 ml). The combined organic layers were washed with brine (100 ml), dried over Na₂SO₄, and concentrated at reduced pressure (40° C./20 mbar). Following NMR the residue consisted of a 1:1-mixture (9.75 g) of the product ethyl 5-methyl-5-(trifluoromethyl)-4,5-dihydroisoxazole-3-carboxylate and the starting material (Z)-ethyl 2-chloro-2-(hydroxyimino)acetate. The crude product was engaged in the next step without further purification.

Synthesis of the Intermediate Alcohol XIX-1

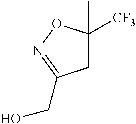

A solution of ethyl 5-methyl-5-(trifluoromethyl)-4,5-dihydroisoxazole-3-carboxylate (2.2 g, 5.86 mmol) in ethanol (12 ml) was treated at 15-25° C. in portions with sodium borohydride (485 mg, 12.31 mmol). The reaction was followed by TLC. After 4 h the reaction mixture was quenched at 0° C. with a saturated solution of NH₄Cl (10 ml) and stirred for 10 min. Thereafter the mixture was diluted with H₂O (10 ml) and EtOAc (50 ml). The aqueous layer was separated and extracted with EtOAc (2×50 ml). The combined organic layers were washed with brine (10 ml), dried over Na₂SO₄, and concentrated at reduced pressure. The crude product was purified by chromatography on silica gel (eluent: heptane-EtOAc; gradient: 0-60% EtOAc) and the (5-methyl-5-(trifluoromethyl)-4,5-dihydroisoxazol-3-yl)methanol was obtained as a colorless amorphous material (1.0 g).

Synthesis of the Intermediate Fluoro Derivative XX-1

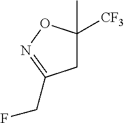

Under an atmosphere of nitrogen a suspension of (5-methyl-5-(trifluoromethyl)-4,5-dihydroisoxazol-3-yl)methanol (1.0 g, 5.46 mmol, Eq) in DCM (20 ml) was treated dropwise at −75° C. with bis(2-methoxyethyl)aminosulfur trifluoride (Deoxofluor®). The yellow reaction mixture was stirred at −75° C. for 30 min, then left to warm to rt and stirred for 16 h. For the workup the reaction mixture was treated dropwise with an ice-cold, saturated solution of NaHCO₃ while the temperature was kept at 5-10° C. After complete addition the mixture was left to warm to rt and stirring was continued for 50 min. Thereafter the mixture was diluted with H₂O (20 ml) and DCM (20 ml). The aqueous layer separated and extracted twice with DCM (50 ml). The combined organic layers were washed with 1 N HCl (20 ml), dried over Na₂SO₄, and concentrated at reduced pressure (40° C./100 mbar). The crude product 3-(fluoromethyl)-5-methyl-5-(trifluoromethyl)-4,5-dihydroisoxazole (yellow oil, 98% yield) was pure enough to be engaged in the next step without further purification.

Synthesis of the Intermediate Isoxazolidine XXII-1

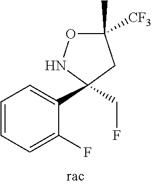 [SMILES: C[C@@]1(C(F)(F)F)C[C@@](CF)(c2ccccc2F)NO1]

A solution of 1-bromo-2-fluorobenzene (1.7 g, 1.06 ml, 9.72 mmol, eq: 1.80) in toluene (10 ml) and THF (3.5 ml) was cooled to −75° C., and n-BuLi (1.6 M in hexane; 5.91 ml, 9.45 mmol, eq: 1.75) was added dropwise via syringe within 12 min keeping the temperature between −75° C. and −72° C. After complete addition the reaction mixture was stirred at −78° C. for 20 min. A freshly prepared solution of 3-(fluoromethyl)-5-methyl-5-(trifluoromethyl)-4,5-dihydroisoxazole (XX-1) (1 g, 5.4 mmol, eq: 1.00) and BF₃.Et₂O (1.61 g, 1.44 ml, 11.3 mmol, eq: 2.10) in toluene (3 ml) and THF (3 ml) was added dropwise via syringe pump and using an insulated cannula to the aforementioned solution keeping the temperature between −75° C. and −73° C. After complete addition the reaction mixture was stirred at −76° C. for 45 min, then it was quenched with ethanol (2.49 g, 3.15 ml, 54.0 mmol, eq: 10.00) under cooling with ice. Thereafter the ice bath was removed and the mixture was allowed to warm to 10° C. before it was poured slowly into a saturated solution of NaHCO₃ (100 ml) and stirred at rt for 1 h. The mixture was diluted with toluene (20 ml) and H₂O (20 ml). The aqueous phase was separated and extracted twice with EtOAc (2×100 ml). The combined organic layers were washed with brine (50 ml), dried over Na₂SO₄, and concentrated at reduced pressure. After chromatography on silica gel (eluent: heptane-EtOAc; gradient: 0-35% EtOAc) the product (3S,5S)- and (3R,5R)-3-(fluoromethyl)-3-(2-fluorophenyl)-5-methyl-5-(trifluoromethyl)-isoxazolidine (XXII-1) was not obtained in pure form (511 mg, light yellow oil) and was engaged in the next step without further purification. The relative configuration was tentatively assigned retrospectively based on NMR-analysis of intermediate VIIIc-1.

Synthesis of the Intermediate Aminoalcohol VIIb-1

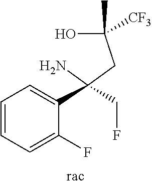 [SMILES: C[C@](O)(C[C@@](N)(CF)c1ccccc1F)C(F)(F)F]

A solution of (3S,5S)- and (3R,5R)-3-(fluoromethyl)-3-(2-fluorophenyl)-5-methyl-5-(trifluoromethyl)isoxazolidine (XXII-1) (200 mg, 711 mol) in ethanol (4 ml) was treated with ammonium formate (366 mg, 5.69 mmol) and palladium (10% on carbon; 49.2 mg). The progress of the reaction was followed by TLC. After 16 h at rt the incomplete reaction was concentrated at reduced pressure, the residue was diluted with EtOAc (30 ml) ethylacetate and extracted with a saturated solution of NaHCO₃ (10 ml). The aqueous layer was washed twice with EtOAc (2×15 ml), the combined organic layers washed with brine (10 ml), dried over Na₂SO₄, and concentrated at reduced pressure. The residue containing the product (2S,4S)- and (2R,4R)-4-amino-1,1,1,5-tetrafluoro-4-(2-fluorophenyl)-2-methylpentan-2-ol (VIIb-1) (light yellow oil, 75 mg) was engaged in the next step without further purification.

Synthesis of the Intermediate Oxazine VIIIc-1

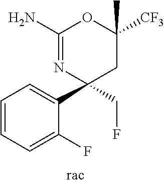 [SMILES: C[C@@]1(C(F)(F)F)C[C@@](CF)(c2ccccc2F)N=C(N)O1]

Starting from (2S,4S)- and (2R,4R)-4-amino-1,1,1,5-tetrafluoro-4-(2-fluorophenyl)-2-methylpentan-2-ol (VIIb-1) and following General Procedure D, the reaction with cyanogen bromide yielded the product (4S,6S)- and (4R,6R)-4-(fluoromethyl)-4-(2-fluorophenyl)-6-methyl-6-(trifluoromethyl)-5,6-dihydro-4H-1,3-oxazin-2-amine (VIIIc-1) after chromatography on silica gel (eluent: heptane-EtOAc; gradient: 0-50% EtOAc) as a yellow oil (13% yield). MS: m/z=309.5 [M+H]⁺. The relative configuration was tentatively assigned based on NMR-analysis of intermediate VIIIc-1.

Synthesis of the Intermediate Nitro-Oxazine IXa-1

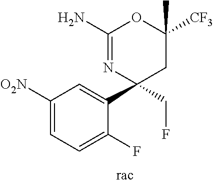 [SMILES: C[C@@]1(C(F)(F)F)C[C@@](CF)(c2cc([N+](=O)[O-])ccc2F)N=C(N)O1]

Starting from (4S,6S)- and (4R,6R)-4-(fluoromethyl)-4-(2-fluorophenyl)-6-methyl-6-(trifluoromethyl)-5,6-dihydro-4H-1,3-oxazin-2-amine (VIIIc-1) and following General Procedure E, the nitration yielded the product (4S,6S)- and (4R,6R)-4-(2-fluoro-5-nitrophenyl)-4-(fluoromethyl)-6-methyl-6-(trifluoromethyl)-5,6-dihydro-4H-1,3-oxazin-2-amine (IXa-1) after chromatography on silica gel (eluent: heptane-EtOAc; gradient: 0-40% EtOAc) as a light yellow solid (41% yield). MS: m/z=354.4 [M+H]⁺.

Synthesis of the Intermediate Aniline Xa-1

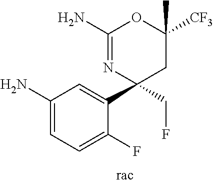 [SMILES: C[C@@]1(C(F)(F)F)C[C@@](CF)(c2cc(N)ccc2F)N=C(N)O1]

Starting from (4S,6S)- and (4R,6R)-4-(2-fluoro-5-nitrophenyl)-4-(fluoromethyl)-6-methyl-6-(trifluoromethyl)-5,6-dihydro-4H-1,3-oxazin-2-amine (IXa-1), and following General Procedure F, the reduction yielded the product (4S,6S)- and (4R,6R)-4-(5-amino-2-fluorophenyl)-4-(fluoromethyl)-6-methyl-6-(trifluoromethyl)-5,6-dihydro-4H-1,3-oxazin-2-amine (Xa-1) as an off-white solid (95% yield). The crude product was engaged in the next step without further purification.

The following compounds were prepared following the general procedures and, depending on the reaction and purification conditions, they were isolated in either the free base form or as a salt.

General Procedure J: Synthesis of Compounds of Formula I-1 and I-4

A solution of the carboxylic acid (0.23 mmol) in methanol (5 ml) was cooled to 0° C. 4-(4,6-Dimethoxy[1.3.5]triazin-2-yl)-4-methylmorpholinium chloride hydrate* (DMTMM) (80 mg, 0.27 mmol) was added and the solution was stirred at 0° C. for 30 minutes. Thereafter, a solution of the intermediate aniline (0.21 mmol) in methanol (5 ml) was added dropwise at 0° C. via syringe. The reaction mixture was stirred at 23° C. for 18-60 hours. For the workup, the reaction mixture was concentrated at reduced pressure, then poured into a solution of Na₂CO₃ (1M) followed by the extraction with DCM. The organic layer was separated, washed with brine and dried over Na₂SO₄. Removal of the solvent left the crude product which was purified by chromatography on silica gel using a mixture of DCM-MeOH or heptane-EtOAc or by preparative HPLC to give the pure amides.

* The chloride salt can be replaced by the corresponding tetrafluoroborate.

### Example 1

### N-(3-((4R,5R,6R)-2-Amino-5-fluoro-4-methyl-6-(trifluoromethyl)-5,6-dihydro-4H-1,3-oxazin-4-yl)-4-fluorophenyl)-5-cyanopicolinamide

Starting with (4R,5R,6R)-4-(5-amino-2-fluorophenyl)-5-fluoro-4-methyl-6-(trifluoromethyl)-5,6-dihydro-4H-1,3-oxazin-2-amine (X-2), the reaction with 5-cyano-2-pyridinecarboxylic acid and DMTMM as the condensating agent yielded the title compound after chromatography on silica gel (eluent: heptane-EtOAc; gradient: 0-80% EtOAc) as a white solid (67% yield). H NMR (300 MHz, CDCl₃) δ ppm 9.86 (br s, 1H), 8.88 (d, J=1.0 Hz, 1H), 8.43 (d, J=8.3 Hz, 1H), 8.20 (dd, J=8.1, 2.0 Hz, 1H), 7.88 (dt, J=8.6, 3.4 Hz, 1H), 7.81 (dd, J=6.9, 2.6 Hz, 1H), 7.11 (dd, J=11.5, 8.9 Hz, 1H), 4.97 (dd, J=49.4, 7.7 Hz, 1H), 4.56-4.72 (m, 1H), 3.79-4.80 (br s, 2H), 1.72 (br s, 3H). MS: m/z=440.5 (M+H)⁺.

### Example 2

### N-(3-((4R,5R,6S)-2-Amino-5-fluoro-4-methyl-6-(trifluoromethyl)-5,6-dihydro-4H-1,3-oxazin-4-yl)-4-fluorophenyl)-5-cyanopicolinamide

Starting with (4R,5R,6S)-4-(5-amino-2-fluorophenyl)-5-fluoro-4-methyl-6-(trifluoromethyl)-5,6-dihydro-4H-1,3-oxazin-2-amine (X-1), the reaction with 5-cyano-2-pyridinecarboxylic acid and DMTMM as the condensating agent yielded the title compound after chromatography on silica gel (eluent: heptane-EtOAc; gradient: 0-75% EtOAc) as a light yellow foam (83% yield). ¹H NMR (300 MHz, DMSO-d₆) δ ppm 10.92 (s, 1H), 9.20 (dd, J=1.9, 0.9 Hz, 1H), 8.58 (dd, J=8.3, 2.0 Hz, 1H), 8.28 (dd, J=8.3, 0.8 Hz, 1H), 7.86-7.95 (m, 2H), 7.23 (dd, J=11.8, 8.8 Hz, 1H), 6.16 (s, 2H), 5.28 (d, J=49.7 Hz, 1H), 4.53 (ddd, J=29.9, 12.7, 6.7 Hz, 1H), 1.53 (br s, 3H). MS: m/z=440.5 (M+H)⁺.

### Example 3

### N-(3-((4R,5R,6R)-2-Amino-5-fluoro-4-methyl-6-(trifluoromethyl)-5,6-dihydro-4H-1,3-oxazin-4-yl)-4-fluorophenyl)-5-chloropicolinamide

Starting with (4R,5R,6R)-4-(5-amino-2-fluorophenyl)-5-fluoro-4-methyl-6-(trifluoromethyl)-5,6-dihydro-4H-1,3-oxazin-2-amine (X-2), the reaction with 5-chloro-2-pyridinecarboxylic acid and DMTMM as the condensating agent yielded the title compound after chromatography on silica gel (eluent: heptane-EtOAc; gradient: 0-80% EtOAc) as a white solid (52% yield). H NMR (300 MHz, CDCl₃) δ ppm 9.83 (br s, 1H), 8.56 (d, J=1.8 Hz, 1H), 8.24 (d, J=8.5 Hz, 1H), 7.84-7.92 (m, 2H), 7.79 (dd, J=6.9, 2.6 Hz, 1H), 7.09 (dd, J=11.6, 8.8 Hz, 1H), 4.98 (dd, J=49.4, 7.7 Hz, 1H), 4.64 (dquind, J=12.6, 6.5, 6.5, 6.5, 6.5, 1.0 Hz, 1H), 1.72 (br s, 3H). MS: m/z=449.5 (M+H)⁺.

### Example 4

### N-(3-((4R,5R,6S)-2-Amino-5-fluoro-4-methyl-6-(trifluoromethyl)-5,6-dihydro-4H-1,3-oxazin-4-yl)-4-fluorophenyl)-5-chloropicolinamide

Starting with (4R,5R,6S)-4-(5-amino-2-fluorophenyl)-5-fluoro-4-methyl-6-(trifluoromethyl)-5,6-dihydro-4H-1,3-oxazin-2-amine (X-1), the reaction with 5-chloro-2-pyridinecarboxylic acid and DMTMM as the condensating agent yielded the title compound after chromatography on silica gel (eluent: heptane-EtOAc; gradient: 0-60% EtOAc) as a white foam (81% yield). ¹H NMR (300 MHz, DMSO-d₆) δ ppm 10.74 (s, 1H), 8.79 (dd, J=2.2, 0.8 Hz, 1H), 8.18-8.22 (m, 1H), 8.13-8.17 (m, 1H), 7.85-7.92 (m, 2H), 7.22 (dd, J=11.8, 9.4 Hz, 1H), 6.15 (s, 2H), 5.28 (d, J=50.1 Hz, 1H), 4.53 (dq, J=30.3, 6.5 Hz, 1H), 1.53 (t, J=2.0 Hz, 3H). MS: m/z=449.3 (M+H)⁺.

### Example 5

### N-(3-((4R,5R,6S)-2-Amino-5-fluoro-4-methyl-6-(trifluoromethyl)-5,6-dihydro-4H-1,3-oxazin-4-yl)-4-fluorophenyl)-5-methoxypyrazine-2-carboxamide

Starting with (4R,5R,6S)-4-(5-amino-2-fluorophenyl)-5-fluoro-4-methyl-6-(trifluoromethyl)-5,6-dihydro-4H-1,3-oxazin-2-amine (X-1), the reaction with 5-methoxypyrazine-2-carboxylic acid and DMTMM as the condensating agent yielded the title compound after chromatography on silica gel (eluent: heptane-EtOAc; gradient: 0-75% EtOAc) as a white solid (78% yield). ¹H NMR (300 MHz, DMSO-d₆) δ ppm 10.58 (s, 1H), 8.89 (d, J=1.4 Hz, 1H), 8.41 (d, J=1.2 Hz, 1H), 7.82-7.92 (m, 2H), 7.21 (dd, J=11.8, 8.8 Hz, 1H), 6.14 (s, 2H), 5.28 (d, J=49.9 Hz, 1H), 4.43-4.61 (m, 1H), 4.02 (s, 3H), 1.53 (t, J=1.6 Hz, 3H). MS: m/z=446.4 (M+H)+.

### Example 6

### N-(3-((4R,5R,6S)-2-Amino-5-fluoro-4-methyl-6-(trifluoromethyl)-5,6-dihydro-4H-1,3-oxazin-4-yl)-4-fluorophenyl)-5-(difluoromethyl)pyrazine-2-carboxamide

Starting with (4R,5R,6S)-4-(5-amino-2-fluorophenyl)-5-fluoro-4-methyl-6-(trifluoromethyl)-5,6-dihydro-4H-1,3-oxazin-2-amine (X-1), the reaction with 5-(difluoromethyl)pyrazine-2-carboxylic acid [J. M. Ellard et al.⁴⁷] and DMTMM as the condensating agent yielded the title compound after chromatography on silica gel (eluent: heptane-EtOAc; gradient: 0-70% EtOAc) as a white foam (76% yield). H NMR (300 MHz, DMSO-d₆) δ ppm 10.99 (s, 1H), 9.39 (d, J=1.2 Hz, 1H), 9.09 (d, J=0.8 Hz, 1H), 7.86-7.97 (m, 2H), 7.25 (dd, J=11.8, 8.9 Hz, 1H), 7.25 (t, 54.1 Hz, 1H), 6.16 (s, 2H), 5.29 (dd, J=49.2, 0.6 Hz, 1H), 4.43-4.62 (m, 1H), 1.53 (t, J=1.8 Hz, 3H). MS: m/z=466.4 (M+H)⁺.

### Example 7

### N-(3-((4R,5R,6S)-2-Amino-5-fluoro-4-methyl-6-(trifluoromethyl)-5,6-dihydro-4H-1,3-oxazin-4-yl)-4-fluorophenyl)-5-(but-2-ynyloxy)pyrazine-2-carboxamide

Starting with (4R,5R,6S)-4-(5-amino-2-fluorophenyl)-5-fluoro-4-methyl-6-(trifluoromethyl)-5,6-dihydro-4H-1,3-oxazin-2-amine (X-1), the reaction with 5-(but-2-ynyloxy)pyrazine-2-carboxylic acid [G. Csjernyik et al.⁴⁸] and DMTMM as the condensating agent yielded the title compound after chromatography on silica gel (eluent: heptane-EtOAc; gradient: 0-70% EtOAc) as a white solid (66% yield). ¹H NMR (300 MHz, DMSO-d₆) δ ppm 10.60 (s, 1H), 8.89 (d, J=1.2 Hz, 1H), 8.45 (d, J=1.4 Hz, 1H), 7.82-7.92 (m, 2H), 7.21 (dd, J=11.8, 8.8 Hz, 1H), 6.15 (s, 2H), 5.28 (d, J=49.7 Hz, 1H), 5.09 (q, J=2.4 Hz, 2H), 4.43-4.61 (m, 1H), 1.85 (t, J=2.4 Hz, 3H), 1.52 (br s, 3H). MS: m/z=484.4 (M+H)⁺.

### Example 8

### N-(3-((4R,5R,6S)-2-Amino-4-methyl-5-(2,2,2-trifluoroethoxy)-6-(trifluoromethyl)-5,6-dihydro-4H-1,3-oxazin-4-yl)-4-fluorophenyl)-5-cyanopicolinamide

Starting with (4R,5R,6S)-4-(5-amino-2-fluorophenyl)-4-methyl-5-(2,2,2-trifluoroethoxy)-6-(trifluoromethyl)-5,6-dihydro-4H-1,3-oxazin-2-amine (X-4), the reaction with 5-cyano-2-pyridinecarboxylic acid and DMTMM as the condensating agent yielded the title compound after chromatography on silica gel (eluent: heptane-EtOAc; gradient: 0-66% EtOAc) as a white solid (68% yield). H NMR (300 MHz, CDCl₃) δ ppm 9.87 (s, 1H), 8.90 (dd, J=2.0, 0.8 Hz, 1H), 8.43 (dd, J=8.1, 0.8 Hz, 1H), 8.21 (dd, J=8.1, 2.0 Hz, 1H), 8.00-8.06 (m, 1H), 7.51 (dd, J=6.9, 2.8 Hz, 1H), 7.13 (dd, J=11.5, 8.9 Hz, 1H), 4.44 (s, 1H), 4.33 (br s, approx. 2H), 4.02-4.17 (m, 3H), 1.67 (d, J=1.0 Hz, 3H). MS: m/z=520.5 (M+H)⁺.

### Example 9

### N-(3-((4R,5R,6R)-2-Amino-5-fluoro-4,5-dimethyl-6-(trifluoromethyl)-5,6-dihydro-4H-1,3-oxazin-4-yl)-4-fluorophenyl)-5-cyanopicolinamide

Starting with (4R,5R,6R)-4-(5-amino-2-fluorophenyl)-5-fluoro-4,5-dimethyl-6-(trifluoromethyl)-5,6-dihydro-4H-1,3-oxazin-2-amine (X-3), the reaction with 5-cyano-2-pyridinecarboxylic acid and DMTMM as the condensating agent yielded the title compound after chromatography on silica gel (eluent: heptane-EtOAc; gradient: 0-50% EtOAc) as a light yellow solid (43% yield). ¹H NMR (300 MHz, CDCl₃) δ ppm 9.86 (br s, 1H), 8.88 (d, J=1.2 Hz, 1H), 8.43 (d, J=8.1 Hz, 1H), 8.20 (dd, J=8.1, 2.0 Hz, 1H), 7.88-8.00 (m, 2H), 7.12 (dd, J=11.4, 8.8 Hz, 1H), 4.79-4.92 (m, 1H), 4.32 (br s, 2H approx.), 1.80 (t, J=1.8 Hz, 3H), 1.16 (d, J=22.4 Hz, 3H). MS: m/z=454.5 (M+H)⁺.

### Example 10

### N-(3-((4S,6S)- and (4R,6R)-2-Amino-4-(fluoromethyl)-6-methyl-6-(trifluoro-methyl)-5,6-dihydro-4H-1,3-oxazin-4-yl)-4-fluorophenyl)-5-cyanopicolinamide

Starting with (4S,6S)- and (4R,6R)-4-(5-amino-2-fluorophenyl)-4-(fluoromethyl)-6-methyl-6-(trifluoromethyl)-5,6-dihydro-4H-1,3-oxazin-2-amine (Xa-1), the reaction with 5-cyano-2-pyridinecarboxylic acid and DMTMM as the condensating agent yielded the title compound after chromatography on silica gel (eluent: heptane-EtOAc; gradient: 0-40% EtOAc) as a white solid (71% yield). ¹H NMR (300 MHz, CDCl₃) δ ppm 9.88 (s, 1H), 8.90 (s, 1H), 8.43 (d, J=8.1 Hz, 1H), 8.21 (dd, J=8.2, 1.9 Hz, 1H), 8.07 (dt, J=9.0, 3.5 Hz, 1H), 7.72 (dd, J=6.9, 2.8 Hz, 1H), 7.12 (dd, J=11.3, 8.9 Hz, 1H), 4.44 (ddd, J=63.6, 47.6, 8.5 Hz, 2H), 4.35-4.48 (br s, 2H), 2.69 (d, J=13.9 Hz, 1H), 2.36 (d, J=13.9 Hz, 1H), 1.09 (s, 3H). MS: m/z=454.4 (M+H)⁺.

General Procedure K: Synthesis of Compounds of Formula I-2, I-3 and I-8

A solution of the DMTr-protected amine XIII (76.6 μmol) in dichloromethane (2 ml) was treated at rt with trifluoroacetic acid (89.2 mg, 59.9 μl, 766 μmol), and the mixture was stirred for 2-15 hours. After evaporation at reduced pressure, the residue was purified by chromatography on silica gel or a silica-NH₂ phase using a mixture of DCM-MeOH or heptane-EtOAc or by preparative HPLC to give the pure amines I-2, I-3 and I-8.

### Example 11

### (4S,6S)-4-(2-fluoro-5-(pyrimidin-5-yl)phenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(2-fluoro-5-(pyrimidin-5-yl)phenyl)-4-methyl-6-(trifluoromethyl)-5,6-dihydro-4H-1,3-oxazin-2-amine (XIII-1), the product (4S,6S)-4-(2-fluoro-5-(pyrimidin-5-yl)phenyl)-4-methyl-6-(trifluoromethyl)-5,6-dihydro-4H-1,3-oxazin-2-amine was obtained after chromatography on silica-NH₂ gel (eluent: heptane-EtOAc; gradient: 0-66% EtOAc) as a white foam (81% yield). ¹H NMR (300 MHz, DMSO-d₆) δ ppm 9.20 (s, 1H), 9.04 (s, 2H), 7.77 (ddd, J=8.4, 4.5, 2.6 Hz, 1H), 7.66 (dd, J=7.9, 2.4 Hz, 1H), 7.37 (dd, J=12.0, 8.4 Hz, 1H), 5.95 (br s, 2H), 4.25-4.39 (m, 1H), 2.62 (dd, J=13.4, 2.7 Hz, 1H), 1.86 (t, J=13.0 Hz, 1H), 1.54 (d, J=0.6 Hz, 3H). MS: m/z=355.5 [M+H]⁺.

### Example 12

### 5-(3-((4S,6S)-2-Amino-4-methyl-6-(trifluoromethyl)-5,6-dihydro-4H-1,3-oxazin-4-yl)-4-fluorophenyl)nicotinonitrile

Starting with 5-(3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)nicotinonitrile (XIII-2), the product 5-(3-((4S,6S)-2-amino-4-methyl-6-(trifluoromethyl)-5,6-dihydro-4H-1,3-oxazin-4-yl)-4-fluorophenyl)nicotinonitrile was obtained after chromatography on silica-NH₂ gel (eluent: heptane-EtOAc; gradient: 0-66% EtOAc) as a white foam (80% yield). ¹H NMR (300 MHz, DMSO-d₆) δ ppm 9.07 (d, J=2.2 Hz, 1H), 9.02 (d, J=2.0 Hz, 1H), 8.53 (t, J=2.1 Hz, 1H), 7.77 (ddd, J=8.4, 4.5, 2.4 Hz, 1H), 7.67 (dd, J=7.8, 2.5 Hz, 1H), 7.37 (dd, J=12.1, 8.5 Hz, 1H), 5.95 (br s, 2H), 4.23-4.38 (m, 1H), 2.62 (dd, J=13.3, 2.6 Hz, 1H), 1.86 (t, J=13.0 Hz, 1H), 1.87 (t, J=12.9 Hz, 1H), 1.54 (d, J=0.8 Hz, 3H). MS: m/z=379.5 [M+H]⁺.

### Example 13

### (4S,6S)-4-(5-(5-Chloropyridin-3-yl)-2-fluorophenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(5-(5-chloropyridin-3-yl)-2-fluorophenyl)-4-methyl-6-(trifluoromethyl)-5,6-dihydro-4H-1,3-oxazin-2-amine (XIII-3), the product (4S,6S)-4-(5-(5-chloropyridin-3-yl)-2-fluorophenyl)-4-methyl-6-(trifluoromethyl)-5,6-dihydro-4H-1,3-oxazin-2-amine was obtained after chromatography on silica gel (eluent: heptane-EtOAc; gradient: 0-33% EtOAc) as a white solid (91% yield). ¹H NMR (300 MHz, DMSO-d₆) δ ppm 8.76 (d, J=2.0 Hz, 1H), 8.64 (d, J=2.4 Hz, 1H), 8.12 (t, J=2.1 Hz, 1H), 7.74 (ddd, J=8.4, 4.5, 2.4 Hz, 1H), 7.65 (dd, J=7.8, 2.5 Hz, 1H), 7.34 (dd, J=12.1, 8.5 Hz, 1H), 5.96 (br s, 2H), 4.31 (dqd, J=12.4, 6.2, 6.2, 6.2, 2.3 Hz, 1H), 2.62 (dd, J=13.4, 2.7 Hz, 1H), 1.86 (t, J=13.0 Hz, 1H), 1.54 (d, J=0.8 Hz, 3H). MS: m/z=388.5 [M+H]⁺.

### Example 14

### (4S,6S)-4-(2-Fluoro-5-(5-(prop-1-ynyl)pyridin-3-yl)phenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(2-fluoro-5-(5-(prop-1-ynyl)pyridin-3-yl)phenyl)-4-methyl-6-(trifluoromethyl)-5,6-dihydro-4H-1,3-oxazin-2-amine (XIII-4), the product (4S,6S)-4-(2-fluoro-5-(5-(prop-1-ynyl)pyridin-3-yl)phenyl)-4-methyl-6-(trifluoromethyl)-5,6-dihydro-4H-1,3-oxazin-2-amine was obtained after chromatography on silica-NH₂ gel (eluent: heptane-EtOAc; gradient: 0-33% EtOAc) as a white foam (91% yield). ¹H NMR (300 MHz, CDCl₃) δ ppm 8.59 (d, J=1.8 Hz, 1H), 8.50 (d, J=1.2 Hz, 1H), 7.74 (s, 1H), 7.51 (dd, J=7.7, 2.2 Hz, 1H), 7.37 (ddd, J=8.0, 4.4, 2.5 Hz, 1H), 7.08 (dd, J=11.7, 8.5 Hz, 1H), 4.24 (br s, 2H approx.), 3.95 (dqd, J=12.2, 6.0, 6.0, 6.0, 2.6 Hz, 1H), 2.76 (dd, J=13.6, 2.3 Hz, 1H), 2.02 (s, 3H), 1.84 (t, J=13.1 Hz, 1H), 1.60 (s, 3H). MS: m/z=392.5 [M+H]⁺.

### Example 15

### (4S,6S)-4-(5-(6-Chloropyrazin-2-yl)-2-fluorophenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(5-(6-chloropyrazin-2-yl)-2-fluorophenyl)-4-methyl-6-(trifluoromethyl)-5,6-dihydro-4H-1,3-oxazin-2-amine (XIII-5), the product (4S,6S)-4-(5-(6-chloropyrazin-2-yl)-2-fluorophenyl)-4-methyl-6-(trifluoromethyl)-5,6-dihydro-4H-1,3-oxazin-2-amine was obtained after chromatography on silica gel (eluent: heptane-EtOAc; gradient: 0-25% EtOAc) as a white foam (91% yield). ¹H NMR (300 MHz, CDCl₃) δ ppm 8.91 (s, 1H), 8.51 (s, 1H), 7.99-8.06 (m, 2H), 7.17-7.25 (m, 1H), 3.98-4.12 (m, 1H), 2.85 (dd, J=13.8, 2.7 Hz, 1H), 1.95 (t, J=13.1 Hz, 1H), 1.70 (s, 3H), H₂N-signal not detectable. MS: m/z=389.6 [M+H]⁺.

### Example 16

### (4S,6S)-4-(5-(1-(Difluoromethyl)-1H-pyrazol-4-yl)-2-fluorophenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(5-(1-(difluoromethyl)-1H-pyrazol-4-yl)-2-fluorophenyl)-4-methyl-6-(trifluoromethyl)-5,6-dihydro-4H-1,3-oxazin-2-amine (XIII-6), the product (4S,6S)-4-(5-(1-(difluoromethyl)-1H-pyrazol-4-yl)-2-fluorophenyl)-4-methyl-6-(trifluoromethyl)-5,6-dihydro-4H-1,3-oxazin-2-amine was obtained after chromatography on silica-NH₂ gel (eluent: heptane-EtOAc; gradient: 0-33% EtOAc) as a white solid (80% yield). ¹H NMR (300 MHz, DMSO-d₆) δ ppm 8.53 (s, 1H), 8.10 (s, 1H), 7.85 (t, J=59.1 Hz, 1H), 7.63 (ddd, J=8.4, 4.6, 2.5 Hz, 1H), 7.57 (dd, J=7.9, 2.2 Hz, 1H), 7.23 (dd, J=12.2, 8.4 Hz, 1H), 5.91 (br s, 2H), 4.16-4.32 (m, 1H), 2.61 (dd, J=13.3, 2.6 Hz, 1H), 1.83 (t, J=12.9 Hz, 1H), 1.52 (s, 3H). MS: m/z=393.5 [M+H]⁺.

### Example 17

### (4S,6S)-4-(2,4-Difluoro-5-(pyrimidin-5-yl)phenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(2,4-difluoro-5-(pyrimidin-5-yl)phenyl)-4-methyl-6-(trifluoromethyl)-5,6-dihydro-4H-1,3-oxazin-2-amine (XIII-7), the product (4S,6S)-4-(2,4-difluoro-5-(pyrimidin-5-yl)phenyl)-4-methyl-6-(trifluoromethyl)-5,6-dihydro-4H-1,3-oxazin-2-amine was obtained after chromatography on silica-NH₂ gel (eluent: heptane-EtOAc; gradient: 0-33% EtOAc) as a white foam (79% yield). ¹H NMR (300 MHz, DMSO-d₆) δ ppm 9.24 (s, 1H), 8.96 (d, J=1.2 Hz, 2H), 7.45-7.55 (m, 2H), 5.95 (br s, 2H approx.), 4.32-4.46 (m, 1H), 2.57 (dd, J=13.5, 2.4 Hz, 1H), 1.86 (t, J=13.1 Hz, 1H), 1.53 (s, 3H). MS: m/z=373.5 [M+H]⁺.

### Example 18

### (4S,6S)-4-(2,4-Difluoro-5-(2-fluoropyridin-3-yl)phenyl)-4-methyl-6-(trifluoro-methyl)-5,6-dihydro-4H-1,3-oxazin-2-amine

Starting with (4S,6S)—N-(bis(4-methoxyphenyl)(phenyl)methyl)-4-(2,4-difluoro-5-(2-fluoropyridin-3-yl)phenyl)-4-methyl-6-(trifluoromethyl)-5,6-dihydro-4H-1,3-oxazin-2-amine (XIII-8), the product (4S,6S)-4-(2,4-difluoro-5-(2-fluoropyridin-3-yl)phenyl)-4-methyl-6-(trifluoromethyl)-5,6-dihydro-4H-1,3-oxazin-2-amine was obtained after chromatography on silica-NH₂ gel (eluent: heptane-EtOAc; gradient: 0-33% EtOAc) as a white foam (77% yield). ¹H NMR (300 MHz, CDCl₃) δ ppm 8.26 (dq, J=4.8, 1.0 Hz, 1H), 7.82 (dddd, J=9.4, 7.5, 2.0, 0.9 Hz, 1H), 7.44 (td, J=8.7, 1.0 Hz, 1H), 7.29 (ddd, J=7.3, 4.8, 1.8 Hz, 1H, part of signal concealed by solvent), 6.96 (dd, J=11.6, 9.4 Hz, 1H), 4.09-4.23 (br, 2H approx.), 4.03 (dqd, J=12.2, 5.8, 5.8, 5.8, 2.4 Hz, 1H), 2.77 (dd, J=13.8, 2.7 Hz, 1H), 1.90 (dd, J=13.6, 12.6 Hz, 1H), 1.64 (d, J=1.2 Hz, 3H). MS: m/z=390.5 [M+H]⁺.

### Example 19

### (4S,6S)-4-(2,4-Difluoro-5-(5-(prop-1-ynyl)pyridin-3-yl)phenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(2,4-difluoro-5-(5-(prop-1-ynyl)pyridin-3-yl)phenyl)-4-methyl-6-(trifluoromethyl)-5,6-dihydro-4H-1,3-oxazin-2-amine (XIII-9), the product (4S,6S)-4-(2,4-difluoro-5-(5-(prop-1-ynyl)pyridin-3-yl)phenyl)-4-methyl-6-(trifluoromethyl)-5,6-dihydro-4H-1,3-oxazin-2-amine was obtained after chromatography on silica-NH₂ gel (eluent: heptane-EtOAc; gradient: 0-33% EtOAc) as a colorless viscous oil (76% yield). ¹H NMR (300 MHz, CDCl₃) δ ppm 8.59-8.63 (m, 2H), 7.81 (d, J=1.6 Hz, 1H), 7.46 (t, J=8.9 Hz, 1H), 6.96 (dd, J=11.5, 9.9 Hz, 1H), 4.18 (br s, 2H), 4.01 (dqd, J=12.4, 6.1, 6.1, 6.1, 2.8 Hz, 1H), 2.78 (dd, J=13.7, 2.8 Hz, 1H), 2.10 (s, 3H), 1.90 (dd, J=13.7, 12.7 Hz, 1H), 1.64 (d, J=1.2 Hz, 3H). MS: m/z=410.5 [M+H]⁺.

### Example 20

### (4S,6S)-4-(5-((2-Chloropyridin-4-yl)ethynyl)-2-fluorophenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(5-((2-chloropyridin-4-yl)ethynyl)-2-fluorophenyl)-4-methyl-6-(trifluoromethyl)-5,6-dihydro-4H-1,3-oxazin-2-amine (XVI-1), the product (4S,6S)-4-(5-((2-chloropyridin-4-yl)ethynyl)-2-fluorophenyl)-4-methyl-6-(trifluoromethyl)-5,6-dihydro-4H-1,3-oxazin-2-amine was obtained after chromatography on silica gel (eluent: heptane-EtOAc; gradient: 0-50% EtOAc) as a light white solid (82% yield). ¹H NMR (300 MHz, CDCl₃) δ ppm 8.37 (d, J=5.0 Hz, 1H), 7.58 (dd, J=7.6, 1.9 Hz, 1H), 7.46 (ddd, J=8.2, 4.7, 2.2 Hz, 1H), 7.42 (s, 1H), 7.28 (dd, J=5.0, 1.0 Hz, 1H), 7.08 (dd, J=11.7, 8.5 Hz, 1H), 4.08-4.76 (br, 2H), 3.92-4.07 (m, 1H), 2.78 (dd, J=13.9, 2.4 Hz, 1H), 1.91 (t, J=13.2 Hz, 1H), 1.64 (s, 3H). MS: m/z=412.5 [M+H]⁺.

### Example 21

### 6-((3-((4S,6S)-2-Amino-4-methyl-6-(trifluoromethyl)-5,6-dihydro-4H-1,3-oxazin-4-yl)-4-fluorophenyl)ethynyl)nicotinonitrile

Starting with 6-((3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)ethynyl)nicotinonitrile (XVI-2), the product 6-((3-((4S,6S)-2-amino-4-methyl-6-(trifluoromethyl)-5,6-dihydro-4H-1,3-oxazin-4-yl)-4-fluorophenyl)ethynyl)nicotinonitrile was obtained after chromatography on silica gel (eluent: heptane-EtOAc; gradient: 0-66% EtOAc) as a white foam (82% yield). ¹H NMR (300 MHz, DMSO-d₆) δ ppm 9.05 (dd, J=2.2, 0.8 Hz, 1H), 8.37 (dd, J=8.3, 2.2 Hz, 1H), 7.86 (dd, J=8.3, 0.8 Hz, 1H), 7.59-7.68 (m, 2H), 7.33 (dd, J=12.0, 8.4 Hz, 1H), 6.02 (br s, 2H), 4.23-4.39 (m, 1H), 2.59 (dd, J=13.1, 2.4 Hz, 1H), 1.85 (t, J=12.9 Hz, 1H), 1.50 (s, 3H). MS: m/z=403.6 [M+H]⁺.

### Example 22

### (4S,6S)-4-(2-Fluoro-5-(pyridin-3-ylethynyl)phenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(2-fluoro-5-(pyridin-3-ylethynyl)phenyl)-4-methyl-6-(trifluoromethyl)-5,6-dihydro-4H-1,3-oxazin-2-amine (XVI-3), the product (4S,6S)-4-(2-fluoro-5-(pyridin-3-ylethynyl)phenyl)-4-methyl-6-(trifluoromethyl)-5,6-dihydro-4H-1,3-oxazin-2-amine was obtained after chromatography on silica gel (eluent: heptane-EtOAc; gradient: 0-66% EtOAc) as a white foam (94% yield). H NMR (300 MHz, DMSO-d₆) δ ppm 8.76 (dd, J=2.0, 0.8 Hz, 1H), 8.59 (dd, J=4.8, 1.6 Hz, 1H), 7.99 (dt, J=8.1, 1.8 Hz, 1H), 7.53-7.61 (m, 2H), 7.46 (ddd, J=7.9, 4.9, 0.9 Hz, 1H), 7.29 (dd, J=12.2, 8.2 Hz, 1H), 6.01 (br s, approx. 2H), 2.59 (dd, J=13.5, 2.8 Hz, 1H), 1.84 (t, J=12.8 Hz, 1H), 1.50 (s, 3H). MS: m/z=378.5 [M+H]⁺.

### Example 23

### (4S,6S)-4-(5-((5-Chloropyrimidin-2-yl)ethynyl)-2-fluorophenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(5-((5-chloropyrimidin-2-yl)ethynyl)-2-fluorophenyl)-4-methyl-6-(trifluoromethyl)-5,6-dihydro-4H-1,3-oxazin-2-amine (XVI-4), the product (4S,6S)-4-(5-((5-chloropyrimidin-2-yl)ethynyl)-2-fluorophenyl)-4-methyl-6-(trifluoromethyl)-5,6-dihydro-4H-1,3-oxazin-2-amine was obtained after chromatography on silica gel (eluent: heptane-EtOAc; gradient: 0-50% EtOAc) as a yellow solid (80% yield). ¹H NMR (300 MHz, CDCl₃) δ ppm 8.71 (s, 2H), 7.73 (dd, J=7.8, 2.1 Hz, 1H), 7.58 (ddd, J=8.3, 4.6, 2.2 Hz, 1H), 7.07 (dd, J=11.7, 8.5 Hz, 1H), 4.19 (br s, 2H), 3.96 (dqd, J=12.2, 5.9, 5.9, 5.9, 2.6 Hz, 1H), 2.74 (dd, J=13.7, 2.6 Hz, 1H), 1.88 (dd, J=13.6, 12.6 Hz, 1H), 1.63 (d, J=1.2 Hz, 3H). MS: m/z=413.5 [M+H]⁺.

### Example 24

### (4S,6S)-4-(2-Fluoro-5-((5-methoxypyrimidin-2-yl)ethynyl)phenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(2-fluoro-5-((5-methoxypyrimidin-2-yl)ethynyl)phenyl)-4-methyl-6-(trifluoromethyl)-5,6-dihydro-4H-1,3-oxazin-2-amine (XVI-5), the product (4S,6S)-4-(2-fluoro-5-((5-methoxypyrimidin-2-yl)ethynyl)phenyl)-4-methyl-6-(trifluoromethyl)-5,6-dihydro-4H-1,3-oxazin-2-amine was obtained after chromatography on silica gel (eluent: heptane-EtOAc; gradient: 0-50% EtOAc) as a light yellow foam (87% yield). ¹H NMR (300 MHz, DMSO-d₆) δ ppm 8.83 (s, 2H), 7.50-7.58 (m, 2H), 7.28 (dd, J=11.9, 8.5 Hz, 1H), 5.99 (s, 2H), 4.18-4.33 (m, 1H), 3.97 (s, 3H), 2.59 (dd, J=13.4, 2.5 Hz, 1H), 1.83 (t, J=13.0 Hz, 1H), 1.49 (s, 3H). MS: m/z=409.06 [M+H]⁺.

### Example 25

### (4S,6S)-4-(2-Fluoro-5-((5-methoxypyrazin-2-yl)ethynyl)phenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(2-fluoro-5-((5-methoxypyrazin-2-yl)ethynyl)phenyl)-4-methyl-6-(trifluoromethyl)-5,6-dihydro-4H-1,3-oxazin-2-amine (XVI-6), the product (4S,6S)-4-(2-fluoro-5-((5-methoxypyrazin-2-yl)ethynyl)phenyl)-4-methyl-6-(trifluoromethyl)-5,6-dihydro-4H-1,3-oxazin-2-amine was obtained after chromatography on silica gel (eluent: heptane-EtOAc; gradient: 0-50% EtOAc) as a white solid (82% yield). H NMR (300 MHz, CDCl₃) δ ppm 8.30 (d, J=1.4 Hz, 1H), 8.22 (d, J=1.4 Hz, 1H), 7.63 (dd, J=7.9, 2.2 Hz, 1H), 7.49 (ddd, J=8.3, 4.6, 2.2 Hz, 1H), 7.05 (dd, J=11.9, 8.3 Hz, 1H), 4.19 (br s, 2H), 4.00 (s, 3H), 3.93-4.04 (m, 1H), 2.75 (dd, J=13.6, 2.7 Hz, 1H), 1.88 (dd, J=13.6, 12.6 Hz, 1H), 1.63 (d, J=1.2 Hz, 3H). MS: m/z=409.62 [M+H]⁺.

The following compounds of formula I-5 and I-6 were prepared following General Procedure H.

### Example 26

### 5-(5-((4S,6S)-2-Amino-4-methyl-6-(trifluoromethyl)-5,6-dihydro-4H-1,3-oxazin-4-yl)thiophen-3-yl)nicotinonitrile Hydrochloride

Starting with (4S,6S)-4-(4-bromothiophen-2-yl)-4-methyl-6-(trifluoromethyl)-5,6-dihydro-4H-1,3-oxazin-2-amine (XXIX-1) and 5-cyanopyridin-3-ylboronic acid, the title compound was obtained after chromatography on silica-NH₂ gel (eluent: heptane-EtOAc; gradient: 0-40% EtOAc), treatment with 4 N hydrochloric acid in dioxane, evaporation, and trituration in diethylether as a white solid (26% yield). ¹H NMR (300 MHz, DMSO-d₆) δ ppm 11.05 (s, 1H), 9.15-9.57 (br, 1H approx.), 9.29 (d, J=2.2 Hz, 1H), 8.95 (d, J=2.0 Hz, 1H), 8.70 (t, J=2.0 Hz, 1H), 8.35-8.75 (br, 1H approx.), 8.23 (d, J=1.4 Hz, 1H), 7.83 (d, J=1.6 Hz, 1H), 5.14-5.29 (m, 1H), 2.73-2.81 (m, 1H), 2.58 (m, J=12.5 Hz, 1H), 1.84 (s, 3H). MS: m/z=367.4 [M+H]⁺.

### Example 27

### (4S,6S)-4-(4-(2-Fluoropyridin-3-yl)thiophen-2-yl)-4-methyl-6-(trifluoromethyl)-5,6-dihydro-4H-1,3-oxazin-2-amine Hydrochloride

Starting with (4S,6S)-4-(4-bromothiophen-2-yl)-4-methyl-6-(trifluoromethyl)-5,6-dihydro-4H-1,3-oxazin-2-amine (XXIX-1) and 2-fluoropyridin-3-ylboronic acid, the title compound was obtained after chromatography on silica-NH₂ gel (eluent: heptane-EtOAc; gradient: 0-40% EtOAc), treatment with 4 N hydrochloric acid in dioxane, evaporation, and trituration in diethylether as a white solid (24% yield). ¹H NMR (300 MHz, DMSO-d₆) δ ppm 11.07 (s, 1H), 8.96-9.66 (br, 1H), 8.39-8.94 (br, 1H), 8.31 (ddd, J=10.2, 7.6, 1.9 Hz, 1H), 8.20 (dt, J=4.7, 1.6 Hz, 1H), 7.98 (t, J=1.6 Hz, 1H), 7.64 (t, J=1.2 Hz, 1H), 7.47 (ddd, J=7.5, 4.8, 2.0 Hz, 1H), 5.13-5.27 (m, 1H), 2.81 (dd, J=14.1, 2.4 Hz, 1H), 2.56 (m, J=12.7 Hz, 1H), 1.83 (s, 3H). MS: m/z=360.4 [M+H]⁺.

### Example 28

### (4S,6S)-4-Methyl-6-(trifluoromethyl)-4-(4-(3-(trifluoromethyl)phenyl)thiophen-2-yl)-5,6-dihydro-4H-1,3-oxazin-2-amine

Starting with (4S,6S)-4-(4-bromothiophen-2-yl)-4-methyl-6-(trifluoromethyl)-5,6-dihydro-4H-1,3-oxazin-2-amine (XXIX-1) and 3-(trifluoromethyl)phenylboronic acid, the title compound was obtained after chromatography on silica-NH₂ gel (eluent: heptane-EtOAc; gradient: 0-40% EtOAc) as a colorless waxy solid (53% yield). ¹H NMR (300 MHz, CDCl₃) δ ppm 7.79 (s, 1H), 7.71-7.76 (m, 1H), 7.47-7.57 (m, 2H), 7.38 (d, J=1.4 Hz, 1H), 7.12 (d, J=1.6 Hz, 1H), 4.32 (dqd, J=12.2, 5.9, 5.9, 5.9, 2.6 Hz, 1H), 4.22 (br s, 2H), 2.41 (dd, J=13.3, 2.8 Hz, 1H), 1.99 (dd, J=13.3, 12.3 Hz, 1H), 1.66 (s, 3H). MS: m/z=409.5 [M+H]⁺.

### Example 29

### (4S,6S)-4-Methyl-4-(4-(5-(prop-1-ynyl)pyridin-3-yl)thiophen-2-yl)-6-(trifluoromethyl)-5,6-dihydro-4H-1,3-oxazin-2-amine Hydrochloride

Starting with (4S,6S)-4-(4-bromothiophen-2-yl)-4-methyl-6-(trifluoromethyl)-5,6-dihydro-4H-1,3-oxazin-2-amine (XXIX-1) and 5-(prop-1-ynyl)pyridin-3-ylboronic acid, the title compound was obtained after chromatography on silica-NH₂ gel (eluent: heptane-EtOAc; gradient: 0-40% EtOAc), treatment with 4 N hydrochloric acid in dioxane, evaporation, and trituration in diethylether as a white solid (21% yield). ¹H NMR (600 MHz, DMSO-d₆) δ ppm 11.01 (s, 1H), 9.23-9.52 (br s, 1H), 8.96 (d, J=2.2 Hz, 1H), 8.54 (d, J=1.9 Hz, 1H), 8.39-8.53 (br s, 1H), 8.24 (t, J=2.0 Hz, 1H), 8.15 (d, J=1.5 Hz, 1H), 7.79 (d, J=1.5 Hz, 1H), 5.18-5.25 (m, 1H), 2.79 (dd, J=14.0, 2.2 Hz, 1H), 2.55 (dd, J=14.1, 12.6 Hz, 1H), 2.12 (s, 3H), 1.82 (s, 3H). MS: m/z=380.6 [M+H]⁺.

### Example 30

### (4S,6S)-4-(3-Chloro-5-(5-(prop-1-ynyl)pyridin-3-yl)thiophen-2-yl)-4-methyl-6-(trifluoromethyl)-5,6-dihydro-4H-1,3-oxazin-2-amine

Starting with the crude (4S,6S)-4-(5-bromo-3-chlorothiophen-2-yl)-4-methyl-6-(trifluoromethyl)-5,6-dihydro-4H-1,3-oxazin-2-amine (XXXVII-1) and 5-(prop-1-ynyl)pyridin-3-ylboronic acid, the title compound was obtained after chromatography on silica gel (eluent: heptane-EtOAc; gradient: 0-65% EtOAc) as a light brown foam (19% yield). ¹H NMR (600 MHz, CDCl₃) δ ppm 8.65 (d, J=2.2 Hz, 1H), 8.52 (d, J=1.9 Hz, 1H), 7.77 (t, J=2.1 Hz, 1H), 7.16 (s, 1H), 4.17-4.35 (br, 2H), 4.26 (dqd, J=11.7, 5.9, 5.9, 5.9, 2.9 Hz, 1H), 3.13 (dd, J=13.7, 2.7 Hz, 1H), 2.09 (s, 3H), 1.83 (dd, J=13.7, 12.5 Hz, 1H), 1.71 (s, 3H). MS: m/z=414.1 [M+H]⁺.

### Example 31

### 5-(5-((4S,6S)-2-Amino-4-methyl-6-(trifluoromethyl)-5,6-dihydro-4H-1,3-oxazin-4-yl)-4-chlorothiophen-2-yl)nicotinonitrile

Starting with the crude (4S,6S)-4-(5-bromo-3-chlorothiophen-2-yl)-4-methyl-6-(trifluoromethyl)-5,6-dihydro-4H-1,3-oxazin-2-amine (XXXVII-1) and 5-cyanopyridin-3-ylboronic acid, the title compound was obtained after chromatography on silica gel (eluent: heptane-EtOAc; gradient: 0-65% EtOAc) and HPLC [Gemini NX, 50×4.6 mm; eluent: H₂O (20%+0.05% TEA)/acetonitrile (80%)] as a white foam (11% yield). ¹H NMR (300 MHz, CDCl₃) δ ppm 8.96 (d, J=2.4 Hz, 1H), 8.78 (d, J=1.8 Hz, 1H), 8.03 (t, J=2.1 Hz, 1H), 7.25 (s, 1H), 4.31-4.55 (m, approx. 2H), 4.25 (dqd, J=11.7, 5.9, 5.9, 5.9, 2.8 Hz, 1H), 3.14 (dd, J=13.7, 2.8 Hz, 1H), 1.86 (dd, J=13.6, 12.6 Hz, 1H), 1.71 (s, 3H). MS: m/z=401.1 [M+H]⁺.

### Example 32

### (4S,6S)-4-(3-Chloro-5-(5-chloropyridin-3-yl)thiophen-2-yl)-4-methyl-6-(trifluoromethyl)-5,6-dihydro-4H-1,3-oxazin-2-amine

Starting with the crude (4S,6S)-4-(5-bromo-3-chlorothiophen-2-yl)-4-methyl-6-(trifluoromethyl)-5,6-dihydro-4H-1,3-oxazin-2-amine (XXXVII-1) and 5-chloropyridin-3-ylboronic acid, the title compound was obtained after chromatography on silica gel (eluent: heptane-EtOAc; gradient: 0-65% EtOAc) as a light yellow foam (22% yield). ¹H NMR (600 MHz, CDCl₃) δ ppm 8.66 (d, J=2.0 Hz, 1H), 8.49 (d, J=2.2 Hz, 1H), 7.78 (t, J=2.2 Hz, 1H), 7.19 (s, 1H), 4.17-4.34 (m, 3H), 3.14 (dd, J=13.8, 2.8 Hz, 1H), 1.84 (dd, J=13.7, 12.5 Hz, 1H), 1.71 (s, 3H). MS: m/z=410.0 [M+H]⁺.

### Example 33

### 3-(5-((4S,6S)-2-Amino-4-methyl-6-(trifluoromethyl)-5,6-dihydro-4H-1,3-oxazin-4-yl)-4-chlorothiophen-2-yl)benzonitrile

Starting with the crude (4S,6S)-4-(5-bromo-3-chlorothiophen-2-yl)-4-methyl-6-(trifluoromethyl)-5,6-dihydro-4H-1,3-oxazin-2-amine (XXXVII-1) and 3-cyanophenylboronic acid, the title compound was obtained after chromatography on silica gel (eluent: heptane-EtOAc; gradient: 0-35% EtOAc) as a white foam (33% yield). ¹H NMR (300 MHz, CDCl₃) δ ppm 7.77-7.80 (m, 1H), 7.71-7.75 (m, 1H), 7.57 (dt, J=7.9, 1.4 Hz, 1H), 7.48 (td, J=7.9, 0.6 Hz, 1H), 7.17 (s, 1H), 4.13-4.36 (m, 3H), 3.13 (dd, J=13.7, 2.8 Hz, 1H), 1.84 (dd, J=13.7, 12.5 Hz, 1H), 1.71 (s, 3H). MS: m/z=400.4 [M+H]⁺.

### Example 34

### N-(5-((4S,6S)-2-amino-4-methyl-6-(trifluoromethyl)-5,6-dihydro-4H-1,3-oxazin-4-yl)thiophen-3-yl)-5-chloropicolinamide

### Preparation of the intermediate (4S,6S)-4-(4-aminothiophen-2-yl)-4-methyl-6-(trifluoromethyl)-5,6-dihydro-4H-1,3-oxazin-2-amine (XXXVIII-1)

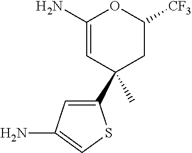

To a solution of (4S,6S)-4-(4-bromothiophen-2-yl)-4-methyl-6-(trifluoromethyl-6-(tfluoomethyl)-5,6-dihydro-4H-1,3-oxazin-2-amine (XXIX-1) (250 mg) in dioxane (5 ml) was added a solution of hydrochloric acid in dioxane (4 M, 0.185 ml) and the solution was evaporated. The residue was dissolved in ethanol (9.5 ml) and water (4.3 ml) and treated subsequently with sodium azide (379 mg), sodium L-ascorbate (58 mg) and copper (I) iodide (56 mg). The mixture was flushed with argon for 5 min, treated with trans-N,N′-dimethylcyclohexane-1,2-diamine (104 mg) and heated to 70° C. for 30 min. The dark green mixture was partitioned between saturated aqueous NaHCO₃ solution and EtOAc, the organic layer was dried and evaporated to give the crude intermediate (4S,6S)-4-(4-azidothiophen-2-yl)-4-methyl-6-(trifluoromethyl)-5,6-dihydro-4H-1,3-oxazin-2-amine (280 mg) as a green oil, which was used in the next step without further purification. MS: m/z=306.4 [M+H]⁺. The crude material was dissolved in methanol (14 ml) and treated with activated zinc (94 mg) and ammonium formate (227 mg) and stirring was continued at 22° C. for 1 h. The mixture was partitioned between saturated aqueous NaHCO₃ solution and EtOAc, the organic layer was dried, evaporated and the residue purified by flash chromatography (NH₂-phase from Biotage, gradient EtOAc in heptane, 0% to 80% EtOAc) to give (4S,6S)-4-(4-aminothiophen-2-yl)-4-methyl-6-(trifluoromethyl)-5,6-dihydro-4H-1,3-oxazin-2-amine (XXXVIII-1) (49 mg) as a yellow oil. MS: m/z=280.4 [M+H]⁺. To a solution of (4S,6S)-4-(4-aminothiophen-2-yl)-4-methyl-6-(trifluoromethyl)-5,6-dihydro-4H-1,3-oxazin-2-amine (XXXVIII-1) (20 mg) and 5-chloro-2-pyridinecarboxylic acid (15 mg) in EtOAc (0.65 ml) was added a solution of 2,4,6-tripropyl-1,3,5,2,4,6-trioxatriphosphorinane-2,4,6-trioxide (T3P, 50% in ethyl acetate, 0.063 ml) and stirring was continued at 22° C. for 4 h. The mixture was partitioned between saturated aqueous NaHCO₃ solution and EtOAc, the organic layer was dried, evaporated and the residue purified by chromatography (NH₂-phase from Biotage, gradient EtOAc in heptane, 0% to 50% EtOAc) to give N-(5-((4S,6S)-2-amino-4-methyl-6-(trifluoromethyl)-5,6-dihydro-4H-1,3-oxazin-4-yl)thiophen-3-yl)-5-chloropicolinamide (Example 34, 30 mg) as a white solid. MS: m/z=419.3 [M+H]⁺.

### Example 35

### N-(5-((4S,6S)-2-Amino-4-methyl-6-(trifluoromethyl)-5,6-dihydro-4H-1,3-oxazin-4-yl)thiophen-3-yl)-5-cyanopicolinamide

To a solution of (4S,6S)-4-(4-aminothiophen-2-yl)-4-methyl-6-(trifluoromethyl)-5,6-dihydro-4H-1,3-oxazin-2-amine (XXXVIII-1) (10 mg) and 5-cyano-2-pyridinecarboxylic acid (7 mg) in EtOAc (0.3 ml) was added a solution of 2,4,6-tripropyl-1,3,5,2,4,6-trioxatriphosphorinane-2,4,6-trioxide (T3P, 50% in ethyl acetate, 0.032 ml) and stirring was continued at 22° C. for 2 h. The mixture was partitioned between saturated aqueous NaHCO₃ solution and EtOAc, the organic layer was dried, evaporated and the residue purified by chromatography (NH₂-phase from Biotage, gradient EtOAc in heptane, 20% to 80% EtOAc) to give the title compound (15 mg) as an off-white solid. MS: m/z=410.4 [M+H]⁺.

### Example 36

### 7-(3-((4S,6S)-2-Amino-4-methyl-6-(trifluoromethyl)-5,6-dihydro-4H-1,3-oxazin-4-yl)-4-fluorophenylamino)-6,7-dihydro-5H-cyclopenta[b]pyridine-3-carbonitrile

To solution of (4S,6S)-4-(5-amino-2-fluorophenyl)-4-methyl-6-(trifluoromethyl)-5,6-dihydro-4H-1,3-oxazin-2-amine (50 mg) prepared as described by H. Hilpert et al., J. Med. Chem., 56, 3980, 2013 and 7-oxo-5,6-dihydrocyclopenta[b]pyridine-3-carbonitrile (33 mg) prepared as described by H. Hilpert et al., WO2012156284A1 in dichloroethane was added at 22° C. acetic acid (21 mg) and decaborane (42 mg) and stirring was continued for 2 h. The mixture was partitioned between aqueous Na₂CO₃ (10%) and dichloromethane, the organic layer was dried, evaporated and the residue purified by prep. HPLC (RP-18, gradient water/acetonitrile) to give the title compound (10 mg) as a pale brown oil. MS: m/z=434.4 [M+H]⁺.

### Examples 37 and 38

### N-(3-((4S,6S)-2-Amino-4-methyl-6-(trifluoromethyl)-5,6-dihydro-4H-1,3-oxazin-4-yl)-2-fluorophenyl)-5-cyanopicolinamide and N-(3-((4S,6S)-2-amino-4-methyl-6-(trifluoromethyl)-5,6-dihydro-4H-1,3-oxazin-4-yl)-2,4-difluorophenyl)-5-cyanopicolinamide

### Preparation of the intermediate (3S,5S)-3-(2,6-difluorophenyl)-3-methyl-5-(trifluoromethyl)isoxazolidine (XXII-2)

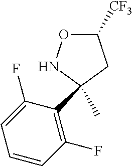

To a stirred solution of the 2-bromo-1,3-difluorobenzene (5.67 g) in di-n-butylether (9 ml) and toluene (27 ml) was added at −78° C. n-BuLi (1.6 M in hexane, 18 ml) over 35 min and stirring was continued at −78° C. for 1 h. A solution of 3-methyl-5-(trifluoromethyl)-4,5-dihydroisoxazole (2.5 g) prepared as described by H. Hilpert et al., J. Med. Chem., 56, 3980, 2013 in di-n-butylether (8 ml) and toluene (12 ml) containing BF₃-Et₂O (4.87 g) was added over 30 min keeping the temperature below −70° C. The mixture was stirred at −78° C. for 1 h, quenched with ethanol (10 ml), warmed to 10° C. and partitioned between saturated aqueous NaHCO₃ and EtOAc. The organic layer was washed with brine, dried, evaporated (45° C./0.1 mbar) and the residue was purified by flash chromatography (SiO₂, gradient EtOAc in heptane, 0% to 30% EtOAc) to give the racemate (2.31 g) as a pale yellow solid. The racemate was resolved by chiral prep. HPLC (Chiralpak AD, 3% isopropanol in n-heptane) to give (3R,5R)-3-(2,6-difluorophenyl)-3-methyl-5-(trifluoromethyl)isoxazolidine (0.71 g) as the faster eluting enantiomer as an off-white solid with positive optical rotation. MS: m/z=268.5 [M+H]⁺. The second fraction contained (3S,5S)-3-(2,6-difluorophenyl)-3-methyl-5-(trifluoromethyl)isoxazolidine (XXII-2) (0.69 g) of the slower eluting desired enantiomer as an off-white solid with negative optical rotation. MS: m/z=268.5 [M+H]⁺.

### Preparation of the intermediate (2S,4S)-4-amino-4-(2,6-difluorophenyl)-1,1,1-trifluoropentan-2-ol (VIIb-2)

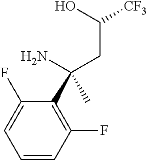

To a solution of (3S,5S)-3-(2,6-difluorophenyl)-3-methyl-5-(trifluoromethyl)isoxazolidine (XXII-2) (661 mg) in ethanol (14 ml) was subsequently added at 22° C. ammonium formate (1.25 g) and Pd/C (132 mg, 10% Pd) and the suspension was stirred at 22° C. for 4.5 h. The suspension was filtered, the filtrate evaporated and the residue partitioned between saturated aqueous NaHCO₃ solution and EtOAc. The organic layer was dried, evaporated and the residue purified by flash chromatography (NH₂-phase from Biotage, gradient EtOAc in heptane, 0% to 100% EtOAc) to give (2S,4S)-4-amino-4-(2,6-difluorophenyl)-1,1,1-trifluoropentan-2-ol (VIIb-2) (360 mg) as a colorless solid. MS: m/z=270.4 [M+H]⁺.

### Preparation of the intermediate (4S,6S)-4-(2,6-difluorophenyl)-4-methyl-6-(trifluoromethyl)-5,6-dihydro-4H-1,3-oxazin-2-amine (VIIIc-2)

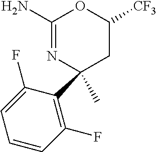

To a solution of (2S,4S)-4-amino-4-(2,6-difluorophenyl)-1,1,1-trifluoropentan-2-ol (VIIb-2) (330 mg) in ethanol (4 ml) was added at 22° C. a solution of BrCN (5 M in acetonitrile, 0.37 ml) and stirring was continued at 75° C. (oil bath temp.) for 20 h. The mixture was evaporated and the residue partitioned between saturated aqueous Na₂CO₃ solution and EtOAc, the organic layer was dried, evaporated and the residue purified by flash chromatography (SiO₂, gradient EtOAc in heptane, 50% to 75% EtOAc) to give (4S,6S)-4-(2,6-difluorophenyl)-4-methyl-6-trifluoromethyl)-5,6-dihydro-4H-1,3-oxazin-2-amine (VIIIc-2) (163 mg) as a colorless oil. MS: m/z=295.4 [M+H]⁺.

### Preparation of the intermediate (4S,6S)-4-(2,6-difluoro-3-nitrophenyl)-4-methyl-6-(trifluoromethyl)-5,6-dihydro-4H-1,3-oxazin-2-amine (IXa-2)

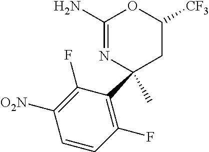

To a solution of (4S,6S)-4-(2,6-difluorophenyl)-4-methyl-6-trifluoromethyl)-5,6-dihydro-4H-1,3-oxazin-2-amine (VIIIc-2) (145 mg) in sulfuric acid (3.8 g) was added at 0° C. red fuming nitric acid (44 mg) and stirring was continued at 0° C. for 30 min. The colorless viscous reaction mixture was added dropwise to an ice/water mixture (1:1, 30 ml) and the pH was adjusted to 6 using aqueous NaOH (4 N, 23 ml). The mixture was partitioned between saturated aqueous Na₂CO₃ solution and EtOAc, the organic layer was dried, evaporated and the residue purified by flash chromatography (SiO₂, preconditioned with heptane/EtOAc/Et₃N, 65/30/5 then heptane/EtOAc 2:1, then purified with gradient of EtOAc in heptane, 20% to 100% EtOAc) to give (4S,6S)-4-(2,6-difluoro-3-nitrophenyl)-4-methyl-6-(trifluoromethyl)-5,6-dihydro-4H-1,3-oxazin-2-amine (IXa-2) (148 mg) as a colorless solid. MS: m/z=340.5 [M+H]⁺.

### Preparation of the intermediates (4S,6S)-4-(3-amino-2-fluorophenyl)-4-methyl-6-(trifluoromethyl)-5,6-dihydro-4H-1,3-oxazin-2-amine (Xa-2) and (4S,6S)-4-(3-amino-2,6-difluorophenyl)-4-methyl-6-(trifluoromethyl)-5,6-dihydro-4H-1,3-oxazin-2-amine (Xa-3)

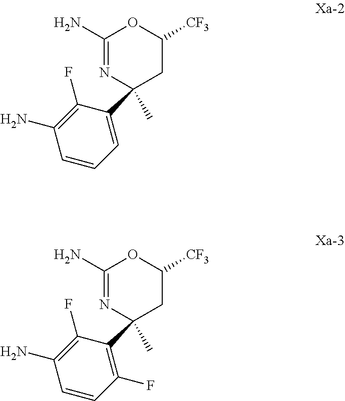

A mixture of (4S,6S)-4-(2,6-difluoro-3-nitrophenyl)-4-methyl-6-(trifluoromethyl)-5,6-dihydro-4H-1,3-oxazin-2-amine (IXa-2) (136 mg) and Et₃N (41 mg) in ethanol (9 ml) containing Pd/C (10%, 21 mg) was hydrogenated at atmospheric pressure and 22° C. for 2 h. The mixture was filtered, the filtrated evaporated and the residue purified by flash chromatography (SiO₂, preconditioned with heptane/EtOAc/Et₃N, 65/30/5 then heptane/EtOAc 2:1, then purified with gradient of EtOAc in heptane, 40% to 100% EtOAc) to give (4S,6S)-4-(3-amino-2-fluorophenyl)-4-methyl-6-(trifluoromethyl)-5,6-dihydro-4H-1,3-oxazin-2-amine (Xa-2) (11 mg) as the faster eluting compound as a pale yellow oil. MS: m/z=292.5 [M+H]⁺. The second fraction contained the slower eluting (4S,6S)-4-(3-amino-2,6-difluorophenyl)-4-methyl-6-(trifluoromethyl)-5,6-dihydro-4H-1,3-oxazin-2-amine (Xa-3) (95 mg) as a pale yellow oil. MS: m/z=310.5 [M+H]⁺.

To a solution of (4S,6S)-4-(3-amino-2-fluorophenyl)-4-methyl-6-(trifluoromethyl)-5,6-dihydro-4H-1,3-oxazin-2-amine (Xa-2) (9 mg) and 5-cyano-2-pyridinecarboxylic acid (6 mg) in EtOAc (0.4 ml) was added a solution of 2,4,6-tripropyl-1,3,5,2,4,6-trioxatriphosphorinane-2,4,6-trioxide (T3P, 50% in ethyl acetate, 0.027 ml) and stirring was continued at 22° C. for 4 h. The mixture was partitioned between saturated aqueous NaHCO₃ solution and EtOAc, the organic layer was dried, evaporated and the residue purified by chromatography (NH₂-phase from Biotage, gradient EtOAc in heptane, 20% to 80% EtOAc) followed by an additional purification on prep. HPLC (gradient of water/acetonitrile) to give N-(3-((4S,6S)-2-amino-4-methyl-6-(trifluoromethyl)-5,6-dihydro-4H-1,3-oxazin-4-yl)-2-fluorophenyl)-5-cyanopiicoinamide (Example 37, 5 mg) as a colorless solid. MS: m/z=422.4 [M+H]⁺.

(4S,6S)-4-(3-amino-2,6-difluorophenyl)-4-methyl-6-(trifluoromethyl)-5,6-dihydro-4H-1,3-oxazin-2-amine (Xa-3) (30 mg) and 5-cyano-2-pyridinecarboxylic acid (19 mg) were coupled according to the procedure given above to give N-(3-((4S,6S)-2-amino-4-methyl-6-(trifluoromethyl)-5,6-dihydro-4H-1,3-oxazin-4-yl)-2,4-difluorophenyl)-5-cyanopicolinamide (Example 38, 32 mg) as a colorless solid. MS: m/z=440.4 [M+H]⁺.

### Example 39

### (4S,6S)-4-(5-(6-Chlorobenzo[d]oxazol-2-yl)-2-fluorophenyl)-4-methyl-6-(trifluoromethyl)-5,6-dihydro-4H-1,3-oxazin-2-amine

### Preparation of the intermediate (4S,6S)—N-(bis(4-methoxyphenyl)(phenyl)methyl)-4-(5-(6-chlorobenzo[d]oxazol-2-yl)-2-fluorophenyl)-4-methyl-6-(trifluoromethyl)-5,6-dihydro-4H-1,3-oxazin-2-amine (XIII-10)

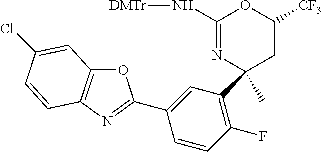

A solution of (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) (50 mg), 2-(5,5-dimethyl-1,3,2-dioxaborinan-2-yl)-5,5-dimethyl-1,3,2-dioxaborinane (19 mg) and potassium acetate (25 mg) in dioxane (1 ml) was flushed with argon for 5 min, treated with bis(triphenylphosphine)palladium(II)dichloride (2.5 mg) and stirring was continued in a sealed tube at 110° C. for 16 h. The mixture was evaporated, the residue partitioned between water and EtOAc, the organic layer was dried and evaporated to give the crude (4S,6S)—N-[bis(4-methoxyphenyl)-phenyl-methyl]-4-[5-(5,5-dimethyl-1,3,2-dioxaborinan-2-yl)-2-fluoro-phenyl]-4-methyl-6-(trifluoromethyl)-5,6-dihydro-1,3-oxazin-2-amine (58 mg) as a yellow oil, which was processed without further purification. A solution of the crude material (50 mg), 2,6-dichlorobenzoxazole (18 mg) and cesium carbonate (9 mg) in THF (2 ml) and water (1 ml) was flushed with argon for 5 min, treated with 1,1′-bis(diphenylphosphino)-ferrocene-palladium(II)dichloride complex with dichloromethane (6 mg) and stirring was continued in a sealed tube at 85° C. for 16 h. The mixture was evaporated and the residue was purified by flash chromatography (SiO₂, gradient EtOAc in heptane, 0% to 50% EtOAc) to give (4S,6S)—N-(bis(4-methoxyphenyl)(phenyl)methyl)-4-(5-(6-chlorobenzo[d]oxazol-2-yl)-2-fluorophenyl)-4-methyl-6-(trifluoromethyl)-5,6-dihydro-4H-1,3-oxazin-2-amine (XIII-10) (28 mg) as a colorless foam.

To a solution of (4S,6S)—N-(bis(4-methoxyphenyl)(phenyl)methyl)-4-(5-(6-chlorobenzo[d]oxazol-2-yl)-2-fluorophenyl)-4-methyl-6-(trifluoromethyl)-5,6-dihydro-4H-1,3-oxazin-2-amine (XIII-10) (28 mg) in dichloromethane (1.3 ml) was added at 22° C. trifluoroacetic acid (0.030 ml) and stirring was continued for 5 h. The mixture was washed with aqueous Na₂CO₃ solution (2 N), the organic layer was dried, evaporated and the residue purified by chromatography (NH₂-phase from Biotage, gradient EtOAc in heptane, 0% to 80% EtOAc) followed by a second chromatography (SiO₂, gradient EtOAc in heptane, 0% to 70% EtOAc) to give (4S,6S)-4-(5-(6-chlorobenzo[d]oxazol-2-yl)-2-fluorophenyl)-4-methyl-6-(trifluoromethyl)-5,6-dihydro-4H-1,3-oxazin-2-amine (Example 39, 11 mg) as a white solid. MS: m/z=428.3 [M+H]⁺.

### Example 40

### (4S,6S)-4-(5-(5-(4-Chlorophenyl)-1,3,4-oxadiazol-2-yl)-2-fluorophenyl)-4-methyl-6-(trifluoromethyl)-5,6-dihydro-4H-1,3-oxazin-2-amine

### Preparation of the intermediate 2-bromo-5-(4-chlorophenyl)-1,3,4-oxadiazole

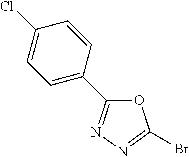 [SMILES: Clc1ccc(-c2nnc(Br)o2)cc1]

To a suspension of 5-(4-chlorophenyl)-1,3,4-oxadiazol-2-amine (391 mg) in acetonitrile (10 ml) was added at 22° C. copper (II) bromide (469 mg) and isoamyl nitrite (469 mg) and stirring was continued for 4 h. The mixture was partitioned between aqueous HCl (1 N) solution and EtOAc, the organic layer was dried, evaporated and the residue purified by chromatography (SiO₂, gradient EtOAc in heptane, 0% to 65% EtOAc) to give 2-bromo-5-(4-chlorophenyl)-1,3,4-oxadiazole (52 mg) as a yellow solid. MS: m/z=261.3 [M+H]⁺.

The coupling of the crude (4S,6S)—N-[bis(4-methoxyphenyl)-phenyl-methyl]-4-[5-(5,5-dimethyl-1,3,2-dioxaborinan-2-yl)-2-fluoro-phenyl]-4-methyl-6-(trifluoromethyl)-5,6-dihydro-1,3-oxazin-2-amine (52 mg) described in example 39 and 2-bromo-5-(4-chlorophenyl)-1,3,4-oxadiazole (24 mg) to give (4S,6S)—N-(bis(4-methoxyphenyl)(phenyl)methyl)-4-(5-(5-(4-chlorophenyl)-1,3,4-oxadiazol-2-yl)-2-fluorophenyl)-4-methyl-6-(trifluoromethyl)-5,6-dihydro-4H-1,3-oxazin-2-amine (23 mg) and deprotection with trifluoroacetic acid was carried out as described for example 39 to give (4S,6S)-4-(5-(5-(4-chlorophenyl)-1,3,4-oxadiazol-2-yl)-2-fluorophenyl)-4-methyl-6-(trifluoromethyl)-5,6-dihydro-4H-1,3-oxazin-2-amine (Example 40, 10 mg) as a colorless solid. MS: m/z=455.5 [M+H]⁺.

### Example 41

### (4S,6S)-4-(4-Fluorobiphenyl-3-yl)-4-methyl-6-(trifluoromethyl)-5,6-dihydro-4H-1,3-oxazin-2-amine

The coupling of the crude (4S,6S)—N-[bis(4-methoxyphenyl)-phenyl-methyl]-4-[5-(5,5-dimethyl-1,3,2-dioxaborinan-2-yl)-2-fluoro-phenyl]-4-methyl-6-(trifluoromethyl)-5,6-dihydro-1,3-oxazin-2-amine (50 mg) described in example 39 and bromo-benzene followed by deprotection with trifluoroacetic acid gave the title compound (5 mg) as a yellow oil. MS: m/z=353.4 [M+H]⁺.

### Example 42

### (4S,6S)-4-Methyl-4-(4-(pyrimidin-5-yl)thiophen-2-yl)-6-(trifluoromethyl)-5,6-dihydro-4H-1,3-oxazin-2-amine

To a solution of (4S,6S)-4-(4-bromothiophen-2-yl)-4-methyl-6-(trifluoromethyl)-5,6-dihydro-4H-1,3-oxazin-2-amine (XXIX-1) (40 mg) in 1,2-dimethoxyethane (0.75 ml) was added subsequently pyrimidine-5-boronic acid (15 mg), triphenylphosphine (8 mg) and an aqueous solution of Na₂CO₃ (2N, 0.15 ml). The mixture was flushed with argon for 5 min, treated with palladium (II) acetate (4 mg) and heated at 100° C. for 16 h. The mixture was evaporated and purified by flash chromatography (NH₂-phase from Biotage, gradient EtOAc in heptane, 0% to 80% EtOAc) followed by another chromatography (SiO₂, gradient MeOH in dichloromethane, 0% to 10% MeOH) to give the title compound (5 mg) as a colorless foam. MS: m/z=343.5 [M+H]⁺.

### Example 43

### 5-(5-((4S,6S)-2-Amino-4-methyl-6-(trifluoromethyl)-5,6-dihydro-4H-1,3-oxazin-4-yl)thiophen-3-yl)pyrimidine-2-carbonitrile

To a solution of (4S,6S)-4-(4-bromothiophen-2-yl)-4-methyl-6-(trifluoromethyl)-5,6-dihydro-4H-1,3-oxazin-2-amine (XXIX-1) (50 mg) in 1,2-dimethoxyethane (1.0 ml) was added subsequently 2-cyanopyrimidine-5-boronic acid pinacolester (34 mg), triphenylphosphine (10 mg) and an aqueous solution of Na₂CO₃ (2N, 0.2 ml). The mixture was flushed with argon for 5 min, treated with palladium(II)acetate (5 mg) and heated at 100° C. for 6 h. The mixture was evaporated and purified by chromatography (NH₂-phase from Biotage, gradient EtOAc in heptane, 0% to 100% EtOAc) followed by another chromatography (SiO₂, gradient MeOH in dichloromethane, 0% to 10% MeOH) to give the title compound (10 mg) as a colorless foam. MS: m/z=368.5 [M+H]⁺.

### Example 44

### 6-(3-((4S,6S)-2-Amino-4-methyl-6-(trifluoromethyl)-5,6-dihydro-4H-1,3-oxazin-4-yl)-4-fluorophenethyl)nicotinonitrile

A mixture of 6-((3-((4S,6S)-2-amino-4-methyl-6-(trifluoromethyl)-5,6-dihydro-4H-1,3-oxazin-4-yl)-4-fluorophenyl)ethynyl)nicotinonitrile (Example 21) (8 mg) in methanol (0.5 ml) and Pt/C (10%, 4 mg) was hydrogenated at atmospheric pressure at 22° C. for 10 min. The mixture was filtered, the filtrated evaporated and the residue purified by flash chromatography (SiO₂, gradient of EtOAc in heptane, 50% to 100% EtOAc) to give the title compound (3 mg) as an off-white solid. MS: m/z=407.6 [M+H]⁺.

### Example 45

### (4S,6S)-4-(5-(6-Chlorobenzo[d]oxazol-2-yl)-2-fluorophenyl)-4-(fluoromethyl)-6-(trifluoromethyl)-5,6-dihydro-4H-1,3-oxazin-2-amine

### Preparation of the intermediate (4S,6S)-4-(5-bromo-2-fluorophenyl)-4-(fluoromethyl)-6-(trifluoromethyl)-5,6-dihydro-4H-1,3-oxazin-2-amine (VIIIb-3)

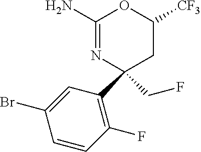

To a dark green solution of tert-butyl nitrite (185 mg) and copper(II)bromide (433 mg) in acetonitrile (5 ml) was added at 65° C. a solution of (4S,6S)-4-(5-amino-2-fluorophenyl)-4-(fluoromethyl)-6-(trifluoromethyl)-5,6-dihydro-4H-1,3-oxazin-2-amine (CAS#1432511-77-7, 500 mg) in acetonitrile (5 ml) over 3 min and stirring was continued for 30 min. The mixture was cooled to 22° C., partitioned between saturated aqueous NaHCO₃ solution and EtOAc, the organic layer was dried, evaporated and the residue purified by flash chromatography (SiO₂, heptane/EtOAc 5:1) to give (4S,6S)-4-(5-bromo-2-fluorophenyl)-4-(fluoromethyl)-6-(trifluoromethyl)-5,6-dihydro-4H-1,3-oxazin-2-amine (VIIIb-3) (182 mg) as a colorless oil. MS: m/z=373.0 and 375.0 [M+H]⁺.

### Preparation of the intermediate (4S,6S)—N-(bis(4-methoxyphenyl)(phenyl)methyl)-4-(5-bromo-2-fluorophenyl)-4-(fluoromethyl)-6-(trifluoromethyl)-5,6-dihydro-4H-1,3-oxazin-2-amine (XII-3)

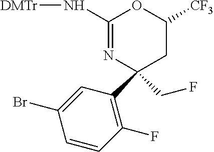

To a solution of (4S,6S)-4-(5-bromo-2-fluorophenyl)-4-(fluoromethyl)-6-(trifluoromethyl)-5,6-dihydro-4H-1,3-oxazin-2-amine (VIIIb-3) (215 mg) and Et₃N (0.16 ml) in dichloromethane (8 ml) was added at 5° C. 4,4′-dimethoxytriphenylmethyl chloride (205 mg) and stirring was continued at 5° C. for 30 min and at 22° C. for 3 h. The mixture was washed with water, the organic layer was dried, evaporated and the residue purified by flash chromatography (SiO₂, gradient of EtOAc in heptane, 0% to 40% EtOAc) to give (4S,6S)—N-(bis(4-methoxyphenyl)(phenyl)methyl)-4-(5-bromo-2-fluorophenyl)-4-(fluoromethyl)-6-(trifluoromethyl)-5,6-dihydro-4H-1,3-oxazin-2-amine (XII-3) (290 mg) as a colorless oil.

### Preparation of the intermediate (4S,6S)—N-(bis(4-methoxyphenyl)(phenyl)methyl)-4-(5-(5,5-dimethyl-1,3,2-dioxaborinan-2-yl)-2-fluorophenyl)-4-(fluoromethyl)-6-(trifluoromethyl)-5,6-dihydro-4H-1,3-oxazin-2-amine (XXXIX-1)

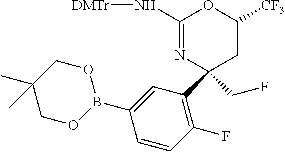

(4S,6S)—N-(Bis(4-methoxyphenyl)(phenyl)methyl)-4-(5-bromo-2-fluorophenyl)-4-(fluoromethyl)-6-(trifluoromethyl)-5,6-dihydro-4H-1,3-oxazin-2-amine (XII-3) was converted to crude (XXXIX-1) according to the method described for Example 39.

### Preparation of the intermediate (4S,6S)—N-(bis(4-methoxyphenyl)(phenyl)methyl)-4-(5-(6-chlorobenzo[d]oxazol-2-yl)-2-fluorophenyl)-4-(fluoromethyl)-6-(trifluoromethyl)-5,6-dihydro-4H-1,3-oxazin-2-amine (XIII-11)

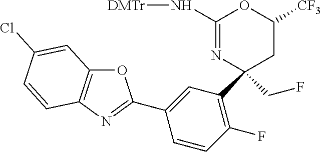

(4S,6S)—N-(bis(4-methoxyphenyl)(phenyl)methyl)-4-(5-(5,5-dimethyl-1,3,2-dioxaborinan-2-yl)-2-fluorophenyl)-4-(fluoromethyl)-6-(trifluoromethyl)-5,6-dihydro-4H-1,3-oxazin-2-amine (XXXIX-1) was coupled with 2,6-dichlorobenzoxazole to (XIII-11) according to the method described for Example 39.

(4S,6S)—N-(bis(4-methoxyphenyl)(phenyl)methyl)-4-(5-(6-chlorobenzo[d]oxazol-2-yl)-2-fluorophenyl)-4-(fluoromethyl)-6-(trifluoromethyl)-5,6-dihydro-4H-1,3-oxazin-2-amine (XIII-11) (42 mg) was deprotected according to the method described for Example 39 to give (4S,6S)-4-(5-(6-chlorobenzo[d]oxazol-2-yl)-2-fluorophenyl)-4-(fluoromethyl)-6-(trifluoromethyl)-5,6-dihydro-4H-1,3-oxazin-2-amine (Example 45, 21 mg) as a colorless solid. MS: m/z=446.1 [M+H]⁺.

### Example 46

### 5-((3-((4S,6S)-2-amino-4-methyl-6-(trifluoromethyl)-5,6-dihydro-4H-1,3-oxazin-4-yl)-4-fluorophenyl)ethynyl)picolinonitrile

### Preparation of the intermediate 5-((3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)ethynyl)picolinonitrile (XVI-7)

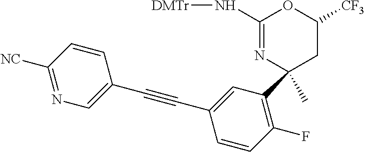

(4S,6S)—N-(Bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (50 mg) (XV) was coupled with 5-((trimethylsilyl)ethynyl)picolinonitrile (20 mg) (prepared as described in A. F. Farahat et al., Bioorg. & Med. Chem., 18, 557, 2010) according to general procedure I to give 5-((3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)ethynyl)picolinonitrile (XVI-7) (26 mg) as a colorless foam.

5-((3-((4S,6S)-2-(Bis(4-methoxyphenyl)(phenyl)methylamino)-4-methyl-6-(trifluoromethyl)-5,6-dihydro-4H-1,3-oxazin-4-yl)-4-fluorophenyl)ethynyl)picolinonitrile (XVI-7) (24 mg) was deprotected according to the method described for Example 39 to give 5-((3-((4S,6S)-2-amino-4-methyl-6-(trifluoromethyl)-5,6-dihydro-4H-1,3-oxazin-4-yl)-4-fluorophenyl)ethynyl)picolinonitrile (Example 46, 9 mg) as a white solid. MS: m/z=403.5 [M+H]⁺.

### Example 47

### (4S,6S)-4-(2-fluoro-5-(5-(5-methyl-1H-pyrazol-3-yl)pyridin-3-yl)phenyl)-4-(fluoromethyl)-6-(trifluoromethyl)-5,6-dihydro-4H-1,3-oxazin-2-amine

Step 1: To a solution of (4S,6S)—N-(bis(4-methoxyphenyl)(phenyl)methyl)-4-(5-(5,5-dimethyl-1,3,2-dioxaborinan-2-yl)-2-fluorophenyl)-4-(fluoromethyl)-6-(trifluoromethyl)-5,6-dihydro-4H-1,3-oxazin-2-amine (Intermediate XXXIX-1, 159 mg) in tetrahydrofuran (8 ml) and water (4 ml) was added at room temperature 3-bromo-5-(5-methyl-1H-pyrazol-3-yl)pyridine (80.1 mg) and cesium carbonate (292 mg). Under argon 1,1′-bis(diphenylphosphino)ferrocene-palladium(II)dichloride dichloromethane complex (18.3 mg) was added and the 2-phase-reaction mixture was stirred in a sealed tube at 85° C. for 3 hours. The aqueous layer was separated and extracted with ethyl acetate. The combined organic layers were dried over Na₂SO₄, filtered and evaporated. The residue was chromatographed (SiO₂, 0-80% EtOAc in heptane) to give (4S,6S)—N-(bis(4-methoxyphenyl)(phenyl)methyl)-4-(2-fluoro-5-(5-(5-methyl-1H-pyrazol-3-yl)pyridin-3-yl)phenyl)-4-(fluoromethyl)-6-(trifluoromethyl)-5,6-dihydro-4H-1,3-oxazin-2-amine (113 mg) as an off-white foam. MS: m/z=798.27 [M+HCOO⁻]⁻

Step 2: To a solution of (4S,6S)—N-(bis(4-methoxyphenyl)(phenyl)methyl)-4-(2-fluoro-5-(5-(5-methyl-1H-pyrazol-3-yl)pyridin-3-yl)phenyl)-4-(fluoromethyl)-6-(trifluoromethyl)-5,6-dihydro-4H-1,3-oxazin-2-amine (112 mg) in dichloromethane (2 ml) was added at rt trifluoroacetic acid (339 mg). The orange solution was stirred at 23° C. for 1 hour. The mixture was evaporated and extracted with ethyl acetate/sat NaHCO₃. The organic layer was washed with water and brine, dried over Na₂SO₄ filtered and evaporated. The residue was chromatographed (SiO2, dichloromethane/MeOH 9:1) to give (4S,6S)-4-(2-fluoro-5-(5-(5-methyl-1H-pyrazol-3-yl)pyridin-3-yl)phenyl)-4-(fluoromethyl)-6-(trifluoromethyl)-5,6-dihydro-4H-1,3-oxazin-2-amine (44 mg) as an off-white foam. MS: m/z=452.15 [M+H]⁺.

### Example 48

### (4S,6S)-4-(5-(5-(1H-tetrazol-5-yl)pyridin-3-yl)-2-fluorophenyl)-4-(fluoromethyl)-6-(trifluoromethyl)-5,6-dihydro-4H-1,3-oxazin-2-amine

Step 1: To a solution of (4S,6S)—N-(bis(4-methoxyphenyl)(phenyl)methyl)-4-(5-(5,5-dimethyl-1,3,2-dioxaborinan-2-yl)-2-fluorophenyl)-4-(fluoromethyl)-6-(trifluoromethyl)-5,6-dihydro-4H-1,3-oxazin-2-amine (Intermediate XXXIX-1, 223 mg) in THF (8.00 ml) and water (4.00 ml) was added at room temperature 3-bromo-5-(1H-tetrazol-5-yl)pyridine (CAS#211943-13-4,107 mg) and cesium carbonate (410 mg). Under argon 1,1′-bis(diphenylphosphino)ferrocene-palladium(II)dichloride dichloromethane complex (25.7 mg) was added and the 2-phase-reaction mixture was stirred in a sealed tube at 85° C. for 3 hours. The aqueous layer was separated and extracted with ethyl acetate. The combined organic layers were dried over Na₂SO₄, filtered and evaporated. The residue was chromatographed (SiO₂, 0-80% MeOH in dichloromethane) to give (4S,6S)-4-(5-(5-(1H-tetrazol-5-yl)pyridin-3-yl)-2-fluorophenyl)-N-(bis(4-methoxyphenyl)(phenyl)methyl)-4-(fluoromethyl)-6-(trifluoromethyl)-5,6-dihydro-4H-1,3-oxazin-2-amine (127 mg) as brown oil.

Step 2: To a solution of (4S,6S)-4-(5-(5-(1H-tetrazol-5-yl)pyridin-3-yl)-2-fluorophenyl)-N-(bis(4-methoxyphenyl)(phenyl)methyl)-4-(fluoromethyl)-6-(trifluoromethyl)-5,6-dihydro-4H-1,3-oxazin-2-amine (127 mg) in dichloromethane (3 ml) was added at room temperature trifluoroacetic acid (976 mg). The orange reaction solution was stirred at 23° C. for 2 hours. The mixture was evaporated and extracted with EtOAc/sat NaHCO₃. The organic layer was washed with water and brine, dried over Na2SO4, filtered and evaporated. The residue was chromatographed with dichloromethane/MeOH 9:1 to give (4S,6S)-4-(5-(5-(1H-tetrazol-5-yl)pyridin-3-yl)-2-fluorophenyl)-4-(fluoromethyl)-6-(trifluoromethyl)-5,6-dihydro-4H-1,3-oxazin-2-amine (4.82 mg) as a colorless oil. MS: m/z=438.4 [M−H]⁻.

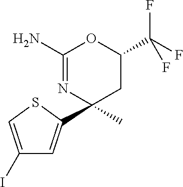 [SMILES: C[C@@]1(c2cc(I)cs2)C[C@@H](C(F)(F)F)OC(N)=N1]

Intermediate (XXXX-49): Starting from (4S,6S)-4-(4-bromothiophen-2-yl)-4-methyl-6-(trifluoromethyl)-5,6-dihydro-4H-1,3-oxazin-2-amine (XXIX-1), the product (4S,6S)-4-(4-iodothiophen-2-yl)-4-methyl-6-(trifluoromethyl)-5,6-dihydro-4H-1,3-oxazin-2-amine (XXXX-49) was obtained after chromatography on silica gel (eluent: heptane-EtOAc; gradient: 0-50% EtOAc) as a light yellow viscous oil (78% yield). MS: m/z=391.4 [M+H]⁺.

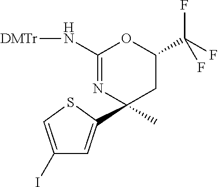

Intermediate (XXXXI-49): Starting from (4S,6S)-4-(4-iodothiophen-2-yl)-4-methyl-6-(trifluoromethyl)-5,6-dihydro-4H-1,3-oxazin-2-amine (XXXX-49), following general procedure G, the product (4S,6S)—N-(bis(4-methoxyphenyl)(phenyl)methyl)-4-(4-iodothiophen-2-yl)-4-methyl-6-(trifluoromethyl)-5,6-dihydro-4H-1,3-oxazin-2-amine (XXXXI-49) was obtained after chromatography on silica gel (eluent: heptane-EtOAc; gradient: 0-20% EtOAc) as a white foam (79% yield). MS: m/z=691.2 [M−H]⁻.

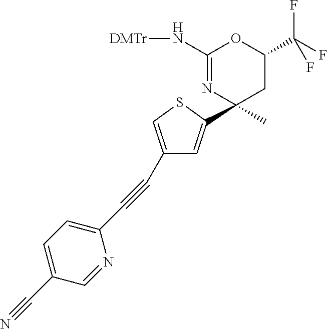

Intermediate (XXXXII-49): Starting from (4S,6S)—N-(bis(4-methoxyphenyl)(phenyl)methyl)-4-(4-iodothiophen-2-yl)-4-methyl-6-(trifluoromethyl)-5,6-dihydro-4H-1,3-oxazin-2-amine (XXXXI-49) and 6-((trimethylsilyl)ethynyl)nicotinonitrile, following general procedure I, the product 6-((5-((4S,6S)-2-(bis(4-methoxyphenyl)(phenyl)methylamino)-4-methyl-6-(trifluoromethyl)-5,6-dihydro-4H-1,3-oxazin-4-yl)thiophen-3-yl)ethynyl)nicotinonitrile (XXXXII-49) was obtained after chromatography on silica gel (eluent: heptane-EtOAc; gradient: 0-33% EtOAc) as a light yellow foam (67% yield). MS: m/z=691.7 [M−H]⁻.

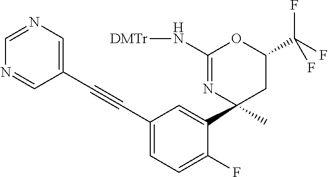

Intermediate (XVI-50): Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 5-((trimethylsilyl)ethynyl)pyrimidine, following general procedure I, the product (4S,6S)—N-(bis(4-methoxyphenyl)(phenyl)methyl)-4-(2-fluoro-5-(pyrimidin-5-ylethynyl)phenyl)-4-methyl-6-(trifluoromethyl)-5,6-dihydro-4H-1,3-oxazin-2-amine (XVI-50) was obtained after chromatography on silica gel (eluent: heptane-EtOAc; gradient: 0-50% EtOAc) as a light yellow foam (78% yield). MS: m/z=679.5 [M−H]⁻.

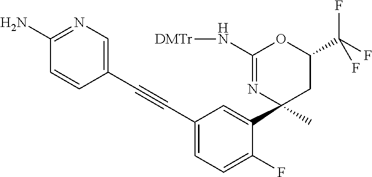

Intermediate (XVI-51): Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 5-((trimethylsilyl)ethynyl)pyridin-2-amine, following general procedure I, the product (4S,6S)-4-(5-((6-aminopyridin-3-yl)ethynyl)-2-fluorophenyl)-N-(bis(4-methoxyphenyl)(phenyl)methyl)-4-methyl-6-(trifluoromethyl)-5,6-dihydro-4H-1,3-oxazin-2-amine (XVI-51) was obtained after chromatography on silica gel (eluent: heptane-EtOAc; gradient: 20-100% EtOAc) as a light yellow foam (83% yield). MS: m/z=693.8 [M−H]⁻.

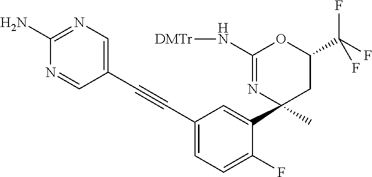

Intermediate (XVI-52): Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 5-((trimethylsilyl)ethynyl)pyrimidin-2-amine, following general procedure I, the product (4S,6S)-4-(5-((2-aminopyrimidin-5-yl)ethynyl)-2-fluorophenyl)-N-(bis(4-methoxyphenyl)(phenyl)methyl)-4-methyl-6-(trifluoromethyl)-5,6-dihydro-4H-1,3-oxazin-2-amine (XVI-52) was obtained after chromatography on silica gel (eluent: heptane-EtOAc; gradient: 0-60% EtOAc) as a light yellow foam (75% yield).

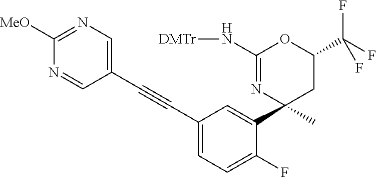

Intermediate (XVI-53): Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 2-methoxy-5-((trimethylsilyl)ethynyl)pyrimidine, following general procedure I, the product (4S,6S)—N-(bis(4-methoxyphenyl)(phenyl)methyl)-4-(2-fluoro-5-((2-methoxypyrimidin-5-yl)ethynyl)phenyl)-4-methyl-6-(trifluoromethyl)-5,6-dihydro-4H-1,3-oxazin-2-amine (XVI-53) was obtained after chromatography on silica gel (eluent: heptane-EtOAc; gradient: 0-50% EtOAc) as a light yellow foam (75% yield). MS: m/z=709.3 [M−H]⁻.

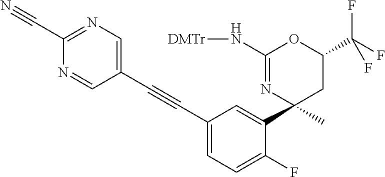

Intermediate (XVI-54): Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 5-((trimethylsilyl)ethynyl)pyrimidine-2-carbonitrile, following general procedure I, the product 5-((3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)ethynyl)pyrimidine-2-carbonitrile (XVI-54) was obtained after chromatography on silica gel (eluent: heptane-EtOAc; gradient: 0-20% EtOAc) as a light yellow oil (22% yield). MS: m/z=704.5 [M−H]⁻.

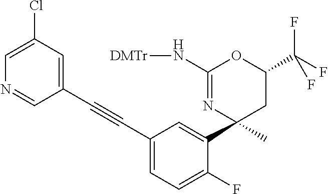

Intermediate (XVI-55): Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 3-chloro-5-((trimethylsilyl)ethynyl)pyridine, following general procedure I, the product (4S,6S)—N-(bis(4-methoxyphenyl)(phenyl)methyl)-4-(5-((5-chloropyridin-3-yl)ethynyl)-2-fluorophenyl)-4-methyl-6-(trifluoromethyl)-5,6-dihydro-4H-1,3-oxazin-2-amine (XVI-55) was obtained after chromatography on silica gel (eluent: heptane-EtOAc; gradient: 0-20% EtOAc) as a light yellow foam (73% yield).

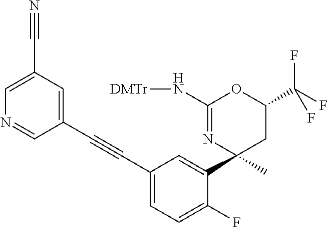

Intermediate (XVI-56): Starting from (4S,6S)—N-(bis(4-methoxyphenyl)(phenyl)methyl)-4-(2-fluoro-5-iodophenyl)-4-methyl-6-(trifluoromethyl)-5,6-dihydro-4H-1,3-oxazin-2-amine (XV) and 5-((trimethylsilyl)ethynyl)nicotinonitrile, following general procedure I, the product 5-((3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)ethynyl)nicotinonitrile (XVI-56) was obtained after chromatography on silica gel (eluent: heptane-EtOAc; gradient: 0-30% EtOAc) as a light yellow foam (73% yield). MS: m/z=703.4 [M−H]⁻.

### Example 49

### 6-((5-((4S,6S)-2-amino-4-methyl-6-(trifluoromethyl)-5,6-dihydro-4H-1,3-oxazin-4-yl)thiophen-3-yl)ethynyl)nicotinonitrile

Starting with 6-((5-((4S,6S)-2-(bis(4-methoxyphenyl)(phenyl)methylamino)-4-methyl-6-(trifluoromethyl)-5,6-dihydro-4H-1,3-oxazin-4-yl)thiophen-3-yl)ethynyl)nicotinonitrile (XXXXII-49) and following general procedure K, the title compound was obtained after chromatography on silica gel (eluent: heptane-EtOAc; gradient: 25-66% EtOAc) as a light yellow foam (73% yield). ¹H NMR (300 MHz, CDCl₃) δ ppm 8.86 (dd, J=0.91, 2.12 Hz, 1H), 7.93 (dd, J=2.22, 8.28 Hz, 1H), 7.56-7.59 (m, 2H), 7.00 (d, J=1.41 Hz, 1H), 4.27 (tdd, J=2.98, 5.98, 12.08 Hz, 1H), 2.36 (dd, J=2.83, 13.52 Hz, 1H), 1.99 (dd, J=12.51, 13.52 Hz, 1H), 1.63 (s, 3H). MS: m/z=391.4 [M+H]⁺.

### Example 50

### (4S,6S)-4-(2-fluoro-5-(pyrimidin-5-ylethynyl)phenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(2-fluoro-5-(pyrimidin-5-ylethynyl)phenyl)-4-methyl-6-(trifluoromethyl)-5,6-dihydro-4H-1,3-oxazin-2-amine (XVI-50) and following general procedure K, the title compound was obtained after chromatography on silica gel (eluent: heptane-EtOAc; gradient: 0-80% EtOAc) as a white foam (78% yield). ¹H NMR (300 MHz, DMSO-d₆) δ ppm 9.20 (s, 1H), 9.03 (s, 2H), 7.55-7.64 (m, 2H), 7.31 (dd, J=8.28, 12.11 Hz, 1H), 6.00 (br s, 2H), 4.23-4.32 (m, 1H), 2.59 (dd, J=2.62, 13.52 Hz, 1H), 1.84 (t, J=13.12 Hz, 1H), 1.50 (s, 3H). MS: m/z=379.6 [M+H]⁺.

### Example 51

### (4S,6S)-4-(5-((6-aminopyridin-3-yl)ethynyl)-2-fluorophenyl)-4-methyl-6-(trifluoromethyl)-5,6-dihydro-4H-1,3-oxazin-2-amine

Starting with (4S,6S)-4-(5-((6-aminopyridin-3-yl)ethynyl)-2-fluorophenyl)-N-(bis(4-methoxyphenyl)(phenyl)methyl)-4-methyl-6-(trifluoromethyl)-5,6-dihydro-4H-1,3-oxazin-2-amine (XVI-51) and following general procedure K, the title compound was obtained after chromatography on silica gel (eluent: heptane-EtOAc; gradient: 0-66% EtOAc) as a white foam (68% yield). ¹H NMR (300 MHz, DMSO-d₆) δ ppm 8.10 (d, J=1.82 Hz, 1H), 7.50 (dd, J=2.32, 8.58 Hz, 1H), 7.41-7.46 (m, 2H), 7.21 (dd, J=9.08, 12.11 Hz, 1H), 6.45 (d, J=8.68 Hz, 1H), 6.40 (s, 2H), 5.97 (br s, 2H), 4.18-4.30 (m, 1H), 2.58 (dd, J=2.52, 13.42 Hz, 1H), 1.82 (t, J=13.02 Hz, 1H), 1.49 (s, 3H). MS: m/z=393.4 [M+H]⁺.

### Example 52

### (4S,6S)-4-(5-((2-aminopyrimidin-5-yl)ethynyl)-2-fluorophenyl)-4-methyl-6-(trifluoromethyl)-5,6-dihydro-4H-1,3-oxazin-2-amine

Starting with (4S,6S)-4-(5-((2-aminopyrimidin-5-yl)ethynyl)-2-fluorophenyl)-N-(bis(4-methoxyphenyl)(phenyl)methyl)-4-methyl-6-(trifluoromethyl)-5,6-dihydro-4H-1,3-oxazin-2-amine (XVI-52) and following general procedure K, the title compound was obtained after chromatography on silica gel (eluent: heptane-EtOAc; gradient: 25-100% EtOAc) as a white solid (88% yield). H NMR (300 MHz, DMSO-d₆) δ ppm 8.42 (s, 2H), 7.45-7.50 (m, 2H), 7.24 (dd, J=8.88, 12.11 Hz, 1H), 7.13 (s, 2H), 5.98 (br s, 2H), 4.19-4.29 (m, 1H), 2.58 (dd, J=2.72, 13.42 Hz, 1H), 1.82 (t, J=13.12 Hz, 1H), 1.49 (s, 3H). MS: m/z=394.6 [M+H]⁺.

### Example 53

### (4S,6S)-4-[2-fluoro-5-[2-(2-methoxypyrimidin-5-yl)ethynyl]phenyl]-4-methyl-6-(trifluoromethyl)-5,6-dihydro-1,3-oxazin-2-amine

Starting with (4S,6S)—N-(bis(4-methoxyphenyl)(phenyl)methyl)-4-(2-fluoro-5-((2-methoxypyrimidin-5-yl)ethynyl)phenyl)-4-methyl-6-(trifluoromethyl)-5,6-dihydro-4H-1,3-oxazin-2-amine (XVI-53) and following general procedure K, the title compound was obtained after chromatography on silica gel (eluent: heptane-EtOAc; gradient: 25-100% EtOAc) as a white foam (88% yield). ¹H NMR (300 MHz, DMSO-d₆) δ ppm 8.84 (s, 2H), 7.52-7.58 (m, 2H), 7.28 (dd, J=8.38, 12.21 Hz, 1H), 6.00 (br s, 2H), 4.21-4.30 (m, 1H), 3.97 (s, 3H), 2.59 (dd, J=2.52, 13.42 Hz, 1H), 1.83 (t, J=13.02 Hz, 1H), 1.49 (s, 3H). MS: m/z=409.6 [M+H]⁺.

### Example 54

### 5-[2-[3-[(4S,6S)-2-amino-4-methyl-6-(trifluoromethyl)-5,6-dihydro-1,3-oxazin-4-yl]-4-fluorophenyl]ethynyl]pyrimidine-2-carbonitrile

Starting with 5-((3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)ethynyl)pyrimidine-2-carbonitrile (XVI-54) and following general procedure K, the title compound was obtained after chromatography on silica gel (eluent: heptane-EtOAc; gradient: 0-80% EtOAc) as a light yellow foam (51% yield). ¹H NMR (300 MHz, CDCl₃) δ ppm 8.91 (s, 2H), 7.62 (dd, J=2.12, 7.77 Hz, 1H), 7.50 (ddd, J=2.22, 4.54, 8.38 Hz, 1H), 7.11 (dd, J=8.38, 11.61 Hz, 1H), 3.96-4.03 (m, 1H), 2.78 (dd, J=2.52, 13.83 Hz, 1H), 1.91 (t, J=13.22 Hz, 1H), 1.64 (d, J=1.01 Hz, 3H). MS: m/z=404.6 [M+H]⁺.

### Example 55

### (4S,6S)-4-(5-((5-chloropyridin-3-yl)ethynyl)-2-fluorophenyl)-4-methyl-6-(trifluoromethyl)-5,6-dihydro-4H-1,3-oxazin-2-amine

Starting with (4S,6S)—N-(bis(4-methoxyphenyl)(phenyl)methyl)-4-(5-((5-chloropyridin-3-yl)ethynyl)-2-fluorophenyl)-4-methyl-6-(trifluoromethyl)-5,6-dihydro-4H-1,3-oxazin-2-amine (XVI-55) and following general procedure K, the title compound was obtained after chromatography on silica gel (eluent: heptane-EtOAc; gradient: 0-80% EtOAc) as a white foam (82% yield). ¹H NMR (300 MHz, DMSO-d₆) δ ppm 8.72 (d, J=1.61 Hz, 1H), 8.66 (d, J=2.42 Hz, 1H), 8.21 (dd, J=1.82, 2.42 Hz, 1H), 7.55-7.62 (m, 2H), 7.30 (dd, J=8.17, 12.21 Hz, 1H), 6.00 (br s, 2H), 4.24-4.31 (m, 1H), 2.59 (dd, J=2.42, 13.32 Hz, 1H), 1.83 (t, J=12.92 Hz, 1H), 1.50 (s, 3H). MS: m/z=412.5 [M+H]⁺.

### Example 56

### 5-[2-[3-[(4S,6S)-2-amino-4-methyl-6-(trifluoromethyl)-5,6-dihydro-1,3-oxazin-4-yl]-4-fluorophenyl]ethynyl]pyridine-3-carbonitrile

Starting with 5-((3-((4S,6S)-2-(bis(4-methoxyphenyl)(phenyl)methylamino)-4-methyl-6-(trifluoromethyl)-5,6-dihydro-4H-1,3-oxazin-4-yl)-4-fluorophenyl)ethynyl)nicotinonitrile (XVI-56) and following general procedure K, the title compound was obtained after chromatography on silica gel (eluent: heptane-EtOAc; gradient: 0-80% EtOAc) as a white foam (83% yield). ¹H NMR (300 MHz, DMSO-d₆) δ ppm 9.03 (d, J=2.02 Hz, 2H), 8.59 (t, J=1.92 Hz, 1H), 7.56-7.63 (m, 2H), 7.32 (dd, J=8.28, 12.11 Hz, 1H), 6.01 (br s, 2H), 4.22-4.33 (m, 1H), 2.59 (dd, J=2.32, 13.22 Hz, 1H), 1.84 (t, J=13.02 Hz, 1H), 1.50 (s, 3H). MS: m/z=403.2 [M+H]⁺.

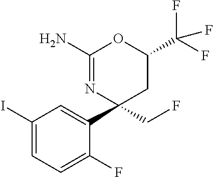 [SMILES: NC1=N[C@](CF)(c2cc(I)ccc2F)C[C@@H](C(F)(F)F)O1]

Intermediate (XIV-57): (4S,6S)-4-(5-amino-2-fluorophenyl)-4-(fluoromethyl)-6-(trifluoromethyl)-5,6-dihydro-4H-1,3-oxazin-2-amine (CAS#1432511-77-7, 100 mg) was dissolved at room temperature under argon in acetonitrile (4.2 ml) and 1 M aqueous HCl (3.23 ml) and cooled to −10° C. Sodium nitrite (24.5 mg) and cooled water (1.7 ml) were added. After stirring for 30 min at −10° C. potassium iodide (268 mg) was added and stirring was continued for 4 h at a temperature between −10 and −5° C. After addition of saturated aqueous sodium bicarbonate solution, the mixture was extracted twice with EtOAc. The org. layers were washed with water, 0.1 M aqueous sodium thiosulfate, dried over sodium sulphate, evaporated and dried at the high vacuo. The crude material was purified by preparative TLC (silica gel, 2.0 mm, EtOAc) to give (4S,6S)-4-(2-fluoro-5-iodophenyl)-4-(fluoromethyl)-6-(trifluoromethyl)-5,6-dihydro-4H-1,3-oxazin-2-amine (Intermediate (XIV-57), 133 mg) as a light yellow solid. MS: m/z=421.1 [M+H]⁺.

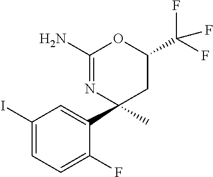 [SMILES: C[C@@]1(c2cc(I)ccc2F)C[C@@H](C(F)(F)F)OC(N)=N1]

Alternative synthesis of Intermediate (XIV): A solution of (4S,6S)-4-(2-fluorophenyl)-4-methyl-6-(trifluoromethyl)-5,6-dihydro-4H-1,3-oxazin-2-amine (CAS#1432511-74-4, 175 mg) in dichloromethane (8.8 ml) was cooled under argon in an ice bath. Trifluoromethanesulfonic acid (1.9 g) was added and the solution was allowed to warm to room temperature. N-Iodosuccinimide (171 mg) was added at once and the mixture was stirred for 3 h. The dark purple mixture was added dropwise to a sat. aqueous NaHCO3 solution (50 ml). The aqueous layer was separated and extracted once more with dichloromethane. The organic layers were washed with 0.1M aqueous sodiumthiosulphate solution, dried over MgSO4, filtered and concentrated in vacuo. The crude material was purified by flash chromatography (silica gel, 0% to 100% EtOAc in n-heptane) to give (4S,6S)-4-(2-fluoro-5-iodophenyl)-4-methyl-6-(trifluoromethyl)-5,6-dihydro-4H-1,3-oxazin-2-amine (Intermediate (XIV), 216 mg) as an off-white solid. MS: m/z=403.1 [M+H]⁺.

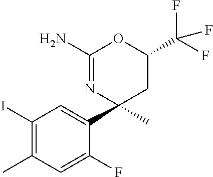 [SMILES: Cc1cc(F)c([C@]2(C)C[C@@H](C(F)(F)F)OC(N)=N2)cc1I]

Intermediate (XIV-69):

Step 1: A stirred solution of 1-bromo-2-fluoro-4-methylbenzene (26.8 g) in toluene (600 ml) and THF (600 ml) was cooled under argon to −100° C. Within 10 min, a 1.6 M solution of n-butyllithium in hexane (79.7 ml) was added dropwise at a rate that keeps the temperature between −95 and −98° C. After additional 15 min stirring at −95° C. a mixture of 3-methyl-5-(trifluoromethyl)-4,5-dihydroisoxazole (10.84 g) in toluene (60.0 ml) and THF (11.0 ml) mixed with boron trifluoride diethyl etherate (20.1 g) was added within 3-5 min at a rate that keeps the temperature below −90° C. Stirring was continued for 30 min at a temperature between −85 and −90° C. 60 ml saturated aqueous ammonium chloride solution, 100 ml water and 200 ml EtOAc were added and the temperature was raised to 0° C. The layers were separated, the aqueous layer was extracted once more with EtOAc. The org. layers were washed with water/brine, dried over sodium sulphate, evaporated and dried at the high vacuo for 10 min. The crude material was purified by flash chromatography (silica gel, 0% to 25% EtOAc in heptane) to give (3SR,5SR)-3-(2-fluoro-4-methylphenyl)-3-methyl-5-(trifluoromethyl)-1,2-oxazolidine (10.2 g) as a yellow liquid. MS: m/z=264.2 [M+H]⁺.

Step 2: (3SR,5SR)-3-(2-fluoro-4-methylphenyl)-3-methyl-5-(trifluoromethyl)-1,2-oxazolidine (9.97 g) was separated into the enantiomers by chiral chromatography (Chiralpak AD, n-heptane/EtOH 95:5) to give (−)-(3R,5R)-3-(2-fluoro-4-methylphenyl)-3-methyl-5-(trifluoromethyl)isoxazolidine (3.34 g), MS: m/z=264.2 [M+H]⁺, and (+)-(3S,5S)-3-(2-fluoro-4-methylphenyl)-3-methyl-5-(trifluoromethyl)isoxazolidine (3.26 g), MS: m/z=264.2 [M+H]⁺.

Step 3: To a solution of (3S,5S)-3-(2-fluoro-4-methylphenyl)-3-methyl-5-(trifluoromethyl)isoxazolidine (333 mg) in ethanol (7.4 ml) was added at r.t. under argon ammonium formate (638 mg) followed by palladium (10% on carbon, 67.3 mg). The suspension was stirred at 25° C. for 2 h. The catalyst was filtered off, washed three times with ethanol and the filtrate was evaporated. The semi-solid residue was treated with saturated aqueous NaHCO₃ solution and extracted three times with EtOAc. The organic layers were washed once with brine, dried and evaporated to give (2S,4S)-4-amino-1,1,1-trifluoro-4-(2-fluoro-4-methylphenyl)pentan-2-ol (333 mg) as a colorless solid. MS: m/z=266.2 [M+H]⁺.

Step 4: (2S,4S)-4-amino-1,1,1-trifluoro-4-(2-fluoro-4-methylphenyl)pentan-2-ol (320 mg) was dissolved under argon in THF (6.8 ml). After addition of benzoyl isothiocyanate (197 mg) the reaction mixture was stirred for 5 h at room temperature. The reaction mixture was concentrated to dryness and re-dissolved in acetonitrile (6.8 ml). After addition of triethylamine (12.2 mg) and

N,N′-dicyclohexylcarbodiimide (274 mg) the mixture was stirred 4 days at room temperature. The solid was filtered off, the filtrate was evaporated and purified by chromatography (silica gel, 0% to 100% EtOAc in n-heptane) to give N-((4S,6S)-4-(2-fluoro-4-methylphenyl)-4-methyl-6-(trifluoromethyl)-5,6-dihydro-4H-1,3-oxazin-2-yl)benzamide (380 mg) as a colorless amorphous solid. MS: m/z=395.3 [M+H]⁺.

Step 5: N-((4S,6S)-4-(2-fluoro-4-methylphenyl)-4-methyl-6-(trifluoromethyl)-5,6-dihydro-4H-1,3-oxazin-2-yl)benzamide (4.17 g) was combined at r.t. under argon with THF (53.0 ml) and Methanol (53.0 ml). Sodium hydroxide (2 M in water, 31.7 ml) was added and the reaction mixture was stirred for 19 h at 70° C. The reaction mixture was cooled down to r.t. and was evaporated. Water was added and extracted with EtOAc. The organic layers were combined, dried over MgSO₄ and evaporated. The crude material was purified by flash chromatography (silica gel, 0% to 100% EtOAc in heptane) to give (4S,6S)-4-(2-fluoro-4-methylphenyl)-4-methyl-6-(trifluoromethyl)-5,6-dihydro-4H-1,3-oxazin-2-amine (1.71 g) as a colorless semisolid. MS: m/z=291.2 [M+H]⁺.

Step 6: A solution of (4S,6S)-4-(2-fluoro-4-methylphenyl)-4-methyl-6-(trifluoromethyl)-5,6-dihydro-4H-1,3-oxazin-2-amine (1.7 g) in dichloromethane (85.4 ml) was cooled under argon in an ice bath. Trifluoromethanesulfonic acid (17.6 g) was added and the solution was allowed to warm to room temperature. N-Iodosuccinimide (1.58 g) was added at once and the mixture was stirred 2 days at rt. The mixture was added dropwise to a sat. aqueous NaHCO₃ solution (250 ml).

The aqueous layer was separated and extracted once more with dichloromethane. The organic layers were washed with 0.1M sodiumthiosulphate solution, dried over MgSO₄, filtered and concentrated in vacuo. The crude material was purified by flash chromatography (silica gel, 0% to 100% EtOAc in n-heptane) to give (4S,6S)-4-(2-fluoro-5-iodo-4-methylphenyl)-4-methyl-6-(trifluoromethyl)-5,6-dihydro-4H-1,3-oxazin-2-amine (Intermediate (XIV-69), 1.95 g) as an off-white solid. MS: m/z=417.1 [M+H]⁺.

### Example 57

### 6-((3-((4S,6S)-2-Amino-4-(fluoromethyl)-6-(trifluoromethyl)-5,6-dihydro-4H-1,3-oxazin-4-yl)-4-fluorophenyl)ethynyl)-5-methoxynicotinonitrile

(4S,6S)-4-(2-Fluoro-5-iodophenyl)-4-(fluoromethyl)-6-(trifluoromethyl)-5,6-dihydro-4H-1,3-oxazin-2-amine (Intermediate (XIV-57), 60 mg), 5-methoxy-6-((trimethylsilyl)ethynyl)nicotinonitrile (42.8 mg), bis(triphenylphosphine)palladiumdichloride (7.02 mg), triethylamine (72.3 mg), copper(I) iodide (1.09 mg) and tetramethylammonium fluoride (18.6 mg) were combined under argon with THF (1.4 ml) and the reaction mixture was stirred in a sealed tube at 65° C. for 18 h. The reaction mixture was diluted with EtOAc, filtered through a glassfibre filter, evaporated and purified by chromatography (silica gel, 0% to 100% EtOAc in heptane) followed by preparative TLC purification (silica gel, 1.0 mm, 1:1 Heptane/AcOEt) to give 6-((3-((4S,6S)-2-amino-4-(fluoromethyl)-6-(trifluoromethyl)-5,6-dihydro-4H-1,3-oxazin-4-yl)-4-fluorophenyl)ethynyl)-5-methoxynicotinonitrile (46 mg) as a light brown solid. MS: m/z=451.2 [M+H]⁺.

### Example 58

### (4S,6S)-4-(5-((1-Ethyl-1H-pyrazol-4-yl)ethynyl)-2-fluorophenyl)-4-(fluoromethyl)-6-(trifluoromethyl)-5,6-dihydro-4H-1,3-oxazin-2-amine

In analogy to Example 57, (4S,6S)-4-(2-Fluoro-5-iodophenyl)-4-(fluoromethyl)-6-(trifluoromethyl)-5,6-dihydro-4H-1,3-oxazin-2-amine (Intermediate (XIV-57) was coupled with 1-ethyl-4-((trimethylsilyl)ethynyl)-1H-pyrazole (prepared by coupling of 1-ethyl-4-iodo-1H-pyrazole with ethynyltrimethylsilane) to give (4S,6S)-4-(5-((1-ethyl-1H-pyrazol-4-yl)ethynyl)-2-fluorophenyl)-4-(fluoromethyl)-6-(trifluoromethyl)-5,6-dihydro-4H-1,3-oxazin-2-amine as an off-white amorphous solid. MS: m/z=413.2 [M+H]⁺.

### Example 59

### 6-((3-((4S,6S)-2-Amino-4-(fluoromethyl)-6-(trifluoromethyl)-5,6-dihydro-4H-1,3-oxazin-4-yl)-4-fluorophenyl)ethynyl)-5-chloronicotinonitrile

In analogy to Example 57, (4S,6S)-4-(2-Fluoro-5-iodophenyl)-4-(fluoromethyl)-6-(trifluoromethyl)-5,6-dihydro-4H-1,3-oxazin-2-amine (Intermediate (XIV-57) was coupled with 5-chloro-6-((trimethylsilyl)ethynyl)nicotinonitrile (prepared by coupling of 5,6-dichloronicotinonitrile with ethynyltrimethylsilane) to give 6-((3-((4S,6S)-2-amino-4-(fluoromethyl)-6-(trifluoromethyl)-5,6-dihydro-4H-1,3-oxazin-4-yl)-4-fluorophenyl)ethynyl)-5-chloronicotinonitrile as light brown solid. MS: m/z=455.2 [M+H]⁺.

### Example 60

### 4-((3-((4S,6S)-2-Amino-4-(fluoromethyl)-6-(trifluoromethyl)-5,6-dihydro-4H-1,3-oxazin-4-yl)-4-fluorophenyl)ethynyl)benzonitrile

In analogy to Example 57, (4S,6S)-4-(2-Fluoro-5-iodophenyl)-4-(fluoromethyl)-6-(trifluoromethyl)-5,6-dihydro-4H-1,3-oxazin-2-amine (Intermediate (XIV-57) was coupled with 4-ethynylbenzonitrile to give 4-((3-((4S,6S)-2-amino-4-(fluoromethyl)-6-(trifluoromethyl)-5,6-dihydro-4H-1,3-oxazin-4-yl)-4-fluorophenyl)ethynyl)benzonitrile as yellow solid. MS: m/z=420.2 [M+H]⁺.

### Example 61

### (4S,6S)-4-(5-((5-Chloropyridin-3-yl)ethynyl)-2-fluorophenyl)-4-(fluoromethyl)-6-(trifluoromethyl)-5,6-dihydro-4H-1,3-oxazin-2-amine

In analogy to Example 57, (4S,6S)-4-(2-Fluoro-5-iodophenyl)-4-(fluoromethyl)-6-(trifluoromethyl)-5,6-dihydro-4H-1,3-oxazin-2-amine (Intermediate (XIV-57) was coupled with 3-chloro-5-((trimethylsilyl)ethynyl)pyridine (prepared by coupling of 3-chloro-5-iodopyridine with ethynyltrimethylsilane) to give (4S,6S)-4-(5-((5-chloropyridin-3-yl)ethynyl)-2-fluorophenyl)-4-(fluoromethyl)-6-(trifluoromethyl)-5,6-dihydro-4H-1,3-oxazin-2-amine as off-white solid. MS: m/z=430.2 [M+H]⁺.

### Example 62

### 6-((3-((4S,6S)-2-Amino-4-(fluoromethyl)-6-(trifluoromethyl)-5,6-dihydro-4H-1,3-oxazin-4-yl)-4-fluorophenyl)ethynyl)nicotinonitrile

In analogy to Example 57, (4S,6S)-4-(2-Fluoro-5-iodophenyl)-4-(fluoromethyl)-6-(trifluoromethyl)-5,6-dihydro-4H-1,3-oxazin-2-amine (Intermediate (XIV-57) was coupled with 6-((trimethylsilyl)ethynyl)nicotinonitrile to give 6-((3-((4S,6S)-2-amino-4-(fluoromethyl)-6-(trifluoromethyl)-5,6-dihydro-4H-1,3-oxazin-4-yl)-4-fluorophenyl)ethynyl)nicotinonitrile as off-white solid. MS: m/z=421.2 [M+H]⁺.

### Example 63

### (4S,6S)-4-[5-[2-(5-Chloropyrimidin-2-yl)ethynyl]-2-fluorophenyl]-4-(fluoromethyl)-6-(trifluoromethyl)-5,6-dihydro-1,3-oxazin-2-amine

In analogy to Example 57, (4S,6S)-4-(2-Fluoro-5-iodophenyl)-4-(fluoromethyl)-6-(trifluoromethyl)-5,6-dihydro-4H-1,3-oxazin-2-amine (Intermediate (XIV-57) was coupled with 5-chloro-2-((trimethylsilyl)ethynyl)pyrimidine to give (4S,6S)-4-[5-[2-(5-chloropyrimidin-2-yl)ethynyl]-2-fluorophenyl]-4-(fluoromethyl)-6-(trifluoromethyl)-5,6-dihydro-1,3-oxazin-2-amine as off-white solid. MS: m/z=431.2 [M+H]⁺.

### Example 64

### (4S,6S)-4-(2-Fluoro-5-((3-methylisothiazol-5-yl)ethynyl)phenyl)-4-(fluoromethyl)-6-(trifluoromethyl)-5,6-dihydro-4H-1,3-oxazin-2-amine

In analogy to Example 57, (4S,6S)-4-(2-Fluoro-5-iodophenyl)-4-(fluoromethyl)-6-(trifluoromethyl)-5,6-dihydro-4H-1,3-oxazin-2-amine (Intermediate (XIV-57) was coupled with 3-methyl-5-((trimethylsilyl)ethynyl)isothiazole (prepared by coupling of 5-iodo-3-methylisothiazole with ethynyltrimethylsilane) to give (4S,6S)-4-(2-fluoro-5-((3-methylisothiazol-5-yl)ethynyl)phenyl)-4-(fluoromethyl)-6-(trifluoromethyl)-5,6-dihydro-4H-1,3-oxazin-2-amine as colorless solid. MS: m/z=416.2 [M+H]⁺.

### Example 65

### (4S,6S)-4-(2-Fluoro-5-((3-methylisothiazol-5-yl)ethynyl)phenyl)-4-methyl-6-(trifluoromethyl)-5,6-dihydro-4H-1,3-oxazin-2-amine

In analogy to Example 57, (4S,6S)-4-(2-fluoro-5-iodophenyl)-4-methyl-6-(trifluoromethyl)-5,6-dihydro-4H-1,3-oxazin-2-amine (Intermediate (XIV) was coupled with 3-methyl-5-((trimethylsilyl)ethynyl)isothiazole to give (4S,6S)-4-(2-fluoro-5-((3-methylisothiazol-5-yl)ethynyl)phenyl)-4-methyl-6-(trifluoromethyl)-5,6-dihydro-4H-1,3-oxazin-2-amine as off-white solid. MS: m/z=398.2 [M+H]⁺.

### Example 66

### 4-((3-((4S,6S)-2-Amino-4-methyl-6-(trifluoromethyl)-5,6-dihydro-4H-1,3-oxazin-4-yl)-4-fluorophenyl)ethynyl)benzonitrile

In analogy to Example 57, (4S,6S)-4-(2-fluoro-5-iodophenyl)-4-methyl-6-(trifluoromethyl)-5,6-dihydro-4H-1,3-oxazin-2-amine (Intermediate (XIV) was coupled with 4-ethynylbenzonitrile to give 4-((3-((4S,6S)-2-amino-4-methyl-6-(trifluoromethyl)-5,6-dihydro-4H-1,3-oxazin-4-yl)-4-fluorophenyl)ethynyl)benzonitrile as off-white solid. MS: m/z=402.2 [M+H]⁺.

### Example 67

### 2-((3-((4S,6S)-2-Amino-4-methyl-6-(trifluoromethyl)-5,6-dihydro-4H-1,3-oxazin-4-yl)-4-fluorophenyl)ethynyl)thiazole-5-carbonitrile

In analogy to Example 57, (4S,6S)-4-(2-fluoro-5-iodophenyl)-4-methyl-6-(trifluoromethyl)-5,6-dihydro-4H-1,3-oxazin-2-amine (Intermediate (XIV) was coupled with 2-((trimethylsilyl)ethynyl)thiazole-5-carbonitrile (prepared by coupling of 2-chlorothiazole-5-carbonitrile with ethynyltrimethylsilane) to give 2-((3-((4S,6S)-2-amino-4-methyl-6-(trifluoromethyl)-5,6-dihydro-4H-1,3-oxazin-4-yl)-4-fluorophenyl)ethynyl)thiazole-5-carbonitrile as off-white solid. MS: m/z=409.2 [M+H]⁺.

### Example 68

### (4S,6S)-4-(5-((4-chloro-1-(difluoromethyl)-1H-pyrazol-3-yl)ethynyl)-2-fluorophenyl)-4-methyl-6-(trifluoromethyl)-5,6-dihydro-4H-1,3-oxazin-2-amine

Step 1: To a solution of 4-chloro-1-(difluoromethyl)-1H-pyrazole-3-carbaldehyde (CAS#1357098-11-3, 500 mg) and dimethyl 1-diazo-2-oxopropylphosphonate (638 mg) in methanol (12 ml) at 0° C. was added K₂CO₃ (842 mg). The ice bath was removed and the mixture was allowed to warm to room temperature and stirred for 3 h. The mixture was poured into sat. aqueous NaCl solution and extracted with EtOAc. The organic layer was dried over Na₂SO₄. Removal of the solvent in vacuum left a light brown oil. The crude material was purified by flash chromatography (silica gel, 0% to 40% EtOAc in heptane) to give 4-chloro-1-(difluoromethyl)-3-ethynyl-1H-pyrazole (265 mg) as a colorless liquid.

Step 2: In analogy to Example 57, (4S,6S)-4-(2-fluoro-5-iodophenyl)-4-methyl-6-(trifluoromethyl)-5,6-dihydro-4H-1,3-oxazin-2-amine (Intermediate (XIV) was coupled with 4-chloro-1-(difluoromethyl)-3-ethynyl-1H-pyrazole to give (4S,6S)-4-(5-((4-chloro-1-(difluoromethyl)-1H-pyrazol-3-yl)ethynyl)-2-fluorophenyl)-4-methyl-6-(trifluoromethyl)-5,6-dihydro-4H-1,3-oxazin-2-amine as light brown solid. MS: m/z=451.2 [M+H]⁺.

### Example 69

### 6-((5-((4S,6S)-2-Amino-4-methyl-6-(trifluoromethyl)-5,6-dihydro-4H-1,3-oxazin-4-yl)-4-fluoro-2-methylphenyl)ethynyl)-5-chloronicotinonitrile

In analogy to Example 57, (4S,6S)-4-(2-fluoro-5-iodo-4-methylphenyl)-4-methyl-6-(trifluoromethyl)-5,6-dihydro-4H-1,3-oxazin-2-amine (Intermediate (XIV-69) was coupled with 5-chloro-6-((trimethylsilyl)ethynyl)nicotinonitrile to give 6-((5-((4S,6S)-2-amino-4-methyl-6-(trifluoromethyl)-5,6-dihydro-4H-1,3-oxazin-4-yl)-4-fluoro-2-methylphenyl)ethynyl)-5-chloronicotinonitrile as colorless solid. MS: m/z=451.2 [M+H]⁺.

### Example 70

### 6-((5-((4S,6S)-2-Amino-4-methyl-6-(trifluoromethyl)-5,6-dihydro-4H-1,3-oxazin-4-yl)-4-fluoro-2-methylphenyl)ethynyl)nicotinonitrile

In analogy to Example 57, (4S,6S)-4-(2-fluoro-5-iodo-4-methylphenyl)-4-methyl-6-(trifluoromethyl)-5,6-dihydro-4H-1,3-oxazin-2-amine (Intermediate (XIV-69) was coupled with 6-((trimethylsilyl)ethynyl)nicotinonitrile to give 6-((5-((4S,6S)-2-amino-4-methyl-6-(trifluoromethyl)-5,6-dihydro-4H-1,3-oxazin-4-yl)-4-fluoro-2-methylphenyl)ethynyl)nicotinonitrile as off-white solid. MS: m/z=417.2 [M+H]⁺.

### Example 71

### (4S,6S)-4-(5-((4-Chloro-1-(difluoromethyl)-1H-pyrazol-3-yl)ethynyl)-2-fluoro-4-methylphenyl)-4-methyl-6-(trifluoromethyl)-5,6-dihydro-4H-1,3-oxazin-2-amine

In analogy to Example 57, (4S,6S)-4-(2-fluoro-5-iodo-4-methylphenyl)-4-methyl-6-(trifluoromethyl)-5,6-dihydro-4H-1,3-oxazin-2-amine (Intermediate (XIV-69) was coupled with 4-chloro-1-(difluoromethyl)-3-ethynyl-1H-pyrazole to give (4S,6S)-4-(5-((4-chloro-1-(difluoromethyl)-1H-pyrazol-3-yl)ethynyl)-2-fluoro-4-methylphenyl)-4-methyl-6-(trifluoromethyl)-5,6-dihydro-4H-1,3-oxazin-2-amine as off-white solid. MS: m/z=465.2 [M+H]⁺.

### Example 72

### (4S,6S)-4-(5-((5-Chloropyrimidin-2-yl)ethynyl)-2-fluoro-4-methylphenyl)-4-methyl-6-(trifluoromethyl)-5,6-dihydro-4H-1,3-oxazin-2-amine

In analogy to Example 57, (4S,6S)-4-(2-fluoro-5-iodo-4-methylphenyl)-4-methyl-6-(trifluoromethyl)-5,6-dihydro-4H-1,3-oxazin-2-amine (Intermediate (XIV-69) was coupled with 5-chloro-2-((trimethylsilyl)ethynyl)pyrimidine to give (4S,6S)-4-(5-((5-chloropyrimidin-2-yl)ethynyl)-2-fluoro-4-methylphenyl)-4-methyl-6-(trifluoromethyl)-5,6-dihydro-4H-1,3-oxazin-2-amine as off-white solid. MS: m/z=427.2 [M+H]⁺.

### Example 73

### (4S,6S)-4-(2-Fluoro-5-(4-fluoropyridin-3-yl)-4-methylphenyl)-4-methyl-6-(trifluoromethyl)-5,6-dihydro-4H-1,3-oxazin-2-amine

(4S,6S)-4-(2-Fluoro-5-iodo-4-methylphenyl)-4-methyl-6-(trifluoromethyl)-5,6-dihydro-4H-1,3-oxazin-2-amine (Intermediate (XIV-69), 80 mg), 4-fluoro-3-(4,4,5,5-tetramethyl-1,3,2-dioxaborolan-2-yl)pyridine (55.7 mg) and cesium carbonate (188 mg) were dissolved in THF (2.2 ml) and water (1.1 ml). Argon was bubbled through the solution. After addition of [1,1′-bis(diphenylphosphino)ferrocene]dichloropalladium(II) (7.03 mg) the reaction mixture was stirred 6 h at 90° C. in a sealed tube. The reaction mixture was filtered through glass fiber paper, the filter cake was washed with water and EOAc, the layers were separated and the aqueous layer was extracted with EtOAc. The org. layers were evaporated and concentrated to dryness. The crude material was purified by preparative TLC (silica gel, 2.0 mm, EtOAc) followed by a second preparative TLC purification (silica gel, 1.0 mm, 9:1 dichloromethane/MeOH) to give (4S,6S)-4-(2-fluoro-5-(4-fluoropyridin-3-yl)-4-methylphenyl)-4-methyl-6-(trifluoromethyl)-5,6-dihydro-4H-1,3-oxazin-2-amine (18.2 mg) as off-white solid. MS: m/z=386.2 [M+H]⁺.

### Example 74

### 5′-((4S,6S)-2-Amino-4-methyl-6-(trifluoromethyl)-5,6-dihydro-4H-1,3-oxazin-4-yl)-2,4′-difluoro-2′-methylbiphenyl-3-carbonitrile

In analogy to Example 73, (4S,6S)-4-(2-fluoro-5-iodo-4-methylphenyl)-4-methyl-6-(trifluoromethyl)-5,6-dihydro-4H-1,3-oxazin-2-amine (Intermediate (XIV-69)) was coupled with 3-cyano-2-fluorophenylboronic acid to give 5′-((4S,6S)-2-amino-4-methyl-6-(trifluoromethyl)-5,6-dihydro-4H-1,3-oxazin-4-yl)-2,4′-difluoro-2′-methylbiphenyl-3-carbonitrile as off-white solid. MS: m/z=410.2 [M+H]⁺.

### Example 75

### (4S,6S)-4-(5-((4-Chlorophenyl)ethynyl)-2-fluorophenyl)-4-methyl-6-(trifluoromethyl)-5,6-dihydro-4H-1,3-oxazin-2-amine

In analogy to Example 57, (4S,6S)-4-(2-fluoro-5-iodophenyl)-4-methyl-6-(trifluoromethyl)-5,6-dihydro-4H-1,3-oxazin-2-amine (Intermediate (XIV) was coupled with 1-chloro-4-ethynylbenzene to give (4S,6S)-4-(5-((4-chlorophenyl)ethynyl)-2-fluorophenyl)-4-methyl-6-(trifluoromethyl)-5,6-dihydro-4H-1,3-oxazin-2-amine as off-white solid. MS: m/z=411.2 [M+H]⁺.

### Example 76

### (4S,6S)-4-[5-[2-(6-Chloropyridin-3-yl)ethynyl]-2-fluorophenyl]-4-methyl-6-(trifluoromethyl)-5,6-dihydro-1,3-oxazin-2-amine

In analogy to Example 57, (4S,6S)-4-(2-fluoro-5-iodophenyl)-4-methyl-6-(trifluoromethyl)-5,6-dihydro-4H-1,3-oxazin-2-amine (Intermediate (XIV) was coupled with 2-chloro-5-((trimethylsilyl)ethynyl)pyridine (CAS#263012-81-3) to give (4S,6S)-4-[5-[2-(6-chloropyridin-3-yl)ethynyl]-2-fluorophenyl]-4-methyl-6-(trifluoromethyl)-5,6-dihydro-1,3-oxazin-2-amine as off-white solid. MS: m/z=412.2 [M+H]⁺.

### Example 77

### 6-(4-(3-((4S,6S)-2-Amino-4-methyl-6-(trifluoromethyl)-5,6-dihydro-4H-1,3-oxazin-4-yl)-4-fluorophenyl)-1H-1,2,3-triazol-1-yl)nicotinonitrile

Step 1: (4S,6S)-4-(2-Fluoro-5-iodophenyl)-4-methyl-6-(trifluoromethyl)-5,6-dihydro-4H-1,3-oxazin-2-amine (Intermediate (XIV, 500 mg), trimethylsilylacetylene (244 mg), bis(triphenylphosphine)palladium (II) chloride (61.1 mg), copper (I) iodide (9.5 mg) and triethylamine (377 mg) were combined under argon with THF (12.3 ml) and stirred for 5.5 h at 65° C. in a sealed tube. The reaction mixture was diluted with EtOAc, filtered through a glassfiber filter and concentrated to dryness. The crude material was purified by flash chromatography (silica gel, 0% to 100% EtOAc in n-heptane) to give (4S,6S)-4-(2-fluoro-5-((trimethylsilyl)ethynyl)phenyl)-4-methyl-6-(trifluoromethyl)-5,6-dihydro-4H-1,3-oxazin-2-amine (446 mg) as a light brown solid which could be further purified by stirring of a dichloromethane solution of the compound with 3-mercaptopropyl ethyl sulfide silica (90A, low-cross linking, Phosphonics SPM32f, Strem), filtration and concentration to dryness. MS: m/z=373.2 [M+H]⁺.

Step 2: A solution of (4S,6S)-4-(2-Fluoro-5-((trimethylsilyl)ethynyl)phenyl)-4-methyl-6-(trifluoromethyl)-5,6-dihydro-4H-1,3-oxazin-2-amine (100 mg) in dichloromethane (1.14 ml) was cooled to 0° C. Tetrabutylammonium fluoride (1 M in THF, 0.295 ml) was added and the mixture was stirred at 0° C. for 1 h. The reaction mixture was diluted with dichloromethane and washed with water. The combined organic layers were dried over sodium sulphate, filtered and concentrated to dryness to give (4S,6S)-4-(5-ethynyl-2-fluorophenyl)-4-methyl-6-(trifluoromethyl)-5,6-dihydro-4H-1,3-oxazin-2-amine (71 mg, not totally pure) which was used in the next step without further purification. MS: m/z=301.1 [M+H]⁺.

Step 3:

(4S,6S)-4-(5-Ethynyl-2-fluorophenyl)-4-methyl-6-(trifluoromethyl)-5,6-dihydro-4H-1,3-oxazin-2-amine (65 mg) was combined with toluene (1.78 ml) at room temperature under argon, then 6-azidonicotinonitrile (31.4 mg) and copper(I) trifluoromethanesulfonate benzene complex (10.9 mg) were added and stirred 4 days at room temperature. EtOAc was added and the mixture was filtered through a glassfiber filter and concentrated to dryness. The crude material was purified by preparative TLC (silica gel, 2.0 mm, AcOEt) followed by flash chromatography (silica gel, 25 g, 0% to 100% EtOAc in n-heptane) followed by preparative TLC (silica gel, 1.0 mm, 9:1 dichloromethane/MeOH) to give 6-(4-(3-((4S,6S)-2-amino-4-methyl-6-(trifluoromethyl)-5,6-dihydro-4H-1,3-oxazin-4-yl)-4-fluorophenyl)-1H-1,2,3-triazol-1-yl)nicotinonitrile (16.6 mg) as off-white solid. MS: m/z=446.2 [M+H]⁺.
¹Hardy et al., The amyloid hypothesis of Alzheimer's disease: progress and problems on the road to therapeutics, Science. 2002 Jul. 19; 297(5580):353-6
²Selkoe, Cell biology of the amyloid beta-protein precursor and the mechanism of Alzheimer's disease, Annu Rev Cell Biol. 1994; 10:373-403
³Vassar et al., Beta-secretase cleavage of Alzheimer's amyloid precursor protein by the transmembrane aspartic protease BACE, Science. 1999 Oct. 22; 286(5440):735
⁴Luo et al., Mice deficient in BACE1, the Alzheimer's beta-secretase, have normal phenotype and abolished beta-amyloid generation, Nat Neurosci. 2001 March; 4(3):231
⁵Roberds et al., BACE knockout mice are healthy despite lacking the primary beta-secretase activity in brain: implications for Alzheimer's disease therapeutics, Hum Mol Genet. 2001 Jun. 1; 10(12):1317-24
⁶McConlogue et al., Partial reduction of BACE1 has dramatic effects on Alzheimer plaque and synaptic pathology in APP Transgenic Mice. J Biol Chem. 2007 Sep. 7; 282(36):26326
⁷Vattemi G. et al., Lancet. 2001 Dec. 8; 358(9297):1962-4
⁸Barbiero L. et al., Exp Neurol. 2003 August; 182(2):335-45
⁹Sugimoto I. et al., J Biol Chem. 2007 Nov. 30; 282(48):34896-903
¹⁰Desnues B. et al., Clin Vaccine Immunol. 2006 February; 13(2):170-8
¹¹Gatchel J. R. et al., Proc Natl Acad Sci USA 2008 Jan. 29; 105(4):1291-6
¹²Greenberg S. A. et al., Ann Neurol. 2005 May; 57(5):664-78
¹³Greenberg S. A. et al., Neurol 2005 May; 57(5):664-78
¹⁴Lagos D. et al, Blood, 2007 Feb. 15; 109(4):1550-8
¹⁵E-MEXP-2576, http://www.ebi.ac.uk/microarray-as/aer/result?queryFor=PhysicalArrayDesign&aAccession=A-MEXP-258
¹⁶Ungethuem U. et al, GSE2053
¹⁷Koistinen H. et al., Muscle Nerve. 2006 October; 34(4):444-50
¹⁸Li Q. X. et al, Aging Cell. 2006 April; 5(2):153-65
¹⁹Kim Y. J. et al., Neurobiol Dis. 2006 May; 22(2):346-56. Epub 2006 Jan. 19
²⁰Hodges A. et al., Hum Mol Genet. 2006 Mar. 15; 15(6):965-77. Epub 2006 Feb. 8
²¹Kihara Y. et al, Proc Natl Acad Sci USA. 2009 Dec. 22; 106(51):21807-12
²²Talantov D. et al, Clin Cancer Res. 2005 Oct. 15; 11(20):7234-42
²³Basset C. et al., Scand J Immunol. 2000 March; 51(3):307-11
²⁴Grewal P. K. et al, Mol Cell Biol. 2006, July; 26(13):4970-81
²⁵Hedlund M. et al, Cancer Res. 2008 Jan. 15; 68(2):388-94
²⁶Kondoh K. et al., Breast Cancer Res Treat. 2003 March; 78(1):37-44
²⁷Hoffmeister A. et al, JOP. 2009 Sep. 4; 10(5):501-6
²⁸Woodard-Grice A. V. et al., J Biol Chem. 2008 Sep. 26; 283(39):26364-73. Epub 2008 Jul. 23
²⁹Toegel S. et al, Osteoarthritis Cartilage. 2010 February; 18(2):240-8. Epub 2009 Sep. 22
³⁰Lichtenthaler S. F. et al., J Biol Chem. 2003 Dec. 5; 278(49):48713-9. Epub 2003 Sep. 24
³¹Merten M. et al., Z Kardiol. 2004 November; 93(11):855-63
³²Maugeri N. et al., Srp Arh Celok Lek. 2010 January; 138 Suppl 1:50-2
³³Kiljański J. et al, Thyroid. 2005 July; 15(7):645-52
³⁴WO2013110622, WO2012168175, WO2012168164 and WO2012156284
³⁵Biochem. Pharmacol. (1973) 22:3099
³⁶T. W. Greene and P. G. M. Wuts, “Protective Groups in Organic Synthesis”, 2nd ed., John Wiley & Sons, Inc., New York, N. Y., 1991, chapter 7; E. Haslam, “Protective Groups in Organic Chemistry”, J. G. W. McOmie, Ed., Plenum Press, New York, N. Y., 1973, Chapter 5, and T. W. Greene, “Protective Groups in Organic Synthesis”, John Wiley and Sons, New York, N. Y., 1981
³⁷IUPAC—Compendium of Chemical Terminology, 2nd, A. D. McNaught & A. Wilkinson (Eds). Blackwell Scientific Publications, Oxford (1997)
³⁸T. P. Tang & J. A. Ellman, J. Org. Chem. 1999, 64, 12
³⁹A. Klapars and S. L. Buchwald in JACS 2002, 124(50), 14844
⁴⁰A. Klapars and S. L. Buchwald in JACS 2002, 124(50), 14844
⁴¹US20120258962
⁴²WO2011138293
⁴³WO2009134617
⁴⁴Andrew W. Stamford, Jack D. Scott, Sarah W. Li, Suresh Babu, Dawit Tadesse, Rachael Hunter, Yusheng Wu, Jeffrey Misiaszek, Jared N. Cumming, Eric J. Gilbert, Chunli Huang, Brian A. McKittrick, Liwu Hong, Tao Guo, Zhaoning Zhu, Corey Strickland, Peter Orth, Johannes H. Voigt, Matthew E. Kennedy, Xia Chen, Reshma Kuvelkar, Robert Hodgson, Lynn A. Hyde, Kathleen Cox, Leonard Favreau, Eric M. Parker, and William J. Greenlee, ACS Med. Chem. Lett. (2012), 3(11), 897-902
⁴⁵Ute F. Röhrig, Somi Reddy Majjigapu, Aurélien Grosdidier, Sylvian Bron, Vincent Stroobant, Luc Pilotte, Didier Colau, Pierre Vogel, Benoît J. Van den Eynde, Vincent Zoete, and Olivier Michielin, J. Med. Chem. 2012, 55, 5270-5290
⁴⁶Farahat A A, Kumar A, Say M, Barghash A E D M, Goda F E, Eisa H M, Wenzler T, Brun R, Liu Y, Mickelson L, Wilson W D, Boykin D W (2010) Synthesis, DNA binding, fluorescence measurements and antiparasitic activity of DAPI related diamidines, Bioorg. Med. Chem. 2010, 18(2), 557-566
⁴⁷WO2011009898
⁴⁸WO2012087237

1. A compound of formula I, 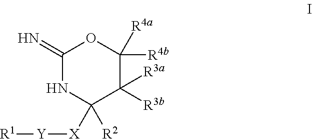
wherein
Y is a bond and X is selected from the group consisting of 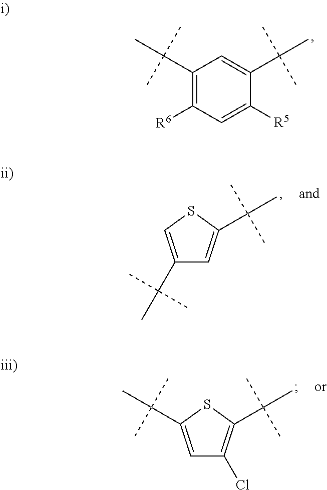
Y is selected from the group consisting of 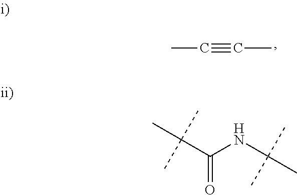
and X is 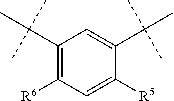
iii) —(CH₂)ₙ— with n=1, 2 or 3,
iv) —NH—, and
or Y is 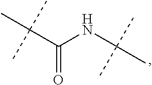
and X is 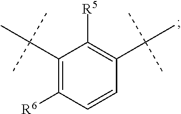
R¹ is selected from the group consisting of
i) heteroaryl, and
ii) heteroaryl, substituted by one or two substituents individually selected from the group consisting of amino, cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl, C₁₋₆-alkyl, aryl, which aryl is optionally substituted by cyano, halogen, halogen-C₁₋₆-alkyl, halogen-C₁₋₆-alkoxy, C₁₋₆-alkoxy or C₁₋₆-alkyl, and heteroaryl, which heteroaryl is optionally substituted by cyano, halogen, halogen-C₁₋₆-alkyl, halogen-C₁₋₆-alkoxy, C₁₋₆-alkoxy or C₁₋₆-alkyl,
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iv) aryl, when R^{4a} is halogen-C₁₋₆-alkyl;
R² is selected from the group consisting of
i) C₁₋₆-alkyl, and
ii) halogen-C₁₋₆-alkyl;
R^{3a} is selected from the group consisting of
i) hydrogen and
ii) C₁₋₆-alkyl;
R^{3b} is selected from the group consisting of
i) hydrogen,
ii) halogen, and
iii) halogen-C₁₋₆-alkoxy;
R^{4a} is selected from the group consisting of
i) halogen-C₁₋₆-alkyl, and
ii) C₁₋₆-alkyl;
R^{4b} is selected from the group consisting of
i) hydrogen and
ii) C₁₋₆-alkyl;
R⁵ is halogen;
R⁶ is selected from the group consisting of
i) hydrogen and
ii) halogen;
or pharmaceutically acceptable salts thereof.

2. The compound according to claim 1, wherein
Y is a bond and X is selected from the group consisting of 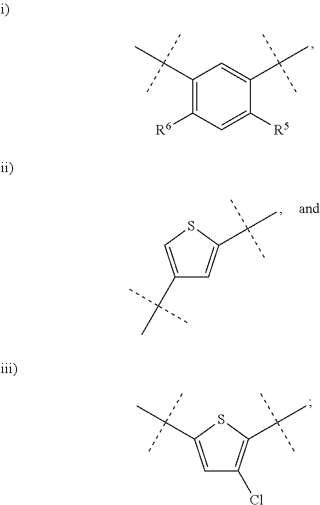
or
Y is selected from the group consisting of
i) —C≡C— and 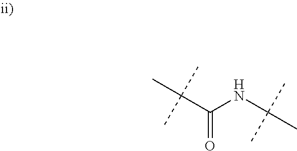
and X is 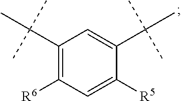
R¹ is selected from the group consisting of
i) heteroaryl, and
ii) heteroaryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl.
R² is selected from the group consisting of
i) C₁₋₆-alkyl, and
ii) halogen-C₁₋₆-alkyl;
R^{3a} is selected from the group consisting of
i) hydrogen and
ii) C₁₋₆-alkyl;
R^{3b} is selected from the group consisting of
i) hydrogen,
ii) halogen, and
iii) halogen-C₁₋₆-alkoxy;
R^{4a} is selected from the group consisting of
i) halogen-C₁₋₆-alkyl, and
ii) C₁₋₆-alkyl;
R^{4b} is selected from the group consisting of
i) hydrogen and
ii) C₁₋₆-alkyl;
R⁵ is halogen;
R⁶ is selected from the group consisting of
i) hydrogen and
ii) halogen;
or a pharmaceutically acceptable salt thereof.

3. The compound according to claim 1 of formula I′: 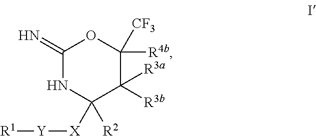
wherein X, Y, R¹, R², R^{3a}, R^{3b}, R^{4a} and R^{4b} are as defined in claim 1.

4. The compound according to claim 1 of formula Ia′: 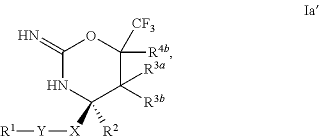
wherein X, Y, R¹, R², R^{3a}, R^{3b}, R^{4a} and R^{4b} are as defined in claim 1.

5. The compound according to claim 1, wherein Y is a bond and X is selected from the group consisting of

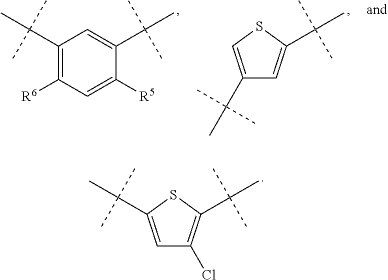

6. The compound according to claim 1, wherein Y is —C≡C—.

7. The compound according to claim 1, wherein R⁵ is fluoro.

8. The compound according to claim 1, wherein R⁶ is hydrogen.

9. The compound according to claim 1, wherein R¹ is selected from the group consisting of
i) heteroaryl, and
ii) heteroaryl, substituted by one or two substituents individually selected from the group consisting of amino, cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl, C₁₋₆-alkyl, aryl, which aryl is optionally substituted by cyano, halogen, halogen-C₁₋₆-alkyl, halogen-C₁₋₆-alkoxy, C₁₋₆-alkoxy or C₁₋₆-alkyl, and heteroaryl, which heteroaryl is optionally substituted by cyano, halogen, halogen-C₁₋₆-alkyl, halogen-C₁₋₆-alkoxy, C₁₋₆-alkoxy or C₁₋₆-alkyl,
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iv) aryl, when R^{4a} is halogen-C₁₋₆-alkyl;

10. The compound according to claim 1, wherein R¹ is selected from the group consisting of
i) heteroaryl,
ii) heteroaryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl, and
iii) aryl, substituted by one or two substituents individually selected from the group consisting of cyano, halogen, halogen-C₁₋₆-alkyl, halogen-C₁₋₆-alkoxy, C₁₋₆-alkoxy, C₂₋₆-alkynyl-C₁₋₆-alkoxy, C₂₋₆-alkynyl, C₁₋₆-alkoxy-C₁₋₆-alkyl and C₁₋₆-alkyl.

11. The compound according to claim 1, wherein R¹ is selected from the group consisting of phenyl, 1H-pyrazolyl, pyridinyl, pyrazinyl and pyrimidinyl, each unsubstituted or individually substituted by difluoromethyl, chloro, fluoro, cyano, trifluoromethyl, prop-1-ynyl, but-2-ynyloxy or methoxy.

12. The compound according to claim 1, wherein R¹ is phenyl substituted by cyano or trifluoromethyl.

13. The compound according to claim 1, wherein R¹ is 1H-pyrazolyl substituted by difluoromethyl.

14. The compound according to claim 1, wherein R¹ is pyridinyl, unsubstituted or substituted by cyano, chloro, fluoro or prop-1-ynyl.

15. The compound according to claim 1, wherein R¹ is pyrazinyl substituted by but-2-ynyloxy, methoxy, difluoromethyl or chloro.

16. The compound according to claim 1, wherein R¹ is pyrimidinyl, unsubstituted or substituted by chloro or methoxy.

17. The compound according to claim 1, wherein R² is methyl.

18. The compound according to claim 1, wherein R² is —CH₂F.

19. The compound according to claim 1, wherein R^{3a} is hydrogen.

20. The compound according to claim 1, wherein R^{3a} is methyl.

21. The compound according to claim 1, wherein R^{3b} is hydrogen.

22. The compound according to claim 1, wherein R^{3b} is —OCH₂CF₃.

23. The compound according to claim 1, wherein R^{3b} is fluoro.

24. The compound according to claim 1, wherein R^{4a} is —CF₃.

25. The compound according to claim 1, wherein R^{4a} is methyl.

26. The compound according to claim 1, wherein R^{4b} is hydrogen.

27. The compound according to claim 1, wherein R^{4b} is methyl.

28. The compound according to claim 1, wherein said compound is selected from the group consisting of
(4S,6S)-4-(2,4-Difluoro-5-(2-fluoropyridin-3-yl)phenyl)-4-methyl-6-(trifluoro-methyl)-5,6-dihydro-4H-1,3-oxazin-2-amine,
(4S,6S)-4-(2,4-Difluoro-5-(5-(prop-1-ynyl)pyridin-3-yl)phenyl)-4-methyl-6-(trifluoromethyl)-5,6-dihydro-4H-1,3-oxazin-2-amine,
(4S,6S)-4-(2,4-Difluoro-5-(pyrimidin-5-yl)phenyl)-4-methyl-6-(trifluoromethyl)-5,6-dihydro-4H-1,3-oxazin-2-amine,
(4S,6S)-4-(2-fluoro-5-((3-methylisothiazol-5-yl)ethynyl)phenyl)-4-(fluoromethyl)-6-(trifluoromethyl)-5,6-dihydro-4H-1,3-oxazin-2-amine,
(4S,6S)-4-(2-fluoro-5-((3-methylisothiazol-5-yl)ethynyl)phenyl)-4-methyl-6-(trifluoromethyl)-5,6-dihydro-4H-1,3-oxazin-2-amine,
(4S,6S)-4-(2-Fluoro-5-((5-methoxypyrazin-2-yl)ethynyl)phenyl)-4-methyl-6-(trifluoromethyl)-5,6-dihydro-4H-1,3-oxazin-2-amine,
(4S,6S)-4-(2-Fluoro-5-((5-methoxypyrimidin-2-yl)ethynyl)phenyl)-4-methyl-6-(trifluoromethyl)-5,6-dihydro-4H-1,3-oxazin-2-amine,
(4S,6S)-4-(2-fluoro-5-(4-fluoropyridin-3-yl)-4-methylphenyl)-4-methyl-6-(trifluoromethyl)-5,6-dihydro-4H-1,3-oxazin-2-amine,
(4S,6S)-4-(2-fluoro-5-(5-(5-methyl-1H-pyrazol-3-yl)pyridin-3-yl)phenyl)-4-(fluoromethyl)-6-(trifluoromethyl)-5,6-dihydro-4H-1,3-oxazin-2-amine,
(4S,6S)-4-(2-Fluoro-5-(5-(prop-1-ynyl)pyridin-3-yl)phenyl)-4-methyl-6-(trifluoromethyl)-5,6-dihydro-4H-1,3-oxazin-2-amine,
(4S,6S)-4-(2-Fluoro-5-(pyridin-3-ylethynyl)phenyl)-4-methyl-6-(trifluoromethyl)-5,6-dihydro-4H-1,3-oxazin-2-amine,
(4S,6S)-4-(2-fluoro-5-(pyrimidin-5-yl)phenyl)-4-methyl-6-(trifluoromethyl)-5,6-dihydro-4H-1,3-oxazin-2-amine,
(4S,6S)-4-(2-fluoro-5-(pyrimidin-5-yethynyl)phenyl)-4-methyl-6-(trifluoromethyl)-5,6-dihydro-4H-1,3-oxazin-2-amine,
(4S,6S)-4-(3-Chloro-5-(5-(prop-1-ynyl)pyridin-3-yl)thiophen-2-yl)-4-methyl-6-(trifluoromethyl)-5,6-dihydro-4H-1,3-oxazin-2-amine,
(4S,6S)-4-(3-Chloro-5-(5-chloropyridin-3-yl)thiophen-2-yl)-4-methyl-6-(trifluoromethyl)-5,6-dihydro-4H-1,3-oxazin-2-amine,
(4S,6S)-4-(4-(2-Fluoropyridin-3-yl)thiophen-2-yl)-4-methyl-6-(trifluoromethyl)-5,6-dihydro-4H-1,3-oxazin-2-amine,
(4S,6S)-4-(4-fluorobiphenyl-3-yl)-4-methyl-6-(trifluoromethyl)-5,6-dihydro-4H-1,3-oxazin-2-amine,
(4S,6S)-4-(5-((1-ethyl-1H-pyrazol-4-yl)ethynyl)-2-fluorophenyl)-4-(fluoromethyl)-6-(trifluoromethyl)-5,6-dihydro-4H-1,3-oxazin-2-amine,
(4S,6S)-4-(5-((2-aminopyrimidin-5-yl)ethynyl)-2-fluorophenyl)-4-methyl-6-(trifluoromethyl)-5,6-dihydro-4H-1,3-oxazin-2-amine,
(4S,6S)-4-(5-((2-Chloropyridin-4-yl)ethynyl)-2-fluorophenyl)-4-methyl-6-(trifluoromethyl)-5,6-dihydro-4H-1,3-oxazin-2-amine,
(4S,6S)-4-(5-((4-chloro-1-(difluoromethyl)-1H-pyrazol-3-yl)ethynyl)-2-fluorophenyl)-4-methyl-6-(trifluoromethyl)-5,6-dihydro-4H-1,3-oxazin-2-amine,
(4S,6S)-4-(5-((4-chloro-1-(difluoromethyl)-1H-pyrazol-3-yl)ethynyl)-2-fluoro-4-methylphenyl)-4-methyl-6-(trifluoromethyl)-5,6-dihydro-4H-1,3-oxazin-2-amine,
(4S,6S)-4-(5-((4-chlorophenyl)ethynyl)-2-fluorophenyl)-4-methyl-6-(trifluoromethyl)-5,6-dihydro-4H-1,3-oxazin-2-amine,
(4S,6S)-4-(5-((5-chloropyridin-3-yl)ethynyl)-2-fluorophenyl)-4-(fluoromethyl)-6-(trifluoromethyl)-5,6-dihydro-4H-1,3-oxazin-2-amine,
(4S,6S)-4-(5-((5-chloropyridin-3-yl)ethynyl)-2-fluorophenyl)-4-methyl-6-(trifluoromethyl)-5,6-dihydro-4H-1,3-oxazin-2-amine,
(4S,6S)-4-(5-((5-chloropyrimidin-2-yl)ethynyl)-2-fluoro-4-methylphenyl)-4-methyl-6-(trifluoromethyl)-5,6-dihydro-4H-1,3-oxazin-2-amine,
(4S,6S)-4-(5-((5-Chloropyrimidin-2-yl)ethynyl)-2-fluorophenyl)-4-methyl-6-(trifluoromethyl)-5,6-dihydro-4H-1,3-oxazin-2-amine,
(4S,6S)-4-(5-((6-aminopyridin-3-yl)ethynyl)-2-fluorophenyl)-4-methyl-6-(trifluoromethyl)-5,6-dihydro-4H-1,3-oxazin-2-amine,
(4S,6S)-4-(5-(1-(Difluoromethyl)-1H-pyrazol-4-yl)-2-fluorophenyl)-4-methyl-6-(trifluoromethyl)-5,6-dihydro-4H-1,3-oxazin-2-amine,
(4S,6S)-4-(5-(5-(1H-tetrazol-5-yl)pyridin-3-yl)-2-fluorophenyl)-4-(fluoromethyl)-6-(trifluoromethyl)-5,6-dihydro-4H-1,3-oxazin-2-amine,
(4S,6S)-4-(5-(5-(4-chiorophenyl)-1,3,4-oxadiazol-2-yl)-2-fluorophenyl)-4-methyl-6-(trifluoromethyl)-5,6-dihydro-4H-1,3-oxazin-2-amine,
(4S,6S)-4-(5-(5-Chloropyridin-3-yl)-2-fluorophenyl)-4-methyl-6-(trifluoromethyl)-5,6-dihydro-4H-1,3-oxazin-2-amine,
(4S,6S)-4-(5-(6-chlorobenzo[d]oxazol-2-yl)-2-fluorophenyl)-4-(fluoromethyl)-6-(trifluoromethyl)-5,6-dihydro-4H-1,3-oxazin-2-amine,
(4S,6S)-4-(5-(6-chlorobenzo[d]oxazol-2-yl)-2-fluorophenyl)-4-methyl-6-(trifluoromethyl)-5,6-dihydro-4H-1,3-oxazin-2-amine,
(4S,6S)-4-(5-(6-Chloropyrazin-2-yl)-2-fluorophenyl)-4-methyl-6-(trifluoromethyl)-5,6-dihydro-4H-1,3-oxazin-2-amine,
(4S,6S)-4-[2-fluoro-5-[2-(2-methoxypyrimidin-5-yl)ethynyl]phenyl]-4-methyl-6-(trifluoromethyl)-5,6-dihydro-1,3-oxazin-2-amine,
(4S,6S)-4-[5-[2-(5-chloropyrimidin-2-yl)ethynyl]-2-fluorophenyl]-4-(fluoromethyl)-6-(trifluoromethyl)-5,6-dihydro-1,3-oxazin-2-amine,
(4S,6S)-4-[5-[2-(6-chloropyridin-3-yl)ethynyl]-2-fluorophenyl]-4-methyl-6-(trifluoromethyl)-5,6-dihydro-1,3-oxazin-2-amine,
(4S,6S)-4-Methyl-4-(4-(5-(prop-1-ynyl)pyridin-3-yl)thiophen-2-yl)-6-(trifluoromethyl)-5,6-dihydro-4H-1,3-oxazin-2-amine,
(4S,6S)-4-methyl-4-(4-(pyrimidin-5-yl)thiophen-2-yl)-6-(trifluoromethyl)-5,6-dihydro-4H-1,3-oxazin-2-amine,
(4S,6S)-4-Methyl-6-(trifluoromethyl)-4-(4-(3-(trifluoromethyl)phenyl)thiophen-2-yl)-5,6-dihydro-4H-1,3-oxazin-2-amine,
2-((3-((4S,6S)-2-amino-4-methyl-6-(trifluoromethyl)-5,6-dihydro-4H-1,3-oxazin-4-yl)-4-fluorophenyl)ethynyl)thiazole-5-carbonitrile,
3-(5-((4S,6S)-2-Amino-4-methyl-6-(trifluoromethyl)-5,6-dihydro-4H-1,3-oxazin-4-yl)-4-chlorothiophen-2-yl)benzonitrile,
4-((3-((4S,6S)-2-amino-4-(fluoromethyl)-6-(trifluoromethyl)-5,6-dihydro-4H-1,3-oxazin-4-yl)-4-fluorophenyl)ethynyl)benzonitrile,
4-((3-((4S,6S)-2-amino-4-methyl-6-(trifluoromethyl)-5,6-dihydro-4H-1,3-oxazin-4-yl)-4-fluorophenyl)ethynyl)benzonitrile,
5-((3-((4S,6S)-2-amino-4-methyl-6-(trifluoromethyl)-5,6-dihydro-4H-1,3-oxazin-4-yl)-4-fluorophenyl)ethynyl)picolinonitrile,
5′-((4S,6S)-2-amino-4-methyl-6-(trifluoromethyl)-5,6-dihydro-4H-1,3-oxazin-4-yl)-2,4′-difluoro-2′-methylbiphenyl-3-carbonitrile,
5-(3-((4S,6S)-2-Amino-4-methyl-6-(trifluoromethyl)-5,6-dihydro-4H-1,3-oxazin-4-yl)-4-fluorophenyl)nicotinonitrile,
5-(5-((4S,6S)-2-Amino-4-methyl-6-(trifluoromethyl)-5,6-dihydro-4H-1,3-oxazin-4-yl)thiophen-3-yl)nicotinonitrile,
5-(5-((4S,6S)-2-Amino-4-methyl-6-(trifluoromethyl)-5,6-dihydro-4H-1,3-oxazin-4-yl)-4-chlorothiophen-2-yl)nicotinonitrile,
5-(5-((4S,6S)-2-amino-4-methyl-6-(trifluoromethyl)-5,6-dihydro-4H-1,3-oxazin-4-yl)thiophen-3-yl)pyrimidine-2-carbonitrile,
5-[2-[3-[(4S,6S)-2-amino-4-methyl-6-(trifluoromethyl)-5,6-dihydro-1,3-oxazin-4-yl]-4-fluorophenyl]ethynyl]pyrimidine-2-carbonitrile,
5-[2-[3-[(4S,6S)-2-amino-4-methyl-6-(trifluoromethyl)-5,6-dihydro-1,3-oxazin-4-yl]-4-fluorophenyl]ethynyl]pyridine-3-carbonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)-5-methoxynicotinonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)-5-chloronicotinonitrile,
6-((3-((4S,6S)-2-amino-4-(fluoromethyl)-6-(trifluoromethyl)-5,6-dihydro-4H-1,3-oxazin-4-yl)-4-fluorophenyl)ethynyl)nicotinonitrile,
6-((3-((4S,6S)-2-Amino-4-methyl-6-(trifluoromethyl)-5,6-dihydro-4H-1,3-oxazin-4-yl)-4-fluorophenyl)ethynyl)nicotinonitrile,
6-((5-((4S,6S)-2-amino-4-methyl-6-(trifluoromethyl)-5,6-dihydro-4H-1,3-oxazin-4-yl)thiophen-3-yl)ethynyl)nicotinonitrile,
6-((5-((4S,6S)-2-amino-4-methyl-6-(trifluoromethyl)-5,6-dihydro-4H-1,3-oxazin-4-yl)-4-fluoro-2-methylphenyl)ethynyl)-5-chloronicotinonitrile,
6-((5-((4S,6S)-2-amino-4-methyl-6-(trifluoromethyl)-5,6-dihydro-4H-1,3-oxazin-4-yl)-4-fluoro-2-methylphenyl)ethynyl)nicotinonitrile,
6-(3-((4S,6S)-2-amino-4-methyl-6-(trifluoromethyl)-5,6-dihydro-4H-1,3-oxazin-4-yl)-4-fluorophenethyl)nicotinonitrile,
6-(4-(3-((4S,6S)-2-amino-4-methyl-6-(trifluoromethyl)-5,6-dihydro-4H-1,3-oxazin-4-yl)-4-fluorophenyl)-1H-1,2,3-triazol-1-yl)nicotinonitrile,
7-(3-((4S,6S)-2-amino-4-methyl-6-(trifluoromethyl)-5,6-dihydro-4H-1,3-oxazin-4-yl)-4-fluorophenylamino)-6,7-dihydro-5H-cyclopenta[b]pyridine-3-carbonitrile,
N-(3-((4R,5R,6R)-2-Amino-5-fluoro-4,5-dimethyl-6-(trifluoromethyl)-5,6-dihydro-4H-1,3-oxazin-4-yl)-4-fluorophenyl)-5-cyanopicolinamide,
N-(3-((4R,5R,6R)-2-Amino-5-fluoro-4-methyl-6-(trifluoromethyl)-5,6-dihydro-4H-1,3-oxazin-4-yl)-4-fluorophenyl)-5-cyanopicolinamide,
N-(3-((4R,5R,6R)-2-Amino-5-fluoro-4-methyl-6-(trifluoromethyl)-5,6-dihydro-4H-1,3-oxazin-4-yl)-4-fluorophenyl)-5-chloropicolinamide,
N-(3-((4R,5R,6S)-2-Amino-4-methyl-5-(2,2,2-trifluoroethoxy)-6-(trifluoromethyl)-5,6-dihydro-4H-1,3-oxazin-4-yl)-4-fluorophenyl)-5-cyanopicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-cyanopicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-chloropicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-methoxypyrazine-2-carboxamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-(difluoromethyl)pyrazine-2-carboxamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-(but-2-ynyloxy)pyrazine-2-carboxamide,
N-(3-((4S,6S)- and (4R,6R)-2-Amino-4-(fluoromethyl)-6-methyl-6-(trifluoro-methyl)-5,6-dihydro-4H-1,3-oxazin-4-yl)-4-fluorophenyl)-5-cyanopicolinamide,
N-(3-((4S,6S)-2-amino-4-methyl-6-(trifluoromethyl)-5,6-dihydro-4H-1,3-oxazin-4-yl)-2-fluorophenyl)-5-cyanopicolinamide,
N-(3-((4S,6S)-2-amino-4-methyl-6-(trifluoromethyl)-5,6-dihydro-4H-1,3-oxazin-4-yl)-2,4-difluorophenyl)-5-cyanopicolinamide,
N-(5-((4S,6S)-2-amino-4-methyl-6-(trifluoromethyl)-5,6-dihydro-4H-1,3-oxazin-4-yl)thiophen-3-yl)-5-chloropicolinamide, and
N-(5-((4S,6S)-2-amino-4-methyl-6-(trifluoromethyl)-5,6-dihydro-4H-1,3-oxazin-4-yl)thiophen-3-yl)-5-cyanopicolinamide,
or a pharmaceutically acceptable salt thereof.

29. The compound according to claim 1, wherein said compound is selected from the group consisting of:
N-(3-((4R,5R,6R)-2-Amino-5-fluoro-4-methyl-6-(trifluoromethyl)-5,6-dihydro-4H-1,3-oxazin-4-yl)-4-fluorophenyl)-5-cyanopicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-cyanopicolinamide,
N-(3-((4R,5R,6R)-2-Amino-5-fluoro-4-methyl-6-(trifluoromethyl)-5,6-dihydro-4H-1,3-oxazin-4-yl)-4-fluorophenyl)-5-chloropicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-chloropicolinamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-methoxypyrazine-2-carboxamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-(difluoromethyl)pyrazine-2-carboxamide,
N-(3-((4R,5R,6S)-2-Amino-5-fluoro-4-methyl-6-(trifluoromethyl)-5,6-dihydro-4H-1,3-oxazin-4-yl)-4-fluorophenyl)-5-(but-2-ynyloxy)pyrazine-2-carboxamide,
N-(3-((4S,6S)- and (4R,6R)-2-Amino-4-(fluoromethyl)-6-methyl-6-(trifluoro-methyl)-5,6-dihydro-4H-1,3-oxazin-4-yl)-4-fluorophenyl)-5-cyanopicolinamide,
(4S,6S)-4-(2-fluoro-5-(pyrimidin-5-yl)phenyl)-4-methyl-6-(trifluoromethyl)-5,6-dihydro-4H-1,3-oxazin-2-amine,
(4S,6S)-4-(5-(5-Chloropyridin-3-yl)-2-fluorophenyl)-4-methyl-6-(trifluoromethyl)-5,6-dihydro-4H-1,3-oxazin-2-amine,
(4S,6S)-4-(2-Fluoro-5-(5-(prop-1-ynyl)pyridin-3-yl)phenyl)-4-methyl-6-(trifluoromethyl)-5,6-dihydro-4H-1,3-oxazin-2-amine,
(4S,6S)-4-(5-(6-Chloropyrazin-2-yl)-2-fluorophenyl)-4-methyl-6-(trifluoromethyl)-5,6-dihydro-4H-1,3-oxazin-2-amine,
(4S,6S)-4-(5-(1-(Difluoromethyl)-1H-pyrazol-4-yl)-2-fluorophenyl)-4-methyl-6-(trifluoromethyl)-5,6-dihydro-4H-1,3-oxazin-2-amine,
(4S,6S)-4-(5-((2-Chloropyridin-4-yl)ethynyl)-2-fluorophenyl)-4-methyl-6-(trifluoromethyl)-5,6-dihydro-4H-1,3-oxazin-2-amine,
(4S,6S)-4-(2-Fluoro-5-(pyridin-3-ylethynyl)phenyl)-4-methyl-6-(trifluoromethyl)-5,6-dihydro-4H-1,3-oxazin-2-amine,
(4S,6S)-4-(5-((5-Chloropyrimidin-2-yl)ethynyl)-2-fluorophenyl)-4-methyl-6-(trifluoromethyl)-5,6-dihydro-4H-1,3-oxazin-2-amine,
(4S,6S)-4-(2-Fluoro-5-((5-methoxypyrimidin-2-yl)ethynyl)phenyl)-4-methyl-6-(trifluoromethyl)-5,6-dihydro-4H-1,3-oxazin-2-amine,
(4S,6S)-4-(2-Fluoro-5-((5-methoxypyrazin-2-yl)ethynyl)phenyl)-4-methyl-6-(trifluoromethyl)-5,6-dihydro-4H-1,3-oxazin-2-amine,
(4S,6S)-4-(4-(2-Fluoropyridin-3-yl)thiophen-2-yl)-4-methyl-6-(trifluoromethyl)-5,6-dihydro-4H-1,3-oxazin-2-amine,
(4S,6S)-4-Methyl-6-(trifluoromethyl)-4-(4-(3-(trifluoromethyl)phenyl)thiophen-2-yl)-5,6-dihydro-4H-1,3-oxazin-2-amine,
(4S,6S)-4-Methyl-4-(4-(5-(prop-1-ynyl)pyridin-3-yl)thiophen-2-yl)-6-(trifluoromethyl)-5,6-dihydro-4H-1,3-oxazin-2-amine,
(4S,6S)-4-(3-Chloro-5-(5-(prop-1-ynyl)pyridin-3-yl)thiophen-2-yl)-4-methyl-6-(trifluoromethyl)-5,6-dihydro-4H-1,3-oxazin-2-amine,
(4S,6S)-4-(3-Chloro-5-(5-chloropyridin-3-yl)thiophen-2-yl)-4-methyl-6-(trifluoromethyl)-5,6-dihydro-4H-1,3-oxazin-2-amine,
N-(3-((4R,5R,6S)-2-Amino-4-methyl-5-(2,2,2-trifluoroethoxy)-6-(trifluoromethyl)-5,6-dihydro-4H-1,3-oxazin-4-yl)-4-fluorophenyl)-5-cyanopicolinamide,
(4S,6S)-4-(2,4-Difluoro-5-(pyrimidin-5-yl)phenyl)-4-methyl-6-(trifluoromethyl)-5,6-dihydro-4H-1,3-oxazin-2-amine,
(4S,6S)-4-(2,4-Difluoro-5-(2-fluoropyridin-3-yl)phenyl)-4-methyl-6-(trifluoro-methyl)-5,6-dihydro-4H-1,3-oxazin-2-amine,
(4S,6S)-4-(2,4-Difluoro-5-(5-(prop-1-ynyl)pyridin-3-yl)phenyl)-4-methyl-6-(trifluoromethyl)-5,6-dihydro-4H-1,3-oxazin-2-amine,
N-(3-((4R,5R,6R)-2-Amino-5-fluoro-4,5-dimethyl-6-(trifluoromethyl)-5,6-dihydro-4H-1,3-oxazin-4-yl)-4-fluorophenyl)-5-cyanopicolinamide,
5-(3-((4S,6S)-2-Amino-4-methyl-6-(trifluoromethyl)-5,6-dihydro-4H-1,3-oxazin-4-yl)-4-fluorophenyl)nicotinonitrile,
6-((3-((4S,6S)-2-Amino-4-methyl-6-(trifluoromethyl)-5,6-dihydro-4H-1,3-oxazin-4-yl)-4-fluorophenyl)ethynyl)nicotinonitrile,
5-(5-((4S,6S)-2-Amino-4-methyl-6-(trifluoromethyl)-5,6-dihydro-4H-1,3-oxazin-4-yl)thiophen-3-yl)nicotinonitrile,
5-(5-((4S,6S)-2-Amino-4-methyl-6-(trifluoromethyl)-5,6-dihydro-4H-1,3-oxazin-4-yl)-4-chlorothiophen-2-yl)nicotinonitrile, and
3-(5-((4S,6S)-2-Amino-4-methyl-6-(trifluoromethyl)-5,6-dihydro-4H-1,3-oxazin-4-yl)-4-chlorothiophen-2-yl)benzonitrile,
or a pharmaceutically acceptable salt thereof.

30-32. (canceled)

33. A pharmaceutical composition, comprising a therapeutically effective amount of a compound according to claim 1 and a pharmaceutically acceptable carrier and/or a pharmaceutically acceptable auxiliary substance.

34-35. (canceled)

36. A method for the treatment of Alzheimer's disease, amyotrophic lateral sclerosis (ALS), arterial thrombosis, autoimmune/inflammatory diseases, cancer such as breast cancer, cardiovascular diseases such as myocardial infarction and stroke, dermatomyositis, Down's Syndrome, gastrointestinal diseases, Glioblastoma multiforme, Graves Disease, Huntington's Disease, inclusion body myositis (IBM), inflammatory reactions, Kaposi Sarcoma, Kostmann Disease, lupus erythematosus, macrophagic myofasciitis, juvenile idiopathic arthritis, granulomatous arthritis, malignant melanoma, multiple mieloma, rheumatoid arthritis, Sjogren syndrome, SpinoCerebellar Ataxia 1, SpinoCerebellar Ataxia 7, Whipple's Disease or Wilson's Disease, comprising the step of administering a therapeutically effective amount of a compound according to claim 1 to a human being or animal in need thereof.

37. (canceled)
